(19) United States
(12) Patent Application Publication
(10) Pub. No.: US 2024/0141021 A1
(43) Pub. Date: May 2, 2024

(54) ANTI-INFLUENZA ANTIBODIES AND COMBINATIONS THEREOF

(71) Applicants: Vir Biotechnology, Inc., San Francisco, CA (US); Humabs BioMed SA, Bellinzona (CH)

(72) Inventors: Davide CORTI, Bellinzona (CH); Matteo Samuele PIZZUTO, Bellinzona (CH); Andrea MINOLA, Bellinzona (CH); Elisabetta CAMERONI, Bellinzona (CH); Fabrizia ZATTA, Bellinzona (CH); Gyorgy SNELL, San Francisco, CA (US); Elena FERRI, San Francisco, CA (US)

(21) Appl. No.: 18/253,386

(22) PCT Filed: Nov. 19, 2021
(86) PCT No.: PCT/US2021/060166

§ 371 Date: May 17, 2023

Related U.S. Application Data
(60) Provisional application No. 63/117,454, filed on Nov. 23, 2020.
(60) Provisional application No. 63/125,892, filed on Dec. 15, 2020.
(60) Provisional application No. 63/197,254, filed on Jun. 4, 2021.
(60) Provisional application No. 63/261,464, filed on Sep. 21, 2021.

(51) Int. Cl. C07K 16/10; A61K 39/00; A61P 31/16

(52) U.S. Cl. CPC C07K 16/1018; A61P 31/16; A61K 2039/507; C07K 2317/31; C07K 2317/33; C07K 2317/34; C07K 2317/41; C07K 2317/52; C07K 2317/72; C07K 2317/76

(57) ABSTRACT

The present disclosure relates, in part, to anti-influenza antibodies (and antigen binding fragments thereof) and combinations thereof for preventing and treating influenza infection. Presently disclosed combinations provide surprising synergistic effects and can potently prevent, inhibit, or neutralize an influenza infection, such as an influenza A virus (IAV) infection an influenza B virus (IBV) infection, or both.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_415WO_SEQUENCE_LISTING.txt. The text file is 174 KB, was created on Nov. 16, 2021, and is being submitted electronically via EFS-Web.

### BACKGROUND

Influenza is an infectious disease which spreads around the world in yearly outbreaks, resulting per year in about three million to about five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually symptoms begin about two days after exposure to the virus. Complications of influenza may include pneumonia, sinus infections, and worsening of previous health problems such as asthma or heart failure, sepsis or exacerbation of chronic underlying disease.

Influenza is caused by influenza virus, an antigenically and genetically diverse group of viruses of the family Orthomyxoviridae that contains a negative-sense, single-stranded, segmented RNA genome. Of the four types of influenza virus (A, B, C and D), three types (A, B and C) are known to affect humans. Influenza viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 contains H1, H2, H5, H6, H8, H9, H11, H12, H13, H16 and H17 subtypes, and group 2 includes H3, H4, H7, H10, H14 and H15 subtypes. While all subtypes are present in birds, mostly H1, H2 and H3 subtypes cause disease in humans. H5, H7 and H9 subtypes are causing sporadic severe infections in humans and may generate a new pandemic. Influenza A viruses continuously evolve generating new variants, a phenomenon called antigenic drift. As a consequence, antibodies produced in response to past viruses may be poorly- or non-protective against new drifted viruses. A consequence is that new vaccines have to be produced every year against H1 and H3 viruses that are predicted to emerge, a process that is very costly, and not always efficient. The same applies to the production of a H5 influenza vaccine.

HA is a major surface protein of influenza A virus, and is the primary target of neutralizing antibodies that are induced by infection or vaccination. Without wishing to be bound by theory, HA is responsible for binding the virus to cells with sialic acid on the membranes, such as cells in the upper respiratory tract or erythrocytes. In addition, HA mediates the fusion of the viral envelope with the endosome membrane, after the pH has been reduced. HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha helical coiled coil structure and primarily forms the “stem” or “stalk” region of HA, while the HA1 region is a small globular domain containing a mix of a/0 structures (“head” region of HA). The globular HA head region mediates binding to the sialic acid receptor, while the HA stem mediates the subsequent fusion between the viral and cellular membranes that is triggered in endosomes by the low pH. While the immunodominant HA globular head domain has high plasticity with distinct antigenic sites undergoing constant antigenic drift, the HA stem region is relatively conserved among subtypes. Current influenza vaccines mostly induce an immune response against the immunodominant and variable HA head region, which evolves faster than the stem region of HA (Kirkpatrick E, Qiu X, Wilson P C, Bahl J, Krammer F. The influenza virus hemagglutinin head evolves faster than the stalk domain. Sci Rep. 2018 Jul. 11; 8(1):10432). Therefore, a particular influenza vaccine usually confers protection for no more than a few years and annual re-development of influenza vaccines is required.

There are at least 11 different neuraminidase subtypes (N1 through N11, respectively (cdc.gov/flu/about/viruses/types.htm)). Neuraminidases function in viral mobility and spread by catalyzing hydrolysis of sialic acid residues on virions prior to release from an infected host cell, and on target cell surface glycoproteins. Drugs designed to inhibit neuraminidase (NAIs) have been developed (e.g., oseltamivir, zanamivir, peramivir, laninamivir), though naturally acquired mutations of IAV subtypes have reduced susceptibility to current NAIs (Hussain et al., Infection and Drug Resistance 10:121-134 (2017).

New modalities for treating or preventing influenza virus infections are needed.

### BRIEF DESCRIPTION OF THE DRAWINGS

FIGS. 1A-1F show in vitro neutralization of influenza virus by an anti-NA (neuraminidase) monoclonal antibody combined with an anti-HA (hemagglutinin) monoclonal antibody. Anti-NA monoclonal antibodies “FNI3” (VH: SEQ ID NO.:72; VL: SEQ ID NO.:78) and “FNI9” (VH: SEQ ID NO.:132; VL: SEQ ID NO.:138), and anti-HA monoclonal antibodies “FM08” (VH: SEQ ID NO.:43; VL: SEQ ID NO.:44; see also Kallewaard et al. Cell 166(3):596-608 (2016), FIG. 1A) and “FHF11” (VH: SEQ ID NO.:2; VL: SEQ ID NO.:8) were evaluated using a fluorescence-based assay for sialidase inhibition that measures cleavage of the 2′-(4-Methylumbelliferyl)-α-D-N-acetylneuraminic acid (MUNANA). Inhibition of H1N1 Cal/09 sialidase activity by FM08+FNI3 (FIG. 1A), FM08+FNI9 (FIG. 1B), FHF11+FNI9 (FIG. 1C); and inhibition of H3N2 HK/68 sialidase activity by FM08 & FNI3 (FIG. 1D), FM08 & FNI9 (FIG. 1E), FHF11 & FNI9 (FIG. 1F) are shown. Heatmaps depict neutralization (%) at μg/ml (top panels; antibody concentrations are shown on x and y axes) and Synergy/Antagonism scores (bottom panels, reflecting increased or decreased neutralization of the antibody combination versus the combined effects of the single antibodies (e.g., effect of FM08+FNI9 versus effect of FM08 alone+effect of FNI9 alone) at the indicated concentration). Effects of single antibodies are shown in the left-most column and the bottom row of the upper graph in each of FIGS. 1A-1F.

FIGS. 2A-2C show in vitro neutralization of influenza virus by an anti-NA monoclonal antibody combined with an anti-HA monoclonal antibody. Anti-NA monoclonal antibodies “FNI9” (VH: SEQ ID NO.:132; VL: SEQ ID NO.:138), “FNI17” (VH: SEQ ID NO.:192; VL: SEQ ID NO.:198), and “FNI19” (VH: SEQ ID NO.:204; VL: SEQ ID NO.:210) and anti-HA monoclonal antibody “FM08” (VH: SEQ ID NO.:43; VL: SEQ ID NO.:44; see also Kallewaard et al. Cell 166(3):596-608 (2016), FIG. 1A) were evaluated by nucleoprotein staining. Inhibition of H3N2 A/Hong Kong/1/1968 sialidase activity by FM08+FN19 (FIG. 2A), FM08+FNI17 (FIG. 2B), and FM08+FNI19 (FIG. 2C) are shown. Heatmaps depict neutralization (%) at μg/ml (top panels; antibody concentrations are shown on x and y axes) and Synergy/Antagonism scores (bottom panels, reflecting increased or decreased neutralization of the antibody combination versus the combined effects of the single antibodies (e.g., effect of FM08+FNI9 versus effect of FM08 alone+effect of FN19 alone) at the indicated concentration). Synergy matrix and score were generated using MacSynergyII. “1:1” indicates the ratio of anti-NA to anti-HA monoclonal antibody. Effects of single antibodies are shown in the left-most column and the bottom row of the upper graph in each of FIGS. 2A-2C.

FIGS. 3A and 3B show activation of FcγRIIIa (FIG. 3A; F158 allele) and FcγRIIa (FIG. 3B; H131 allele) by anti-NA FNI3 and FNI9, engineered anti-HA monoclonal antibody “FHF1_v9” (VH: SEQ ID NO.:37; VL: SEQ ID NO.:8), and combinations thereof. Activation was measured using a NFAT-mediated Luciferase reporter in engineered Jurkat cells following contact with A549 cells pre-infected with H1N1 A/PR/8/34. Activation by a comparator antibody “FM08_LS” (FM08 bearing M428L/N434S mutations) and a negative control antibody against an irrelevant antigen, “K-” was also measured.

FIGS. 4A-4B show activation of FcγRIIIa by anti-NA monoclonal antibody “1G01-LS” (1G01 is described by Stadlbauer et al. (Science 366(6464):499-504 (2019); see FIG. 1B; the VH and VL amino acid sequences of antibody 1G01 therein, as well as those of 1E01 and 1G04 in FIG. 1B of Stadlbauer et al., are incorporated herein by reference), and in these experiments bore M428L and N434S Fc mutations), anti-HA FM08-LS, and a combination of both. Activation was measured using an NFAT-mediated Luciferase reporter in engineered Jurkat cells following contact with A549 cells pre-infected with H1N1 A/PR/8/34 (FIG. 4A; Multiplicity of Infection (MOI)=6) and H3N2 A/Aichi/2/68 (FIG. 4B; MOI=18). Activation by a negative control antibody (FY1-LALA) was also measured.

FIGS. 5A-5B show activation of FcγRIIa by anti-NA 1G01-LS, anti-HA FM08-LS, and a combination of both. Activation was measured using an NFAT-mediated luciferase reporter in engineered Jurkat cells following contact with A549 cells pre-infected with H1N1 A/PR/8/34 (FIG. 5A; MOI=6) and H3N2 A/Aichi/2/68 (FIG. 5B; MOI=6). Activation by a negative control antibody (FY1-LALA) was also measured.

FIGS. 6A-6B show the design of an in vivo study evaluating prophylactic activity of a combination of an anti-NA antibody with an anti-HA antibody in BALB/c mice infected with IAV A/Puerto Rico/8/34. 1G01 was used as the anti-NA antibody and FM08 was used as the anti-HA antibody. FIG. 6A shows the dosing and virus strains used in the study. FIG. 6B shows the timeline and endpoints of the study.

FIGS. 7A-7L show measurements of body weight over fifteen days in BALB/c mice that were infected with H1N1 A/Puerto Rico/8/34 following pre-treatment with anti-NA 1G01, anti-HA FM08, or the combination of 1G01 and FM08. Antibody was administered at 1 mg/kg, 0.5 mg/kg, 0.25 mg/kg, or 0.125 mg/kg, one day prior to infection with a LD90 (90% lethal dose) of A/Puerto Rico/8/34. Body weight of mice administered a vehicle control was also measured (left graph in each figure). Data are shown as follows: 1 mg/kg 1G01 (FIG. 7A), 1 mg/kg FM08 (FIG. 7B), 1 mg/kg 1G01+1 mg/kg FM08 (FIG. 7C), 0.5 mg/kg 1G01 (FIG. 7D), 0.5 mg/kg FM08 (FIG. 7E), 0.5 mg/kg 1G01+0.5 mg/kg FM08 (FIG. 7F); 0.25 mg/kg 1G01 (FIG. 7G), 0.25 mg/kg FM08 (FIG. 7H), 0.25 mg/kg 1G01+0.25 mg/kg FM08 (FIG. 7I); 0.125 mg/kg 1G01 (FIG. 7J), 0.125 mg/kg FM08-rIgG (FIG. 7K), 0.125 mg/kg 1G01+0.125 mg/kg FM08 (FIG. 7L).

FIGS. 8A-8B show area-under-the-curve negative peaks compared with IgG in serum from area-under-the-curve analyses of body weight loss in BALB/c mice infected with A/Puerto Rico/8/34 following treatment with 1G01, FM08, or 1G01 and FM08 combined. Negative area-under-the-curve peaks are graphed by amount of each mAb (FIG. 8A) or amount of total antibody (FIG. 8B) administered, in mg/kg.

FIGS. 9A-9C show Compusyn software readouts of area-under-the-curve analyses of body weight loss in BALB/c mice infected with A/Puerto Rico/8/34 following treatment with 1G01 and FM08 combined. The (FIG. 9A) dose-effect curve, isobologram (i.e. equi-effective curve, FIG. 9B), and (FIG. 9C) combination index for quantitative definition of synergism, additive effect, and antagonism are shown.

FIGS. 10A and 10B show quantification of human IgG in serum of BALB/c mice 24-hours post-antibody injection and immediately prior to infection with a LD90 (90% lethal dose) of A/Puerto Rico/8/34. BALB/c mice were intravenously injected with 1G01, FM08, or 1G01 and FM08 at 1 mg/kg, 0.5 mg/kg, 0.25 mg/kg, or 0.125 mg/kg for each antibody. FIG. 10A shows human IgG in serum at 24-hours post-antibody injection reported as μg/ml. FIG. 10B shows H1N1 negative area-under-the-curve peaks compared with IgG in serum and EC50 (half maximal effective concentration) values from area-under-the-curve analyses of body weight loss in BALB/c mice infected with A/Puerto Rico/8/34 (FIGS. 8A-8B).

FIG. 11 shows survival over fifteen days in BALB/c mice infected with A/Puerto Rico/8/34 following treatment with 1G01, FM08, or a combination of 1G01 and FM08. Survival in mice pre-treated with a vehicle control was also measured.

FIGS. 12A-12B show survival over fifteen days in BALB/c mice infected with A/Puerto Rico/8/34 following treatment with (FIG. 12A) 0.25 mg/kg 1G01, 0.25 mg/kg FM08, or 0.25 mg/kg 1G01+0.25 mg/kg FM08; or (FIG. 12B) 0.25 mg/kg 1G01, 0.25 mg/kg FM08, and 0.125 mg/kg 1G01+0.125 mg/kg FM08. Survival in mice pre-treated with a vehicle control was also measured.

FIG. 13 shows the design of a DVD (Dual Variable Domain) bi-specific antibody, “FNI17-L-FM08-DVDIg1-LS”, containing anti-NA (FNI17) and anti-HA (FM08) antigen-binding domains.

FIGS. 14A-14B show in vitro inhibition of sialidase activity by FNI17-FM08-DVDIg1-LS. Comparator test articles were FNI17 mAb alone, FNI17+FM08 mAbs, or FM08 mAb alone against H1N1 Cal/09 (FIG. 14A) and H3N2 HK/68 (FIG. 14B). Calculated IC50 values (nM) are shown below the graph in each figure.

FIGS. 15A-15B show in vitro neutralization of H5 and H7 pseudotyped viruses by FM08-FNI9-DVDIg1-LS, FNI9-FM08-DVDIg1-LS, FM08-FNI17-DVDIg1-LS, and FNI17-FM08-DVDIg1-LS. Data for comparator antibody FM08 is also shown. FIG. 15A shows neutralization of H5/VN1194 pp. FIG. 15B shows neutralization of H7/IT/99 pp. Calculated IC50 values (nM) are shown below the graph in each figure.

FIGS. 16A-16B show antibody activation of FcγRIIIa (FIG. 16A; F158 allele) and FcγRIIa (FIG. 16B; H131 allele). Activation was measured using an NFAT-mediated luciferase reporter in engineered Jurkat cells. FM08-FNI17-DVDIg1-LS and FNI17-FM08-DVDIg1-LS were tested, along with comparator antibodies FM08_LS, FHF12-LS, FHF11-v9-LS, and a negative control antibody (FY1-LALA).

FIG. 17 shows the dosing and treatment groups of an in vivo study to evaluate prophylactic activity of FNI17-FM08-DVDIg1_LS (“DVD Format”) in BALB/c mice infected with H1N1/PR8/8/34. Four treatment p (test articles, “TA 1-TA-4”) were evaluated, FM08_LS (TA 1, “mAb-08”), FNI1 7_LS (TA-2, “mAb-17”), FM08_LS+FNI17_LS (TA 3, “mAb-08+mAb-17”), and FNI17-FM08-DVDIg1-LS (TA 4, “DVD Format”).

FIGS. 18A-18D show measurements of body weight over fifteen days in BALB/c mice that were infected with Influenza virus following pre-treatment with FM08_LS (TA 1, “mAb-08”), FNI17_LS (TA 2, “mAb-17”), FM08_LS+FNI17_LS (TA 3, “mAb-08+mAb-1T”), and FNI17-FM08-DVD-LS (TA 4, “DVD Format”). Antibody was administered at 1 mg/kg (FIG. 18A), 0.5 mg/kg (FIG. 18B), 0.25 mg/kg (FIG. 18C), or 0.125 mg/kg (FIG. 18D), one day prior to infection with a LD90 (90% lethal dose) of H1N1/PR8/8/34. Mice in the FNI17-FM08-DVD-LS (TA 4, “DVD Format”) treatment group received an equivalent number of molecules corresponding with the body weight dosage (mg/kg) dosage of TA 1-TA 3.

FIGS. 19A-19B show survival over fifteen days in BALB/c mice infected with H1N1 PR8/8/34 and pre-treated with FM08_LS or FNI17_LS (FIG. 19A); or FM08_LS+FNI17_LS or FNI17-FM08-DVD-LS (FIG. 19B) at different doses.

FIG. 20 shows body weight loss from day 0 to 14 post-infection (reported as negative area-under-the-curve peak values) in mice infected with IAV following pre-treatment with FNI17_LS, FM08_LS, FNI17_LS and FM08_LS, or a FNI17/FM08_LS dual-variable-domain antibody (DVD). Body weight loss in mice pre-treated with a vehicle control was also measured.

In the left graph, for the 1 mg/kg dose (left-most set of five bars), the left-to-right order of the bars corresponds to the top-to-bottom orientation in the figure key (i.e., Vehicle is the left-most bar in the 1 mg/kg quadrant; FNI17/FM08_LS DVD is right-most bar). At the other doses, the left-to-right order of the bars corresponds to the top-to-bottom orientation of the figure key beginning with FNI17 (i.e., FNI17 is the left-most bar in the 0.5 mg/kg quadrant; FNI17/FM08_LS DVD is the right-most bar). In the smaller graph at right, the bars are (from left to right): Vehicle; FNI17+FM08_LS; FNI17; FM08_LS.

### DETAILED DESCRIPTION

The present disclosure relates, in part, to anti-influenza antibodies (and antigen-binding fragments thereof), polynucleotides that encode the anti-influenza antibodies and antigen-binding fragments thereof, and combinations thereof for preventing and treating influenza infection.

Presently disclosed combinations provide surprising synergistic effects and can potently prevent, inhibit, or neutralize an influenza infection, such as an influenza A virus (IAV) infection an influenza B virus (IBV) infection, or both. Presently disclosed combinations can have improved breadth and potency against human and animal-circulating IAV strains, can provide improved function against Monoclonal Antibody-Resistant Mutants (MARMs) and/or viral isolates, can reduce the risk of escape mutants, can promote an endogenous immune response against influenza, have low-to-no non-specific activity (e.g. against healthy subject tissue), are effective against seasonal IAV and/or IBV, are effective against animal IAVs, and/or possess favorable pharmacokinetic properties.

In certain aspects, provided herein combinations and compositions that comprise an anti-hemagglutinin (HA) antibody, or an antigen-binding fragment thereof, and an anti-neuraminidase (NA) antibody, or an antigen-binding fragment thereof, or a polynucleotide or polynucleotides that encode the anti-HA and anti-NA antibodies or antigen-binding fragments thereof, and uses of the same for preventing or treating an influenza infection, as well as for the preparation of a medicament for preventing or treating an influenza infection. Also provided are methods for treating or preventing an influenza infection, wherein the methods comprise administering to a subject an effective amount of an anti-HA antibody (or an antigen-binding fragment thereof) and an anti-NA antibody (or an antigen-binding fragment thereof), or administering an anti-HA antibody (or an antigen-binding fragment thereof), or a polynucleotide or polynucleotides encoding the same, to a subject who has received, will receive, or is receiving an anti-NA antibody (or an antigen-binding fragment thereof), or administering an anti-NA antibody (or an antigen-binding fragment thereof) to a subject who has received, will receive, or is receiving an anti-HA antibody (or an antigen-binding fragment thereof), or a polynucleotide or polynucleotides encoding the same.

Also provided are multispecific antibodies or antigen-binding fragments thereof that comprise an anti-HA binding domain and an anti-NA binding domain, as well as related compositions and uses.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (GIn or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90°/%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA, also referred to as deoxyribonucleic acid), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 748-756; doi.org/10.1021/acscentsci.1c00197 (2021), and van Hoecke and Roose, J. Translational Med 17:54 (2019); https://doi.org/10.1186/s12967-019-1804-8, which modified nucleosides and mRNA features are incorporated herein by reference. Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications arm introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geirts et al., Mol. Ther. 8:108, 2003: Mates et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning. A Laboratory Manual 2d ed. (Cold Spring Harbor Laboratory, 1989).

In the context of an influenza infection, a “host” refers to a cell or a subject infected with the influenza.

“Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in an influenza NA antigen, such as present in a virion, or expressed or presented on the surface of a cell infected by the influenza.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, Combinations, and Compositions

Anti-HA and anti-NA antibodies are disclosed herein, and have utility in presently disclosed combinations, compositions, uses, and methods. Provided embodiments include an antibody, or an antigen-binding fragment thereof, that is capable of binding to an influenza A virus (IAV) hemagglutinin (HA) and neutralizing infection by the IAV, and/or an antibody, or an antigen-binding fragment thereof, that is capable of binding to a neuraminidase (NA) from: (i) an IAV, wherein the IAV comprises a Group 1 IAV, a Group 2 IAV, or both; and (ii) an influenza B virus (IBV), and is capable of neutralizing infection and/or inhibiting sialidase activity by the IAV and/or the IBV.

In some embodiments, a composition, combination, or therapy comprises an anti-NA antibody or antigen-binding fragment and an anti-HA antibody or antigen-binding fragment at a ratio of 1:1, 1:1.5, 1:2, 1:2.5, 1:3, 1:4, 1:5, 1:10, 10:1, 5:1, 4:1, 3:1, 2.5:1, or 2:1.

In certain embodiments, an antibody or antigen-binding fragment of the present disclosure associates with or unites with a HA or NA while not significantly associating or uniting with any other molecules or components in a sample.

In certain embodiments, an antibody or antigen-binding fragment of the present disclosure specifically binds to a IAV HA or NA. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, biolayer interferometry, and surface plasmon resonance (Biacore®) analysis (see, e.g., Scatchard et al., Ann. N.Y. Acad Sci. 51:660, 1949, Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a influenza HA and/or NA antigen in a host cell (e.g., by transfection) and immunostaining the (e.g., fixed, or fixed and permeabilized) host cell with antibody and analyzing binding by flow cytometry (e.g., using a ZE5 Cell Analyzer (BioRad®) and FlowJo software (TreeStar). In some embodiments, positive binding can be defined by differential staining by antibody of influenza HA and/or NA-expressing cells versus control (e.g., mock) cells.

In some embodiments an antibody or antigen-binding fragment of the present disclosure binds to an influenza HA or NA protein, as measured using biolayer interferometry, or by surface plasmon resonance.

Certain characteristics of presently disclosed antibodies or antigen-binding fragments may be described using IC50 or EC50 values. In certain embodiments, the IC50 is the concentration of a composition (e.g., antibody) that results in half-maximal inhibition of the indicated biological or biochemical function, activity, or response. In certain embodiments, the EC50 is the concentration of a composition that provides the half-maximal response in the assay. In some embodiments, e.g., for describing the ability of a presently disclosed antibody or antigen-binding fragment to neutralize infection by influenza, IC50 and EC50 are used interchangeably.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., DVD-Igs (e.g., U.S. Pat. No. 8,258,268, which formats are incorporated herein by reference in their entirety), bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows. FR1-LCDR1-FR2-LCDR2-FR3-LCDR3-FR4. In general, the VH and the VL together form the antigen-binding site through their respective CDRs. In certain embodiments, one or more CDRs do not contact antigen and/or do not contribute energetically to antigen binding.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme or system, such as the Kabat, Chothia, EU, IMGT, Contact, North, Martin, and AHo numbering schemes (see, e.g., Kabat et al., “Sequences of Proteins of Immunological Interest, US Dept. Health and Human Services, Public Health Service National Institutes of Health, 1991, 5^{th} ed.: Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001); North et al. J Mol Biol. (2011) 406:228-56; doi:10.1016/j.jmb.2010.10.030; Abhinandan and Martin, Mol Immunol. (2008) 45:3832-9. 10.1016/j.molimm.2008.05.022). The antibody and CDR numbering systems of these references are incorporated herein by reference. Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody of the present disclosure is capable of neutralizing infection by influenza. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment can be capable of preventing and/or neutralizing an influenza infection in an in vitro model of infection and/or in an in vivo animal model of infection and/or in a human. In certain embodiments, the antibody, or antigen-binding fragment thereof, is human, humanized, or chimeric.

In some embodiments, CDRs are according to the IMGT numbering system.

In certain embodiments, (1) the anti-HA antibody or antigen-binding fragment comprises a heavy chain variable domain (VH) comprising a complementarity determining region (CDR)H1, a CDRH2, and a CDRH3, and a light chain variable domain (VL) comprising a CDRL1, a CDRL2, and a CDRL3: (1)(i) the CDRH1 comprises or consists of the amino acid sequence of any one of SEQ ID NOs.: 3, 32, or 15, or a functional variant thereof comprising one, two, or three acid substitutions, one or more of which substitutions is optionally a conservative substitution and/or is a substitution to a germline-encoded amino acid; and/or (1)(ii) the CDRH2 comprises or consists of the amino acid sequence of any one of SEQ ID NOs.: 4, 29, 35, 16, or 42, or a functional variant thereof comprising one, two, or three amino acid substitutions, one or more of which substitutions is optionally a conservative substitution and/or is a substitution to a germline-encoded amino acid; and/or (1)(iii) the CDRH3 comprises or consists of the amino acid sequence of any one of SEQ ID NOs.: 5 or 17, or a functional variant thereof comprising one, two, or three amino acid substitutions, one or more of which substitutions is optionally a conservative substitution and/or is a substitution to a germline-encoded amino acid; and/or (1)(iv) the CDRL1 comprises or consists of the amino acid sequence of any one of SEQ ID NOs.: 9 or 21, or a functional variant thereof comprising one, two, or three amino acid substitutions, one or more of which substitutions is optionally a conservative substitution and/or is a substitution to a germline-encoded amino acid; and/or (1)(v) the CDRL2 optionally comprises or consists of the amino acid sequence of any one of SEQ ID NOs.: 10 or 22, or a functional variant thereof comprising one, two, or three amino acid substitutions, one or more of which substitutions is optionally a conservative substitution and/or is a substitution to a germline-encoded amino acid; and/or (1)(vi) the CDRL3 comprises or consists of the amino acid sequence of any one of SEQ ID NOs.: 11 or 23, or a functional variant thereof comprising having one, two, or three amino acid substitutions, one or more of which substitutions is optionally a conservative substitution and/or is a substitution to a germline-encoded amino acid; and/or (2) the anti-NA antibody or antigen-binding fragment comprises a VH comprising a CDRH1, a CDRH2, and a CDRH3, and a VL comprising a CDRL1, a CDRL2, and a CDRL3, wherein the CDRs are determined according to the IMGT numbering system, and wherein: (2)(i) optionally, the CDRH1 comprises or consists of the amino acid sequence set forth in any one of SEQ ID NOs.: 49, 61, 73, 85, 97, 109, 121, 133, 145, 157, 169, 181, 193, or 205, or a functional variant thereof comprising one, two, or three acid substitutions, one or more of which substitutions is optionally a conservative substitution and/or is a substitution to a germline-encoded amino acid; (2)(ii) optionally, the CDRH2 comprises or consists of the amino acid sequence set forth in any one of SEQ ID NOs.: 50, 62, 74, 86, 98, 110, 122, 134, 146, 158, 170, 182, 194, or 206, or a functional variant thereof comprising one, two, or three amino acid substitutions, one or more of which substitutions is optionally a conservative substitution and/or is a substitution to a germline-encoded amino acid: (2)(iii) the CDRH3 comprises or consists of the amino acid sequence set forth in any one of SEQ ID NOs.: 51, 63, 75, 218, 87, 99, 111, 123, 135, 230, 147, 159, 171, 183, 195, or 207, or a functional variant thereof comprising one, two, or three amino acid substitutions, one or more of which substitutions is optionally a conservative substitution and/or is a substitution to a germline-encoded amino acid; (2)(iv) optionally, the CDRL1 comprises or consists of the amino acid sequence set forth in any one of SEQ ID NOs.: 55, 67, 79, 91, 103, 115, 127, 139, 151, 163, 175, 187, 199, or 211, or a functional variant thereof comprising one, two, or three amino acid substitutions, one or more of which substitutions is optionally a conservative substitution and/or is a substitution to a germline-encoded amino acid; (2)(v) optionally, the CDRL2 comprises or consists of the amino acid sequence set forth in any one of SEQ ID NOs.: 56, 68, 80, 92, 104, 116, 128, 140, 152, 164, 176, 188, 200, or 212, or a functional variant thereof comprising one, two, or three amino acid substitutions, one or more of which substitutions is optionally a conservative substitution and/or is a substitution to a germline-encoded amino acid; and/or (2)(vi) optionally, the CDRL3 comprises or consists of the amino acid sequence set forth in any one of SEQ ID NOs.: 57, 69, 81, 221, 224, 227, 93, 105, 117, 129, 141, 233, 239, 153, 165, 177, 189, 201, 236, or 213, or a functional variant thereof comprising having one, two, or three amino acid substitutions, one or more of which substitutions is optionally a conservative substitution and/or is a substitution to a germline-encoded amino acid.

In further embodiments, (1) the anti-HA antibody or antigen-binding fragment comprises CDRH1, CDRH2, CDRH3, CDRL1, CDRL2, and CDRL3 amino acid sequences of SEQ ID NOs.: (1)(i) 3-5 and 9-11, respectively; (1)(ii) 3, 29, 5 and 9-11, respectively; (1)(iii) 32, 4, 5 and 9-11, respectively; (1)(iv) 3, 35, 5 and 9-11, respectively; (1)(v) 32, 35, 5, and 9-11, respectively; (1)(vi) 15-17 and 21-23, respectively; or (1)(vii) 15, 42, 17 and 21-23, respectively; and/or (2) the anti-NA antibody or antigen-binding fragment comprises CDRH1, CDRH2, CDRH3, CDRL1, CDRL2, and CDRL3 amino acid sequences of SEQ ID NOs.: (2)(i) 49-51 and 55-57, respectively; (2)(ii) 61-63 and 67-69, respectively; (2)(iii) 73-75 and 79-81, respectively; (2)(iv) 73, 74, 218, and 79-81, respectively; (2)(v) 73-75, 79, 80, and 221, respectively; (2)(vi) 73-75, 79, 80, and 224, respectively; (2)(vii) 73-75, 79, 80, and 227, respectively; (2)(viii) 73, 74, 218, 79, 80, and 221, respectively; (2)(ix) 73, 74, 218, 79, 80, and 224, respectively; (2)(x) 73, 74, 218, 79, 80, and 227, respectively; (2)(xi) 85-87 and 91-93, respectively; (2)(xii) 97-99 and 103-105, respectively; (2)(xiii) 109-111 and 115-117, respectively; (2)(xiv) 121-123 and 127-129, respectively; (2)(xv) 133-135 and 139-141, respectively; (2)(xvi) 133, 134, 230 and 139-141, respectively; (2)(xvii) 133-135, 139, 141, and 233, respectively; (2)(xviii) 133-135, 139, 141, and 236, respectively; (2)(xix) 133-135, 139, 141, and 239, respectively; (2)(xx) 133, 134, 184, 139, 141, and 233, respectively; (2)(xxi) 133, 134, 184, 139, 141, and 236, respectively; (2)(xxii) 133, 134, 184, 139, 141, and 239, respectively; (2)(xxiii) 145-147 and 151-153, respectively; (2)(xxiv) 157-159 and 163-165, respectively; (2)(xxv) 169-171 and 175-177, respectively; (2)(xxvi) 181-183 and 187-189, respectively; (2)(xxvii) 193-195 and 199-201, respectively; or (2)(xxviii) 205-207 and 211-213, respectively.

In some embodiments, (1) the anti-HA antibody or antigen-binding fragment comprises CDRH1, CDRH2, CDRH3, CDRL1, CDRL2, and CDRL3 amino acid sequences of SEQ ID NOs.: (1)(i) 3-5 and 9-1l, respectively; (1)(ii) 3, 29, 5 and 9-11, respectively; (1)(iii) 32, 4, 5 and 9-11, respectively; (1)(iv) 3, 35, 5 and 9-11, respectively; (1)(v) 32, 35, 5, and 9-11, respectively; (1)(vi) 15-17 and 21-23, respectively; or (1)(vii) 15, 42, 17 and 21-23, respectively; and/or (2) the anti-NA antibody or antigen-binding fragment comprises CDRH1, CDRH2, CDRH3, CDRL1, CDRL2, and CDRL3 amino acid sequences of SEQ ID NOs.: (2)(i) 73-75 and 79-81, respectively; (2)(ii) 73, 74, 218, and 79-81, respectively; (2)(iii) 73-75, 79, 80, and 221, respectively; (2)(iv) 73-75, 79, 80, and 224, respectively; (2)(v) 73-75, 79, 80, and 227, respectively; (2)(vi) 73, 74, 218, 79, 80, and 221, respectively; (2)(vii) 73, 74, 218, 79, 80, and 224, respectively; (2)(viii) 73, 74, 218, 79, 80, and 227, respectively; (2)(ix) 133-135 and 139-141, respectively; (2)(x) 133, 134, 230 and 139-141, respectively; (2)(xi) 133-135, 139, 141, and 233, respectively; (2)(xii) 133-135, 139, 141, and 236, respectively; (2)(xiii) 133-135, 139, 141, and 239, respectively; (2)(xiv) 133, 134, 184, 139, 141, and 233, respectively; (2)(xv) 133, 134, 184, 139, 141, and 236, respectively; or (2)(xvi) 133, 134, 184, 139, 141, and 239, respectively.

In certain embodiments, an antibody or antigen-binding fragment is provided that comprises CDRs of in a VH sequence according to any one of SEQ ID NOs.: 2, 14, 26, 171, 38, 50, 62, 74, 86, 183, 98, 110, 122, 134, 146, and 158, and in a VL sequence according to any one of SEQ ID NOs.: 26, 36, 46, 56, 66, 76, 86, 96, 8, 20, 32, 44, 56, 68, 80, 92, 104, 116, 128, 140, 152, 174, 177, 180, 186, 189, 192, and 164, as determined using any known CDR numbering method, including the Kabat, Chothia, EU, IMGT, Martin (Enhanced Chothia), Contact, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software (www.chemcomp.com).

In some embodiments, the anti-HA antibody or antigen-binding fragment comprises CDRH1, CDRH2, CDRH3 of the VH amino sequence set forth in SEQ ID NO.:43, and CDRL1, CDRL2, and CDRL3 of the VL amino acid sequence set forth in SEQ ID NO.:44. In further embodiments, the anti-HA antibody or antigen-binding fragment comprises the VH set forth in SEQ ID NO.:43 and the VL set forth in SEQ ID NO.:44.

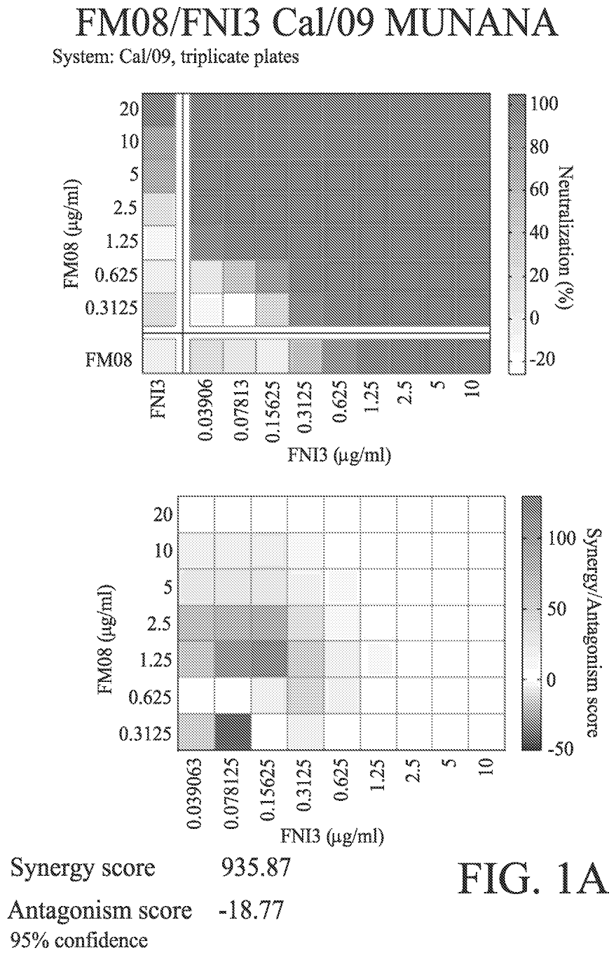

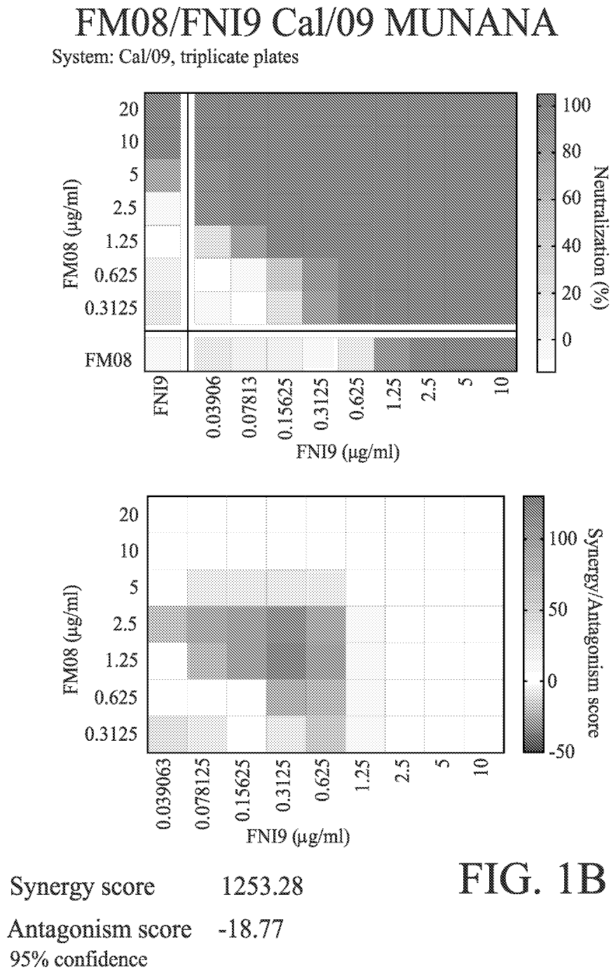

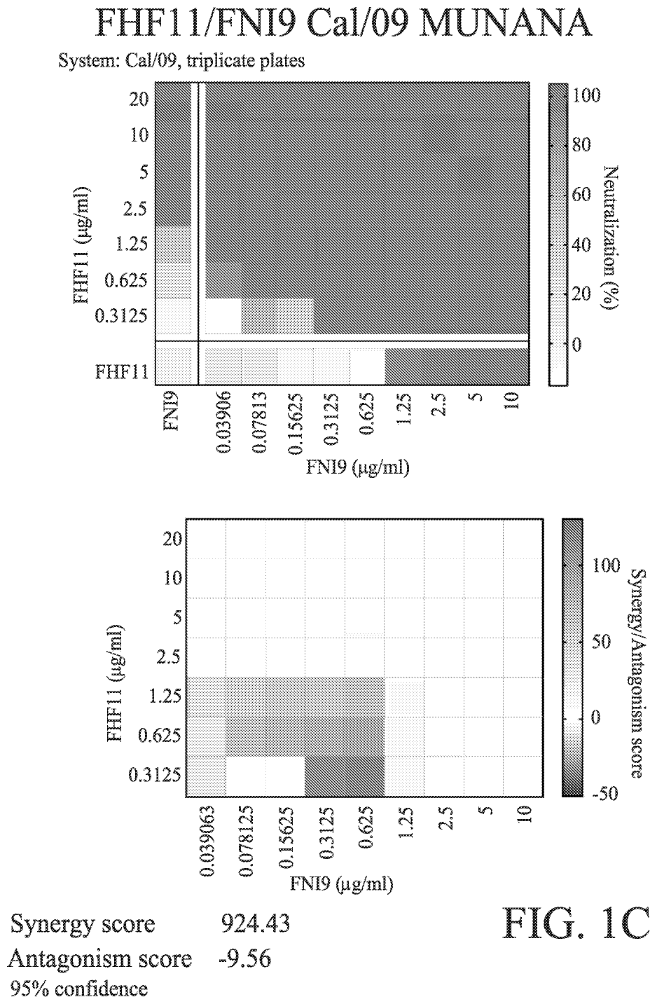

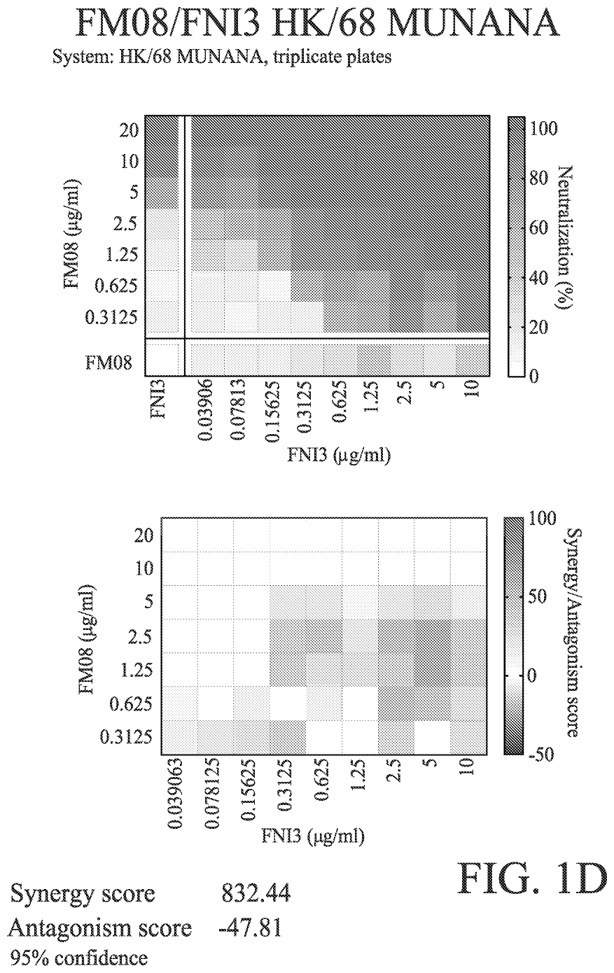

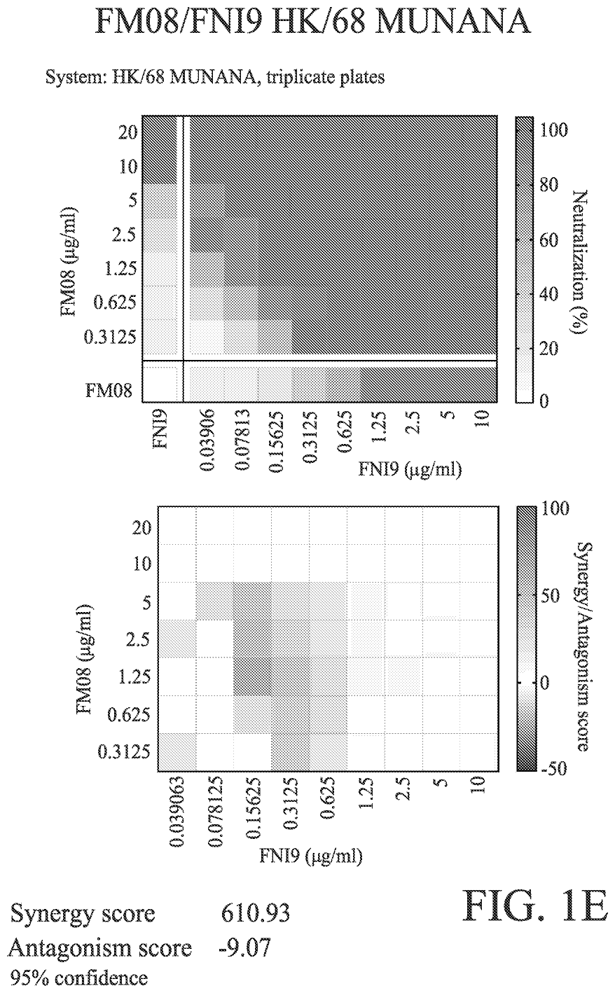

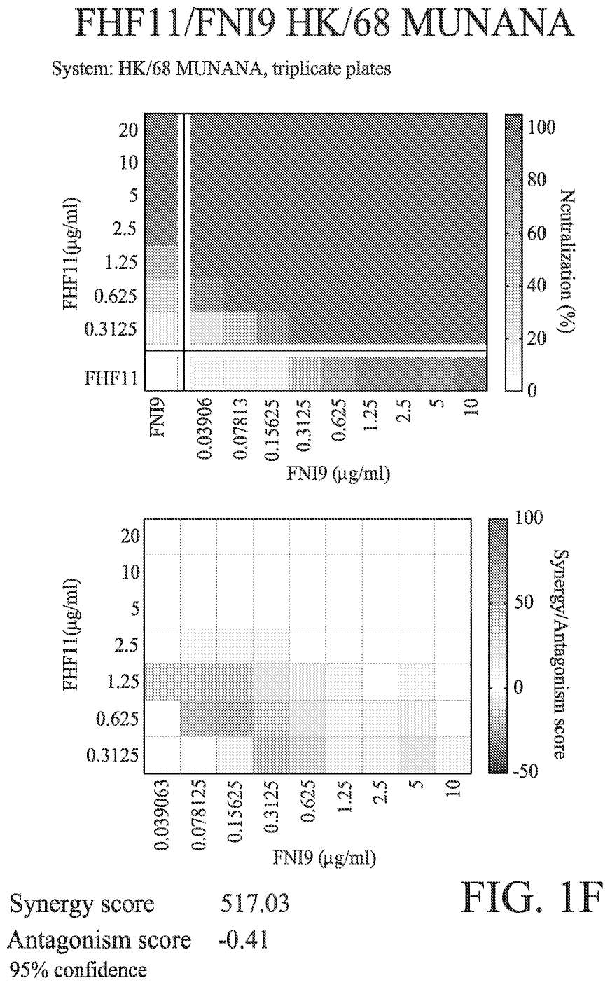

In some embodiments, the anti-NA antibody or antigen-binding fragment comprises CDRH1, CDRH2, CDRH3, CDRL1, CDRL2, and CDRL3 of antibody 1G01, as shown in FIG. 1B of Stadlebaeur et al., Science 366(6464):466-504 (2019), which amino acid sequences are incorporated herein by reference. In further embodiments, the anti-NA antibody or antigen-binding fragment comprises the VH and the VL of antibody 1G01, as shown in FIG. 1B of Stadlebaeur et al., Science 366(6464):466-504 (2019), which amino acid sequences are incorporated herein by reference.

In certain embodiments, (1) the anti-HA antibody or antigen-binding fragment comprises (1)(i) a VH comprising or consisting of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 26, 28, 31, 34, 37, 14, 39 41, and 43 wherein sequence variation with reference to SEQ ID NO.: 2, 26, 28, 31, 34, 37, 14, 39 41, or 43, respectively, is optionally comprised in one or more framework region and/or sequence variation comprises one or more substitution to a germline-encoded amino acid; and/or (1)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 8, 20, or 44, wherein sequence variation with respect to SEQ ID NO.: 8, 20, or 44, respectively, is optionally comprised in one or more framework regions and/or sequence variation comprises one or more substitution to a germline-encoded amino acid; and/or (2) the anti-NA antibody or antigen-binding fragment comprises (2)(i) a VH comprising or consisting of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 48, 60, 72, 171, 84, 96, 108, 120, 132, 229, 144, 156, 168, 180, 192, 204, 241, 245, and 249 wherein sequence variation with reference to SEQ ID NO.: 48, 60, 72, 171, 84, 96, 108, 120, 132, 229, 144, 156, 168, 180, 192, 204, 241, 245, and 249 respectively, is optionally comprised in one or more framework region and/or sequence variation comprises one or more substitution to a germline-encoded amino acid; and/or (2)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 54, 66, 78, 90, 102, 114, 126, 138, 150, 162, 174, 186, 198, 220, 223, 226, 232, 235, 238, 210, 243, 247, and 251 wherein sequence variation with respect to SEQ ID NO.: 54, 66, 78, 90, 102, 114, 126, 138, 150, 162, 174, 186, 198, 220, 223, 226, 232, 235, 238, 210, 243, 247, and 251 respectively, is optionally comprised in one or more framework regions and/or sequence variation comprises one or more substitution to a germline-encoded amino acid.

In particular embodiments, the VH is encoded by or is derived from VH6-1, DH3-3, and/or JH6.

In some embodiments, (1) the VH and the VL of the anti-HA antibody or antigen-binding fragment comprise or consist of the amino acid sequences according to SEQ ID NOs.: (1)(i) 2 and 8, respectively; (1)(ii) 26 and 8, respectively; (1)(iii) 28 and 8, respectively; (1)(iv) 31 and 8, respectively; (1)(v) 34 and 8, respectively; (1)(vi) 37 and 8, respectively; (1)(vii) 14 and 20, respectively; (1)(viii) 39 and 20, respectively; (1)(ix) 41 and 20, respectively; or (1)(x) 43 and 44, respectively; and/or (2) the VH and the VL of the anti-NA antibody or antigen-binding fragment comprise or consist of the amino acid sequences according to SEQ ID NOs.: (2)(i) 48 and 54, respectively; (2)(ii) 60 and 66, respectively; (2)(iii) 72 and 78 or 220 or 223, respectively; (2)(vi) 72 and 226, respectively; (2)(vii) 217 and 78, respectively; (2)(viii) 217 and 220, respectively; (2)(ix) 217 and 223, respectively; (2)(x) 217 and 226, respectively; (2)(xi) 84 and 90, respectively; (2)(xii) 96 and 102, respectively; (2)(xiii) 108 and 114, respectively; (2)(xiv) 120 and 126, respectively; (2)(xv) 132 and 138, respectively; (2)(xvi) 132 and 232, respectively; (2)(xvii) 132 and 235, respectively; (2)(xviii) 132 and 238, respectively; (2)(xix) 229 and 138, respectively; (2)(xx) 229 and 232, respectively; (2)(xxi) 229 and 235, respectively; (2)(xxii) 229 and 238, respectively; (2)(xxiii) 144 and 150, respectively; (2)(xxiv) 156 and 162, respectively; (2)(xxv) 168 and 174, respectively; (2)(xxvi) 180 and 186, respectively; (2)(xxvii) 192 and 198, respectively; (2)(xxviii) 204 and 210, respectively; (2)(xxix) 241 and 243, respectively; (2)(xxx) 245 and 247, respectively; or (2)(xxxi) 249 and 251, respectively.

In particular embodiments, (1) the VH and the VL of the anti-HA antibody or antigen-binding fragment comprise or consist of the amino acid sequences according to SEQ ID NOs.: (1)(i) 2 and 8, respectively; (1)(ii) 26 and 8, respectively; (1)(iii) 28 and 8, respectively; (1)(iv) 31 and 8, respectively; (1)(v) 34 and 8, respectively; (1)(vi) 37 and 8, respectively; (1)(vii) 14 and 20, respectively; (1)(viii) 39 and 20, respectively; (1)(ix) 41 and 20, respectively; or (1)(x) 43 and 44, respectively; and/or (2) the VH and the VL of the anti-NA antibody or antigen-binding fragment comprise or consist of the amino acid sequences according to SEQ ID NOs.: (2)(i) 72 and 78 or 220 or 223, respectively; (2)(ii) 72 and 226, respectively; (2)(iii) 217 and 78, respectively; (2)(iv) 217 and 220, respectively; (2)(v) 132 and 138, respectively; (2)(vi) 132 and 232, respectively; (2)(vii) 132 and 235, respectively; (2)(viii) 132 and 238, respectively; (2)(ix) 229 and 138, respectively; (2)(x) 229 and 232, respectively; (2)(xi) 229 and 235, respectively; (2)(xii) 229 and 238, respectively; (2)(xiii) 217 and 223, respectively; (2)(xiv) 217 and 226, respectively; (2)(xv) 241 and 243, respectively; (2)(xvi) 245 and 247, respectively; or (2)(xvii) 249 and 251, respectively.

In certain embodiments, the NA is a N1, a N2, and/or a N9.

In certain embodiments, the antibody or antigen-binding fragment is capable of binding to: (1) a NA epitope that comprises any one or more of the following amino acids (N1 NA numbering): R368, R293, E228, E344, S247, D198, D151, R118; and/or (2) a NA epitope that comprises any one or more of the following amino acids (N2 NA numbering): R371, R292, E227, E344, S247, D198, D151, R118. It will be understood that the antibodies and antigen-binding fragments may also bind to influenza neuraminidases which may not follow N1 or N2 amino acid numbering conventions; amino acids of these epitopes may correspond to herein-indicated N1 or N2 amino acid residues, such as by being the same amino acid residue at an equivalent (e.g., by alignment, 3-D structure, conservation, or combinations of these) but differently numbered, position in the NA. Accordingly, reference to N1 or N2 numbering will be understood as the amino acid corresponding to the enumerated amino acid.

An example showing N1 vs N2 position numbering (using H1N1_California.07.2009 and H3N2_NewYork.392.2004) is provided in Table 2.

In certain embodiments, the antibody or antigen-binding fragment is capable of binding to: (1) a NA epitope that comprises the amino acids R368, R293, E228, D151, and RI 18 (N1 NA numbering); and/or (2) a NA epitope that comprises the amino acids R371, R292, E227, D151, and R118 (N2 NA numbering).

In certain embodiments, the antibody or antigen-binding fragment is capable of binding to an epitope comprised in or comprising a NA active site (as described herein, the NA active site comprises functional amino acids that form the catalytic core and directly contact sialic acid, as well as structural amino acids that form the active site framework), wherein, optionally, the NA active site comprises the following amino acids (N2 numbering): R118, D151, R152, R224, E276, R292, R371, Y406, E119, R156, W178, S179, D/N198, I222, E227, H274, E277, D293, E425. In certain embodiments, R118, D151, R152, R224, E276, R292, R371, and Y406 form the catalytic core and directly contact sialic acid. In certain embodiments, E119, R156, W178, S179, D/N198, 1222, E227, H274, E277, D293, and E425 form the active site framework.

In certain embodiments, the epitope comprises or further comprises any one or more of the following NA amino acids (N2 numbering): E344, E227, S247, and D198.

In certain embodiments, the antibody or antigen-binding fragment is capable of binding to a NA comprising a S245N amino acid mutation and/or a E221D amino acid mutation (N2 numbering).

In certain embodiments, the NA comprises an IBV NA. In certain embodiments, the antibody or antigen-binding fragment is capable of binding to an IBV NA epitope that comprises any one or more of the following amino acids (IBV numbering; e.g., as for FluB Victoria and FluB Yamagata): R116, D149, E226, R292, and R374. In some embodiments, the epitope comprises the amino acids R116, D149, E226, R292, and R374.

In certain embodiments, (i) the Group 1 IAV NA comprises a H1N1 and/or a H5N1; (ii) the Group 2 IAV NA comprises a H3N2 and/or a H7N9; and/or
(iii) the IBV NA comprises one or more of: B/Lee/10/1940 (Ancestral);
B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); and B/Jiangsu/10/2003 (Yamagata).

In certain embodiments, the anti-HA antibody or antigen-binding fragment is capable of binding to any one or more of the following IAV subtypes: H1, H2, H3, H4, H5, H8, H9, H10, H11, H12, H13, H14, H15, H17, and H18.

In certain embodiments, the anti-HA antibody or antigen-binding fragment is capable of neutralizing infection by: (i) a H1N1 IAV, wherein, optionally, the H1N1 IAV comprises any one or more of: A/California/07/2009, A/PR/8/34, and A/Solomon Islands/3/06; and (ii) a H3N2 IAV, wherein, optionally, the H3N2 IAV comprises any one or more of: A/Aichi/2/68, A/Brisbane/10/07, and A/Hong Kong/68 (i) a Group 1 IAV, wherein, optionally, the Group 1 IAV comprises or is a H5 IAV, wherein, further optionally, the H5 IAV comprises or is H5/VN/11/94 pp; and (ii) a Group 2 IAV, wherein, optionally, the Group 2 IAV comprises or is a H7 IAV, wherein, further optionally, the H7 IAV comprises or is H7/IT/99 pp, wherein, optionally, neutralization of infection is as determined using a virus pseudotyped with the IAV.

In certain embodiments, the HA comprises (i) a H1 HA, which optionally comprises any one or more of: A/England/195/2009; A/Brisbane/59/2007; A/Solomon Islands/3/2006; A/New Caledonia/20/99; A/Texas/36/1991; A/Taiwan/01/1986; A/New Jersey/8/1976; A/Albany/12/1951; A/Fort Monmouth/1/1947; A/New York/I/1918; A/Puerto Rico/8/34; and A/California/07/2009; (ii) a H2 HA, optionally comprising A/Japan/305/1957; (iii) a H5 HA, optionally comprising A/Vietnam/i 194/2004; and (iv) a H9 HA, optionally comprising A/Hong Kong/1073/99.

In any of the presently disclosed combinations, compositions, methods, and uses, (i) the Group 1 IAV NA can comprise a N1, a N4, a N5, and/or a N8; and/or (ii) the Group 2 IAV NA can comprise a N2, a N3, a N6, a N7, and/or a N9. In certain embodiments, (i) the N1 is a N1 from any one or more of: A/California/07/2009, A/California/07/2009 I23R/H275Y, A/Swine/Jiangsu/J004/2018, A/Stockholm/18/2007, A/Brisbane/02/2018, A/Michigan/45/2015, A/Mississippi/3/2001, A/Netherlands/603/2009, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011; (v) the N2 is a N2 from any one or more of: A/Washington/01/2007, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, and A/Victoria/361/2011; (vi) the N3 is from A/Canada/rv504/2004; (v) the N6 is from A/swine/Ontario/01911/1/99; (vi) the N7 is from A/Netherlands/078/03; and/or (vii) the N9 is from A/Anhui/2013.

In any of the presently disclosed combinations, compositions, methods, and uses, the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/I/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; and B/Phuket/3073/2013.

In any of the presently disclosed combinations, compositions, methods, and uses, the NA is a N1, a N2, and/or a N9.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3.

In any of the presently disclosed embodiments, an antibody or antigen-binding fragment of the present disclosure may comprise any one or more of CL, a CH1, a CH2, and a CH3. In certain embodiments, a CL comprises an amino acid sequence having 90%, 91%, 92%, 93%, 94%, 95%, 96%, 97%, 98%, 99%, or 100% identity to the amino acid sequence of SEQ ID NO.:254. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.:252, SEQ ID NO.:253, SEQ ID NO.:280, or SEQ ID NO.:281. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment—linker—light chain Fab fragment) or (light chain Fab fragment—linker—heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the V_{H} and V_{L} antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (Bird et al., Science 242:423-426 (1988)) and the pentamer Gly-Gly-Gly-Gly-Ser when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

ScFvs can be constructed using any combination of the VH and VL sequences or any combination of the CDRH1, CDRH2, CDRH3, CDRL1, CDRL2, and CDRL3 sequences disclosed herein.

In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a influenza NA antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations.

In certain embodiments, an antibody or antigen-binding fragment of the present disclosure is monospecific (e.g., binds to a single epitope) or is multispecific (e.g., binds to multiple epitopes and/or target molecules). In some embodiments, a multispecific antibody or antigen-binding fragment comprises a binding domain specific for an HA antigen and a binding domain specific for an NA antigen. For example, a binding domain comprising CDRs and/or a VH and a VL from any anti-HA antibody disclosed herein and a binding domain comprising CDRs and/or a VH and a VL from any anti-NA antibody disclosed herein can be used in a multispecific antibody. Antibodies and antigen binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different NA epitope, and a bispecific or multispecific antibody or antigen-binding fragment as provided herein can, in some embodiments, comprise a further NA-specific binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In particular embodiments, a bispecific antibody is provided in a DVD-Ig format. In further embodiments, the DVD-Ig format bispecific antibody comprises a binding domain that is capable of specifically binding to an HA antigen and a binding domain that is capable of specifically binding to a NA antigen. In still further embodiments, the binding domain that is capable of specifically binding to the HA antigen comprises CDRs from, and/or comprises a VH and a VL according to, the variable region amino acid sequences set forth in SEQ ID NOs.:43 and 44, respectively. In certain embodiments, the binding domain that is capable of specifically binding to the NA antigen comprises CDRs from, and/or a VH and a VL according to, the variable region amino acid sequences set forth in SEQ ID NOs.:72 and 78, respectively, or in SEQ ID NOs.:132 and 138, respectively, or in SEQ ID NOs.:192 and 198, respectively, or in SEQ ID NOs.:204 and 210, respectively, or in SEQ ID NOs.:241 and 243, respectively. It will be understood that the anti-HA binding domain and the anti-NA binding domain can be present in any orientation or arrangement in the DVD-Ig bispecific antibody, e.g., the anti-HA binding domain can be disposed N-terminal of the anti-NA binding domain, or the anti-NA binding domain can be disposed N-terminal of the anti-HA binding domain.

In other embodiments, a bispecific antibody is provided in an IEI-Ig format. In further embodiments, the IEI-Ig format bispecific antibody comprises a binding domain that is capable of specifically binding to an HA antigen and a binding domain that is capable of specifically binding to a NA antigen. In still further embodiments, the binding domain that is capable of specifically binding to the HA antigen comprises CDRs from, and/or a VH and a VL according to, the variable region amino acid sequences set forth in SEQ ID NOs.:43 and 44, respectively. In certain embodiments, the binding domain that is capable of specifically binding to the NA antigen comprises CDRs from, and/or comprises a VH and a VL according to, the variable region amino acid sequences set forth in SEQ ID NOs.:72 and 78, respectively, or in SEQ ID NOs.:132 and 138, respectively, or in SEQ ID NOs.:192 and 198, respectively, or in SEQ ID NOs.:204 and 210, respectively, or in SEQ ID NOs.:241 and 243, respectively. It will be understood that the anti-HA binding domain and the anti-NA binding domain can be present in any orientation or arrangement in the IEI-Ig bispecific antibody; e.g., the anti-HA binding domain can be disposed in the VH-CH1 (or VL-CL1) elbow region of an anti-NA Fab, or the anti-NA binding domain can be disposed in the VH-CH1 (or VL-CL1) elbow region of an anti-HA Fab.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides). Antibody “effector functions” refer to those biological activities attributable to the Fc region (a native sequence Fc region or amino acid sequence variant Fc region) of an antibody, and vary with the antibody isotype. Examples of antibody effector functions include. C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P33 IS, E333A, S239D/A330L/I332E, P257I/Q311, K326W/E333S, S239D/I332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcER, for IgA as FcaR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with higher affinity, and the “R131” variant, which binds to IgG1 Fc with low affinityer. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with lower affinity, and the “V158” variant, which binds to IgG1 Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239 (EU numbering).

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V: T307A: E380A (EU numbering). In certain embodiments, a half-life-extending mutation comprises M428L/N434S (also referred to herein as “MLNS”, “LS”, “_LS”, and “-LS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes a Fc moiety that comprises the substitution mutations M428L/N434S. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D (e.g., comprises S at 239). In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., H_{C} domains.

A “human antibody” is an antibody containing only sequences that are present in an antibody that is produced by a human (i.e., sequences that are encoded by human antibody-encoding genes). However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, various pharmacokinetic (“PK”) parameters are used to describe or characterize the antibodies or antigen-binding fragments provided herein. Details regarding collection of antibody serum concentrations for purpose of evaluating PK parameters are described in association with the Examples herein. The term “t_{1/2}” or “half-life” refers to the elimination half-life of the antibody or antigen-binding fragment included in the pharmaceutical composition administered to a subject. The term “Cₗₐₛₜ” generally refers to the last measurable plasma concentration (i.e., subsequent thereto, the substance is not present at a measurable concentration in plasma).

In any of the presently disclosed embodiments, an antibody or antigen-binding fragment can comprise the CH1-CH3 amino acid sequence set forth in SEQ ID NO.:252 and/or the CH1-CH3 amino acid sequence set forth in SEQ ID NO.:253 and/or the CH1-CH3 amino acid sequence set forth in SEQ ID NO.:280 and/or the CH1-CH3 amino acid sequence set forth in SEQ ID NO.:251.

In any of the presently disclosed embodiments, an antibody or antigen-binding fragment can comprise the CL amino acid sequence set forth in SEQ ID NO.:254.

In some embodiments, an antibody comprises the heavy chain amino acid sequence set forth in SEQ ID NO.:255. In certain embodiments, the antibody further comprises the light chain amino acid sequence set forth in SEQ ID NO.:257.

In some embodiments, an antibody comprises the heavy chain amino acid sequence set forth in SEQ ID NO.:256. In certain embodiments, the antibody further comprises the light chain amino acid sequence set forth in SEQ ID NO.:257.

In some embodiments, an antibody comprises the heavy chain amino acid sequence set forth in SEQ ID NO.:270 or 272. In certain embodiments, the antibody further comprises the light chain amino acid sequence set forth in SEQ ID NO.:271 or 273.

In certain embodiments, an anti-NA antibody or antigen-binding fragment comprises the heavy chain amino acid sequence set forth in SEQ ID NO.:255 and the light chain amino acid sequence set forth in SEQ ID NO.:257, and an anti-HA antibody or antigen-binding fragment comprises the heavy chain amino acid sequence set forth in SEQ ID NO.:270 or 272 and the light chain amino acid sequence set forth in SEQ ID NO.:271 or 273.

In some embodiments, an anti-NA antibody or antigen-binding fragment comprises the heavy chain amino acid sequence set forth in SEQ ID NO.:256 and the light chain amino acid sequence set forth in SEQ ID NO.:257, and an anti-HA antibody or antigen-binding fragment comprises the heavy chain amino acid sequence set forth in SEQ ID NO.:270 or 272 and the light chain amino acid sequence set forth in SEQ ID NO.:271 or 273.

### Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain). In certain embodiments, the polynucleotide is codon-optimized for expression in a host cell (e.g., a human cell, or a CHO cell). Once a coding sequence is known or identified, codon optimization can be performed using known techniques and tools, e.g., using the GenScript® OptimiumGene™ tool, or the like). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In any of the presently disclosed embodiments, the polynucleotide can comprise deoxyribonucleic acid (DNA) or ribonucleic acid (RNA). In some embodiments, the RNA comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

Vectors are also provided, wherein the vectors comprise or contain a polynucleotide as disclosed herein (e.g., a polynucleotide that encodes an antibody or antigen-binding fragment that binds to IAV HA or to IAV NA and/or to IBV NA). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH1, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a therapy according to the present disclosure comprises delivering to a subject a single nucleic acid molecule that encodes (1) an anti-HA antibody or an antigen-binding fragment thereof, and (2) an anti-NA antibody or an antigen-binding fragment thereof.

In some embodiments, a therapy according to the present disclosure comprises delivering to a subject a first polynucleotide that encodes an anti-HA antibody or an antigen-binding fragment thereof, and a second polynucleotide that an anti-HA antibody or an antigen-binding fragment thereof.

In some embodiments, a therapy according to the present disclosure comprises delivering to a subject a (1) first polynucleotide that encodes a VH, a VH+CH1, or a heavy chain of an anti-HA antibody or an antigen-binding fragment thereof, (2) a second polynucleotide that encodes the cognate VL, VL+CL, or light chain of the anti-HA antibody or an antigen-binding fragment thereof, (3) a third polynucleotide that encodes a VH, a VH+CH1, or a heavy chain of an anti-NA antibody or an antigen-binding fragment thereof, and (4) a fourth polynucleotide that encodes the cognate VL, VL+CL, or light chain of the anti-NA antibody or an antigen-binding fragment thereof. In some embodiments, a polynucleotide (e.g., mRNA) is provided that encodes a heavy chain and a light chain of an antibody or antigen-binding fragment thereof. In some embodiments, a polynucleotide (e.g., mRNA) is provided that encodes two heavy chains and two light chains of an antibody or antigen-binding fragment thereof. See, e.g. Li, J Q., Zhang, Z R., Zhang, H Q. et al. Intranasal delivery of replicating mRNA encoding neutralizing antibody against SARS-CoV-2 infection in mice. Sig Transduct Target Ther 6, 369 (2021). https.//doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody or antigen-binding fragment thereof. In some embodiments, a method is provided that comprises delivering to a subject such a polynucleotide or replicon.

In a further aspect, the present disclosure also provides a host cell expressing an antibody or antigen-binding fragment according to the present disclosure, or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells, such as human B cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation), or a lack thereof, on the antibody or antigen-binding fragment that is not present in a native state of the antibody or antigen-binding fragment (or in a native state of a parent antibody from which the antibody or antigen binding fragment was engineered or derived). Such a PTM, or a lack thereof, may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a host cell can comprise one or more post-translational modification, or can include fewer post-translational modification(s), such that it is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise a presently disclosed anti-HA antibody or antigen-binding fragment, and a presently disclosed anti-NA antibody or antigen-binding fragment, or a polynucleotide encoding the same (e.g., the antibody or antigen-binding fragment, or components of these) in any combination, and can further comprise a pharmaceutically acceptable carrier, excipient, or diluent. Such compositions, as well as carriers, excipients, and diluents, are discussed in further detail herein.

In certain embodiments, a composition comprises one or more vector or polynucleotide that encodes an anti-HA antibody or antigen binding fragment, an anti-NA antibody or antigen-binding fragment, or both. In some embodiments, a composition comprises a first vector comprising a first plasmid, and a second vector comprising a second plasmid, wherein the first plasmid comprises a polynucleotide encoding a heavy chain, VH, or VH+CH1, and a second plasmid comprises a polynucleotide encoding the cognate light chain, VL, or VL+CL of the antibody or antigen-binding fragment thereof.

In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanoned Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

### Methods and Uses

Also provided herein are methods for using an antibody or antigen-binding fragment, polynucleotide, composition, or combination of the present disclosure to treat a subject that has, is believed to have, or is at risk for having an infection by influenza. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of an influenza infection (i.e., in a statistically significant manner). In certain embodiments, therapeutic or prophylactic/preventive benefit includes a reduced duration of hospitalization for treatment of an influenza infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, combination, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient alone.

When referring to an antibody combination, antibody composition, polynucleotide combination, or polynucleotide composition, a therapeutically effective amount refers to the combined amounts of active ingredients that results in a therapeutic effect, whether administered serially, sequentially, or simultaneously.

Accordingly, in certain embodiments, methods are provided for treating an influenza infection in a subject, wherein the methods comprise administering to the subject an effective amount of an antibody, antigen-binding fragment, polynucleotide, vector, host cell, combination, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with an influenza infection. These include, but are not limited to, age, occupation, general health, pre-existing health conditions, locale, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are can be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a subject treated according to the present disclosure has received a vaccine for influenza and the vaccine is determined to be ineffective, e.g., by post-vaccine infection or symptoms in the subject, by clinical diagnosis or scientific or regulatory consensus.

Prophylaxis of infection with influenza virus refers in particular to prophylactic settings, wherein the subject was not diagnosed with infection with influenza virus (either no diagnosis was performed or diagnosis results were negative) and/or the subject does not show or experience symptoms of infection with influenza virus. Prophylaxis of infection with influenza virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza virus is also particularly useful in subjects at greater risk acquiring influenza virus infection, e.g., due to increased exposure, for example subjects working or staying in public areas, in particular health care workers.

In certain embodiments, treatment is administered as peri-exposure or pre-exposure prophylaxis. In certain embodiments, treatment is administered as pos-exposure prophylaxis.

In therapeutic settings, in contrast, the subject is typically infected with influenza virus, diagnosed with influenza virus infection, and/or showing symptoms of influenza virus infection. Of note, the terms “treatment” and “therapy”/“therapeutic” of influenza virus infection can refer to (complete) cure as well as attenuation/reduction of influenza virus infection and/or related symptoms (e.g., attenuation/reduction of severity of infection and/or symptoms, number of symptoms, duration of infection and/or symptoms, or any combination thereof).

It will be understood that reference herein to a reduced number and/or severity of symptoms, which reduction results from administration of a presently disclosed therapy, describes a comparison with a reference subject who did not receive a disclosed therapy. A reference subject can be, for example, (i) the same subject during an earlier period of time (e.g., a prior influenza A virus season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof; and/or immunosuppressive condition or lack thereof: or (iii) a typical subject within a population (e.g., local, regional, or national, including of a same or similar age or age range and/or general state of health) during an influenza virus season. Prophylaxis can be determined by, for example, the failure to develop a diagnosed influenza infection and/or the lack of symptoms associated with influenza infection during a part of a full influenza season, or over a full influenza season.

In certain embodiments, the methods provided herein include administering a therapy according to the present disclosure to a subject at immediate risk of influenza infection. An immediate risk of influenza infection typically occurs during an influenza epidemic. Influenza viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza epidemic is high during November, December, January, February and March, while in the southern hemisphere the risk of an influenza epidemic is high during May, June, July, August and September.

In some embodiments, the subject has received, is receiving, or will receive an antiviral agent. In some embodiments, the antiviral agent comprises a neuraminidase inhibitor, an influenza polymerase inhibitor, or both. In certain embodiments, the antiviral agent comprises oseltamivir, lanamivir, peramivir, zanamivir, baloxavir, or any combination thereof.

Typical routes of administering the presently disclosed compositions include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding or polynucleotide in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat an influenza virus infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the anti-HA antibody or antigen-binding fragment, polynucleotide, vector, host cell, or composition and/or the anti-NA antibody or antigen-binding fragment, polynucleotide, vector, host cell, or composition to the subject at 2, 3, 4, 5, 6, 7, 8, 9, 10 times, or more.

In certain embodiments, a method comprises administering the anti-HA antibody or antigen-binding fragment or polynucleotide or vector or host cell or composition and/or the anti-NA antibody or antigen-binding fragment or polynucleotide or vector or host cell or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the anti-HA antibody or antigen-binding fragment or polynucleotide or vector or host cell or composition and/or the anti-NA antibody or antigen-binding fragment or polynucleotide or vector or host cell or composition at least one time prior to the subject being infected by influenza.

Combinations or compositions comprising an antibody, antigen-binding fragment (e.g., comprising an anti-HA antibody or antigen-binding fragment and an anti-NA antibody or antigen-binding fragment), polynucleotide, vector, or host cell of the present disclosure may also be administered simultaneously with, prior to, or after administration of one or more other therapeutic agents, such as, for example, a neuraminidase inhibitor, e.g., oseltamivir, zanamivir, peramivir, or laninamivir. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an anti-HA antibody or antigen-binding fragment and an anti-NA antibody or antigen-binding fragment can be administered to the patient together in a single oral dosage composition such as a tablet or capsule, or each agent administered in separate oral dosage formulations. Similarly, an anti-HA antibody or antigen-binding fragment and an anti-NA antibody or antigen-binding fragment can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, the anti-HA antibody or antigen-binding fragment or polynucleotide is administered simultaneous to, or within 1 minute, within 5 minutes, within 15 minutes, within 30 minutes, within 1 hour, within 6 hours, within 12 hours, within 24 hours, within 36 hours, within 2 to 5 days, within 1-2 weeks, within 1 month, within 2 months, within 3 months, within 4 months, within 5 months, or within 6 months, of the anti-NA antibody or antigen-binding fragment or polynucleotide.

In some embodiments, the anti-NA antibody or antigen-binding fragment or polynucleotide is administered simultaneous to, or within 1 minute, within 5 minutes, within 15 minutes, within 30 minutes, within 1 hour, within 6 hours, within 12 hours, within 24 hours, within 36 hours, within 2 to 5 days, within 1-2 weeks, within 1 month, within 2 months, within 3 months, within 4 months, within 5 months, or within 6 months, of the anti-HA antibody or antigen-binding fragment or polynucleotide.

In certain embodiments, the anti-HA antibody or antigen-binding fragment or polynucleotide and the anti-NA antibody or antigen-binding fragment or polynucleotide are formulated together. In certain embodiments, the anti-HA antibody or antigen-binding fragment or polynucleotide and the anti-NA antibody or antigen-binding fragment are formulated separately or polynucleotide.

In certain embodiments, the anti-HA antibody or antigen-binding fragment or polynucleotide and the anti-NA antibody or antigen-binding fragment or polynucleotide are administered in a sequence. In some embodiments, an antibody (or one or more nucleic acid, host cell, vector, or composition or combination) is administered to a subject who has previously received one or more anti-inflammatory agent and/or one or more antiviral agent. In some embodiments, the antiviral is a neuramidase inhibitor (NAI), such as, for example, oseltamivir, zanamivir, peramivir, or laninamivir. In some embodiments, one or more anti-inflammatory agent and/or one or more antiviral agent is administered to a subject who has previously received an antibody (or one or more nucleic acid, host cell, vector, or composition). In some embodiments, the antiviral is a neuramidase inhibitor (NAI), such as, for example, oseltamivir, zanamivir, peramivir, or laninamivir.

In a related aspect, uses of the presently disclosed antibodies, antigen-binding fragments, vectors, host cells, and compositions (e.g., in the diagnosis, prophylaxis, and/or treatment of an influenza infection, in the manufacture of a medicament for preventing or treating an influenza infection) are provided.

The present disclosure also provides the following non-limiting embodiments.

Embodiment 1. A combination comprising: (1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof, and (2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof.

Embodiment 2. A composition comprising: (1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof; and (2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof, and, optionally, a pharmaceutically acceptable carrier, excipient, or diluent.

Embodiment 3. The combination of Embodiment 1 or the composition of Embodiment 2, for use in a method of treating or preventing an influenza (IAV, IBV, or both) infection in a subject, wherein the method comprises administering an effective amount of the composition or combination, respectively, to the subject.

Embodiment 4. The combination of Embodiment 1 or the composition of Embodiment 2, for use in the manufacture of a medicament for treating or preventing an influenza (IAV, IBV, or both) infection in a subject.

Embodiment 5. An antibody, or an antigen-binding fragment thereof, that is capable of binding to an influenza A virus (IAV) hemagglutinin (HA) and neutralizing infection by the IAV, or a polynucleotide encoding the anti-HA antibody or antigen-binding fragment thereof, for use in a method of treating or preventing an influenza infection in a subject, wherein the method comprises administering an effective amount of the anti-HA antibody or antigen-binding fragment thereof to a subject who has received, is receiving, or will receive (1) an effective amount of an antibody, or an antigen-binding fragment thereof, that is capable of binding to a neuraminidase (NA) from: (i) an IAV, wherein the IAV comprises a Group 1 IAV, a Group 2 IAV, or both; and (ii) an influenza B virus (IBV), and is capable of neutralizing infection and/or inhibiting sialidase activity by the IAV and/or the IBV, or (2) a polynucleotide encoding the anti-NA antibody or antigen-binding fragment thereof.

Embodiment 6. An antibody, or an antigen-binding fragment thereof, that is capable of binding to a neuraminidase (NA) from: (i) an influenza A virus (IAV), wherein the IAV comprises a Group 1 IAV, a Group 2 IAV, or both; and (ii) an influenza B virus (IBV), and is capable of neutralizing infection and/or inhibiting sialidase activity by the IAV and/or the IBV, or a polynucleotide encoding the anti-NA antibody or antigen-binding fragment thereof, for use in a method of treating or preventing an influenza (IAV, IBV, or both) infection in a subject, wherein the method comprises administering an effective amount of the anti-NA antibody or antigen-binding fragment thereof to a subject who has received, is receiving, or will receive an an effective amount of (a) an antibody, or an antigen-binding fragment thereof, that is capable of binding to an IAV hemagglutinin (HA) and neutralizing infection by the IAV, or (b) a polynucleotide encoding the anti-HA antibody or antigen-binding fragment thereof.

Embodiment 7. A method of treating or preventing an influenza (IAV, IBV, or both) infection in a subject, the method comprising administering to the subject an effective amount of: (1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof; and (2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof.

Embodiment 8. A method of treating or preventing an influenza (IAV, IBV, or both) infection in a subject, the method comprising administering to the subject an effective amount of (1) an antibody, or an antigen-binding fragment thereof, that is capable of binding to an influenza A virus (IAV) hemagglutinin (HA) and neutralizing infection by the IAV, or (2) a polynucleotide encoding the anti-HA antibody or antigen-binding fragment thereof, wherein the subject has received, is receiving, or will receive (a) an antibody, or an antigen-binding fragment thereof, that is capable of binding to a neuraminidase (NA) from: (i) an IAV, wherein the IAV comprises a Group 1 IAV, a Group 2 IAV, or both; and (ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof.

Embodiment 9. A method of treating or preventing an influenza infection in a subject, the method comprising administering to the subject an effective amount of (1) an antibody, or an antigen-binding fragment thereof, that is capable of binding to a neuraminidase (NA) from: (i) an influenza A virus (IAV), wherein the IAV comprises a Group 1 IAV, a Group 2 IAV, or both; and (ii) an influenza B virus (IBV), and is capable of neutralizing infection and/or inhibiting sialidase activity by the IAV and/or the IBV, or (2) a polynucleotide encoding the anti-NA antibody or antigen-binding fragment thereof, wherein the subject has received, is receiving, or will receive (a) an antibody, or an antigen-binding fragment thereof, that is capable of binding to an IAV hemagglutinin (HA) and neutralizing infection by the IAV, or (b) a polynucleotide encoding the anti-HA antibody or antigen-binding fragment thereof.

Embodiment 10. The combination of Embodiment 1, the composition of Embodiment 2, the combination or composition for use of any one of Embodiments 3 and 4, the antibody or antigen-binding fragment or polynucleotide for use of any one of Embodiments 5 and 6, or the method of any one of Embodiments 7-9, wherein: (1) the anti-HA antibody or antigen-binding fragment comprises (1)(i) a VH comprising or consisting of an amino acid sequence having at least 80% (e.g., 80%, 85%, 9%, 91%, 92%, 93%, 94%, 95%, 96%, 97%, 98%, 99%, or more) identity to the amino acid sequence set forth in any one of SEQ ID NOs.:43, 2, 26, 28, 31, 34, 37, 14, 39, and 41, wherein sequence variation with reference to SEQ ID NO.: 43, 2, 26, 28, 31, 34, 37, 14, 39, and 41, respectively, is optionally comprised in one or more framework region and/or sequence variation comprises one or more substitution to a germline-encoded amino acid; and/or (1)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 44, 8, and 20, wherein sequence variation with respect to SEQ ID NO.:44, 8, and 20, respectively, is optionally comprised in one or more framework regions and/or sequence variation comprises one or more substitution to a germline-encoded amino acid; and/or (2) the anti-NA antibody or antigen-binding fragment comprises (2)(i) a VH comprising or consisting of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.:241, 48, 60, 72, 171, 84, 96, 108, 120, 132, 229, 144, 156, 168, 180, 192, 204, 245, 249, 258, and 261, wherein sequence variation with reference to SEQ ID NO.: 241, 48, 60, 72, 171, 84, 96, 108, 120, 132, 229, 144, 156, 168, 180, 192, 204, 245, 249, 258, 261, respectively, is optionally comprised in one or more framework region and/or sequence variation comprises one or more substitution to a germline-encoded amino acid; and/or (2)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.:243, 54, 66, 78, 90, 102, 114, 126, 138, 150, 162, 174, 186, 198, 220, 223, 226, 232, 235, 238, 210, 247, 251, 259, and 263, wherein sequence variation with respect to SEQ ID NO.: 243, 54, 66, 78, 90, 102, 114, 126, 138, 150, 162, 174, 186, 198, 220, 223, 226, 232, 235, 238, 210, 247, 251, 259, and 263, respectively, is optionally comprised in one or more framework regions and/or sequence variation comprises one or more substitution to a germline-encoded amino acid wherein, preferably, the anti-HA antibody or antigen-binding fragment comprises a VH and a VL comprising or consisting of an amino acid sequence having at least 80% identity to the amino acid sequences set forth in SEQ ID NOs.: 43 and 44, respectively, and the anti-NA antibody or antigen-binding fragment comprises a VH and a VL comprising or consisting of an amino acid sequence having at least 80% identity to the amino acid sequences set forth in SEQ ID NOs.: (4)(i) 241 and 243; and/or (3) the anti-HA antibody or antigen-binding fragment comprises a VH and a VL comprising the HCDRS and the LCDRs, respectively, of the VH and VL amino acid sequences set forth in SEQ ID NOs.: (3)(i) 43 and 44, respectively; (3)(ii) 26 and 8, respectively; (3)(iii) 2 and 8, respectively; (3)(iv) 31 and 8, respectively; (3)(v) 34 and 8, respectively; (3)(vi) 37 and 8, respectively; (3)(vii) 14 and 20, respectively; (3)(viii) 39 and 20, respectively; (3)(ix) 41 and 20, respectively; or (1)(x) 28 and 8, respectively, wherein, optionally, the HCDRs and the LCDRs are according to IMGT numbering; and/or (4) the anti-NA antibody or antigen-binding fragment comprises a VH and a VL comprising the HCDRS and the LCDRs, respectively, of the VH and VL amino acid sequences set forth in SEQ ID NOs.: (4)(i) 241 and 243, respectively; (4)(ii) 60 and 66, respectively; (4)(iii) 72 and 78 or 220 or 223, respectively; (4)(vi) 72 and 226, respectively; (4)(vii) 217 and 78, respectively; (4)(viii) 217 and 220, respectively; (4)(ix) 217 and 223, respectively; (4)(x) 217 and 226, respectively; (4)(xi) 84 and 90, respectively; (4)(xii) 96 and 102, respectively; (4)(xiii) 108 and 114, respectively; (4)(xiv) 120 and 126, respectively; (4)(xv) 132 and 138, respectively; (4)(xvi) 132 and 232, respectively; (4)(xvii) 132 and 235, respectively; (4)(xviii) 132 and 238, respectively; (4)(xix) 229 and 138, respectively; (4)(xx) 229 and 232, respectively; (4)(xxi) 229 and 235, respectively; (4)(xxii) 229 and 238, respectively; (4)(xxiii) 144 and 150, respectively; (4)(xxiv) 156 and 162, respectively; (4)(xxv) 168 and 174, respectively; (4)(xxvi) 180 and 186, respectively; (4)(xxvii) 192 and 198, respectively; (4)(xxviii) 204 and 210, respectively; (4)(xxix) 48 and 54, respectively; (4)(xxx) 245 and 247, respectively; (4)(xxxi) 249 and 251, respectively; (4)(xxxii) 258 and 259, respectively; or (4)(xxxi) 261 and 263, respectively, wherein, optionally, the HCDRs and the LCDRs are according to IMGT numbering, wherein, preferably, the anti-HA antibody or antigen-binding fragment comprises a VH and a VL comprising the HCDRS and the LCDRs, respectively, of the VH and VL amino acid sequences set forth in SEQ ID NOs.: 43 and 44, respectively, and the anti-NA antibody or antigen-binding fragment comprises a VH and a VL comprising the HCDRS and the LCDRs, respectively, of the VH and VL amino acid sequences set forth in SEQ ID NOs.: (4)(i) 241 and 243.

Embodiment 11. The combination of Embodiment 1 or 10, the composition of Embodiment 2 or 10, the combination or composition for use of any one of Embodiments 3, 4, and 10, the antibody or antigen-binding fragment or polynucleotide for use of any one of Embodiments 5, 6, and 10, or the method of any one of Embodiments 7-10, wherein: (1) the VH and the VL of the anti-HA antibody or antigen-binding fragment comprise or consist of the amino acid sequences according to SEQ ID NOs.: (1)(i) 43 and 44, respectively; (1)(ii) 26 and 8, respectively; (1)(iii) 2 and 8, respectively; (1)(iv) 31 and 8, respectively; (1)(v) 34 and 8, respectively; (1)(vi) 37 and 8, respectively; (1)(vii) 14 and 20, respectively; (1)(viii) 39 and 20, respectively; (1)(ix) 41 and 20, respectively; or (1)(x) 28 and 8, respectively; and/or (2) the VH and the VL of the anti-NA antibody or antigen-binding fragment comprise or consist of the amino acid sequences according to SEQ ID NOs.: (2)(i) 241 and 243, respectively; (2)(ii) 60 and 66, respectively; (2)(iii) 72 and 78 or 220 or 223, respectively; (2)(vi) 72 and 226, respectively; (2)(vii) 217 and 78, respectively; (2)(viii) 217 and 220, respectively; (2)(ix) 217 and 223, respectively; (2)(x) 217 and 226, respectively; (2)(xi) 84 and 90, respectively; (2)(xii) 96 and 102, respectively; (2)(xiii) 108 and 114, respectively; (2)(xiv) 120 and 126, respectively; (2)(xv) 132 and 138, respectively; (2)(xvi) 132 and 232, respectively; (2)(xvii) 132 and 235, respectively; (2)(xviii) 132 and 238, respectively; (2)(xix) 229 and 138, respectively; (2)(xx) 229 and 232, respectively; (2)(xxi) 229 and 235, respectively; (2)(xxii) 229 and 238, respectively; (2)(xxiii) 144 and 150, respectively; (2)(xxiv) 156 and 162, respectively; (2)(xxv) 168 and 174, respectively; (2)(xxvi) 180 and 186, respectively; (2)(xxvii) 192 and 198, respectively; (2)(xxviii) 204 and 210, respectively; (2)(xxix) 48 and 54, respectively; (2)(xxx) 245 and 247, respectively; (2)(xxxi) 249 and 251, respectively; (2)(xxxii) 258 and 259, respectively; or (2)(xxxiii) 261 and 263, respectively, wherein, preferably, the VH and the VL of the anti-HA antibody or antigen-binding fragment comprise or consist of the amino acid sequences according to SEQ ID NOs.: 43 and 44, respectively the VH and the VL of the anti-NA antibody or antigen-binding fragment comprise or consist of the amino acid sequences according to SEQ ID NOs.: 241 and 243, respectively.

Embodiment 12. The combination of Embodiment 1, 10, or 11, the composition of Embodiment 2, 10, or 11, the combination or composition for use of any one of Embodiments 3, 4, 10, and 11, the antibody or antigen-binding fragment or polynucleotide for use of any one of Embodiments 5, 6, 10, and 11, or the method of any one of Embodiments 7-11, wherein: (1) the VH and the VL of the anti-HA antibody or antigen-binding fragment comprise or consist of the amino acid sequences according to SEQ ID NOs.: (1)(i) 43 and 44, respectively, respectively; (1)(ii) 26 and 8, respectively; (1)(iii) 28 and 8, respectively; (1)(iv) 31 and 8, respectively; (1)(v) 34 and 8, respectively; (1)(vi) 37 and 8, respectively; (1)(vii) 14 and 20, respectively; (1)(viii) 39 and 20, respectively; (1)(ix) 41 and 20, respectively; or (1)(x) 2 and 8, respectively; and/or (2) the VH and the VL of the anti-NA antibody or antigen-binding fragment comprise or consist of the amino acid sequences according to SEQ ID NOs.: (2)(i) 241 and 243, respectively, respectively; (2)(ii) 72 and 226, respectively; (2)(iii) 217 and 78, respectively: (2)(iv) 217 and 220, respectively; (2)(v) 132 and 138, respectively: (2)(vi) 132 and 232, respectively; (2)(vii) 132 and 235, respectively; (2)(viii) 132 and 238, respectively; (2)(ix) 229 and 138, respectively; (2)(x) 229 and 232, respectively; (2)(xi) 229 and 235, respectively; (2)(xii) 229 and 238, respectively: (2)(xiii) 217 and 223, respectively; (2)(xiv) 217 and 226, respectively; (2)(xv) 72 and 78 or 220 or 223; (2)(xvi) 245 and 247, respectively; or (2)(xvii) 249 and 251, respectively, wherein, preferably, the VH and the VL of the anti-HA antibody or antigen-binding fragment comprise or consist of the amino acid sequences according to SEQ ID NOs.: 43 and 44, respectively the VH and the VL of the anti-NA antibody or antigen-binding fragment comprise or consist of the amino acid sequences according to SEQ ID NOs.:241 and 243, respectively.

Embodiment 13. The combination of any one of Embodiments 1 and 10-12, the composition of any one of Embodiments 2 and 10-12, the combination or composition for use of Embodiment 3, 4, 10, 11, or 12, the antibody or antigen-binding fragment or polynucleotide for use of Embodiment 5, 6, 10, 11, or 12, or the method of any one of Embodiments 7-12, wherein: (1) the anti-HA antibody or antigen-binding fragment comprises a heavy chain variable domain (VH) comprising a complementarity determining region (CDR)H1, a CDRH2, and a CDRH3, and a light chain variable domain (VL) comprising a CDRL1, a CDRL2, and a CDRL3, wherein the CDRs are optionally according to the IMGT numbering system, and wherein: (1)(i) the CDRH1 comprises or consists of the amino acid sequence of any one of SEQ ID NOs.: 274, 3, 32, and 15, or a functional variant thereof comprising one, two, or three acid substitutions, one or more of which substitutions is optionally a conservative substitution and/or is a substitution to a germline-encoded amino acid; and/or (1)(ii) the CDRH2 comprises or consists of the amino acid sequence of any one of SEQ ID NOs.: 275, 4, 29, 35, 16, and 42, or a functional variant thereof comprising one, two, or three amino acid substitutions, one or more of which substitutions is optionally a conservative substitution and/or is a substitution to a germline-encoded amino acid; and/or (1)(iii) the CDRH3 comprises or consists of the amino acid sequence of any one of SEQ ID NOs.: 276, 5, and 17, or a functional variant thereof comprising one, two, or three amino acid substitutions, one or more of which substitutions is optionally a conservative substitution and/or is a substitution to a germline-encoded amino acid; and/or (1)(iv) the CDRL1 comprises or consists of the amino acid sequence of any one of SEQ ID NOs.:277, 9, and 21, or a functional variant thereof comprising one, two, or three amino acid substitutions, one or more of which substitutions is optionally a conservative substitution and/or is a substitution to a germline-encoded amino acid; and/or (1)(v) the CDRL2 optionally comprises or consists of the amino acid sequence of any one of SEQ ID NOs.: 278, 10, and 22, or a functional variant thereof comprising one, two, or three amino acid substitutions, one or more of which substitutions is optionally a conservative substitution and/or is a substitution to a germline-encoded amino acid; and/or (1)(vi) the CDRL3 comprises or consists of the amino acid sequence of any one of SEQ ID NOs.: 279, 11, and 23, or a functional variant thereof comprising having one, two, or three amino acid substitutions, one or more of which substitutions is optionally a conservative substitution and/or is a substitution to a germline-encoded amino acid; and/or (2) the anti-NA antibody or antigen-binding fragment comprises a VH comprising a CDRH1, a CDRH2, and a CDRH3, and a VL comprising a CDRL1, a CDRL2, and a CDRL3, wherein: (2)(i) optionally, the CDRH1 comprises or consists of the amino acid sequence set forth in any one of SEQ ID NOs.: 193, 49, 61, 73, 85, 97, 109, 121, 133, 145, 157, 169, 181, 205, and 264, or a functional variant thereof comprising one, two, or three acid substitutions, one or more of which substitutions is optionally a conservative substitution and/or is a substitution to a germline-encoded amino acid; (2)(ii) optionally, the CDRH2 comprises or consists of the amino acid sequence set forth in any one of SEQ ID NOs.: 194, 50, 62, 74, 86, 98, 110, 122, 134, 146, 158, 170, 182, 206, and 265, or a functional variant thereof comprising one, two, or three amino acid substitutions, one or more of which substitutions is optionally a conservative substitution and/or is a substitution to a germline-encoded amino acid; (2)(iii) the CDRH3 comprises or consists of the amino acid sequence set forth in any one of SEQ ID NOs.: 195, 51, 63, 75, 218, 87, 99, 111, 123, 135, 230, 147, 159, 171, 183, 207, and 266, or a functional variant thereof comprising one, two, or three amino acid substitutions, one or more of which substitutions is optionally a conservative substitution and/or is a substitution to a germline-encoded amino acid; (2)(iv) optionally, the CDRL1 comprises or consists of the amino acid sequence set forth in any one of SEQ ID NOs.: 199, 55, 67, 79, 91, 103, 115, 127, 139, 151, 163, 175, 187, 211, and 267, or a functional variant thereof comprising one, two, or three amino acid substitutions, one or more of which substitutions is optionally a conservative substitution and/or is a substitution to a germline-encoded amino acid; (2)(v) optionally, the CDRL2 comprises or consists of the amino acid sequence set forth in any one of SEQ ID NOs.: 200, 56, 68, 80, 92, 104, 116, 128, 140, 152, 164, 176, 188, 212, and 268, or a functional variant thereof comprising one, two, or three amino acid substitutions, one or more of which substitutions is optionally a conservative substitution and/or is a substitution to a germline-encoded amino acid; and/or (2)(vi) optionally, the CDRL3 comprises or consists of the amino acid sequence set forth in any one of SEQ ID NOs.: 201, 57, 69, 81, 221, 224, 227, 93, 105, 117, 129, 141, 233, 239, 153, 165, 177, 189, 236, 213, and 269, or a functional variant thereof comprising having one, two, or three amino acid substitutions, one or more of which substitutions is optionally a conservative substitution and/or is a substitution to a germline-encoded amino acid.

Embodiment 14. The combination of any one of Embodiments 1 and 10-13, the composition of any one of Embodiments 2 and 10-13, the combination or composition for use of Embodiment 3, 4, 10, 11, 12, or 13, the antibody or antigen-binding fragment or polynucleotide for use of Embodiment 5, 6, 10, 11, 12, or 13, or the method of any one of Embodiments 7-13, wherein: (1) the anti-HA antibody or antigen-binding fragment comprises CDRH1, CDRH2, CDRH3, CDRL1, CDRL2, and CDRL3 amino acid sequences of SEQ ID NOs.: (1)(i) 274-279, respectively; (1)(ii) 3, 29, 5 and 9-11, respectively; (1)(iii) 32, 4, 5 and 9-11, respectively; (1)(iv) 3, 35, 5 and 9-11, respectively; (1)(v) 32, 35, 5, and 9-11, respectively; (1)(vi) 15-17 and 21-23, respectively; (1)(vii) 15, 42, 17 and 21-23, respectively; or (1)(viii) 3-5 and 9-11, respectively; and/or (2) the anti-NA antibody or antigen-binding fragment comprises CDRH1, CDRH2, CDRH3, CDRL1, CDRL2, and CDRL3 amino acid sequences of SEQ ID NOs.: (2)(i) 193-195 and 199-201, respectively; (2)(ii) 61-63 and 67-69, respectively; (2)(iii) 73-75 and 79-81, respectively; (2)(iv) 73, 74, 218, and 79-81, respectively; (2)(v) 73-75, 79, 80, and 221, respectively; (2)(vi) 73-75, 79, 80, and 224, respectively; (2)(vii) 73-75, 79, 80, and 227, respectively; (2)(viii) 73, 74, 218, 79, 80, and 221, respectively; (2)(ix) 73, 74, 218, 79, 80, and 224, respectively; (2)(x) 73, 74, 218, 79, 80, and 227, respectively; (2)(xi) 85-87 and 91-93, respectively; (2)(xii) 97-99 and 103-105, respectively; (2)(xiii) 109-111 and 115-117, respectively; (2)(xiv) 121-123 and 127-129, respectively; (2)(xv) 133-135 and 139-141, respectively; (2)(xvi) 133, 134, 230 and 139-141, respectively; (2)(xvii) 133-135, 139, 141, and 233, respectively; (2)(xviii) 133-135, 139, 141, and 236, respectively; (2)(xix) 133-135, 139, 141, and 239, respectively; (2)(xx) 133, 134, 184, 139, 141, and 233, respectively; (2)(xxi) 133, 134, 230, 139, 141, and 236, respectively; (2)(xxii) 133, 134, 230, 139, 141, and 239, respectively; (2)(xxiii) 145-147 and 151-153, respectively; (2)(xxiv) 157-159 and 163-165, respectively; (2)(xxv) 169-171 and 175-177, respectively; (2)(xxvi) 181-183 and 187-189, respectively; (2)(xxvii) 49-51 and 55-57, respectively; (2)(xxviii) 205-207 and 211-213, respectively; or (2)(xxix) 264-266 and 267-269, respectively.

Embodiment 15. The combination of Embodiment 13 or 14, the composition of Embodiment 13 or 14, the combination or composition for use of Embodiment 13 or 14, the antibody or antigen-binding fragment or polynucleotide for use of Embodiment 13 or 14, or the method of Embodiment 13 or 14, wherein: (1) the anti-HA antibody or antigen-binding fragment comprises CDRH1, CDRH2, CDRH3, CDRL1, CDRL2, and CDRL3 amino acid sequences of SEQ ID NOs.: (1)(i) 274-279, respectively; (1)(ii) 3, 29, 5 and 9-11, respectively; (1)(iii) 32, 4, 5 and 9-11, respectively; (1)(iv) 3, 35, 5 and 9-11, respectively; (1)(v) 32, 35, 5, and 9-11, respectively; (1)(vi) 15-17 and 21-23, respectively; (1)(vii) 15, 42, 17 and 21-23, respectively; or (1)(viii) 3-5 and 9-11, respectively; and/or (2) the anti-NA antibody or antigen-binding fragment comprises CDRH1, CDRH2, CDRH3, CDRL1, CDRL2, and CDRL3 amino acid sequences of SEQ ID NOs.: (2)(i) 193-195 and 199-201, respectively; (2)(ii) 73, 74, 218, and 79-81, respectively; (2)(iii) 73-75, 79, 80, and 221, respectively; (2)(iv) 73-75, 79, 80, and 224, respectively; (2)(v) 73-75, 79, 80, and 227, respectively; (2)(vi) 73, 74, 218, 79, 80, and 221, respectively; (2)(vii) 73, 74, 218, 79, 80, and 224, respectively; (2)(viii) 73, 74, 218, 79, 80, and 227, respectively; (2)(ix) 133-135 and 139-141, respectively; (2)(x) 133, 134, 230 and 139-141, respectively; (2)(xi) 133-135, 139, 141, and 233, respectively; (2)(xii) 133-135, 139, 141, and 236, respectively; (2)(xiii) 133-135, 139, 141, and 239, respectively; (2)(xiv) 133, 134, 184, 139, 141, and 233, respectively; (2)(xv) 133, 134, 184, 139, 141, and 236, respectively; (2)(xvi) 133, 134, 184, 139, 141, and 239, respectively; (2)(xvii) 264-266 and 267-296, respectively; or (2)(xviii) 73-75 and 79-81, respectively.

Embodiment 16. The combination of any one of Embodiments 1 and 10-15, the composition of one of Embodiments 2 and 10-15, the combination or composition for use of any one of Embodiments 3, 4, and 10-15, the antibody or antigen-binding fragment or polynucleotide for use of any one of Embodiments 5, 6, and 10-15, or the method of any one of Embodiments 7-15, wherein: (i) the Group 1 IAV NA comprises a N1, a N4, a N5, and/or a N8; and/or (ii) the Group 2 IAV NA comprises a N2, a N3, a N6, a N7, and/or a N9.

Embodiment 17. The combination of Embodiment 16, the composition of Embodiment 16, the combination or composition for use Embodiment 16, the antibody or antigen-binding fragment or polynucleotide for use Embodiment 16, or the method of Embodiment 16, wherein: (i) the N1 is a N1 from any one or more of: A/California/07/2009, A/California/07/2009 I23R/H275Y, A/Swine/Jiangsu/J004/2018, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/G4/SW/Shangdong/1207/2016, A/G4/SW/Henan/SN13/2018, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011; (v) the N2 is a N2 from any one or more of: A/Washington/01/2007, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011; A/HongKong/2671/2019, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, and A/Brisbane/10/2007; (vi) the N3 is from A/Canada/rv504/2004; (v) the N6 is from A/swine/Ontario/01911/1/99; (vi) the N7 is from A/Netherlands/078/03; and/or (vii) the N9 is a N9 from any one or more of: A/Anhui/2013 and A/Hong Kong/56/2015.

Embodiment 18. The combination of any one of Embodiments 1 and 10-17, the composition of any one of Embodiments 2 and 10-17, the combination or composition for use of any one of Embodiments 3, 4, and 10-17, the antibody or antigen-binding fragment or polynucleotide for use of any one of Embodiments 5, 6, and 10-17, or the method of any one of Embodiments 7-17, wherein the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Texas/06/2011 (Yamagata); B/Perth/211/2011; B/HongKong/05/1972; B/Phuket/3073/2013; B/Harbin/7/1994 (Victoria); and B/Washington/02/2019 (Victoria).

Embodiment 19. The combination of any one of Embodiments 1 and 10-18, the composition of any one of Embodiments 2 and 10-18, the combination or composition for use of any one of Embodiments 3, 4, and 10-18, the antibody or antigen-binding fragment or polynucleotide for use of any one of Embodiments 5, 6, and 10-18, or the method of any one of Embodiments 7-18, wherein the NA is a N1, a N2, and/or a N9.

Embodiment 20. The combination of any one of Embodiments 1 and 10-19, the composition of any one of Embodiments 2 and 10-19, the combination or composition for use of any one of Embodiments 3, 4, and 10-19, the antibody or antigen-binding fragment or polynucleotide for use of any one of Embodiments 5, 6, and 10-19, or the method of any one of Embodiments 7-19, wherein the anti-NA antibody or antigen-binding fragment is capable of binding to: (1) (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118; and/or (2) (i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering); and/or (3) an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, I222, E227, H274, E277, D293, E425; and/or (4) an IBV NA epitope that comprises: (i) any one or more of the following amino acids: R116, D149, E226, R292, and R374; or (ii) the amino acids R116, D149, E226, R292, and R374.

Embodiment 21. The combination of Embodiment 20, the composition of Embodiment 20, the combination or composition for use of Embodiment 20, the antibody or antigen-binding fragment or polynucleotide for use of Embodiment 20, or the method of Embodiment 20, wherein the anti-NA antibody or antigen binding fragment is capable of binding to: (1) an epitope that further comprises any one or more of the following NA amino acids (N2 numbering): E344, E227, S247, and D198; and/or (2) a NA comprising a S245N amino acid mutation and/or a E221D amino acid mutation.

Embodiment 22. The combination of any one of Embodiments 1 and 10-21, the composition of any one of Embodiments 2 and 10-21, the combination or composition for use of any one of Embodiments 3, 4, and 10-21, the antibody or antigen-binding fragment or polynucleotide for use of any one of Embodiments 5, 6, and 10-21, or the method of any one of Embodiments 7-21, wherein the anti-NA antibody or antigen-binding fragment is capable of binding to a NA comprising a S245N amino acid mutation and/or a E221D amino acid mutation.

Embodiment 23. The combination of any one of Embodiments 1 and 10-22, the composition of any one of Embodiments 2 and 10-22, the combination or composition for use of any one of Embodiments 3, 4, and 10-22, the antibody or antigen-binding fragment or polynucleotide for use of any one of Embodiments 5, 6, and 10-22, or the method of any one of Embodiments 7-22, wherein: (i) the Group 1 IAV NA comprises a H1N1 and/or a H5N1; (ii) the Group 2 IAV NA comprises a H3N2 and/or a H7N9; and/or (iii) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

Embodiment 24. The combination of any one of Embodiments 1 and 10-23, the composition of any one of Embodiments 2 and 10-23, the combination or composition for use of any one of Embodiments 3, 4, and 10-23, the antibody or antigen-binding fragment or polynucleotide for use of any one of Embodiments 5, 6, and 10-23, or the method of any one of Embodiments 7-23, wherein the anti-HA antibody or antigen-binding fragment is capable of binding to any one or more of the following IAV subtypes: H1, H2, H3, H4, H5, H8, H9, H10, H11, H12, H13, H14, H15, H17, and H18.

Embodiment 25. The combination of any one of Embodiments 1 and 10-24, the composition of any one of Embodiments 2 and 10-24, the combination or composition for use of any one of Embodiments 3, 4, and 10-24, the antibody or antigen-binding fragment or polynucleotide for use of any one of Embodiments 5, 6, and 10-24, or the method of any one of Embodiments 7-24, wherein the anti-HA antibody or antigen-binding fragment is capable of neutralizing infection by: (i) a H1N1 IAV, wherein, optionally, the H1N1 IAV comprises any one or more of: A/California/07/2009, A/PR/8/34, and A/Solomon Islands/3/06; and (ii) a H3N2 IAV, wherein, optionally, the H3N2 IAV comprises any one or more of: A/Aichi/2/68, A/Brisbane/10/07, and A/Hong Kong/68 (i) a Group 1 IAV, wherein, optionally, the Group 1 IAV comprises or is a H5 IAV, wherein, further optionally, the H5 IAV comprises or is H5/VN/l 1/94 pp; and (ii) a Group 2 IAV, wherein, optionally, the Group 2 IAV comprises or is a H7 IAV, wherein, further optionally, the H7 IAV comprises or is H7/IT/99 pp, wherein, optionally, neutralization of infection is as determined using a virus pseudotyped with the IAV.

Embodiment 26. The combination of any one of Embodiments 1 and 10-25, the composition of any one of Embodiments 2 and 10-25, the combination or composition for use of any one of Embodiments 3, 4, and 10-25, the antibody or antigen-binding fragment or polynucleotide for use of any one of Embodiments 5, 6, and 10-25, or the method of any one of Embodiments 7-25, wherein the anti-HA antibody or antigen-binding fragment is capable of binding to one or more of (i)-(iv): (i) a H1 HA, which optionally comprises any one or more of: A/England/195/2009; A/Brisbane/59/2007; A/Solomon Islands/3/2006; A/New Caledonia/20/99; A/Texas/36/1991; A/Taiwan/01/1986; A/New Jersey/8/1976; A/Albany/I2/1951; A/Fort Monmouth/1/1947; A/New York/1/1918; A/Puerto Rico/8/34; and A/California/07/2009; (ii) a H2 HA, optionally comprising A/Japan/305/1957; (iii) a H5 HA, optionally comprising A/Vietnam/1194/2004; and (iv) a H9 HA, optionally comprising A/Hong Kong/1073/99.

Embodiment 27. The combination of any one of Embodiments 1 and 10-26, the composition of any one of Embodiments 2 and 10-26, the combination or composition for use of any one of Embodiments 3, 4, and 10-26, the antibody or antigen-binding fragment or polynucleotide for use of any one of Embodiments 5, 6, and 10-26, or the method of any one of Embodiments 7-26, wherein the anti-HA antibody or antigen-binding fragment, the anti-NA antibody or antigen-binding fragment, or both, is capable of activating a human FcγRIIIa (optionally a F158 allele).

Embodiment 28. The combination of Embodiment 27, the composition of of Embodiment 27, the combination or composition for use of Embodiment 27, the antibody or antigen-binding fragment or polynucleotide for use of Embodiment 27, or the method of Embodiment 27, wherein activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV and/or a IBV.

Embodiment 29. The combination of Embodiment 27 or 28, the composition of Embodiment 27 or 28, the combination or composition for use of Embodiment 27 or 28, the antibody or antigen-binding fragment or polynucleotide for use of Embodiment 27 or 28, or the method of Embodiment 27 or 28, wherein activation is as determined following an incubation (optionally, for about 23 hours) of the antibody or antigen-binding fragment with the target cell infected with a H1N1 and/or a H3N2 IAV, wherein, optionally, the H1N1 IAV is A/PR8/34 and, further optionally, comprises a multiplicity of infection (MOI) of 6 and/or wherein the H3N2 IAV is A/Aichi/68 and, further optionally, comprises a MOI of 18.

Embodiment 30. The combination of any one of Embodiments 1 and 10-29, the composition of any one of Embodiments 2 and 10-29, the combination or composition for use of any one of Embodiments 3, 4, and 10-29, the antibody or antigen-binding fragment or polynucleotide for use of any one of Embodiments 5, 6, and 10-29, or the method of any one of Embodiments 7-29, wherein the IAV and/or the IBV is antiviral-resistant, wherein, optionally, the antiviral is oseltamivir.

Embodiment 31. The combination of any one of Embodiments 1 and 10-30, the composition of any one of Embodiments 2 and 10-30, the combination or composition for use of any one of Embodiments 3, 4, and 10-30, the antibody or antigen-binding fragment or polynucleotide for use of any one of Embodiments 5, 6, and 10-30, or the method of any one of Embodiments 7-, wherein the IAV comprises a N1 NA that comprises the amino acid mutation(s): H275Y; E119D+H275Y; S247N+H275Y; 1222V; and/or N294S wherein, optionally, the IAV comprises CA09 or A/Aichi.

Embodiment 32. The combination of any one of Embodiments 1 and 10-31, the composition of any one of Embodiments 2 and 10-31, the combination or composition for use of any one of Embodiments 3, 4, and 10-31, the antibody or antigen-binding fragment or polynucleotide for use of any one of Embodiments 5, 6, and 10-32, or the method of any one of Embodiments 7-31, wherein the IAV comprises a N2 NA that comprises the amino acid mutation(s) E119V, Q136K, and/or R292K.

Embodiment 33. The combination of any one of Embodiments 1 and 10-32, the composition of any one of Embodiments 2 and 10-32, the combination or composition for use of any one of Embodiments 3, 4, and 10-32, the antibody or antigen-binding fragment or polynucleotide for use of any one of Embodiments 5, 6, and 10-32, or the method of any one of Embodiments 7-32, wherein the anti-HA antibody or antigen-binding fragment, the anti-NA antibody or antigen binding fragment, or both, is/are capable of preventing weight loss in a subject infected by the IAV and/or IBV, optionally for (i) up to 15 days, or (ii) more than 15 days, following administration of an effective amount of the antibody or antigen-binding fragment.

Embodiment 34. The combination of any one of Embodiments 1 and 10-33, the composition of any one of Embodiments 2 and 10-33, the combination or composition for use of any one of Embodiments 3, 4, and 10-33, the antibody or antigen-binding fragment or polynucleotide for use of any one of Embodiments 5, 6, and 10-33, or the method of any one of Embodiments 7-33, wherein the anti-HA antibody or antigen-binding fragment, the anti-NA antibody or antigen binding fragment, or both, is/are capable of preventing a loss in body weight of greater than 25%, 20%, 15%, 10%, or 5% in a subject having an IAV infection and/or an IBV infection, as determined by reference to the subject's body weight just prior to the IAV and/or IBV infection.

Embodiment 35. The combination of any one of Embodiments 1 and 10-34, the composition of any one of Embodiments 2 and 10-34, the combination or composition for use of any one of Embodiments 3, 4, and 10-34, the antibody or antigen-binding fragment or polynucleotide for use of any one of Embodiments 5, 6, and 10-34, or the method of any one of Embodiments 7-34, wherein the anti-HA antibody or antigen-binding fragment, the anti-NA antibody or antigen binding fragment, or both, is/are capable extending survival of a subject having an IAV infection and/or an IBV infection.

Embodiment 36. The combination of any one of Embodiments 1 and 10-35, the composition of any one of Embodiments 2 and 10-35, the combination or composition for use of any one of Embodiments 3, 4, and 10-35, the antibody or antigen-binding fragment or polynucleotide for use of any one of Embodiments 5, 6, and 10-35, or the method of any one of Embodiments 7-35, wherein the anti-HA antibody or antigen-binding fragment, the anti-NA antibody or antigen binding fragment, or both, is/are a IgG, IgA, IgM, IgE, or IgD isotype.

Embodiment 37. The combination of any one of Embodiments 1 and 10-36, the composition of any one of Embodiments 2 and 10-36, the combination or composition for use of any one of Embodiments 3, 4, and 10-36, the antibody or antigen-binding fragment or polynucleotide for use of any one of Embodiments 5, 6, and 10-36, or the method of any one of Embodiments 7-36, wherein the anti-HA antibody or antigen-binding fragment, the anti-NA antibody or antigen binding fragment, or both, is/are an IgG isotype selected from IgG1, IgG2, IgG3, and IgG4.

Embodiment 38. The combination of any one of Embodiments 1 and 10-37, the composition of any one of Embodiments 2 and 10-37, the combination or composition for use of any one of Embodiments 3, 4, and 10-37, the antibody or antigen-binding fragment or polynucleotide for use of any one of Embodiments 5, 6, and 10-37, or the method of any one of Embodiments 7-37, wherein the anti-HA antibody or antigen-binding fragment, the anti-NA antibody or antigen binding fragment, or both, comprises or comprise a human antibody, a monoclonal antibody, a purified antibody, a single chain antibody, a Fab, a Fab′, a F(ab′)2, or Fv.

Embodiment 39. The combination of any one of Embodiments 1 and 10-38, the composition of any one of Embodiments 2 and 10-38, the combination or composition for use of any one of Embodiments 3, 4, and 10-38, the antibody or antigen-binding fragment or polynucleotide for use of any one of Embodiments 5, 6, and 10-38, or the method of any one of Embodiments 7-38, wherein the anti-HA antibody or antigen-binding fragment, the anti-NA antibody or antigen binding fragment, or both, is a multi-specific antibody or antigen-binding fragment, wherein, optionally, the multi-specific antibody or antigen-binding fragment comprises a bispecific antibody or antigen-binding fragment.

Embodiment 40. The combination of any one of Embodiments 1 and 10-39, the composition of any one of Embodiments 2 and 10-39, the combination or composition for use of any one of Embodiments 3, 4, and 10-39, the antibody or antigen-binding fragment or polynucleotide for use of any one of Embodiments 5, 6, and 10-39, or the method of any one of Embodiments 7-39, wherein the anti-HA antibody or antigen-binding fragment, the anti-NA antibody or antigen binding fragment, or both, comprises a Fc polypeptide or a fragment thereof.

Embodiment 41. The combination of Embodiment 40, the composition of Embodiment 40, the combination or composition for use of Embodiment 40, the antibody or antigen-binding fragment or polynucleotide for use of Embodiment 40, or the method of Embodiment 40, wherein the Fc polypeptide or fragment thereof comprises: (i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or (ii) a mutation that increases binding affinity to a human FcγR (e.g., as measured using surface plasmon resonance (SPR) (e.g., Biacore, e.g., T200 instrument, using manufacturer's protocols)) as compared to a reference Fc polypeptide that does not comprise the mutation.

Embodiment 42. The combination of Embodiment 41, the composition of Embodiment 41, the combination or composition for use of Embodiment 41, the antibody or antigen-binding fragment or polynucleotide for use of Embodiment 41, or the method of Embodiment 41, wherein the mutation that increases binding affinity to a human FcRn comprises: M428L; N434S; N434H; N434A; N434S; M252Y; S254T; T256E; T250Q; P257I: Q311I; D376V: T307A: E380A: or any combination thereof.

Embodiment 43. The combination of Embodiment 41 or 42, the composition of Embodiment 41 or 42, the combination or composition for use of Embodiment 41 or 42, the antibody or antigen-binding fragment or polynucleotide for use of Embodiment 41 or 42, or the method of Embodiment 41 or 42, wherein the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 44. The combination of any one of Embodiments 41-43, the composition of any one of Embodiments 41-43, the combination or composition for use of any one of Embodiments 41-43, the antibody or antigen-binding fragment or polynucleotide for use of any one of Embodiments 41-43, or the method of any one of Embodiments 41-43, wherein the mutation that increases binding affinity to a human FcRn comprises M428L/N434S.

Embodiment 45. The combination of any one of Embodiments 41-44, the composition of any one of Embodiments 41-44, the combination or composition for use of any one of Embodiments 41-44, the antibody or antigen-binding fragment or polynucleotide for use of any one of Embodiments 41-44, or the method of any one of Embodiments 41-44, wherein the mutation that enhances binding to a FcγR comprises S239D; I332E; A330L; G236A; or any combination thereof.

Embodiment 46. The combination of any one of Embodiments 41-45, the composition of any one of Embodiments 41-45, the combination or composition for use of any one of Embodiments 41-45, the antibody or antigen-binding fragment or polynucleotide for use of any one of Embodiments 41-45, or the method of any one of Embodiments 41-45, wherein the mutation that enhances binding to a FcγR comprises: (i) S239D/I332E; (ii) S239D/A330L/I332E; (iii) G236A/S239D/I332E; or (iv) G236A/A330L/I332E, wherein the Fc polypeptide or fragment thereof optionally comprises Ser at position 239.

Embodiment 47. The combination of any one of Embodiments 1 and 10-46, the composition of any one of Embodiments 2 and 10-46, the combination or composition for use of any one of Embodiments 3, 4, and 10-46, the antibody or antigen-binding fragment or polynucleotide for use of any one of Embodiments 5, 6, and 10-46, or the method of any one of Embodiments 7-46, wherein the anti-HA antibody or antigen-binding fragment, the anti-NA antibody or antigen binding fragment, or both, comprises a mutation that alters glycosylation, wherein the mutation that alters glycosylation comprises N297A, N297Q, or N297G, and/or which is aglycosylated and/or afucosylated.

Embodiment 48. The combination of any one of Embodiments 1 and 10-47, the composition of any one of Embodiments 2 and 10-47, the combination or composition for use of any one of Embodiments 3, 4, and 10-47, the antibody or antigen-binding fragment or polynucleotide for use of any one of Embodiments 5, 6, and 10-47, or the method of any one of Embodiments 7-47, wherein the treatment and/or prevention comprises post-exposure prophylaxis.

Embodiment 49. The combination of any one of Embodiments 1 and 10-48, the composition of any one of Embodiments 2 and 10-48, the combination or composition for use of any one of Embodiments 3, 4, and 10-48, the antibody or antigen-binding fragment or polynucleotide for use of any one of Embodiments 5, 6, and 10-48, or the method of any one of Embodiments 7-48, the wherein subject has received, is receiving, or will receive an antiviral, wherein, optionally, the antiviral comprises a neuraminidase inhibitor, an influenza polymerase inhibitor, or both.

Embodiment 50. The combination of any one of Embodiments 1 and 10-49, the composition of any one of Embodiments 2 and 10-49, the combination or composition for use of any one of Embodiments 3, 4, and 10-49, the antibody or antigen-binding fragment or polynucleotide for use of any one of Embodiments 5, 6, and 10-49, or the method of any one of Embodiments 7-49, wherein the antibody or antigen-binding fragment comprises: (i) a CH1-CH3 comprising or consisting of the amino acid sequence set forth in SEQ ID NO.:252; (ii) a CH1-CH3 comprising or consisting of the amino acid sequence set forth in SEQ ID NO.:253; (iii) a CL comprising or consisting of the amino acid sequence set forth in SEQ ID NO.:254; or (iv) any combination of (i)-(iii).

Embodiment 51. The combination of any one of Embodiments 1 and 10-50, the composition of any one of Embodiments 2 and 10-50, the combination or composition for use of any one of Embodiments 3, 4, and 10-50, the antibody or antigen-binding fragment or polynucleotide for use of any one of Embodiments 5, 6, and 10-50, or the method of any one of Embodiments 7-50, wherein the anti-NA antibody or antigen-binding fragment comprises: (1) a heavy chain comprising or consisting of the amino acid sequence set forth in SEQ ID NO.:255; and (2) a light chain comprising or consisting of the amino acid sequence set forth in SEQ ID NO.:257, and/or wherein the anti-HA antibody or antigen-binding fragment comprises: (1) a heavy chain comprising or consisting of the amino acid sequence set forth in SEQ ID NO.:270 or 272; and (2) a light chain comprising or consisting of the amino acid sequence set forth in SEQ ID NO.:271 or 273.

Embodiment 52. The combination of any one of Embodiments 1 and 10-50, the composition of any one of Embodiments 2 and 10-51, the combination or composition for use of any one of Embodiments 3, 4, and 10-51, the antibody or antigen-binding fragment or polynucleotide for use of any one of Embodiments 5, 6, and 10-51, or the method of any one of Embodiments 7-51, wherein the anti-NA antibody or antigen-binding fragment comprises: (1) a heavy chain comprising or consisting of the amino acid sequence set forth in SEQ ID NO.:256; and (2) a light chain comprising or consisting of the amino acid sequence set forth in SEQ ID NO.:257, and/or wherein the anti-HA antibody or antigen-binding fragment comprises: (1) a heavy chain comprising or consisting of the amino acid sequence set forth in SEQ ID NO.:270 or 272; and (2) a light chain comprising or consisting of the amino acid sequence set forth in SEQ ID NO.:271 or 273.

Embodiment 53. The combination of any one of Embodiments 1 and 10-51, the composition of any one of Embodiments 2 and 10-51, the combination or composition for use of any one of Embodiments 3, 4, and 10-51, the antibody or antigen-binding fragment or polynucleotide for use of any one of Embodiments 5, 6, and 10-51, or the method of any one of Embodiments 7-51, wherein the anti-NA antibody or antigen-binding fragment comprises: (1) two heavy chains, each comprising or consisting of the amino acid sequence set forth in SEQ ID NO.:255; and (2) two light chains, each comprising or consisting of the amino acid sequence set forth in SEQ ID NO.:257, and/or wherein the anti-HA antibody or antigen-binding fragment comprises: (1) two heavy chains, each comprising or consisting of the amino acid sequence set forth in SEQ ID NO.:270 or 272; and (2) two light chains, each comprising or consisting of the amino acid sequence set forth in SEQ ID NO.:271 or 273.

Embodiment 54. The combination of any one of Embodiments 1 and 10-50, the composition of any one of Embodiments 2 and 10 and 52, the combination or composition for use of any one of Embodiments 3, 4, and 10-50 and 52, the antibody or antigen-binding fragment or polynucleotide for use of any one of Embodiments 5, 6, and 10-50 and 52, or the method of any one of Embodiments 7-50 and 52, wherein the anti-NA antibody or antigen-binding fragment comprises: (1) two heavy chains, each comprising or consisting of the amino acid sequence set forth in SEQ ID NO.:256; and (2) two light chains, each comprising or consisting of the amino acid sequence set forth in SEQ ID NO.:257, and/or wherein the anti-HA antibody or antigen-binding fragment comprises: (1) two heavy chains, each comprising or consisting of the amino acid sequence set forth in SEQ ID NO.:270 or 272; and (2) two light chains, each comprising or consisting of the amino acid sequence set forth in SEQ ID NO.:271 or 273.

Embodiment 55. The combination of any one of Embodiments 50-54, the composition of any one of Embodiments 50-54, the combination or composition for use of any one of Embodiments 50-54, the antibody or antigen-binding fragment or polynucleotide for use of any one of Embodiments 50-54, or the method of any one of Embodiments 50-54, wherein the antiviral comprises oseltamivir, zanamivir, lanimivir, peramivir, baloxavir, or any combination thereof.

Embodiment 56. The combination of any one of Embodiments 1 and 10-55, the composition of any one of Embodiments 2 and 10-55, the combination or composition for use of any one of Embodiments 3, 4, and 10-55, the antibody or antigen-binding fragment or polynucleotide for use of any one of Embodiments 5, 6, and 10-55, or the method of any one of Embodiments 7-55, wherein: (i) the IAV comprises a Group 1 IAV, a Group 2 IAV, or both, wherein, optionally, the Group 1 IAV NA comprises a N1, a N4, a N5, and/or a N8; and/or the Group 2 IAV NA comprises a N2, a N3, a N6, a N7, and/or a N9, wherein, further optionally, the N1 is from A/California/07/2009, is from A/California/07/2009 123R/H275Y, is from A/Swine/Jiangsu/J004/2018, is from A/Stockholm/18/2007, is from A/Brisbane/02/2018, is from A/Michigan/45/2015, is from A/Mississippi/3/2001, is from A/Netherlands/603/2009, is from A/Netherlands/602/2009, is from A/Vietnam/1203/2004, is from A/G4/SW/Shangdong/1207/2016, is from A/G4/SW/Henan/SN13/2018, and/or is from A/New Jersey/8/1976; the N4 is from A/mallard duck/Netherlands/30/2011; the N5 is from A/aquatic bird/Korea/CN5/2009; the N8 is from A/harbor seal/New Hampshire/179629/2011; the N2 is from A/Washington/01/2007, is from A/HongKong/68, is from A/HongKong/2671/2019, is from A/South Australia/34/2019, is from A/Switzerland/8060/2017, is from A/Singapore/INFIMH-16-0019/2016, is from A/Switzerland/9715293/2013, is from A/Leningrad/134/17/57, is from A/Florida/4/2006, is from A/Netherlands/823/1992, is from A/Norway/466/2014, is from A/Texas/50/2012, is from A/Victoria/361/2011, is from A/SW/Mexico/SG1444/2011, is from A/Aichi/2/1968, is from A/Bilthoven/21793/1972, is from A/Netherlands/233/1982, is from A/Shanghai/11/1987, is from A/Nanchang/933/1995, is from A/Fukui/45/2004, A/Brisbane/10/2007, is from A/Tanzania/205/2010; the N3 is from A/Canada/rv504/2004; the N6 is from A/swine/Ontario/01911/1/99; the N7 is from A/Netherlands/078/03; and/or the N9 is from A/Anhui/2013, is from A/Hong Kong/56/2015 and/or (ii) the IBV NA is from: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/I/2010 (Yamagata); B/Yamanashi/166/1998 (Yamagata); B/Brisbane/33/2008 (Victoria); B/Colorado/06/2017 (Victoria); B/Hubei-wujiang/158/2009 (Yamagata); B/Massachusetts/02/2012 (Yamagata); B/Netherlands/234/2011; B/Perth/211/2001 (Yamagata); B/Phuket/3073/2013 (Yamagata); B/Texas/06/2011 (Yamagata); B/HongKong/05/1972; B/Harbin/7/1994 (Victoria); B/Washington/02/2019 (Victoria); B/Perth/211/2011; or any combination thereof.

Embodiment 57. A multispecific antibody or antigen binding fragment thereof, comprising: (i) an antigen-binding domain that is capable of binding to an influenza A virus (IAV) hemagglutinin (HA); and (ii) an antigen-binding domain that is capable of binding to a neuraminidase (NA) from: 2(i) an IAV, wherein the IAV comprises a Group 1 IAV, a Group 2 IAV, or both; and 2(ii) an influenza B virus (IBV).

Embodiment 58. The multispecific antibody or antigen-binding fragment of Embodiment 57, comprising a dual variable domain immunoglobulin (DVD-Ig) format.

Embodiment 59. The multispecific antibody or antigen-binding fragment of Embodiment 57 or 58, comprising an Insert-in-Elbow-Ig (IEI-Ig) format.

Embodiment 60. The multispecific antibody of any one of Embodiments 57-59, wherein: (1) the anti-HA antigen-binding domain comprises CDRH1, CDRH2, CDRH3, CDRL1, CDRL2, and CDRL3 amino acid sequences of SEQ ID NOs.: (1)(i) 274-279, respectively; (1)(ii) 3, 29, 5 and 9-11, respectively; (1)(iii) 32, 4, 5 and 9-11, respectively; (1)(iv) 3, 35, 5 and 9-11, respectively; (1)(v) 32, 35, 5, and 9-11, respectively; (1)(vi) 15-17 and 21-23, respectively; (1)(vii) 15, 42, 17 and 21-23, respectively; (1)(vii) or 3-5 and 9-11, respectively, or as set forth in the variable domain amino acid sequences of SEQ ID NOs.:43 and 44, respectively; and/or (2) the anti-NA antigen-binding domain comprises CDRH1, CDRH2, CDRH3, CDRL1, CDRL2, and CDRL3 amino acid sequences of SEQ ID NOs.: (2)(i) 193-195 and 199-201, respectively; (2)(ii) 61-63 and 67-69, respectively; (2)(iii) 73-75 and 79-81, respectively; (2)(iv) 73, 74, 218, and 79-81, respectively; (2)(v) 73-75, 79, 80, and 221, respectively; (2)(vi) 73-75, 79, 80, and 224, respectively; (2)(vii) 73-75, 79, 80, and 227, respectively; (2)(viii) 73, 74, 218, 79, 80, and 221, respectively; (2)(ix) 73, 74, 218, 79, 80, and 224, respectively; (2)(x) 73, 74, 218, 79, 80, and 227, respectively; (2)(xi) 85-87 and 91-93, respectively; (2)(xii) 97-99 and 103-105, respectively; (2)(xiii) 109-111 and 115-117, respectively; (2)(xiv) 121-123 and 127-129, respectively; (2)(xv) 133-135 and 139-141, respectively; (2)(xvi) 133, 134, 230 and 139-141, respectively; (2)(xvii) 133-135, 139, 141, and 233, respectively; (2)(xviii) 133-135, 139, 141, and 236, respectively; (2)(xix) 133-135, 139, 141, and 239, respectively; (2)(xx) 133, 134, 184, 139, 141, and 233, respectively; (2)(xxi) 133, 134, 184, 139, 141, and 236, respectively; (2)(xxii) 133, 134, 184, 139, 141, and 239, respectively; (2)(xxiii) 145-147 and 151-153, respectively; (2)(xxiv) 157-159 and 163-165, respectively; (2)(xxv) 169-171 and 175-177, respectively; (2)(xxvi) 181-183 and 187-189, respectively; (2)(xxvii) 49-51 and 55-57, respectively; (2)(xxviii) 205-207 and 211-213, respectively; or (2)(xxvix) 264-266 and 267-296, respectively.

Embodiment 61. The multispecific antibody or antigen-binding fragment of any one of Embodiments 57-60, wherein: (1) the anti-HA antigen-binding domain comprises (1)(i) a VH comprising or consisting of an amino acid sequence having at least 80% (e.g., 80%, 85%, 90/6, 91%, 92%, 93%, 94%, 95%, 96%, 97%, 98%, 99%, or more) identity to the amino acid sequence set forth in any one of SEQ ID NOs.: 43, 2, 26, 28, 31, 34, 37, 14, 39, and 41, wherein sequence variation with reference to SEQ ID NO.: 43, 2, 26, 28, 31, 34, 37, 14, 39, or 41, respectively, is optionally comprised in one or more framework region and/or sequence variation comprises one or more substitution to a germline-encoded amino acid; and/or (1)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 44, 8, and 20 or 44, wherein sequence variation with respect to SEQ ID NO:44, 8, or 20, respectively, is optionally comprised in one or more framework regions and/or sequence variation comprises one or more substitution to a germline-encoded amino acid; and/or (2) the anti-NA antigen-binding domain comprises (2)(i) a VH comprising or consisting of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 241, 48, 60, 72, 171, 84, 96, 108, 120, 132, 229, 144, 156, 168, 180, 192, 204, 245, 249, 258, and 261, wherein sequence variation with reference to SEQ ID NO.: 241, 48, 60, 72, 171, 84, 96, 108, 120, 132, 229, 144, 156, 168, 180, 192, 204, 245, and 249, 258, and 261, respectively, is optionally comprised in one or more framework region and/or sequence variation comprises one or more substitution to a germline-encoded amino acid; and/or (2)(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 243, 54, 66, 78, 90, 102, 114, 126, 138, 150, 162, 174, 186, 198, 220, 223, 226, 232, 235, 238, 210, 247, 251, 259, and 263, wherein sequence variation with respect to SEQ ID NO.: 243, 54, 66, 78, 90, 102, 114, 126, 138, 150, 162, 174, 186, 198, 220, 223, 226, 232, 235, 238, 210, 247, 251, 259, and 263, respectively, is optionally comprised in one or more framework regions and/or sequence variation comprises one or more substitution to a germline-encoded amino acid.

Embodiment 62. The multispecific antibody or antigen-binding fragment of any one of Embodiments 57-61, wherein: (1) the VH and the VL of the anti-HA antigen-binding domain comprise or consist of the amino acid sequences according to SEQ ID NOs.: (1)(i) 2 and 8, respectively; (1)(ii) 43 and 44, respectively; (1)(iii) 28 and 8, respectively; (1)(iv) 31 and 8, respectively; (1)(v) 34 and 8, respectively; (1)(vi) 37 and 8, respectively; (1)(vii) 14 and 20, respectively; (1)(viii) 39 and 20, respectively; (1)(ix) 41 and 20, respectively; or (1)(x) 26 and 8, respectively; and/or (2) the VH and the VL of the anti-NA antigen-binding domain comprise or consist of the amino acid sequences according to SEQ ID NOs.: (2)(i) 243 and 243, respectively; (2)(ii) 60 and 66, respectively; (2)(iii) 72 and 78 or 220 or 223, respectively; (2)(vi) 72 and 226, respectively: (2)(vii) 217 and 78, respectively; (2)(viii) 217 and 220, respectively; (2)(ix) 217 and 223, respectively; (2)(x) 217 and 226, respectively; (2)(xi) 84 and 90, respectively; (2)(xii) 96 and 102, respectively; (2)(xiii) 108 and 114, respectively; (2)(xiv) 120 and 126, respectively; (2)(xv) 132 and 138, respectively; (2)(xvi) 132 and 232, respectively; (2)(xvii) 132 and 235, respectively; (2)(xviii) 132 and 238, respectively; (2)(xix) 229 and 138, respectively; (2)(xx) 229 and 232, respectively; (2)(xxi) 229 and 235, respectively; (2)(xxii) 229 and 238, respectively; (2)(xxiii) 144 and 150, respectively; (2)(xxiv) 156 and 162, respectively; (2)(xxv) 168 and 174, respectively; (2)(xxvi) 180 and 186, respectively; (2)(xxvii) 192 and 198, respectively; (2)(xxviii) 204 and 210, respectively; (2)(xxix) 48 and 54, respectively; (2)(xxx) 245 and 247, respectively; (2)(xxxi) 249 and 251, respectively; (2)(xxxii) 258 and 259, respectively; or (2)(xxxiii) 261 and 263, respectively.

Embodiment 63. The multispecific antibody or antigen-binding fragment of any one of Embodiments 57-62, comprising: (i) a CH1-CH3 comprising or consisting of the amino acid sequence set forth in SEQ ID NO.:252; (ii) a CH1-CH3 comprising or consisting of the amino acid sequence set forth in SEQ ID NO.:253; (iii) a CL comprising or consisting of the amino acid sequence set forth in SEQ ID NO.:254; or (iv) any combination of (i)-(iii).

Embodiment 64. The multispecific antibody or antigen-binding fragment of any one of Embodiments 57-63, comprising: (1) a heavy chain comprising or consisting of the amino acid sequence set forth in SEQ ID NO.:255; and (2) a light chain comprising or consisting of the amino acid sequence set forth in SEQ ID NO.:257.

Embodiment 65. The multispecific antibody or antigen-binding fragment of any one of Embodiments 57-64, comprising: (1) a heavy chain comprising or consisting of the amino acid sequence set forth in SEQ ID NO.:256; and (2) a light chain comprising or consisting of the amino acid sequence set forth in SEQ ID NO.:257.

Embodiment 66. An isolated polynucleotide encoding the multispecific antibody or antigen-binding fragment of any one of Embodiments 57-65.

Embodiment 67. A vector comprising the polynucleotide of Embodiment 66.

Embodiment 68. A recombinant host cell comprising the isolated polynucleotide of Embodiment 66 and/or the vector of Embodiment 67 and/or that expresses the multispecific antibody or antigen-binding fragment of any one of Embodiments 57-65.

Embodiment 69. A composition comprising the multispecific antibody or antigen-binding fragment of any one of Embodiments 57-65, the polynucleotide of Embodiment 66, the vector of Embodiment 67, and/or the host cell of Embodiment 68, and a pharmaceutically acceptable carrier, excipient, or diluent.

Embodiment 70. A method of preventing or treating an influenza A infection, an influenza B infection, or both, in a subject, the method comprising administering to the subject an effective amount of the multispecific antibody or antigen-binding fragment of any one of Embodiments 57-65, the polynucleotide of Embodiment 66, the vector of Embodiment 67, the host cell of Embodiment 68, and/or the composition of Embodiment 69.

Embodiment 71. The multispecific antibody or antigen-binding fragment of any one of Embodiments 57-65, the polynucleotide of Embodiment 66, the vector of Embodiment 67, the host cell of Embodiment 68, and/or the composition of Embodiment 69, for use in a method of treating or preventing an influenza A infection, an influenza B infection, or both, in a subject.

Embodiment 72. The multispecific antibody or antigen-binding fragment of any one of Embodiments 57-65, the polynucleotide of Embodiment 66, the vector of Embodiment 67, the host cell of Embodiment 68, and/or the composition of Embodiment 69, for use in a method of manufacturing a medicament for the treatment of prevention of an influenza A infection and/or an influenza B infection.

Embodiment 73. The multispecific antibody or antigen-binding fragment of any one of Embodiments 57-65, the polynucleotide of Embodiment 66, the vector of Embodiment 67, the host cell of Embodiment 68, the composition of Embodiment 69, the method of Embodiment 70, or the antibody or antigen-binding fragment for use of any one of Embodiments 71 and 72, wherein the anti-HA antibody or antigen-binding fragment, the anti-NA antibody or antigen binding fragment, or both, is/are capable of preventing a loss in body weight of greater than 25%, 20%, 15%, 10%, or 5% in a subject having an IAV infection and/or an IBV infection, as determined by reference to the subject's body weight just prior to the IAV and/or IBV infection.

Embodiment 74. The multispecific antibody or antigen-binding fragment of any one of Embodiments 57-65, the polynucleotide of Embodiment 66, the vector of Embodiment 67, the host cell of Embodiment 68, the composition of Embodiment 69, the method of Embodiment 70, the antibody or antigen-binding fragment for use of any one of Embodiments 71-73, wherein the anti-HA antibody or antigen-binding fragment, the anti-NA antibody or antigen binding fragment, or both, is/are capable extending survival of a subject having an IAV infection and/or an IIBV infection.

Embodiment 75. A method for treating or preventing an influenza infection in a subject, the method comprising administering to the subject: (1) an anti-HA antibody, or an antigen-binding fragment thereof, that comprises the VH amino acid sequence set forth in SEQ ID NO.:43 and the VL amino acid sequence set forth in SEQ ID NO.:44: and (2) an anti-NA antibody, or an antigen-binding fragment thereof, that comprises the VH amino acid sequence set forth in SEQ ID NO.:241 and the VL amino acid sequence set forth in SEQ ID NO.:243.

Embodiment 76. A method for treating or preventing an influenza infection in a subject, the method comprising administering to the subject a polynucleotide that encodes: (1) an anti-HA antibody, or an antigen-binding fragment thereof, that comprises the VH amino acid sequence set forth in SEQ ID NO.:43 and the VL amino acid sequence set forth in SEQ ID NO.:44, and (2) an anti-NA antibody, or an antigen-binding fragment thereof, that comprises the VH amino acid sequence set forth in SEQ ID NO.:241 and the VL amino acid sequence set forth in SEQ ID NO.:243.

Embodiment 77. A method for treating or preventing an influenza infection in a subject, the method comprising administering to the subject: (1) a polynucleotide encoding an anti-HA antibody, or an antigen-binding fragment thereof, that comprises the VH amino acid sequence set forth in SEQ ID NO.:43 and the VL amino acid sequence set forth in SEQ ID NO.:44; and (2) a polynucleotide encoding an anti-NA antibody, or an antigen-binding fragment thereof, that comprises the VH amino acid sequence set forth in SEQ ID NO.:241 and the VL amino acid sequence set forth in SEQ ID NO.:243.

Embodiment 78. A method for treating or preventing an influenza infection in a subject, the method comprising administering to the subject: (1) an anti-HA antibody, or an antigen-binding fragment thereof, that comprises a VH comprising the CDRH1, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 79. A method for treating or preventing an influenza infection in a subject, the method comprising administering to the subject a polynucleotide that encodes: (1) an anti-HA antibody, or an antigen-binding fragment thereof, that comprises a VH comprising the CDR11, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 80. A method for treating or preventing an influenza infection in a subject, the method comprising administering to the subject: (1) a polynucleotide encoding an anti-HA antibody, or an antigen-binding fragment thereof, that comprises a VH comprising the CDRH1, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) a polynucleotide encoding an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 81. The method of any one of Embodiments 75-80, wherein the antibody or antigen-binding fragment of (1) comprises the heavy chain amino acid sequence of SEQ ID NO.:270 or SEQ ID NO.: 272 and the light amino acid sequence of SEQ ID NO.:271.

Embodiment 82. The method of any one of Embodiments 75-81, wherein the antibody or antigen-binding fragment of (2) comprises the heavy chain amino acid sequence of SEQ ID NO.:255 or SEQ ID NO.:256 and the light chain amino acid sequence of SEQ ID NO.:257.

Embodiment 83. The method of any one of Embodiments 76-82, wherein the polynucleotide, the polynucleotide of (1), and/or the polynucleotide of (2), respectively, comprises mRNA.

Embodiment 84. The method of any one of Embodiments 76-83, wherein the polynucleotide, the polynucleotide of (1), and/or the polynucleotide of (2), respectively, comprises a modified nucleoside, a cap-1 structure, a cap-2 structure, or any combination thereof.

Embodiment 85. The method of Embodiment 84, wherein the polynucleotide, the polynucleotide of (1), and/or the polynucleotide of (2), respectively, comprises a pseudouridine, a N6-methyladenonsine, a 5-methylcytidine, a 2-thiouridine, or any combination thereof.

Embodiment 86. The method of Embodiment 85, wherein the pseudouridine comprises N1-methylpseudouridine.

Embodiment 87. A polynucleotide that encodes. (1) an anti-HA antibody, or an antigen-binding fragment thereof, that comprises the VH amino acid sequence set forth in SEQ ID NO.:43 and the VL amino acid sequence set forth in SEQ ID NO.:44; and (2) an anti-NA antibody, or an antigen-binding fragment thereof, that comprises the VH amino acid sequence set forth in SEQ ID NO.:241 and the VL amino acid sequence set forth in SEQ ID NO.:243.

Embodiment 88. A polynucleotide that encodes: (1) an anti-HA antibody, or an antigen-binding fragment thereof, that comprises a VH comprising the CDRH1, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 89. A composition comprising: (1) a polynucleotide that encodes an anti-HA antibody, or an antigen-binding fragment thereof, that comprises the VH amino acid sequence set forth in SEQ ID NO.:43 and the VL amino acid sequence set forth in SEQ ID NO.:44; and (2) a polynucleotide that an anti-NA antibody, or an antigen-binding fragment thereof, that comprises the VH amino acid sequence set forth in SEQ ID NO.:241 and the VL amino acid sequence set forth in SEQ ID NO.:243.

Embodiment 90. A composition comprising: (1) a polynucleotide that encodes an anti-HA antibody, or an antigen-binding fragment thereof, that comprises a VH comprising the CDRH1, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) a polynucleotide that encodes an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 91. A combination of: (1) a polynucleotide encoding an anti-HA antibody, or an antigen-binding fragment thereof, that comprises the VH amino acid sequence set forth in SEQ ID NO.:43 and the VL amino acid sequence set forth in SEQ ID NO.:44; and (2) a polynucleotide encoding an anti-NA antibody, or an antigen-binding fragment thereof, that comprises the VH amino acid sequence set forth in SEQ ID NO.:241 and the VL amino acid sequence set forth in SEQ ID NO.:243.

Embodiment 92. A combination of: (1) a polynucleotide that encodes an anti-HA antibody, or an antigen-binding fragment thereof, that comprises a VH comprising the CDRH1, CDRH2, and CDRH3 amino acid sequences set forth in SEQ ID NOs.:274-276, respectively, and a VL comprising the CDRL1, CDRL2, and CDRL3 amino acid sequences set forth in SEQ ID NOs.:277-279, respectively; and (2) a polynucleotide that encodes an anti-NA antibody, or an antigen-binding fragment thereof, that comprises a VH comprising the CDRH1, CDRH2, and CDRH3 amino acid sequences set forth in SEQ ID NOs.:193-195, respectively, and a VL comprising the CDRL1, CDRL2, and CDRL3 amino acid sequences set forth in SEQ ID NOs.:199-201, respectively.

Embodiment 93. The polynucleotide of Embodiment 87 or 88, the composition of Embodiment 89 or 90, or the combination of Embodiment 91 or 92, wherein the antibody or antigen-binding fragment of (1) comprises the heavy chain amino acid sequence of SEQ ID NO.:270 or SEQ ID NO.: 272 and the light amino acid sequence of SEQ ID NO.:271.

Embodiment 94. The polynucleotide of Embodiment 87, 88, or 93, the composition of Embodiment 89, 90, or 93, or the combination of Embodiment 91, 92, or 93, wherein the antibody or antigen-binding fragment of (2) comprises the heavy chain amino acid sequence of SEQ ID NO.:255 or SEQ ID NO.:256 and the light chain amino acid sequence of SEQ ID NO.:257.

Embodiment 95. The polynucleotide of Embodiment 87, 88, 93, or 94, the composition of Embodiment 89, 90, 93, or 94, or the combination of any one of Embodiments 91-94, wherein the polynucleotide, the polynucleotide of (1), and/or the polynucleotide of (2), respectively, comprises mRNA.

Embodiment 96. The polynucleotide of Embodiment 87, 88, 93, 94, or 95, the composition of Embodiment 89, 90, 93, 94, or 95, or the combination of any one of Embodiments 91-94, wherein the polynucleotide, the polynucleotide of (1), and/or the polynucleotide of (2), respectively, comprises a modified nucleoside, a cap-1 structure, a cap-2 structure, or any combination thereof.

Embodiment 97. The polynucleotide, composition, or combination of Embodiment 96, wherein the polynucleotide, the polynucleotide of (1), and/or the polynucleotide of (2), respectively, comprises a pseudouridine, a 2-thiouridine, a N6-methyladenonsine, a 5-methylcytidine, or any combination thereof.

Embodiment 98. The polynucleotide, composition, or combination of Embodiment 97, wherein the pseudouridine comprises N1-methylpseudouridine.

TABLE 1
TABLE OF CERTAIN SEQUENCES AND SEQ ID NUMBERS:

| SEQ | | |
|---|---|---|
| ID NO | Sequence | Identifier |
| 1 | CAGGTACAACTGCAGCAGTCAGGTCCAGGACTGG | FHF11 VH (wt-nt) |
| | TGAAGCCCTCGCAGACCCTCTCAGTCACCTGTGGC | |
| | ATCTCCGGGGACAGTGTCTCTAGTCACAGTGCT | |
| | GCTTGGAACTGGATCAGGCAGTCCCCATCGAGAG | |
| | GCCTTGAGTGGCTGGGAAGGACATATTACAGGTC | |
| | CAAGTGGTATAATGATTATGCAGTCTCTGTGAAA | |
| | AGTCGAATAACCATCAATCCAGACACATCCAAGA | |
| | ACCAGTTCTCCCTACAGTTGATCTCTGTGACTCCC | |
| | GAGGACACGGCTGTCTATTACTGTGCAAGAGTGG | |
| | GTGCTATGACTTTTGGACTTCTTACAGGGGGTA | |
| | TGGACGTCTGGGGCCAAGGGACCACGGTCACCGT | |
| | CTCCTCA | |
| 2 | QVQLQQSGPGLVKPSQTLSVTCGISGDSVSSHSAAW | HF11 VH (aa) |
| | NWIRQSPSRGLEWLGRTYYRSKWYNDYAVSVKSRI | |
| | TINPDTSKNQFSLQLISVTPEDTAVYYCARVGAMTF | |
| | GLLTGGMDVWGQGTTVTVSS | |
| 3 | GDSVSSHSAA | FHF11 CDR-H1 (aa) |
| 4 | TYYRSKWYN | FHF11 CDR-H2 (aa) |
| 5 | ARVGAMTFGLLTGGMDV | FHF11 CDR-H3 (aa) |
| 6 | CAGGTGCAGCTGCAGCAGTCTGGACCAGGACTGGTGA | FHF11 VH (co-nt) |
| | AGCCTAGCCAGACCCTGTCTGTGACATGCGGAATCTC | |
| | CGGCGACAGCGTGTCCAGCCACTCCGCCGCTTGGAAC | |
| | TGGATCAGACAGAGCCCATCTAGGGGACTGGAGTGGC | |
| | TGGGAAGGACCTACTATCGGAGCAAGTGGTACAATGA | |
| | CTATGCCGTGTCTGTGAAGTCCAGGATCACCATCAACC | |
| | CAGATACATCCAAGAATCAGTTCAGCCTGCAGCTGAT | |
| | CTCTGTGACCCCCGAGGACACAGCCGTGTACTATTGTG | |
| | CCAGAGTGGGCGCTATGACCTTTGGCCTGCTGACAGG | |
| | CGGAATGGACGTGTGGGGACAGGGAACCACAGTGAC | |
| | AGTGTCTTCC | |
| 7 | GAAATTGTGTTGACGCAGTCTCCAGGCACCCAGT | FHF11 Vk (wt-nt) |
| | CTTTGTCTCCAGGGGAAAGAGCCACCCTCTCCTG | |
| | CAGGGCCAGTCAGAGTCTGAGCCGCAGCTACTT | |
| | AGCCTGGTACCAGCAGAGACCTGGCAAGCCTCCC | |
| | AGGCTCCTCATCTATGGTGCATCCAGCAGGGCCA | |
| | CTGGCATCCCAGACAGGTTCAGTGGCAGTGGGTC | |
| | TGGGACAGACTTCAGTCTCACCATCAGCAGTCTG | |
| | GAGCCTGAAGATTCTGCAATGTATTTCTGTCAGT | |
| | ACTATGGTGATTCACCTCTATTCAGTTTCGGCC | |
| | CAGGGACCAAAGTGGATATCAAAC | |
| 8 | EIVLTQSPGTQSLSPGERATLSCRASQSLSRSYLAW | FHF11 Vk (aa) |
| | YQQRPGKPPRLLIYGASSRATGIPDRESGSGSGTDFS | |
| | LTISSLEPEDSAMYFCQYYGDSPLFSFGPGTKVDIK | |
| 9 | QSLSRSY | FHF11 CDR-L1 (aa) |
| 10 | GAS | FHF11 CDR-L2 (aa) |
| 11 | QYYGDSPLFS | FHF11 CDR-L3 (aa) |
| 12 | GAGATCGTGCTGACCCAGTCTCCTGGCACACAGA | FHF11 Vk (co-nt) |
| | GCCTGTCTCCAGGAGAGAGGGCCACCCTGTCCTG | |
| | CAGGGCTTCCCAGAGCCTGTCTAGGTCCTACCTG | |
| | GCCTGGTATCAGCAGAGACCAGGCAAGCCACCTA | |
| | GGCTGCTGATCTACGGAGCTTCCAGCAGGGCTAC | |
| | AGGCATCCCTGACAGATTCAGCGGCTCTGGCTCC | |
| | GGCACCGATTTTTCCCTGACAATCTCTTCCCTGGA | |
| | GCCAGAGGACTCCGCCATGTATTTCTGTCAGTACT | |
| | ATGGCGATAGCCCACTGTTCTCTTTTGGCCCCGGC | |
| | ACCAAGGTGGACATCAAG | |
| 13 | CAGGTACAACTGCAGCAGTCAGGTCCAGGACTGGTGA | FHF12 VH (wt-nt) |
| | AGCCCTCGCAGACCCTCTCAGTCACCTGTGCCATCTCC | |
| | GGGGACAGTGTCTCTAGTCACAGTGCTGCTTGGAA | |
| | CTGGATCAGGCAGTCCCCATCGAGAGGCCTTGAGTGG | |
| | CTGGGAAGGACATATTACAGGTCCAAGTGGTATAAT | |
| | GATTATGCAGTCTCTGTGAAAAGTCGAATAACCATCA | |
| | ACCCAGACACATCCAAGAACCAGTTCTCCCTACAGCT | |
| | GGTCTCTGTGACTCCCGAGGACACGGCTGTCTATTACT | |
| | GTGCAAGAGTGGGTGCTGCGACTTTTGGAATTCTT | |
| | ACAGGGGGTATGGACGTCTGGGGCCAAGGGACCAC | |
| | GGTCACCGTCTCCTCA | |
| 14 | QVQLQQSGPGLVKPSQTLSVTCAISGDSVSSHSAA | FFHF12 VH (aa) |
| | WNWIRQSPSRGLEWLGRTYYRSKWYNDYAVSVKS | |
| | RITINPDTSKNQFSLQLVSVTPEDTAVYYCARVGAA | |
| | TFGILTGGMDVWGQGTTVTVSS | |
| 15 | GDSVSSHSAA | FHF12 CDR-H1 (aa) |
| 16 | TYYRSKWYN | FHF12 CDR-H2 (aa) |
| 17 | ARVGAATFGILTGGMDV | FHF12 CDR-H3 (aa) |
| 18 | CAGGTGCAGCTGCAGCAGTCTGGACCAGGACTGG | FHF12 VH (co-nt) |
| | TGAAGCCTAGCCAGACCCTGTCTGTGACATGCGCT | |
| | ATCTCCGGCGACAGCGTGTCCAGCCACTCCGCCGC | |
| | TTGGAACTGGATCAGACAGAGCCCATCTAGGGGA | |
| | CTGGAGTGGCTGGGAAGGACCTACTATCGGAGCA | |
| | AGTGGTACAATGACTATGCCGTGTCCGTGAAGTCC | |
| | AGGATCACCATCAACCCAGATACATCCAAGAATC | |
| | AGTTCAGCCTGCAGCTGGTGTCTGTGACCCCCGAG | |
| | GACACAGCCGTGTACTATTGTGCTAGAGTGGGCGC | |
| | CGCTACCTTTGGCATCCTGACAGGCGGAATGGACG | |
| | TGTGGGGACAGGGAACCACAGTGACAGTGTCTTC | |
| | C | |
| 19 | GAAATTGTGTTGACGCAGTCTCCAGGCACCCAGT | FHF12 Vk (wt-nt) |
| | CTTTGTCTCCAGGGGATAGAGCCACCCTCTCCTGC | |
| | AGGGCCAGTCAGAGTCTGAGCAGAAGCTACTTA | |
| | GCCTGGTACCAGCAGAGACCTGGCAAGCCTCCCA | |
| | GGCTCCTCATCTATGGTGCATCCAGCAGGGCCAC | |
| | TGGCATCCCAGACAGGTTCAGTGGCAGTGGGTCT | |
| | GGGACAGACTTCAGTCTCACCATCAGCAGTCTGG | |
| | AGCCTGAAGATTCTGCTATGTATTTCTGTCAGTA | |
| | CTATGGTGATTCACCTCTATTCAGTTTCGGCCC | |
| | TGGGACCAAAGTGGATATCAAAC | |
| 20 | EIVLTQSPGTQSLSPGDRATLSCRASQSLSRSYLAW | FHF12 Vk (aa) |
| | YQQRPGKPPRLLIYGASSRATGIPDRFSGSGSGTDFS | |
| | LTISSLEPEDSAMYFCQYYGDSPLFSFGPGTKVDIK | |
| 21 | QSLSRSY | FHF12 CDR-L1 (aa) |
| 22 | GAS | FHF12 CDR-L2 (aa) |
| 23 | QYYGDSPLFS | FHF12 CDR-L3 (aa) |
| 24 | GAGATCGTGCTGACCCAGTCTCCTGGCACACAGA | FHF12 Vk (co-nt) |
| | GCCTGTCTCCAGGCGACAGGGCCACCCTGTCCTG | |
| | CAGGGCTTCCCAGAGCCTGTCTAGGTCCTACCTG | |
| | GCCTGGTATCAGCAGAGACCAGGCAAGCCACCTA | |
| | GGCTGCTGATCTACGGAGCTTCCAGCAGGGCTAC | |
| | AGGCATCCCTGACAGATTCAGCGGCTCTGGCTCC | |
| | GGCACCGATTTTTCCCTGACAATCTCTTCCCTGGA | |
| | GCCAGAGGACTCCGCCATGTATTTCTGTCAGTACT | |
| | ATGGCGATAGCCCACTGTTCTCTTTTGGCCCCGGC | |
| | ACCAAGGTGGATATCAAG | |
| 25 | CAGGTGCAGCTGCAGCAGTCTGGACCAGGACTGG | FHF11-VH W36F |
| | TGAAGCCTAGCCAGACCCTGTCTGTGACATGCGGA | (nt) |
| | ATCTCCGGCGACAGCGTGTCCAGCCACTCCGCC | |
| | GCTTTCAACTGGATCAGACAGAGCCCATCTAGGG | |
| | GACTGGAGTGGCTGGGAAGGACCTACTATCGGA | |
| | GCAAGTGGTACAATGACTATGCCGTGTCTGTGAA | |
| | GTCCAGGATCACCATCAACCCAGATACATCCAAG | |
| | AATCAGTTCAGCCTGCAGCTGATCTCTGTGACCCC | |
| | CGAGGACACAGCCGTGTACTATTGTGCCAGAGTG | |
| | GGCGCTATGACCTTTGGCCTGCTGACAGGCGG | |
| | AATGGACGTGTGGGGACAGGGAACCACAGTGAC | |
| | AGTGTCTTCC | |
| 26 | QVQLQQSGPGLVKPSQTLSVTCGISGDSVSSHSAAF | FHF11-VH W36F |
| | NWIRQSPSRGLEWLGRTYYRSKWYNDYAVSVKSRI | (aa) |
| | TINPDTSKNQFSLQLISVTPEDTAVYYCARVGAMTF | |
| | GLLTGGMDVWGQGTTVTVSS | |
| 27 | CAGGTGCAGCTGCAGCAGTCTGGACCAGGACTGG | FHF11-VH W59F |
| | TGAAGCCTAGCCAGACCCTGTCTGTGACATGCGGA | (nt) |
| | ATCTCCGGCGACAGCGTGTCCAGCCACTCCGCC | |
| | GCTTGGAACTGGATCAGACAGAGCCCATCTAGGG | |
| | GACTGGAGTGGCTGGGAAGGACCTACTATCGGA | |
| | GCAAGTTCTACAATGACTATGCCGTGTCTGTGAA | |
| | GTCCAGGATCACCATCAACCCAGATACATCCAAG | |
| | AATCAGTTTAGCCTGCAGCTGATCTCTGTGACCCC | |
| | CGAGGACACAGCCGTGTACTATTGTGCCAGAGTG | |
| | GGCGCTATGACCTTCGGCCTGCTGACAGGCGG | |
| | AATGGACGTGTGGGGACAGGGAACCACAGTGAC | |
| | AGTGTCTTCC | |
| 28 | QVQLQQSGPGLVKPSQTLSVTCGISGDSVSSHSAAW | FHF11-VH W59F |
| | NWIRQSPSRGLEWLGRTYYRSKFYNDYAVSVKSRIT | (aa) |
| | INPDTSKNQFSLQLISVTPEDTAVYYCARVGAMTFG | |
| | LLTGGMDVWGQGTTVTVSS | |
| 29 | TYYRSKFYN | FHF11-VH W59F |
| | | CDRH2 (aa) |
| 30 | CAGGTGCAGCTGCAGCAGTCTGGACCAGGACTGG | FHF11v3 VH (nt) |
| | TGAAGCCTAGCCAGACCCTGTCTGTGACATGCGG | |
| | CATCTCCGGCGACAGCGTGTCCAGCTACTCCGC | |
| | CGCTTGGAACTGGATCAGACAGAGCCCATCTAGG | |
| | GGACTGGAGTGGCTGGGAAGGACCTACTATCGG | |
| | AGCAAGTGGTACAATGACTATGCCGTGTCTGTG | |
| | AAGTCCAGGATCACCATCAACCCAGATACATCCA | |
| | AGAATCAGTTCAGCCTGCAGCTGATCTCTGTGAC | |
| | CCCCGAGGACACAGCCGTGTACTATTGTGCCAGA | |
| | GTGGGCGCTATGACCTTTGGCCTGCTGACAGG | |
| | CGGAATGGACGTGTGGGGACAGGGAACCACAGT | |
| | GACAGTGTCTTCC | |
| 31 | QVQLQQSGPGLVKPSQTLSVTCGISGDSVSSYSAAW | FHF11v3 VH (aa) |
| | NWIRQSPSRGLEWLGRTYYRSKWYNDYAVSVKSR | |
| | ITINPDTSKNQFSLQLISVTPEDTAVYYCARVGAMT | |
| | FGLLTGGMDVWGQGTTVTVSS | |
| 32 | GDSVSSYSAA | FHF11v3 CDRH1 |
| | | (aa) |
| 33 | CAGGTGCAGCTGCAGCAGTCTGGACCAGGACTGG | FHF11v6 VH (nt) |
| | TGAAGCCTAGCCAGACCCTGTCTGTGACATGCGG | |
| | AATCTCCGGCGACAGCGTGTCCAGCCACTCCG | |
| | CCGCTTGGAACTGGATCAGACAGAGCCCATCTAG | |
| | GGGACTGGAGTGGCTGGGAAGGACCTACTATCG | |
| | GAGCGGCTGGTACAATGACTATGCCGTGTCTGT | |
| | GAAGTCCAGGATCACCATCAACCCAGATACATCC | |
| | AAGAATCAGTTCAGCCTGCAGCTGATCTCTGTGA | |
| | CCCCCGAGGACACAGCCGTGTACTATTGTGCCAG | |
| | AGTGGGCGCTATGACCTTTGGCCTGCTGACAG | |
| | GCGGAATGGACGTGTGGGGACAGGGAACCACA | |
| | GTGACAGTGTCTTCC | |
| 34 | QVQLQQSGPGLVKPSQTLSVTCGISGDSVSSHSAA | FHF11v6 VH (aa) |
| | WNWIRQSPSRGLEWLGRTYYRSGWYNDYAVSVKS | |
| | RITINPDTSKNQFSLQLISVTPEDTAVYYCARVGAM | |
| | TFGLLTGGMDVWGQGTTVTVSS | |
| 35 | TYYRSGWYN | FHF11v6 CDRH2 |
| | | (aa) |
| 36 | CAGGTGCAGCTGCAGCAGTCTGGACCAGGACTGG | FHF11v9 VH (nt) |
| | TGAAGCCTAGCCAGACCCTGTCTGTGACATGCGGC | |
| | ATCTCCGGCGACAGCGTGTCCAGCTACTCCGCC | |
| | GCTTGGAACTGGATCAGACAGAGCCCATCTAGGG | |
| | GACTGGAGTGGCTGGGAAGGACCTACTATCGGA | |
| | GCGGCTGGTACAATGACTATGCCGTGTCTGTGAA | |
| | GTCCAGGATCACCATCAACCCAGATACATCCAAG | |
| | AATCAGTTCAGCCTGCAGCTGATCTCTGTGACCCC | |
| | CGAGGACACAGCCGTGTACTATTGTGCCAGAGTG | |
| | GGCGCTATGACCTTTGGCCTGCTGACAGGCGG | |
| | AATGGACGTGTGGGGACAGGGAACCACAGTGAC | |
| | AGTGTCTTCC | |
| 37 | QVQLQQSGPGLVKPSQTLSVTCGISGDSVSSYSAAW | FHF11v9 VH (aa) |
| | NWIRQSPSRGLEWLGRTYYRSGWYNDYAVSVKSR | |
| | ITINPDTSKNQFSLQLISVTPEDTAVYYCARVGAMT | |
| | FGLLTGGMDVWGQGTTVTVSS | |
| 38 | CAGGTGCAGCTGCAGCAGTCTGGACCAGGACTGG | FHF12-VH-W36F |
| | TGAAGCCTAGCCAGACCCTGTCTGTGACATGCGC | (nt) |
| | TATCTCCGGCGACAGCGTGTCCAGCCACTCCGC | |
| | CGCTTTCAACTGGATCAGACAGAGCCCATCTAGG | |
| | GGACTGGAGTGGCTGGGAAGGACCTACTATCGG | |
| | AGCAAGTGGTACAATGACTATGCCGTGTCCGTG | |
| | AAGTCCAGGATCACCATCAACCCAGATACATCCA | |
| | AGAATCAGTTCAGCCTGCAGCTGGTGTCTGTGAC | |
| | CCCCGAGGACACAGCCGTGTACTATTGTGCTAGA | |
| | GTGGGCGCCGCTACCTTTGGCATCCTGACAGG | |
| | CGGAATGGACGTGTGGGGACAGGGAACCACAGT | |
| | GACAGTGTCTTCC | |
| 39 | QVQLQQSGPGLVKPSQTLSVTCAISGDSVSSHSAAF | FHF12-VH-W36F |
| | NWIRQSPSRGLEWLGRTYYRSKWYNDYAVSVKSR | (aa) |
| | ITINPDTSKNQFSLQLVSVTPEDTAVYYCARVGAAT | |
| | FGILTGGMDVWGQGTTVTVSS | |
| 40 | CAGGTGCAGCTGCAGCAGTCTGGACCAGGACTGG | FHF12-VH-W59F |
| | TGAAGCCTAGCCAGACCCTGTCTGTGACATGCGC | (nt) |
| | TATCTCCGGCGACAGCGTGTCCAGCCACTCCGC | |
| | CGCTTGGAACTGGATCAGACAGAGCCCATCTAGG | |
| | GGACTGGAGTGGCTGGGAAGGACCTACTATCGG | |
| | AGCAAGTTCTACAATGACTATGCCGTGTCCGTGA | |
| | AGTCCAGGATCACCATCAACCCAGATACATCCAA | |
| | GAATCAGTTCAGCCTGCAGCTGGTGTCTGTGACC | |
| | CCCGAGGACACAGCCGTGTACTATTGTGCTAGAG | |
| | TGGGCGCCGCTACCTTTGGCATCCTGACAGGC | |
| | GGAATGGACGTGTGGGGACAGGGAACCACAGTG | |
| | ACAGTGTCTTCC | |
| 41 | QVQLQQSGPGLVKPSQTLSVTCAISGDSVSSHSAA | FHF12-VH-W59F |
| | WNWIRQSPSRGLEWLGRTYYRSKFYNDYAVSVKS | (aa) |
| | RITINPDTSKNQFSLQLVSVTPEDTAVYYCARVGAA | |
| | TFGILTGGMDVWGQGTTVTVSS | |
| 42 | TYYRSKFYN | FHF12-CDRH2- |
| | | W59F (aa) |
| 43 | QVQLQQSGPGLVKPSQTLSLTCAISGDSVSSYNAVW | FM08 VH |
| | NWIRQSPSRGLEWLGRTYYRSGWYNDYAESVKSRI | |
| | TINPDTSKNQFSLQLNSVTPEDTAVYYCARSGHITVF | |
| | GVNVDAFDMWGQGTMVTVSS | |
| 44 | DIQMTQSPSSLSASVGDRVTITCRTSQSLSSYTHWY | FM08 VL |
| | QQKPGKAPKLLIYAASSRGSGVPSRFSGSGSGTDFT | |
| | LTISSLQPEDFATYYCQQSRTFGQGTKVEIK | |
| 45 | APELLGGPSVFLFPPKPKDTLMISRTPEVTCVVVDVS | WT hIgG1 Fc |
| | HEDPEVKFNWYVDGVEVHNAKTKPREEQYNSTYR | |
| | VVSVLTVLHQDWLNGKEYKCKVSNKALPAPIEKTI | |
| | SKAKGQPREPQVYTLPPSRDELTKNQVSLTCLVKGF | |
| | YPSDIAVEWESNGQPENNYKTTPPVLDSDGSFFLYS | |
| | KLTVDKSRWQQGNVFSCSVMHEALHNHYTQKSLS | |
| | LSPGK | |
| 46 | ESKYGPPCPPCPAPPVAGP | Chimeric hinge |
| | | sequence |
| 47 | CAAGTTCAGCTGGTGCAGTCTGGGGCTGAGGTGA | FNI1 VH (wt-nt) |
| | AGAGGCCTGGGTCCTCGGTGAGGATCTCCTGCAA | |
| | GGCCTCTGGTGACACCTTCAACAACTATGTTCT | |
| | CAGCTGGGTGCGACAGGCCCCTGGACAAGGGCTT | |
| | GAGTGGATGGGGGGAATCATCCCTATCTCTGGT | |
| | ATCCCACATTACGCACAGAAGTTCCAGGGCAGAG | |
| | TCGCAATTATCGCGGACGAATCCGCGAGCACAGT | |
| | CTACATGGAGTTGAGCAGCCTACGATCTGAGGAC | |
| | TCGGCCGTATATTACTGTGCGAGAGCGGTTTCC | |
| | GATTATTTTAATCGAGACCTCGGCTGGGATGAT | |
| | TACTACTTTCCTTTGTGGGGCCAGGGCACCCTGG | |
| | TCACCGTCTCCTCAG | |
| 48 | QVQLVQSGAEVKRPGSSVRISCKASGDTFNNYVLS | FNI1 VH (aa) |
| | WVRQAPGQGLEWMGGIIPISGIPHYAQKFQGRVAII | |
| | ADESASTVYMELSSLRSEDSAVYYCARAVSDYFNR | |
| | DLGWDDYYFPLWGQGTLVTVSS | |
| 49 | GDTFNNYV | FNI1 CDRH1 (aa) |
| 50 | IIPISGIP | FNI1 CDRH2 (aa) |
| 51 | ARAVSDYFNRDLGWDDYYFPL | FNI1 CDRH3 (aa) |
| 52 | CAGGTGCAGCTGGTGCAGTCTGGAGCTGAGGTGAAGA | FNI1 VH (co-nt) |
| | GGCCAGGATCCAGCGTGCGGATCAGCTGCAAGGCTTC | |
| | TGGCGACACCTTCAACAATTACGTGCTGTCCTGGGTG | |
| | AGGCAGGCTCCAGGACAGGGACTGGAGTGGATGGGC | |
| | GGCATCATCCCCATCAGCGGCATCCCTCACTACGCCC | |
| | AGAAGTTTCAGGGCAGGGTGGCCATCATCGCTGACGA | |
| | GTCCGCTAGCACAGTGTATATGGAGCTGTCTTCCCTGA | |
| | GATCTGAGGATTCCGCCGTGTACTATTGTGCCAGAGC | |
| | CGTGTCCGACTATTTCAACCGCGATCTGGGCTGGGAC | |
| | GATTACTATTTTCCACTGTGGGGACAGGGCACCCTGG | |
| | TGACAGTGAGCTCT | |
| 53 | GAAATAGTGATGACGCAGTCTCCAGCCACCCTGT | FNI1 Vk (wt-nt) |
| | CTGTGTCTCCAGGGGAAAGAGCCACCCTCTTCTG | |
| | CAGGGCCAGTCGGAGTGTTAGTGACAACTTAGC | |
| | CTGGTACCAGCAGAAACCTGGCCAGGCTCCCAGG | |
| | CTCCTCATCTTTGGTGCCTCCACCAGGGCCACTG | |
| | GTGTCCCAGCCAGGTTCGGTGGCAGTGGGTCTGG | |
| | GACACAGTTCACTCTCACCATCAGCAGCCTGCAG | |
| | TCTGAAGATTTTGCAGTTTATTACTGTCAGCATT | |
| | ATAATACCTGGCCTCCGTGGACCTTCGGCCAAG | |
| | GGACCAAGGTGGAAATCAAAC | |
| 54 | EIVMTQSPATLSVSPGERATLFCRASRSVSDNLAWY | FNI1 VK (aa) |
| | QQKPGQAPRLLIFGASTRATGVPARFGGSGSGTQFT | |
| | LTISSLQSEDFAVYYCQHYNTWPPWTFGQGTKVEI | |
| | K | |
| 55 | RSVSDN | FNI1 CDRL1 (aa) |
| 56 | GAS | FNI1 CDRL2 (aa) |
| 57 | QHYNTWPPWT | FNI1 CDRL3 (aa) |
| 58 | GAGATCGTGATGACCCAGTCTCCTGCCACACTGT | FNI1 Vk (co-nt) |
| | CCGTGTCCCCAGGCGAGAGGGCCACACTGTTCTG | |
| | CAGGGCTAGCAGGTCCGTGTCCGACAACCTGGCC | |
| | TGGTACCAGCAGAAGCCAGGCCAGGCTCCCAGAC | |
| | TGCTGATCTTTGGAGCTTCCACCAGAGCTACAGG | |
| | CGTGCCAGCTAGGTTCGGAGGAAGCGGATCTGGC | |
| | ACCCAGTTTACCCTGACAATCTCCAGCCTGCAGA | |
| | GCGAGGATTTCGCCGTGTACTATTGTCAGCACTA | |
| | TAATACCTGGCCCCCTTGGACATTTGGCCAGGGC | |
| | ACCAAGGTGGAGATCAAG | |
| 59 | CAGGTTCAGCTGGTGCAGTCTGGGGCTGAGGTGAAGA | FNI2 VH (wt-nt) |
| | GGCCTGGGTCCTCGGTGAGGGTCTCCTGCAAGGCTTC | |
| | TGGAGCCACCTTCAATAACCATGTTCTCACCTGGGT | |
| | GCGACAGGCCCCTGGACAAGGGCTTGAGTGGATGGG | |
| | AGGGATCATCCCTGTCTCTGGAAAAACAACCTACGC | |
| | ACAGAAGTTCCAGGGCAGAGTCGCGATAAGCACGGA | |
| | CGAATCCGCGAGCACAGCCTATATGGAGTTGAGCAGC | |
| | CTGAGATCTGAGGACTCGGCCATATATTACTGTGCGA | |
| | GAGCGGTTTCCGATTACTTTAATCGAGACCTCGGC | |
| | TGGGAAGATTATTACTTTCCGATCTGGGGCCAGGGC | |
| | ACCCTGGTCACCGTCTCTTCAG | |
| 60 | QVQLVQSGAEVKRPGSSVRVSCKASGATFNNHVLT | FNI2 VH (aa) |
| | WVRQAPGQGLEWMGGIIPVSGKTTYAQKFQGRVAI | |
| | STDESASTAYMELSSLRSEDSAIYYCARAVSDYFNR | |
| | DLGWEDYYFPIWGQGTLVTVSS | |
| 61 | GATFNNHV | FNI2 CDRH1 (aa) |
| 62 | IIPVSGKT | FNI2 CDRH2 (aa) |
| 63 | ARAVSDYFNRDLGWEDYYFPI | FNI2 CDRH3 (aa) |
| 64 | CAGGTGCAGCTGGTGCAGTCTGGAGCTGAGGTGA | FNI2 VH (co-nt) |
| | AGAGGCCAGGATCCAGCGTGCGGGTGAGCTGCA | |
| | AGGCTTCTGGAGCTACCTTCAACAATCACGTGCT | |
| | GACATGGGTGAGGCAGGCTCCAGGACAGGGACT | |
| | GGAGTGGATGGGCGGCATCATCCCCGTGTCCGGC | |
| | AAGACCACATACGCCCAGAAGTTTCAGGGCAGG | |
| | GTGGCTATCAGCACCGATGAGTCCGCCAGCACAG | |
| | CTTATATGGAGCTGTCTTCCCTGAGATCTGAGGA | |
| | CTCCGCCATCTACTATTGTGCCAGAGCCGTGTCCG | |
| | ACTACTTCAACCGCGATCTGGGCTGGGAGGACTA | |
| | CTATTTTCCCATCTGGGGCCAGGGCACCCTGGTG | |
| | ACAGTGAGCTCT | |
| 65 | GACGTAGTGATGACGCAGTCTCCAGCCACCCTGT | FNI2 Vk (wt-nt) |
| | CTGTGTCTCCAGGGGAAAGAGCCACCCTCTCCTG | |
| | CAGGGCCAGTCAGAGTGTTAGTAGCAACTTGGC | |
| | CTGGTACCAGCAGAAACCTGGCCAGGCTCCCAGG | |
| | CTCCTCATCTATGGTGCATCCACCAGGGCCACTG | |
| | GTGTCCCAGCCAGGTTCAGTGGCAGTGGGTCTGG | |
| | GACACAGTTCACTCTCACCATCAGCAGCCTGCAG | |
| | TCTGAAGATTTTGCAGTTTATTACTGTCAGCACT | |
| | ATAATAACTGGCCTCCGTGGACGTTCGGCCAAG | |
| | GGACCAAGTTGGAAATCAAAC | |
| 66 | DVVMTQSPATLSVSPGERATLSCRASQSVSSNLAW | FNI2 VK (aa) |
| | YQQKPGQAPRLLIYGASTRATGVPARFSGSGSGTQF | |
| | TLTISSLQSEDFAVYYCQHYNNWPPWTFGQGTKLE | |
| | IK | |
| 67 | QSVSSN | FNI2 CDRL1 (aa) |
| 68 | GAS | FNI2 CDRL2 (aa) |
| 69 | QHYNNWPPWT | FNI2 CDRL3 (aa) |
| 70 | GACGTGGTCATGACCCAGTCTCCTGCCACACTGA | FNI2 Vk (co-nt) |
| | GCGTGTCTCCAGGAGAGAGGGCCACCCTGTCCTG | |
| | CAGGGCTTCCCAGAGCGTGTCCAGCAACCTGGCC | |
| | TGGTACCAGCAGAAGCCAGGCCAGGCTCCCAGGC | |
| | TGCTGATCTATGGAGCTAGCACCAGAGCTACAGG | |
| | CGTGCCAGCTCGCTTCTCTGGATCCGGAAGCGGC | |
| | ACACAGTTTACCCTGACAATCTCTTCCCTGCAGTC | |
| | TGAGGATTTCGCCGTGTACTATTGTCAGCACTAC | |
| | AACAATTGGCCCCCTTGGACCTTTGGCCAGGGCA | |
| | CAAAGCTGGAGATCAAG | |
| 71 | CAGGTTCAGCTGGTGCAGTCGGGGGCTGAGGTGA | FNI3 VH (wt-nt) |
| | AGAGGCCTGGGTCCTCGGTGAAGGTCTCCTGCAA | |
| | GGCTTCTGGAGCCACCTTCAGCAACAATGTTAT | |
| | AGCCTGGGTGCGACAGGCCCCTGGACAAGGGCTT | |
| | GAGTGGATGGGGGGGATCCACCCTATCTCTGCT | |
| | ACAGCAACCTACGCACAGAAGTTCCAGGGCAGAG | |
| | TCGCGATTGCCGCGGACGAATTAACGAGCACAGC | |
| | CTACATGGAGTTGAATGGCCTGAGATCTGAGGAC | |
| | TCGGCCGTGTATTACTGTGCGAGAGCGGGGTCC | |
| | GATTACTTTAATAGAGACCTCGGCTGGGAAAAT | |
| | TACTACTTTGACTCCTGGGGCCAGGGAACCCTGG | |
| | TCACCGTCTCGTCAG | |
| 72 | QVQLVQSGAEVKRPGSSVKVSCKASGATFSNNVIA | FNI3 VH (aa) |
| | WVRQAPGQGLEWMGGIHPISATATYAQKFQGRVA | |
| | IAADELTSTAYMELNGLRSEDSAVYYCARAGSDYF | |
| | NRDLGWENYYFDSWGQGTLVTVSS | |
| 73 | GATFSNNV | FNI3 CDRH1 (aa) |
| 74 | IHPISATA | FNI3 CDRH2 (aa) |
| 75 | ARAGSDYFNRDLGWENYYFDS | FNI3 CDRH3 (aa) |
| 76 | CAGGTGCAGCTGGTGCAGTCCGGAGCTGAGGTGA | FNI3 VH (co-nt) |
| | AGAGGCCAGGATCCAGCGTGAAGGTGTCCTGCAA | |
| | GGCCAGCGGCGCTACCTTCAGCAACAATGTGATC | |
| | GCTTGGGTGAGACAGGCTCCAGGACAGGGACTG | |
| | GAGTGGATGGGAGGAATCCACCCTATCAGCGCCA | |
| | CCGCTACATACGCCCAGAAGTTTCAGGGCAGAGT | |
| | GGCTATCGCCGCTGACGAGCTGACCTCTACAGCC | |
| | TATATGGAGCTGAACGGCCTGCGCAGCGAGGATT | |
| | CCGCCGTGTACTATTGTGCCAGGGCTGGCTCTGA | |
| | CTACTTCAACCGGGATCTGGGCTGGGAGAATTAC | |
| | TATTTTGACTCCTGGGGCCAGGGCACCCTGGTGA | |
| | CAGTGTCTTCC | |
| 77 | GAAATATTGATGACGCAGTCTCCAGCCACCCTGT | FNI3 Vk (wt-nt) |
| | CTGTGTCTCCAGGGGAAAGAGCCACCCTCTCCTG | |
| | CAGGGCCAGTCAGGATGTTAGCGGCAACTTAGC | |
| | CTGGTACCAGCAGAGACCTGGCCAGGCTCCCAGG | |
| | CTCCTTATCTATGGTGCATCCACGAGGGCCACTG | |
| | GTGTCCCAGCCAGGTTCACTGGCGCTGGGTCTGG | |
| | GACAGAGTTCACTCTCACCATCAGCAGCCTGCAG | |
| | TCTGAGGATTTTGCACTTTATTACTGTCAGCACT | |
| | ATAATAACTGGCCTCCGTGGACCTTCGGCCAAG | |
| | GGACCAAGGTGGAAATCAAAC | |
| 78 | EILMTQSPATLSVSPGERATLSCRASQDVSGNLAW | FNI3 Vk (aa) |
| | YQQRPGQAPRLLIYGASTRATGVPARFTGAGSGTEF | |
| | TLTISSLQSEDFALYYCQHYNNWPPWTFGQGTKVE | |
| | IK | |
| 79 | QDVSGN | FNI3 CDRL1 (aa) |
| 80 | GAS | FNI3 CDRL2 (aa) |
| 81 | QHYNNWPPWT | FNI3 CDRL3 (aa) |
| 82 | GAGATCCTGATGACCCAGTCCCCTGCCACACTGTC | FNI3 Vk (co-nt) |
| | CGTGTCCCCAGGAGAGAGGGCCACCCTGAGCTGC | |
| | AGGGCTTCTCAGGACGTGTCCGGCAACCTGGCCT | |
| | GGTACCAGCAGAGACCAGGACAGGCTCCAAGGCT | |
| | GCTGATCTATGGAGCTTCCACCAGGGCTACAGGC | |
| | GTGCCAGCTAGATTCACCGGCGCTGGAAGCGGCA | |
| | CAGAGTTTACCCTGACAATCTCCAGCCTGCAGTCT | |
| | GAGGATTTCGCTCTGTACTATTGTCAGCACTACAA | |
| | CAATTGGCCCCCTTGGACCTTTGGCCAGGGCACAA | |
| | AGGTGGAGATCAAG | |
| 83 | CAGGAGCAGCTGGTACAGTCTGGGGCTGAGGTGA | FNI4 VH (wt-nt) |
| | AGAAGCCGGGGTCCTCGGTGAGGGTCTCCTGCAA | |
| | GGCCTCTGGAGACACCTTCAGCAGATATACTAT | |
| | CAGCTGGGTTCGACAGGCCCCCGGACAAGGACTT | |
| | GAGTGGATGGGAGGGATCATCGCTCTCTCTCGA | |
| | AGAGCGACATACGCACAGAAGTTCCAGGGCAGA | |
| | GTTACCATTACCGCGGACGAATCCGCGACCACAG | |
| | CCTACATACAACTGAGCGGCCTGACATCTGACGA | |
| | CACGGCCGTATATTACTGTGCGAGAGCACACTCC | |
| | GATTACTTTAATAGAGACCTCGGCTGGGAAGAT | |
| | TACTACTTTGACTACTGGGGCCAGGGAACCCTGG | |
| | TCACCGTCTCCTCAG | |
| 84 | QEQLVQSGAEVKKPGSSVRVSCKASGDTFSRYTIS | FNI4 VH (aa) |
| | WVRQAPGQGLEWMGGIIALSRRATYAQKFQGRVT | |
| | ITADESATTAYIQLSGLTSDDTAVYYCARAHSDYFN | |
| | RDLGWEDYYFDYWGQGTLVTVSS | |
| 85 | GDTFSRYT | FNI4 CDRH1 (aa) |
| 86 | IIALSRRA | FNI4 CDRH2 (aa) |
| 87 | ARAHSDYFNRDLGWEDYYFDY | FNI4 CDRH3 (aa) |
| 88 | CAGGAGCAGCTGGTGCAGTCCGGAGCTGAGGTG | FNI4 VH (co-nt) |
| | AAGAAGCCAGGATCCAGCGTGAGAGTGAGCTGC | |
| | AAGGCTTCTGGCGACACCTTCTCTAGATACACAA | |
| | TCTCCTGGGTGCGCCAGGCTCCTGGACAGGGACT | |
| | GGAGTGGATGGGAGGAATCATCGCTCTGAGCAG | |
| | GCGGGCCACCTACGCTCAGAAGTTTCAGGGCCGC | |
| | GTGACCATCACAGCCGATGAGTCTGCCACCACAG | |
| | CTTATATCCAGCTGTCCGGCCTGACCAGCGACGA | |
| | TACAGCCGTGTACTATTGTGCCAGGGCTCACAGC | |
| | GACTACTTCAACCGGGATCTGGGCTGGGAGGACT | |
| | ACTATTTTGATTATTGGGGCCAGGGCACCCTGGT | |
| | GACAGTGTCTTCC | |
| 89 | GAAGTAGTGCTGACGCAGTCTCCAGCCACCCTGT | FNI4 Vk (wt-nt) |
| | CTGTGTCTCTAGGGGAAAGAGCCATCCTCTCCTG | |
| | CAGGGCCAGTCAGAGTGTTAGCACCAACTTAGC | |
| | CTGGTACCAGCAGAGACCTGGCCAGGCTCCCAGG | |
| | CTCCTCATCTCTGGTGCATCCACCAGGGCCACGG | |
| | GTATCCCAGCCAGGTTCAGTGGCAGTGGGTCTGG | |
| | GACAGAGTTCACGCTCACCATCAGCAGCCTGCAG | |
| | TCTGAAGATTTTGCAGTTTATTACTGTCAGCAGT | |
| | ATAATAACTGGCCTCCGTGGACGTTCGGCCAAG | |
| | GGACCAAGGTGGAAATCAGAC | |
| 90 | EVVLTQSPATLSVSLGERAILSCRASQSVSTNLAWY | FNI4 VK (aa) |
| | QQRPGQAPRLLISGASTRATGIPARFSGSGSGTEFTL | |
| | TISSLQSEDFAVYYCQQYNNWPPWTFGQGTKVEIR | |
| 91 | QSVSTN | FNI4 CDRL1 (aa) |
| 92 | GAS | FNI4 CDRL2 (aa) |
| 93 | QQYNNWPPWT | FNI4 CDRL3 (aa) |
| 94 | GAGGTGGTGCTGACCCAGTCCCCTGCCACACTGT | FNI4 Vk (co-nt) |
| | CCGTGTCCCTGGGAGAGAGGGCTATCCTGAGCTG | |
| | CAGGGCTAGCCAGTCCGTGTCCACCAACCTGGCC | |
| | TGGTACCAGCAGAGACCAGGACAGGCTCCAAGG | |
| | CTGCTGATCAGCGGAGCTTCTACCAGGGCTACAG | |
| | GCATCCCAGCCAGATTCAGCGGCTCTGGCTCCGG | |
| | CACAGAGTTTACCCTGACAATCTCCAGCCTGCAG | |
| | TCTGAGGACTTCGCCGTGTACTATTGTCAGCAGT | |
| | ATAACAATTGGCCCCCTTGGACCTTTGGCCAGGG | |
| | CACAAAGGTGGAGATCAGG | |
| 95 | CAGGTGCAGCTGATACAATCTGAGGCTGAGGTGA | FNI5 VH (wt-nt) |
| | AGAAGCCTGGGTCCTCGGTGAGGGTCTCCTGCAA | |
| | GGCTTCTGGAGACACCTTCAGCAAATATACTAT | |
| | CGGCTGGGTGCGACAGGCCCCCGGACAAGGGCTT | |
| | GAGTGGATGGGAGGGATCATCCCTCTCTCTCGA | |
| | ACAGCGACCTACGCACAGAAGTTCCAGGGCAGA | |
| | GTCACGATTACCGCGGACGAATCCACGACCACAG | |
| | TTTACATGCAACTGAGCGGCCTGAGATCTGACGA | |
| | CACGGCCGCATATTACTGTGCGAGAGCACGCTC | |
| | GGATTACTTTAATAGAGACCTCGGCTGGGACG | |
| | ATTACTACTTTGATTACTGGGGCCAGGGAACCC | |
| | TGGTCACCGTCTCCTCAG | |
| 96 | QVQLIQSEAEVKKPGSSVRVSCKASGDTFSKYTIG | FNI5 VH (aa) |
| | WVRQAPGQGLEWMGGIIPLSRTATYAQKFQGRVTI | |
| | TADESTTTVYMQLSGLRSDDTAAYYCARARSDYF | |
| | NRDLGWDDYYFDYWGQGTLVTVSS | |
| 97 | GDTFSKYT | FNI5 CDRH1 (aa) |
| 98 | IIPLSRTA | FNI5 CDRH2 (aa) |
| 99 | ARARSDYFNRDLGWDDYYFDY | FNI5 CDRH3 (aa) |
| 100 | CAGGTGCAGCTGATCCAGAGCGAGGCCGAGGTG | FNI5 VH (co-nt) |
| | AAGAAGCCAGGCTCCAGCGTGAGGGTGAGCTGC | |
| | AAGGCTTCTGGCGACACATTCTCTAAGTACACCA | |
| | TCGGATGGGTGCGGCAGGCTCCAGGACAGGGCCT | |
| | GGAGTGGATGGGCGGCATCATCCCTCTGTCTAGA | |
| | ACAGCCACCTACGCTCAGAAGTTTCAGGGCCGCG | |
| | TGACAATCACCGCTGACGAGTCCACCACAACCGT | |
| | GTATATGCAGCTGTCCGGCCTGAGAAGCGACGAT | |
| | ACAGCCGCTTACTATTGTGCCAGGGCTCGGTCCG | |
| | ACTACTTCAACCGCGATCTGGGCTGGGACGATTA | |
| | CTATTTTGATTATTGGGGCCAGGGCACACTGGTG | |
| | ACCGTGTCTTCC | |
| 101 | GAAATAGTGATGACGCAGTCTCCAGCCAACCTGT | FNI5 Vk (wt-nt) |
| | CTGTGTCTCCAGGGGAAAGAGCCACCCTCTCCTG | |
| | CAGGGCCAGTCAGACTGTTAGCACCAACTTAGC | |
| | CTGGTACCAGCAGAAGCCTGGCCAGGCTCCCAGG | |
| | CTCCTCATCTCTGGTGCATCCACCAGGGCCACTG | |
| | GTATCCCAGCCAGGTTCAGTGGCAGTGGGTCTGG | |
| | GACAGAGTTCACGCTCACCATCAGCAGCCTGCAG | |
| | TCTGAAGATTTTGCAGTTTATTACTGTCAGCAGT | |
| | ATAATAATTGGCCTCCGTGGACGTTCGGCCAAG | |
| | GGACCAAGGTGGAAATCAGAC | |
| 102 | EIVMTQSPANLSVSPGERATLSCRASQTVSTNLAW | FNI5 VK (aa) |
| | YQQKPGQAPRLLISGASTRATGIPARFSGSGSGTEFT | |
| | LTISSLQSEDFAVYYCQQYNNWPPWTFGQGTKVEI | |
| | R | |
| 103 | QTVSTN | FNI5 CDRL1 (aa) |
| 104 | GAS | FNI5 CDRL2 (aa) |
| 105 | QQYNNWPPWT | FNI5 CDRL3 (aa) |
| 106 | GAGATCGTGATGACCCAGTCCCCTGCTAACCTGT | FNI5 Vk (co-nt) |
| | CCGTGTCCCCAGGAGAGAGGGCCACACTGTCCTG | |
| | CCGGGCTAGCCAGACCGTGTCTACAAATCTGGCC | |
| | TGGTACCAGCAGAAGCCAGGACAGGCTCCAAGG | |
| | CTGCTGATCAGCGGAGCTTCTACCAGAGCTACAG | |
| | GCATCCCAGCTCGCTTCAGCGGATCTGGATCCGG | |
| | CACCGAGTTTACCCTGACAATCTCCAGCCTGCAG | |
| | AGCGAGGACTTCGCCGTGTACTATTGTCAGCAGT | |
| | ATAACAATTGGCCCCCTTGGACCTTTGGCCAGGG | |
| | CACAAAGGTGGAGATCAGA | |
| 107 | CAGGTGCAGCTGGTGCAGTCTGGGGCTGAGGTGA | FNI6 VH (wt-nt) |
| | AGAAGCCTGGGTCCTCGGTGAAGGTCTCCTGCAA | |
| | GGCCTCTGGAGGCACCTTCAGTAGTCAAGTTAT | |
| | CAGCTGGGTGCGAGAGGCCCCAGGACAAGGGCT | |
| | TGAGTGGATGGGAGGGATCATTCCTATCACTGG | |
| | AATAGCGAACAACGCACAGAAGTTCCAGGGCAG | |
| | AGTCACGATTACCGCGGACGGATCCACGGGCACA | |
| | GTCTACATGGAGTTGAGCAGCCTGAGATCTGGGG | |
| | ACACGGCCGTCTATTACTGTGCGAGAGCGGGTT | |
| | CGGATTATTTTAATAGAGACCTCGGCTGGGAA | |
| | AATTACTACTTTGAATATTGGGGCCAGGGAACC | |
| | CTGGTCACCGTCTCCTCAG | |
| 108 | QVQLVQSGAEVKKPGSSVKVSCKASGGTFSSQVIS | FNI6 VH (aa) |
| | WVREAPGQGLEWMGGIIPITGIANNAQKFQGRVTI | |
| | TADGSTGTVYMELSSLRSGDTAVYYCARAGSDYF | |
| | NRDLGWENYYFEYWGQGTLVTVSS | |
| 109 | GGTFSSQV | FNI6 CDRH1 (aa) |
| 110 | IIPITGIA | FNI6 CDRH2 (aa) |
| 111 | ARAGSDYFNRDLGWENYYFEY | FNI6 CDRH3 (aa) |
| 112 | CAGGTGCAGCTGGTGCAGAGCGGAGCTGAGGTGA | FNI6 VH (co-nt) |
| | AGAAGCCAGGCTCCAGCGTGAAGGTGTCTTGCAA | |
| | GGCTTCCGGCGGCACCTTCTCTTCCCAGGTCATCT | |
| | CTTGGGTGAGGGAGGCTCCAGGACAGGGACTGGA | |
| | GTGGATGGGCGGCATCATCCCTATCACAGGCATC | |
| | GCCAACAATGCTCAGAAGTTTCAGGGCAGAGTGA | |
| | CCATCACAGCCGACGGCAGCACCGGCACAGTGTA | |
| | CATGGAGCTGAGCTCTCTGCGCTCTGGCGATACCG | |
| | CCGTGTACTATTGTGCCAGGGCTGGCTCCGACTAC | |
| | TTCAACCGGGATCTGGGCTGGGAGAATTACTATTT | |
| | TGAGTATTGGGGCCAGGGCACCCTGGTGACAGTG | |
| | TCCAGC | |
| 113 | GAAATCGTGATGACACAGTCTCCAGCCACCCTGT | FNI6 Vk (wt-nt) |
| | CTGTATCTCCAGGGGAAAGAGCCATCCTCTCCTG | |
| | CAGGGCCAGTCAGAGTGTTAGCACCCACTTAGC | |
| | CTGGTACCAGCAGAAACCTGGCCAGGCTCCCAGA | |
| | CTCCTCGTTTTTGATGCATCCACCAGGGCCACTG | |
| | GTGTCCCAGCCAGATTCGGTGGCAGTGGGTCTGG | |
| | GACAGAGTTCACTCTCACCATCAGCAGCCTGCAG | |
| | TCTGAAGATTCTGCTGTTTATTACTGTCAACACTA | |
| | TAATAACTGGCCTCCGTGGACGTTCGGCCAAGG | |
| | GACCAACGTGGAAATCAGAC | |
| 114 | EIVMTQSPATLSVSPGERAILSCRASQSVSTHLAWY | FNI6 VK (aa) |
| | QQKPGQAPRLLVFDASTRATGVPARFGGSGSGTEFT | |
| | LTISSLQSEDSAVYYCQHYNNWPPWTFGQGINVEI | |
| | R | |
| 115 | QSVSTH | FNI6 CDRL1 (aa) |
| 116 | DAS | FNI6 CDRL2 (aa) |
| 117 | QHYNNWPPWT | FNI6 CDRL3 (aa) |
| 118 | GAGATCGTGATGACCCAGTCTCCTGCCACACTGT | FNI6 Vk (co-nt) |
| | CCGTGTCCCCAGGAGAGAGGGCTATCCTGTCCTG | |
| | CAGGGCTAGCCAGTCCGTGTCCACCCACCTGGCC | |
| | TGGTACCAGCAGAAGCCAGGCCAGGCTCCCAGGC | |
| | TGCTGGTGTTCGACGCTAGCACCAGAGCTACAGG | |
| | CGTGCCAGCTAGGTTCGGAGGAAGCGGATCTGGC | |
| | ACAGAGTTTACCCTGACAATCTCCAGCCTGCAGT | |
| | CCGAGGATTCCGCCGTGTACTATTGTCAGCATTAT | |
| | AACAATTGGCCCCCTTGGACCTTTGGCCAGGGCA | |
| | CAAACGTGGAGATCAGA | |
| 119 | CAAGTGCAGCTGGTGCAGTCTGGGGCTGAGGTGA | FNI7 VH (wt-nt) |
| | AGAAGCCTGGGTCCTCGGTGAAAGTCTCCTGTAA | |
| | GACTTCTGGAGGCACCTTCAATAGGCAAGTTAT | |
| | CAGCTGGGTGCGACAGGCCCCAGGACAAGGACTT | |
| | GAGTGGATGGGAGGGATCCTCCCTCTTACTGGT | |
| | AGAGGGGACGAGGCAGAGAGGTTTCAGGGCAGA | |
| | GTCACCATTACCGCGGACGAATCTGAGAGTACAG | |
| | TCTACATGGACTTGAGCAGCCTGAGATCTGGGGA | |
| | CACGGCCGTCTATTACTGTGCGAGAGCGCGTTC | |
| | GGATTACTTTAATAGAGACCTCGGCTGGGAAA | |
| | ATTACTACTTTGAATCTTGGGGCCAGGGAACCC | |
| | TGGTCACCGTCTCCTCAG | |
| 120 | QVQLVQSGAEVKKPGSSVKVSCKTSGGTFNRQVIS | FNI7 VH |
| | WVRQAPGQGLEWMGGILPLTGRGDEAERFQGRV | (aa) |
| | TITADESESTVYMDLSSLRSGDTAVYYCARARSDY | |
| | FNRDLGWENYYFESWGQGTLVTVSS | |
| 121 | GGTFNRQV | FNI7 CDRH1 (aa) |
| 122 | ILPLTGRG | FNI7 CDRH2 (aa) |
| 123 | ARARSDYFNRDLGWENYYFES | FNI7 CDRH3 (aa) |
| 124 | CAGGTGCAGCTGGTGCAGTCCGGAGCTGAGGTGA | FNI7 VH (co-nt) |
| | AGAAGCCAGGCTCCAGCGTGAAGGTGTCTTGCAA | |
| | GACCTCCGGCGGCACATTCAACAGGCAGGTCATC | |
| | AGCTGGGTGCGGCAGGCTCCAGGACAGGGACTG | |
| | GAGTGGATGGGAGGAATCCTGCCTCTGACCGGCA | |
| | GGGGCGACGAGGCCGAGAGATTTCAGGGCCGCG | |
| | TGACCATCACAGCTGATGAGTCCGAGAGCACCGT | |
| | GTACATGGACCTGTCTTCCCTGAGAAGCGGCGAT | |
| | ACAGCCGTGTACTATTGTGCCAGGGCTCGGTCTG | |
| | ACTATTTCAACCGCGATCTGGGCTGGGAGAATTA | |
| | CTATTTTGAGTCTTGGGGCCAGGGCACCCTGGTG | |
| | ACAGTGAGCTCT | |
| 125 | GAAATCGTGATGACGCAGTCTCCAGCCACCCTGT | FNI7 Vk (wt-nt) |
| | CTGTATCTCCAGGGGAAAGAGCCACCCTCTCCTG | |
| | CAGGGCCAGTCAGAGTGTTAGTACCGACTTAGT | |
| | CTGGTACCAGCAGAAACCTGGCCAGGCTCCCCGG | |
| | CTCCTCATTTATGATGCATCCACTAGGGCCACTG | |
| | GTATCCCAGCCAGGTTCGGTGGCAGGGGGTCTGG | |
| | GACAGAGTTCACTCTCACCATCAGCAGCCTGCAG | |
| | TCTGAAGATTCTGCTGTTTATTACTGTCAGCACT | |
| | ATTCTTACTGGCCTCCGTGGACATTCGGCCAAG | |
| | GGACCAAAGTGGAAATCAATC | |
| 126 | EIVMTQSPATLSVSPGERATLSCRASQSVSTDLVWY | FNI7 VK (aa) |
| | QQKPGQAPRLLIYDASTRATGIPARFGGRGSGTEFT | |
| | LTISSLQSEDSAVYYCQHYSYWPPWTFGQGTKVEI | |
| | N | |
| 127 | QSVSTD | FNI7 CDRL1 (aa) |
| 128 | DAS | FNI7 CDRL2 (aa) |
| 129 | QHYSYWPPWT | FNI7 CDRL3 (aa) |
| 130 | GAGATCGTGATGACCCAGTCCCCTGCCACACTGT | FNI7 Vk (co-nt) |
| | CCGTGTCCCCAGGAGAGAGAGCCACCCTGAGCTG | |
| | CAGGGCTAGCCAGTCCGTGTCCACAGACCTGGTG | |
| | TGGTACCAGCAGAAGCCAGGACAGGCTCCAAGG | |
| | CTGCTGATCTATGATGCCTCTACCAGAGCTACAG | |
| | GCATCCCAGCTAGGTTCGGAGGAAGGGGATCCGG | |
| | CACCGAGTTTACCCTGACAATCTCCAGCCTGCAG | |
| | AGCGAGGACTCCGCCGTGTACTATTGTCAGCACT | |
| | ACAGCTATTGGCCCCCTTGGACCTTCGGCCAGGG | |
| | CACAAAGGTGGAGATCAAC | |
| 131 | CAGGTCCACCTGGTGCAGTCTGGGGCTGAGGTGA | FNI9 VH (wt-nt) |
| | AGGAGCCTGGGTCCTCGGTGACGGTCTCCTGCAA | |
| | GGCATCTGGAGGCAGCTTCAACAACCAGGCTA | |
| | TTAGCTGGGTGCGACAGGCCCCAGGACAAGGCCT | |
| | TGAGTGGATGGGAGGGATCTTCCCTATCTCTGG | |
| | CACACCGACCAGCGCACAGAGGTTCCAGGGCAG | |
| | AGTCACATTTACCGCGGACGAGTCCACGACCACA | |
| | GTCTACATGGATCTGAGCAGCCTGAGATCTGACG | |
| | ACACGGCCGTCTACTACTGTGCGAGAGCGGGTT | |
| | CGGATTACTTTAATAGAGACCTCGGCTGGGAA | |
| | AACTACTACTTTGCGTCCTGGGGCCAGGGAACC | |
| | CTGGTCACCGTCTCCTCAG | |
| 132 | QVHLVQSGAEVKEPGSSVTVSCKASGGSFNNQAIS | FNI9 VH (aa) |
| | WVRQAPGQGLEWMGGIFPISGTPTSAQRFQGRVTF | |
| | TADESTTTVYMDLSSLRSDDTAVYYCARAGSDYFN | |
| | RDLGWENYYFASWGQGTLVTVSS | |
| 133 | GGSFNNQA | FNI9 CDRH1 (aa) |
| 134 | IFPISGTP | FNI9 CDRH2 (aa) |
| 135 | ARAGSDYFNRDLGWENYYFAS | FNI9 CDRH3 (aa) |
| 136 | CAGGTGCACCTGGTGCAGAGCGGAGCTGAGGTG | FNI9 VH (co-nt) |
| | AAGGAGCCAGGATCCAGCGTGACAGTGTCTTGCA | |
| | AGGCTTCCGGCGGCAGCTTCAACAATCAGGCTAT | |
| | CTCCTGGGTGAGGCAGGCTCCAGGACAGGGACTG | |
| | GAGTGGATGGGCGGCATCTTTCCCATCTCTGGCA | |
| | CACCTACCTCCGCCCAGAGGTTCCAGGGAAGGGT | |
| | GACCTTCACCGCTGACGAGAGCACCACAACCGTG | |
| | TACATGGATCTGTCTTCCCTGAGATCTGACGATAC | |
| | CGCCGTGTACTATTGTGCCAGAGCTGGCTCCGAC | |
| | TATTTCAACCGCGATCTGGGCTGGGAGAATTACT | |
| | ATTTTGCTTCCTGGGGCCAGGGCACACTGGTGAC | |
| | CGTGAGCTCT | |
| 137 | GAAATCGTGATGACGCAGTCTCCAGCCACCCTGT | FNI9 Vk (wt-nt) |
| | CTCTATCTTCAGGGGAAAGAGCCACCCTCTCCTG | |
| | CAGGGCCAGTCGGAGTGTTAGTAGCAACTTAGC | |
| | CTGGTACCAGCAGAAACCTGGCCAGGCTCCCAGG | |
| | CTCCTCATTTATGATGCATCCACCAGGGCCACTG | |
| | GTTTTTCAGCCAGGTTCGCTGGCAGTGGGTCTGG | |
| | GACAGAGTTCACTCTCACCATCAGCAGCCTGCAG | |
| | TCTGAAGATTCTGCTATTTATTACTGTCAGCAGT | |
| | ATAATAACTGGCCTCCGTGGACGTTCGGCCAAG | |
| | GGACCAAGGTGGAAATCAAAC | |
| 138 | EIVMTQSPATLSLSSGERATLSCRASRSVSSNLAWY | FN19 VK (aa) |
| | QQKPGQAPRLLIYDASTRATGFSARFAGSGSGTEFT | |
| | LTISSLQSEDSAIYYCQQYNNWPPWTFGQGTKVEI | |
| | K | |
| 139 | RSVSSN | FNI9 CDRL1 (aa) |
| 140 | DAS | FNI9 CDRL2 (aa) |
| 141 | QQYNNWPPWT | FNI9 CDRL3 (aa) |
| 142 | GAGATCGTGATGACCCAGTCCCCAGCCACACTGA | FNI9 Vk (co-nt) |
| | GCCTGTCCAGCGGAGAGAGGGCCACCCTGTCCTG | |
| | CAGGGCTTCCCGGAGCGTGTCTTCCAACCTGGCC | |
| | TGGTACCAGCAGAAGCCAGGCCAGGCTCCCAGAC | |
| | TGCTGATCTATGACGCCTCTACCAGAGCTACAGG | |
| | CTTCTCCGCCAGGTTTGCTGGATCTGGATCCGGCA | |
| | CAGAGTTCACCCTGACAATCAGCTCTCTGCAGAG | |
| | CGAGGATTCTGCTATCTACTATTGTCAGCAGTAC | |
| | AACAATTGGCCCCCTTGGACCTTTGGCCAGGGCA | |
| | CAAAGGTGGAGATCAAG | |
| 143 | CAGGTGCAGCTGGTGCAGTCTGGGGCTGAGGTGA | FNI10 VH (wt-nt) |
| | AGAAGCCTGGGTCCTCGGTGAAAGTCTCCTGCAA | |
| | GGCTTCTGGAGGCACCTTGAGTAGTCAAGTTAT | |
| | TAGCTGGGTGCGACAGGCCCCAGGACAAGGACT | |
| | GGAGTGGATCGGAGGGATCATCCCCACCACTGG | |
| | TACAGGGGGCGCGGCAGAGGGGTTCCAGGGCAG | |
| | AGTCTCCATTTCCGCGGACGAATCCAGGAGCACA | |
| | GTCTACATGGAACTGACCAGCCTGACTTCTGGGG | |
| | ACACGGCCGTCTATTATTGTGCGAGAGCGGTTT | |
| | CGGATTACTTTAATAGAGACCTCGGCTGGGAA | |
| | AATTACTACTTTGAATCTTGGGGCCAGGGAACC | |
| | CTGGTCACCGTCTCCTCAG | |
| 144 | QVQLVQSGAEVKKPGSSVKVSCKASGGTLSSQVIS | FNI10 VH (aa) |
| | WVRQAPGQGLEWIGGIIPTTGTGGAAEGFQGRVSIS | |
| | ADESRSTVYMELTSLTSGDTAVYYCARAVSDYFNR | |
| | DLGWENYYFESWGQGTLVTVSS | |
| 145 | GGTLSSQV | FNI10 CDRH1 (aa) |
| 146 | IIPTTGTG | FNI10 CDRH2 (aa) |
| 147 | ARAVSDYFNRDLGWENYYFES | FNI10 CDRH3 (aa) |
| 148 | CAGGTGCAGCTGGTGCAGAGCGGAGCTGAGGTGA | FNI10 VH (co-nt) |
| | AGAAGCCAGGCTCCAGCGTGAAGGTGTCCTGCAA | |
| | GGCTAGCGGCGGCACCCTGTCTTCCCAGGTCATCT | |
| | CTTGGGTGAGGCAGGCTCCAGGACAGGGACTGGA | |
| | GTGGATCGGCGGCATCATCCCTACCACAGGCACA | |
| | GGCGGAGCTGCTGAGGGATTCCAGGGCAGAGTGT | |
| | CCATCAGCGCCGACGAGTCTCGCTCCACCGTGTAC | |
| | ATGGAGCTGACCAGCCTGACATCTGGCGATACAG | |
| | CCGTGTACTATTGTGCCAGGGCCGTGTCCGACTAT | |
| | TTCAACCGGGATCTGGGCTGGGAGAATTACTATTT | |
| | TGAGTCCTGGGGCCAGGGCACCCTGGTGACAGTG | |
| | AGCTCT | |
| 149 | GAAATCGTGATGACGCAGTCTCCAGCCACCCTGT | FNI10 Vk (wt-nt) |
| | CTGTGTCTCCAGGGGAAAGAGCCACCCTCTCTTG | |
| | CAGGGCCAGTCGGAGTGTTAGTATCAACTTAGC | |
| | CTGGTACCAACAGAAACCTGGCCAGGCTCCCCGG | |
| | CTCCTCATTTATGATGCATCTACGAGGGCCACTG | |
| | GCATCCCAGCCAGGTTCGGTGGCAGGGGGTCTGG | |
| | AACAGAGTTCACTCTCACCATCAGCAGCCTGCAG | |
| | TCTGAAGATTCTGCTGTTTATTACTGTCAGCACT | |
| | ATAATAACTGGCCTCCGTGGACATTCGGCCAAG | |
| | GGACCAGAGTGGAAATCAAAC | |
| 150 | EIVMTQSPATLSVSPGERATLSCRASRSVSINLAWYQ | FNI10 VK (aa) |
| | QKPGQAPRLLIYDASTRATGIPARFGGRGSGTEFTLT | |
| | ISSLQSEDSAVYYCQHYNNWPPWTFGQGTRVEIK | |
| 151 | RSVSIN | FNI10 CDRL1 (aa) |
| 152 | DAS | FNI10 CDRL2 (aa) |
| 153 | QHYNNWPPWT | FNI10 CDRL3 (aa) |
| 154 | GAGATCGTGATGACCCAGTCCCCTGCCACACTGTCCGT | FNI10 Vk (co-nt) |
| | GTCCCCAGGAGAGAGAGCCACCCTGAGCTGCAGGGCT | |
| | AGCAGGTCCGTGTCCATCAACCTGGCCTGGTACCAGCA | |
| | GAAGCCAGGCCAGGCTCCCAGGCTGCTGATCTATGAC | |
| | GCTTCTACCAGGGCTACAGGCATCCCAGCTAGATTCGG | |
| | AGGAAGGGGATCCGGAACAGAGTTTACCCTGACAATC | |
| | TCCAGCCTGCAGAGCGAGGATTCCGCCGTGTACTATTG | |
| | TCAGCACTACAACAATTGGCCACCTTGGACCTTCGGCC | |
| | AGGGAACACGCGTGGAGATCAAG | |
| 155 | CAGGTGCACCTGGTACAGTCTGGGGCTGAGGTGA | FNI12 VH (wt-nt) |
| | AGAAGCCTGGGTCCTCGGTGAGGGTCTCCTGCAA | |
| | GGCTTCTGGAGACTCCTTCAACAAATATGAAGT | |
| | CAGCTGGGTGCGACAGGCCCCCGGACATGGACTT | |
| | GAGTGGATGGGAGGGATCATCCCTCTCTCTCCT | |
| | ATAGCGAGGTACGCAGAGAAATTTCAGGGCAGA | |
| | GTCACGATTACCGCGGACGAATTCACGAGCACGG | |
| | TCTATATACAACTGACCAGCCTGAGATCTGACGAC | |
| | ACGGCCGTATACTACTGTGCGACAACACGTTCG | |
| | GATTACTTTAATAGAGACCTCGGCTGGGAAGAT | |
| | TACTTCTTTGACCACTGGGGCCAGGGAACCCTGG | |
| | TCACCGTCTCCTCAG | |
| 156 | QVHLVQSGAEVKKPGSSVRVSCKASGDSFNKYEVS | FNI12 VH (aa) |
| | WVRQAPGHGLEWMGGIIPLSPIARYAEKFQGRVTIT | |
| | ADEFTSTVYIQLTSLRSDDTAVYYCATTRSDYFNRD | |
| | LGWEDYFFDHWGQGTLVTVSS | |
| 157 | GDSFNKYE | FNI12 CDRH1 (aa) |
| 158 | IIPLSPIA | FNI12 CDRH2 (aa) |
| 159 | ATTRSDYFNRDLGWEDYFFDH | FNI12 CDRH3 (aa) |
| 160 | CAGGTGCACCTGGTGCAGTCTGGCGCCGAGGTGA | FNI12 VH (co-nt) |
| | AGAAGCCAGGCTCCAGCGTGAGGGTGTCCTGCAA | |
| | GGCTAGCGGCGACTCTTTCAACAAGTACGAGGTG | |
| | AGCTGGGTGAGACAGGCTCCAGGACATGGACTGG | |
| | AGTGGATGGGCGGCATCATCCCCCTGTCTCCTATC | |
| | GCCAGATACGCTGAGAAGTTCCAGGGCCGCGTGA | |
| | CCATCACAGCTGATGAGTTTACCTCCACAGTGTAT | |
| | ATCCAGCTGACCTCCCTGAGGAGCGACGATACAG | |
| | CCGTGTACTATTGTGCTACCACAAGGAGCGACTAC | |
| | TTTAATCGGGATCTGGGCTGGGAGGACTATTTCTT | |
| | TGATCACTGGGGCCAGGGCACCCTGGTGACAGTG | |
| | TCTTCC | |
| 161 | GAAATAGTGATGACGCAGTCTCCAGCCACCCTGT | FNI12 Vk (wt-nt) |
| | CTGTGTCTCCAGGGGAAAGAGCCACCCTCTCCTGC | |
| | AGGGCCAGTCAGAGTATTAGCACCAACTTAGCC | |
| | TGGTACCAGCAGAAACCTGGCCAGGCTCCCAGGC | |
| | TCCTCATCTCTGGTGCATCCACCAGGGCCACTGG | |
| | TATCCCAGCCAGGTTCAGTGGCAGTGGGTCTGGG | |
| | ACAGAGTTCACTCTCACCATCAGCAGCCTGCAGTC | |
| | TGAAGATTTTGGAGTTTATTACTGTCAGCACTATA | |
| | ATAACTGGCCTCCGTGGACGTTCGGCCAAGGGA | |
| | CCAAGGTGGAAATCAAAC | |
| 162 | EIVMTQSPATLSVSPGERATLSCRASQSISTNLAWYQ | FNI12 VK (aa) |
| | QKPGQAPRLLISGASTRATGIPARFSGSGSGTEFTLTI | |
| | SSLQSEDFGVYYCQHYNNWPPWTFGQGTKVEIK | |
| 163 | QSISTN | FNI12 CDRL1 (aa) |
| 164 | GAS | FNI12 CDRL2 (aa) |
| 165 | QHYNNWPPWT | FNI12 CDRL3 (aa) |
| 166 | GAGATCGTGATGACCCAGTCCCCTGCCACACTGTC | FNI12 Vk (co-nt) |
| | CGTGTCCCCAGGAGAGAGGGCCACCCTGAGCTGC | |
| | CGGGCTAGCCAGTCTATCTCCACAAACCTGGCCTG | |
| | GTACCAGCAGAAGCCAGGACAGGCTCCAAGGCTG | |
| | CTGATCAGCGGAGCTTCTACCAGAGCTACAGGCA | |
| | TCCCAGCTCGCTTCAGCGGATCTGGATCCGGAACC | |
| | GAGTTTACCCTGACAATCTCCAGCCTGCAGTCTGA | |
| | GGACTTCGGCGTGTACTATTGTCAGCACTATAACA | |
| | ATTGGCCCCCTTGGACCTTTGGCCAGGGCACAAA | |
| | GGTGGAGATCAAG | |
| 167 | CAGGTTCAGCTGGTGCAATCTGGGGCTGAGGTGA | FNI13 VH (wt-nt) |
| | AGAGGCCTGGGTCCTCGGTGAGGGTCTCCTGCAA | |
| | GGGTTCTGGAGACACCTTCAACAACTATGTTAT | |
| | CAGTTGGGTGCGACAGGCCCCTGGCCAAGGGCTT | |
| | GAGTGGATGGGGGGGATCATCCCTATCTTTCAA | |
| | ACACCAAACTACGCAGAGAAGTTCCAGGGCAGA | |
| | GTCGCGATTACCGCGGACGAATCCACGAGCACGG | |
| | CCTACATGGAGTTGAGCAGCCTGAGATCTGAGGA | |
| | CTCGGCCATTTATTACTGTGCGAGAGCGAATTCC | |
| | GATTACTTTAATAGAGACCTCGGCTGGGAAAAT | |
| | TACTACTTTGAAGACTGGGGCCAGGGAACCCTG | |
| | GTCACCGTCTCCTCAG | |
| 168 | QVQLVQSGAEVKRPGSSVRVSCKGSGDTFNNYVIS | FNI13 VH (aa) |
| | WVRQAPGQGLEWMGGIIPIFQTPNYAEKFQGRVAI | |
| | TADESTSTAYMELSSLRSEDSAIYYCARANSDYFNR | |
| | DLGWENYYFEDWGQGTLVTVSS | |
| 169 | GDTFNNYV | FNI13 CDRH1 (aa) |
| 170 | IIPIFQTP | FNI13 CDRH2 (aa) |
| 171 | ARANSDYFNRDLGWENYYFED | FNI13 CDRH3 (aa) |
| 172 | CAGGTGCAGCTGGTGCAGTCCGGAGCTGAGGTGA | FNI13 VH (co-nt) |
| | AGAGGCCAGGATCCAGCGTGCGGGTGAGCTGCAA | |
| | GGGATCTGGCGACACCTTCAACAATTACGTGATC | |
| | AGCTGGGTGAGGCAGGCTCCAGGACAGGGACTGG | |
| | AGTGGATGGGCGGCATCATCCCCATCTTCCAGACC | |
| | CCTAACTACGCTGAGAAGTTTCAGGGCAGGGTGG | |
| | CCATCACAGCTGACGAGTCCACCAGCACAGCCTA | |
| | TATGGAGCTGTCTTCCCTGAGATCTGAGGATTCCG | |
| | CTATCTACTATTGTGCCAGAGCTAACTCTGACTAT | |
| | TTCAATCGCGATCTGGGCTGGGAGAATTACTATTT | |
| | TGAGGATTGGGGCCAGGGCACCCTGGTGACAGTG | |
| | AGCTCT | |
| 173 | GAAAGAGTGATGACGCAGTCTCCAGCCACCCTTT | FNI13 Vk (wt-nt) |
| | CTGTGTCTCCAGGGGGAAGAGCCACCCTCTCCTGC | |
| | AGGGCCAGTCAGAGTGTTGGTAGCAACTTAGCC | |
| | TGGTACCAGCAGAAACCTGGCCAGGCTCCCAGGC | |
| | TCCTCATCTATGATGCTTCTGCCAGGGCCACTGG | |
| | TGTCCCAGCCAGGTTCAGTGGCAGTGGGTCTGGG | |
| | ACAGAGTTCTCTCTCTCCATCAACAGCCTGCAGTC | |
| | TGAAGATTCTGCAGTTTATTACTGTCAGCACTATA | |
| | ATATCTGGCCGCCGTGGACGTTCGGCCAAGGGA | |
| | CCAAGGTGGAAATCAAAC | |
| 174 | ERVMTQSPATLSVSPGGRATLSCRASQSVGSNLAW | FNI13 VK (aa) |
| | YQQKPGQAPRLLIYDASARATGVPARFSGSGSGTEF | |
| | SLSINSLQSEDSAVYYCQHYNIWPPWTFGQGTKVEI | |
| | K | |
| 175 | QSVGSN | FNI13 CDRL1 (aa) |
| 176 | DAS | FNI13 CDRL2 (aa) |
| 177 | QHYNIWPPWT | FNI13 CDRL3 (aa) |
| 178 | GAGAGAGTGATGACCCAGTCTCCTGCTACACTGTC | FNI13 Vk (co-nt) |
| | CGTGAGCCCAGGAGGAAGGGCTACCCTGTCCTGC | |
| | AGGGCTTCTCAGTCCGTGGGAAGCAACCTGGCTT | |
| | GGTACCAGCAGAAGCCAGGCCAGGCCCCCAGACT | |
| | GCTGATCTATGACGCTTCCGCTAGAGCTACCGGCG | |
| | TGCCAGCTCGCTTCAGCGGATCTGGCTCCGGCACA | |
| | GAGTTTAGCCTGTCTATCAACTCCCTGCAGAGCGA | |
| | GGATTCTGCCGTGTACTATTGTCAGCACTACAATA | |
| | TCTGGCCACCTTGGACCTTCGGCCAGGGAACAAA | |
| | GGTGGAGATCAAG | |
| 179 | CAAGTTCAGTTGGTGCAGTCTGGGGCTGAGCTGA | FNI14 VH (wt-nt) |
| | AGCGGCCTGGGTCCTCGGTGAGGATCTCCTGCAA | |
| | GGCCTCTGGTGTCACCTTCAACAAGTATGTTCTC | |
| | AGCTGGGTGCGACTGGCCCCTGGACAAGGGCTTG | |
| | AGTGGATGGGAGGAATCATCCCTATTTCTGGTA | |
| | TACCACATTACGCAGAGAAGTTCCAGGGCAGAGT | |
| | CGCGATTACCGCGGACGAATCCACGAGCACAGTC | |
| | TACATGGAGTTGAGCAGCCTACGATCTGAGGACT | |
| | CGGCCCTATATTACTGTGCGAGAGCGGTCTCCG | |
| | ATTATTTTAATCGGGACCTCGGCTGGGATGATT | |
| | ACTACTTTCCTTTGTGGGGCCACGGCACCCTGGT | |
| | CACCGTCTCCTCAG | |
| 180 | QVQLVQSGAELKRPGSSVRISCKASGVTFNKYVLS | FNI14 VH (aa) |
| | WVRLAPGQGLEWMGGIIPISGIPHYAEKFQGRVAIT | |
| | ADESTSTVYMELSSLRSEDSALYYCARAVSDYFNR | |
| | DLGWDDYYFPLWGHGTLVTVSS | |
| 181 | GVTFNKYV | FNI14 CDRH1 (aa) |
| 182 | IIPISGIP | FNI14 CDRH2 (aa) |
| 183 | ARAVSDYFNRDLGWDDYYFPL | FNI14 CDRH3 (aa) |
| 184 | CAGGTGCAGCTGGTGCAGTCTGGAGCTGAGCTGA | FNI14 VH (co-nt) |
| | AGAGGCCAGGATCCAGCGTGCGGATCAGCTGCAA | |
| | GGCTTCTGGCGTGACCTTCAACAAGTACGTGCTGT | |
| | CCTGGGTGAGGCTGGCTCCAGGACAGGGACTGGA | |
| | GTGGATGGGCGGCATCATCCCCATCAGCGGCATC | |
| | CCTCACTACGCTGAGAAGTTTCAGGGCAGGGTGG | |
| | CCATCACAGCTGACGAGTCCACCAGCACAGTGTA | |
| | TATGGAGCTGTCTTCCCTGAGATCTGAGGATTCCG | |
| | CCCTGTACTATTGTGCCAGAGCCGTGTCCGACTAT | |
| | TTCAATCGCGATCTGGGCTGGGACGATTACTATTT | |
| | TCCCCTGTGGGGCCATGGCACCCTGGTGACAGTG | |
| | AGCTCT | |
| 185 | GAAATAGTGATGACGCAGTCTCCAGCCACCCTGT | FNI14 Vk (wt-nt) |
| | CTGTGTCTCCAGGGGAAAGCGCCACCCTCTTCTGC | |
| | AGGGCCAGTCGGAGTGTTAGTGACAACTTAGCC | |
| | TGGTACCAGCAGAAACCTGGCCAGGCTCCCAGGC | |
| | TCCTCATCTTTGGTGCTTCCACCAGGGCCACTGG | |
| | TGTCCCAGCCAGGTTCGGTGGCAGTGGGTCTGGG | |
| | ACACAGTTCACTCTCACCATCAGCAGCCTGCAGTC | |
| | TGAAGATTTTGCAGTTTATTACTGTCAGCATTATA | |
| | ATAACTGGCCTCCGTGGACGTTCGGCCAAGGGA | |
| | CCAAGGTGGAGATCAAAC | |
| 186 | EIVMTQSPATLSVSPGESATLFCRASRSVSDNLAWY | FNI14 VK (aa) |
| | QQKPGQAPRLLIFGASTRATGVPARFGGSGSGTQFT | |
| | LTISSLQSEDFAVYYCQHYNNWPPWTFGQGTKVEI | |
| | K | |
| 187 | RSVSDN | FNI14 CDRL1 (aa) |
| 188 | GAS | FNI14 CDRL2 (aa) |
| 189 | QHYNNWPPWT | FNI14 CDRL3 (aa) |
| 190 | GAGATCGTGATGACCCAGTCCCCTGCCACACTGTC | FNI14 Vk (co-nt) |
| | CGTGTCCCCAGGAGAGAGCGCCACCCTGTTCTGC | |
| | AGGGCTAGCAGGTCCGTGTCCGACAACCTGGCCT | |
| | GGTACCAGCAGAAGCCAGGCCAGGCTCCCAGGCT | |
| | GCTGATCTTTGGCGCCTCTACCAGAGCTACAGGCG | |
| | TGCCAGCTAGGTTCGGAGGAAGCGGATCTGGCAC | |
| | ACAGTTTACCCTGACAATCTCCAGCCTGCAGTCCG | |
| | AGGATTTCGCCGTGTACTATTGTCAGCACTATAAC | |
| | AATTGGCCCCCTTGGACCTTTGGCCAGGGCACAA | |
| | AGGTGGAGATCAAG | |
| 191 | CAGGTTCAACTGGTGCAGTCTGGGGCTGAGGTGA | FNI17 VH (wt-nt) |
| | AGAGGCCTGGGTCCTCGGTGAAGGTCTCCTGCAA | |
| | GCCTTCCGGAGGCACCTTCAGCAACAATGTTAT | |
| | CAGCTGGGTGCGACAGGCCCCTGGACAAGGGCTT | |
| | GAGTGGATGGGAGGGATCATCCCCACCTCTGGT | |
| | ATAGCAAACTACGCGCAGAAGTTCCAGGGCAGAG | |
| | TCGCGATTATTGCGGACAAATCTACGAGCACAGT | |
| | CTACATGGCGTTGAGCAGCCTGAGATCTGAGGAC | |
| | TCGGCCGTGTATTTCTGTGCCAGAGCGCGGTCCG | |
| | ACTACTTCAATAGAGACCTCGGCTGGGAAGATT | |
| | ACTACTTTGAGAACTGGGGCCAGGGAACCCTGGT | |
| | CACCGTCTCCTCAG | |
| 192 | QVQLVQSGAEVKRPGSSVKVSCKPSGGTFSNNVIS | FNI17 VH (aa) |
| | WVRQAPGQGLEWMGGIIPTSGIANYAQKFQGRVAI | |
| | IADKSTSTVYMALSSLRSEDSAVYFCARARSDYFNR | |
| | DLGWEDYYFENWGQGTLVTVSS | |
| 193 | GGTFSNNV | FNI17 CDRH1 (aa) |
| 194 | IIPTSGIA | FNI17 CDRH2 (aa) |
| 195 | ARARSDYFNRDLGWEDYYFEN | FNI17 CDRH3 (aa) |
| 196 | CAGGTGCAGCTGGTGCAGTCCGGAGCTGAGGTGA | FNI17 VH (co-nt) |
| | AGAGGCCAGGCTCCAGCGTGAAGGTGAGCTGCAA | |
| | GCCTTCTGGCGGCACCTTCTCCAACAATGTGATCA | |
| | GCTGGGTGAGACAGGCTCCAGGACAGGGACTGGA | |
| | GTGGATGGGAGGAATCATCCCCACATCTGGCATC | |
| | GCCAACTACGCTCAGAAGTTTCAGGGCAGGGTGG | |
| | CCATCATCGCTGATAAGTCCACCAGCACAGTGTAT | |
| | ATGGCCCTGTCTTCCCTGAGATCTGAGGACTCCGC | |
| | CGTGTACTTCTGTGCCAGGGCTCGGTCCGACTACT | |
| | TCAACCGCGATCTGGGCTGGGAGGACTACTATTTC | |
| | GAGAATTGGGGCCAGGGCACCCTGGTGACAGTGA | |
| | GCTCT | |
| 197 | GAAATAGTGATGACGCAGTCTCCAGCCACCCTGT | FNI17 Vk (wt-nt) |
| | CTGTGTCTCCAGGGGAAAGAGCCACCCTCTCCTGC | |
| | AGGGCCAGTCAGAGTGTTGGCAGCAGCTTAGTC | |
| | TGGTACCAGCAGAAACCTGGCCAGGCTCCCAGGC | |
| | TCCTCATCTATGGTGCATCCACCAGGGCCACTGG | |
| | TGTCCCAGCCAGGTTCAGTGGCAGTGGGTCTGGG | |
| | ACAGAGTTCACTCTCACCATCAGCAGCCTGCAGTC | |
| | TGAAGATTTTGCAGTTTATTACTGTCAGCACTATA | |
| | ATAACTGGCCTCCGTGGACGTTCGGCCAAGGGA | |
| | CCAAGGTGGAAATCAAAC | |
| 198 | EIVMTQSPATLSVSPGERATLSCRASQSVGSSLVWY | FNI17 VK (aa) |
| | QQKPGQAPRLLIYGASTRATGVPARFSGSGSGTEFTL | |
| | TISSLQSEDFAVYYCQHYNNWPPWTFGQGTKVEIK | |
| 199 | QSVGSS | FNI17 CDRL1 (aa) |
| 200 | GAS | FNI17 CDRL2 (aa) |
| 201 | QHYNNWPPWT | FNI17 CDRL3 (aa) |
| 202 | GAGATCGTGATGACCCAGTCTCCTGCCACACTGA | FNI17 Vk (co-nt) |
| | GCGTGTCTCCAGGAGAGAGGGCCACCCTGTCCTG | |
| | CAGGGCTTCCCAGAGCGTGGGATCCAGCCTGGTG | |
| | TGGTACCAGCAGAAGCCAGGACAGGCTCCAAGGC | |
| | TGCTGATCTATGGAGCTAGCACCAGAGCTACAGG | |
| | CGTGCCAGCTCGCTTCTCTGGATCCGGAAGCGGCA | |
| | CAGAGTTTACCCTGACAATCTCTTCCCTGCAGTCT | |
| | GAGGACTTCGCCGTGTACTATTGTCAGCACTACAA | |
| | CAATTGGCCCCCTTGGACCTTTGGCCAGGGCACAA | |
| | AGGTGGAGATCAAG | |
| 203 | CAAGTTCAGCTGGTGCAGTCTGGGGCTGAGGTGA | FNI19 VH (wt-nt) |
| | AGAGGCCTGGGTCCTCGGTGAGGGTCTCCTGCAA | |
| | GGCTTCTGAAGGCACCTTCAACAAGTATACTCTC | |
| | ACCTGGGTGCGACAGGCCCCTGGACAGGGACTTG | |
| | AGTGGATGGGAGGAATCATCCCTATCTCCGGTA | |
| | TAGCAAACTACGCACAGAAGTTCCAGGGCAGAGT | |
| | CGCGATTACCGCGGACGAATCCACGACCACAGCC | |
| | TACATGGAATTGAGCAGCCTAAGATCTGAAGACT | |
| | CGGCCGTATATTACTGTGCGACAGCGGTCTCCG | |
| | ATTATTTTAATCGAGACCTCGGCTGGGAAGATT | |
| | ACTACTTTCCGTTCTGGGGCCAGGGCACCCTGGT | |
| | CACCGTCGCCTCAG | |
| 204 | QVQLVQSGAEVKRPGSSVRVSCKASEGTFNKYTLT | FNI19 VH (aa) |
| | WVRQAPGQGLEWMGGIIPISGIANYAQKFQGRVAI | |
| | TADESTTTAYMELSSLRSEDSAVYYCATAVSDYFN | |
| | RDLGWEDYYFPFWGQGTLVTVAS | |
| 205 | EGTFNKYT | FNI19 CDRH1 (aa) |
| 206 | IIPISGIA | FNI19 CDRH2 (aa) |
| 207 | ATAVSDYFNRDLGWEDYYFPF | FNI19 CDRH3 (aa) |
| 208 | CAGGTGCAGCTGGTGCAGTCCGGAGCTGAGGTGA | FNI19 VH (co-nt) |
| | AGAGGCCAGGATCCAGCGTGCGGGTGTCCTGCAA | |
| | GGCTAGCGAGGGCACATTCAACAAGTACACACTG | |
| | ACCTGGGTGAGGCAGGCTCCAGGACAGGGACTGG | |
| | AGTGGATGGGCGGCATCATCCCTATCTCTGGCATC | |
| | GCCAATTACGCTCAGAAGTTTCAGGGCAGAGTGG | |
| | CCATCACAGCTGATGAGTCCACCACAACCGCCTAT | |
| | ATGGAGCTGTCTTCCCTGAGAAGCGAGGACTCCG | |
| | CCGTGTACTATTGTGCCACCGCTGTGAGCGACTAT | |
| | TTCAACCGCGATCTGGGCTGGGAGGACTACTATTT | |
| | CCCCTTTTGGGGCCAGGGCACACTGGTGACCGTG | |
| | GCTTCT | |
| 209 | GAAATAGTGATGACGCAGTCTCCAGCCACCCTGT | FNI19 Vk (wt-nt) |
| | CTGTGTCTCCGGGGGCCAGAGCCACCCTCTTCTGC | |
| | AGGGCCAGTCGGAGTGTTAGTGACAACTTAGCC | |
| | TGGTACCAGCAGAAACCTGGCCAGGCTCCCAGGC | |
| | TCCTCATCTTTGGTGCATCCACCAGGGCCACTGG | |
| | TGTCCCAGCCAGGTTCAGTGGAAGTGGGTCTGGG | |
| | ACACAGTTCACTCTCACCATCAGCAGCCTGCAGTC | |
| | CGAAGATTTTGCAGTTTATTACTGTCAGCATTATA | |
| | ATATTTGGCCTCCGTGGACGTTCGGCCAAGGGA | |
| | CCAAGGTGGAGATCAAAC | |
| 210 | EIVMTQSPATLSVSPGARATLFCRASRSVSDNLAWY | FNI19 VK (aa) |
| | QQKPGQAPRLLIFGASTRATGVPARFSGSGSGTQFTL | |
| | TISSLQSEDFAVYYCQHYNIWPPWTFGQGTKVEIK | |
| 211 | RSVSDN | FNI19 CDRL1 (aa) |
| 212 | GAS | FNI19 CDRL2 (aa) |
| 213 | QHYNIWPPWT | FNI19 CDRL3 (aa) |
| 214 | GAGATCGTGATGACCCAGTCCCCTGCTACACTGTC | FNI19 Vk (co-nt) |
| | CGTGTCCCCAGGAGCTAGGGCTACCCTGTTCTGCA | |
| | GGGCTAGCAGGTCCGTGTCCGACAACCTGGCTTG | |
| | GTACCAGCAGAAGCCAGGCCAGGCCCCCAGACTG | |
| | CTGATCTTTGGAGCTAGCACCAGAGCTACAGGCG | |
| | TGCCAGCTCGCTTCAGCGGATCTGGATCCGGCACA | |
| | CAGTTTACCCTGACAATCTCCAGCCTGCAGTCTGA | |
| | GGATTTCGCCGTGTACTATTGTCAGCACTATAATA | |
| | TCTGGCCCCCTTGGACCTTTGGCCAGGGCACAAAG | |
| | GTGGAGATCAAG | |
| 215 | [Reserved] | |
| 216 | CAGGTGCAGCTGGTGCAGTCCGGAGCTGAGGTGA | FNI3-VH-W110F |
| | AGAGGCCAGGATCCAGCGTGAAGGTGTCCTGCAA | (nt) |
| | GGCCAGCGGCGCTACCTTCAGCAACAATGTGAT | |
| | CGCTTGGGTGAGACAGGCTCCAGGACAGGGACTG | |
| | GAGTGGATGGGAGGAATCCACCCTATCAGCGCC | |
| | ACCGCTACATACGCCCAGAAGTTTCAGGGCAGAG | |
| | TGGCTATCGCCGCTGACGAGCTGACCTCTACAGCC | |
| | TATATGGAGCTGAACGGCCTGCGCAGCGAGGATT | |
| | CCGCCGTGTACTATTGTGCCAGGGCTGGCTCTGA | |
| | CTACTTCAACCGGGATCTGGGCTTCGAGAATTA | |
| | CTATTTTGACTCCTGGGGCCAGGGCACCCTGGTG | |
| | ACAGTGTCTTCC | |
| 217 | QVQLVQSGAEVKRPGSSVKVSCKASGATFSNNVIA | FNI3-VH-W110F |
| | WVRQAPGQGLEWMGGIHPISATATYAQKFQGRVAI | (aa) |
| | AADELTSTAYMELNGLRSEDSAVYYCARAGSDYFN | |
| | RDLGFENYYFDSWGQGTLVTVSS | |
| 218 | ARAGSDYFNRDLGFENYYFDS | FNI3-VH-W110F |
| | | CDRH3 (aa) |
| 219 | GAGATCCTGATGACCCAGTCCCCTGCCACACTGTC | FNI3-VK-W94F (nt) |
| | CGTGTCCCCAGGAGAGAGGGCCACCCTGAGCTGC | |
| | AGGGCTTCTCAGGACGTGTCCGGCAACCTGGCC | |
| | TGGTACCAGCAGAGACCAGGACAGGCTCCAAGGC | |
| | TGCTGATCTATGGAGCTTCCACCAGGGCTACAGG | |
| | CGTGCCAGCTAGATTCACCGGCGCTGGAAGCGGC | |
| | ACAGAGTTTACCCTGACAATCTCCAGCCTGCAGTC | |
| | TGAGGATTTCGCTCTGTACTATTGTCAGCACTACA | |
| | ACAATTTTCCCCCTTGGACCTTTGGCCAGGGCAC | |
| | AAAGGTGGAGATCAAG | |
| 220 | EILMTQSPATLSVSPGERATLSCRASQDVSGNLAWY | FNI3-VK-W94F (aa) |
| | QQRPGQAPRLLIYGASTRATGVPARFTGAGSGTEFT | |
| | LTISSLQSEDFALYYCQHYNNFPPWTFGQGTKVEIK | |
| 221 | QHYNNFPPWT | FNI3-VK-W94F |
| | | CDRL3 (aa) |
| 222 | GAGATCCTGATGACCCAGTCCCCTGCCACACTGTC | FNI3-VK-W97F (nt) |
| | CGTGTCCCCAGGAGAGAGGGCCACCCTGAGCTGC | |
| | AGGGCTTCTCAGGACGTGTCCGGCAACCTGGCC | |
| | TGGTACCAGCAGAGACCAGGACAGGCTCCAAGGC | |
| | TGCTGATCTATGGAGCTTCCACCAGGGCTACAGG | |
| | CGTGCCAGCTAGATTCACCGGCGCTGGAAGCGGC | |
| | ACAGAGTTTACCCTGACAATCTCCAGCCTGCAGTC | |
| | TGAGGATTTCGCTCTGTACTATTGTCAGCACTACA | |
| | ACAATTGGCCCCCTTTCACCTTTGGCCAGGGCAC | |
| | AAAGGTGGAGATCAAG | |
| 223 | EILMTQSPATLSVSPGERATLSCRASQDVSGNLAWY | FNI3-VK-W97F (aa) |
| | QQRPGQAPRLLIYGASTRATGVPARFTGAGSGTEFT | |
| | LTISSLQSEDFALYYCQHYNNWPPFTFGQGTKVEIK | |
| 224 | QHYNNWPPFT | FNI3-VK-W97F |
| | | CDRL3 (aa) |
| 225 | GAGATCCTGATGACCCAGTCCCCTGCCACACTGTC | FNI3-VK-W94F- |
| | CGTGTCCCCAGGAGAGAGGGCCACCCTGAGCTGC | W97F (nt) |
| | AGGGCTTCTCAGGACGTGTCCGGCAACCTGGCC | |
| | TGGTACCAGCAGAGACCAGGACAGGCTCCAAGGC | |
| | TGCTGATCTATGGAGCTTCCACCAGGGCTACAGG | |
| | CGTGCCAGCTAGATTCACCGGCGCTGGAAGCGGC | |
| | ACAGAGTTTACCCTGACAATCTCCAGCCTGCAGTC | |
| | TGAGGATTTCGCTCTGTACTATTGTCAGCACTACA | |
| | ACAATTTTCCCCCTTTCACCTTTGGCCAGGGCAC | |
| | AAAGGTGGAGATCAAG | |
| 226 | EILMTQSPATLSVSPGERATLSCRASQDVSGNLAWY | FNI3-VK-W94F- |
| | QQRPGQAPRLLIYGASTRATGVPARFTGAGSGTEFT | W97F (aa) |
| | LTISSLQSEDFALYYCQHYNNFPPFTFGQGTKVEIK | |
| 227 | QHYNNFPPFT | FNI3-VK-W94F- |
| | | W97F CDRL3 (aa) |
| 228 | CAGGTGCACCTGGTGCAGAGCGGAGCTGAGGTGA | FNI9-VH-W110F |
| | AGGAGCCAGGATCCAGCGTGACAGTGTCTTGCAA | (nt) |
| | GGCTTCCGGCGGCAGCTTCAACAATCAGGCTAT | |
| | CTCCTGGGTGAGGCAGGCTCCAGGACAGGGACTG | |
| | GAGTGGATGGGCGGCATCTTTCCCATCTCTGGC | |
| | ACACCTACCTCCGCCCAGAGGTTCCAGGGAAGGG | |
| | TGACCTTCACCGCTGACGAGAGCACCACAACCGT | |
| | GTACATGGATCTGTCTTCCCTGAGATCTGACGATA | |
| | CCGCCGTGTACTATTGTGCCAGAGCTGGCTCCGA | |
| | CTATTTCAACCGCGATCTGGGCTTCGAGAATTA | |
| | CTATTTTGCTTCCTGGGGCCAGGGCACACTGGTG | |
| | ACCGTGAGCTCT | |
| 229 | QVHLVQSGAEVKEPGSSVTVSCKASGGSFNNQAIS | FNI9-VH-W110F |
| | WVRQAPGQGLEWMGGIFPISGTPTSAQRFQGRVTF | (aa) |
| | TADESTTTVYMDLSSLRSDDTAVYYCARAGSDYFN | |
| | RDLGFENYYFASWGQGTLVTVSS | |
| 230 | ARAGSDYFNRDLGFENYYFAS | FNI9-VH-W110F |
| | | CDRH3 (aa) |
| 231 | GAGATCGTGATGACCCAGTCCCCAGCCACACTGA | FNI9-VK-W94F (nt) |
| | GCCTGTCCAGCGGAGAGAGGGCCACCCTGTCCTG | |
| | CAGGGCTTCCCGGAGCGTGTCTTCCAACCTGGC | |
| | CTGGTACCAGCAGAAGCCAGGCCAGGCTCCCAGA | |
| | CTGCTGATCTATGACGCCTCTACCAGAGCTACAG | |
| | GCTTCTCCGCCAGGTTTGCTGGATCTGGATCCGGC | |
| | ACAGAGTTCACCCTGACAATCAGCTCTCTGCAGA | |
| | GCGAGGATTCTGCTATCTACTATTGTCAGCAGTA | |
| | CAACAATTTCCCCCCTTGGACCTTTGGCCAGGG | |
| | CACAAAGGTGGAGATCAAG | |
| 232 | EIVMTQSPATLSLSSGERATLSCRASRSVSSNLAWY | FNI9-VK-W94F (aa) |
| | QQKPGQAPRLLIYDASTRATGFSARFAGSGSGTEFTL | |
| | TISSLQSEDSAIYYCQQYNNFPPWTFGQGTKVEIK | |
| 233 | QQYNNFPPWT | FNI9-VK-W94F |
| | | CDRL3 (aa) |
| 234 | GAGATCGTGATGACCCAGTCCCCAGCCACACTGA | FNI9-VK-W97F (nt) |
| | GCCTGTCCAGCGGAGAGAGGGCCACCCTGTCCTG | |
| | CAGGGCTTCCCGGAGCGTGTCTTCCAACCTGGC | |
| | CTGGTACCAGCAGAAGCCAGGCCAGGCTCCCAGA | |
| | CTGCTGATCTATGACGCCTCTACCAGAGCTACAG | |
| | GCTTCTCCGCCAGGTTTGCTGGATCTGGATCCGGC | |
| | ACAGAGTTCACCCTGACAATCAGCTCTCTGCAGA | |
| | GCGAGGATTCTGCTATCTACTATTGTCAGCAGTA | |
| | CAACAATTGGCCCCCTTTCACCTTTGGCCAGGG | |
| | CACAAAGGTGGAGATCAAG | |
| 235 | EIVMTQSPATLSLSSGERATLSCRASRSVSSNLAWY | FNI9-VK-W97F (aa) |
| | QQKPGQAPRLLIYDASTRATGFSARFAGSGSGTEFTL | |
| | TISSLQSEDSAIYYCQQYNNWPPFTFGQGTKVEIK | |
| 236 | QQYNNWPPFT | FNI9-VK-W97F |
| | | CDRL3 (aa) |
| 237 | GAGATCGTGATGACCCAGTCCCCAGCCACACTGA | FNI9-VK-W94F- |
| | GCCTGTCCAGCGGAGAGAGGGCCACCCTGTCCTG | W97F (nt) |
| | CAGGGCTTCCCGGAGCGTGTCTTCCAACCTGGC | |
| | CTGGTACCAGCAGAAGCCAGGCCAGGCTOCCAGA | |
| | CTGCTGATCTATGACGCCTCTACCAGAGCTACAG | |
| | GCTTCTCCGCCAGGTTTGCTGGATCTGGATCCGGC | |
| | ACAGAGTTCACCCTGACAATCAGCTCTCTGCAGA | |
| | GCGAGGATTCTGCTATCTACTATTGTCAGCAGTA | |
| | CAACAATTTTCCCCCTTTCACCTTTGGCCAGGGC | |
| | ACAAAGGTGGAGATCAAG | |
| 238 | EIVMTQSPATLSLSSGERATLSCRASRSVSSNLAWY | FNI9-VK-W94F- |
| | QQKPGQAPRLLIYDASTRATGFSARFAGSGSGTEFTL | W97F (aa) |
| | TISSLQSEDSAIYYCQQYNNFPPFTFGQGTKVEIK | |
| 239 | QQYNNFPPFT | FNI9-VK-W94F- |
| | | W97F CDRL3 (aa) |
| 240 | CAGGTCCAGCTGGTCCAGAGTGGGGCAGAGGTCA | FNI17-v19-VH (co- |
| | AAGAGCCAGGGTCTTCAGTCACAGTCTCATGCAA | nt) |
| | AGCAAGCGGAGGAACATTTTCCAACAATGTGATC | |
| | AGCTGGGTGAGGCAGGCTCCAGGACAGGGACTGG | |
| | AGTGGATGGGCGGCATCATCCCTACCTCTGGCATC | |
| | GCCAACTACGCTCAGAAGTTCCAGGGCAGAGTGG | |
| | CCATCATCGCTGACAAGTCTACCTCCACAGTGTAT | |
| | ATGGCCCTGTCCAGCCTGAGAAGCGAGGATTCCG | |
| | CCGTGTACTTCTGCGCCAGGGCTCGGTCCGACTAC | |
| | TTCAACCGCGATCTGGGTTGGGAGGACTATTACTT | |
| | TGAAAACTGGGGGCAGGGCACACTGGTCACTGTC | |
| | TCATCAGC | |
| 241 | QVQLVQSGAEVKEPGSSVTVSCKASGGTFSNNVIS | FNI17-v19-VH (aa) |
| | WVRQAPGQGLEWMGGIIPTSGIANYAQKFQGRVAI | |
| | IADKSTSTVYMALSSLRSEDSAVYFCARARSDYFNR | |
| | DLGWEDYYFENWGQGTLVTVSS | |
| 242 | GAAATTGTGATGACCCAGTCTCCAGCCACTCTGTC | FNI17-v19-VK (co- |
| | AGTCTCTCCAGGGGAACGAGCCACTCTGTCATGTC | nt) |
| | GGGCCTCTCAGTCCGTCGGCTCCAGCCTGGCTTGG | |
| | TACCAGCAGAAGCCAGGACAGGCTCCTAGGCTGC | |
| | TGATCTATGGAGCTAGCACCAGGGCTACAGGCGT | |
| | GCCAGCTCGGTTCAGCGGATCTGGATCCGGCACC | |
| | GAGTTTACCCTGACAATCTCTTCCCTGCAGTCTGA | |
| | GGACTTCGCCGTGTACTATTGCCAGCACTACAATA | |
| | ACTGGCCTCCTTGGACATTCGGGCAGGGGACAAA | |
| | AGTCGAGATTAAG | |
| 243 | EIVMTQSPATLSVSPGERATLSCRASQSVGSSLAWY | FNI17-v19-VK (aa) |
| | QQKPGQAPRLLIYGASTRATGVPARFSGSGSGTEFTL | |
| | TISSLQSEDFAVYYCQHYNNWPPWTFGQGTKVEIK | |
| 244 | CAGGTCCAGCTGGTGCAGAGTGGTGCCGAGGTCA | FNI19-v3-VH (co- |
| | AAAAGCCAGGGTCAAGTGTCAAAGTCAGTTGTAA | nt) |
| | AGCATCAGAGGGAACATTCAACAAGTACACAATC | |
| | AGCTGGGTGAGACAGGCTCCAGGACAGGGACTGG | |
| | AGTGGATGGGCGGCATCATCCCTATCTCTGGCATC | |
| | GCCAATTACGCTCAGAAGTTCCAGGGCCGCGTGG | |
| | CCATCACAGCTGACGAGTCCACCACAACCGCCTA | |
| | TATGGAGCTGTCCAGCCTGAGGTCTGAGGATTCCG | |
| | CCGTGTACTATTGCGCCACCGCTGTGAGCGACTAC | |
| | TTCAACCGGGATCTGGGCTGGGAGGACTATTATTT | |
| | TCCATTCTGGGGTCAGGGGACACTGGTCACCGTCT | |
| | CTTCC | |
| 245 | QVQLVQSGAEVKKPGSSVKVSCKASEGTFNKYTIS | FNI19-v3-VH (aa) |
| | WVRQAPGQGLEWMGGIIPISGIANYAQKFQGRVAI | |
| | TADESTTTAYMELSSLRSEDSAVYYCATAVSDYFN | |
| | RDLGWEDYYFPFWGQGTLVTVSS | |
| 246 | GAGATCGTGATGACCCAGTCCCCTGCTACACTGTC | FNI19-v3-VK (co- |
| | CGTGTCCCCAGGAGCTAGGGCTACCCTGTTCTGCA | nt) |
| | GGGCTAGCAGGTCCGTGTCCGACAACCTGGCTTG | |
| | GTACCAGCAGAAGCCAGGCCAGGCCCCCAGACTG | |
| | CTGATCTTTGGAGCTAGCACCAGAGCTACAGGCG | |
| | TGCCAGCTCGCTTCAGCGGATCTGGATCCGGCACA | |
| | CAGTTTACCCTGACAATCTCCAGCCTGCAGTCTGA | |
| | GGATTTCGCCGTGTACTATTGTCAGCACTATAATA | |
| | TCTGGCCCCCTTGGACCTTTGGCCAGGGCACAAAG | |
| | GTGGAGATCAAG | |
| 247 | EIVMTQSPATLSVSPGARATLFCRASRSVSDNLAWY | FNI19-v3-VK (aa) |
| | QQKPGQAPRLLIFGASTRATGVPARFSGSGSGTQFTL | |
| | TISSLQSEDFAVYYCQHYNIWPPWTFGQGTKVEIK | |
| 248 | CAGGTGCACCTGGTGCAGAGCGGAGCTGAGGTGA | FNI9-v5-VH (co-nt) |
| | AGGAGCCAGGATCCAGCGTGACAGTGTCTTGCAA | |
| | GGCTTCCGGCGGCAGCTTCAACAATCAGGCTATCT | |
| | CCTGGGTGAGGCAGGCTCCAGGACAGGGACTGGA | |
| | GTGGATGGGCGGCATCTTTCCCATCTCTGGCACAC | |
| | CTACCTCCGCCCAGAGGTTCCAGGGAAGGGTGAC | |
| | CTTCACCGCTGACGAGAGCACCACAACCGTGTAC | |
| | ATGGATCTGTCTTCCCTGAGATCTGACGATACCGC | |
| | CGTGTACTATTGTGCCAGAGCTGGCTCCGACTATT | |
| | TCAACCGCGATCTGGGCTGGGAGAATTACTATTTT | |
| | GCTTCCTGGGGCCAGGGCACACTGGTGACCGTGA | |
| | GCTCT | |
| 249 | QVHLVQSGAEVKEPGSSVTVSCKASGGSFNNQAIS | FNI9-v5-VH (aa) |
| | WVRQAPGQGLEWMGGIFPISGTPTSAQRFQGRVTF | |
| | TADESTTTVYMDLSSLRSDDTAVYYCARAGSDYFN | |
| | RDLGWENYYFASWGQGTLVTVSS | |
| 250 | GAGATTGTGATGACCCAGTCCCCTGCTACCCTGAG | FNI9-v5-VK (co-nt) |
| | CGTGTCCCCCGGAGAGAGAGCTACCCTGAGTTGC | |
| | CGCGCCAGCCGCAGTGTCTCTGACAACCTGGCTTG | |
| | GTACCAGCAGAAGCCAGGACAGGCTCCTAGGCTG | |
| | CTGATCTATGGCGCCTCCACCAGGGCTACAGGCAT | |
| | CCCAGCTCGGTTCTCTGGATCCGGAAGCGGCACC | |
| | GAGTTTACCCTGACAATCTCCAGCCTGCAGAGCG | |
| | AGGATTTCGCCGTGTACTATTGCCAGCATTACAAC | |
| | ATCTGGCCTCCTTGGACATTCGGTCAGGGAACTAA | |
| | AGTGGAAATTAAG | |
| 251 | EIVMTQSPATLSVSPGERATLSCRASRSVSDNLAWY | FNI9-v5-VK (aa) |
| | QQKPGQAPRLLIYGASTRATGIPARFSGSGSGTEFTL | |
| | TISSLQSEDFAVYYCQHYNIWPPWTFGQGTKVEIK | |
| 252 | ASTKGPSVFPLAPSSKSTSGGTAALGCLVKDYFPEPV | IgHG1*01, G1m3 |
| | TVSWNSGALTSGVHTFPAVLQSSGLYSLSSVVTVPS | CH1-CH3 with |
| | SSLGTQTYICNVNHKPSNTKVDKRVEPKSCDKTHTC | M428L and N434S |
| | PPCPAPELLGGPSVFLFPPKPKDTLMISRTPEVTCVV | mutations and C- |
| | VDVSHEDPEVKFNWYVDGVEVHNAKTKPREEQYN | terminal lysine |
| | STYRVVSVLTVLHQDWLNGKEYKCKVSNKALPAPI | |
| | EKTISKAKGQPREPQVYTLPPSREEMTKNQVSLTCL | |
| | VKGFYPSDIAVEWESNGQPENNYKTTPPVLDSDGSF | |
| | FLYSKLTVDKSRWQQGNVFSCSVLHEALHSHYTQK | |
| | SLSLSPGK | |
| 253 | ASTKGPSVFPLAPSSKSTSGGTAALGCLVKDYFPEPV | IgHG1*01, G1m3 |
| | TVSWNSGALTSGVHTFPAVLQSSGLYSLSSVVTVPS | CH1-CH3 with |
| | SSLGTQTYICNVNHKPSNTKVDKRVEPKSCDKTHTC | M428L and N434S |
| | PPCPAPELLGGPSVFLFPPKPKDTLMISRTPEVTCVV | mutations, without |
| | VDVSHEDPEVKFNWYVDGVEVHNAKTKPREEQYN | C-terminal lysine |
| | STYRVVSVLTVLHQDWLNGKEYKCKVSNKALPAPI | |
| | EKTISKAKGQPREPQVYTLPPSREEMTKNQVSLTCL | |
| | VKGFYPSDIAVEWESNGQPENNYKTTPPVLDSDGSF | |
| | FLYSKLTVDKSRWQQGNVFSCSVLHEALHSHYTQK | |
| | SLSLSPG | |
| 254 | RTVAAPSVFIFPPSDEQLKSGTASVVCLLNNFYPREA | Kappa light chain |
| | KVQWKVDNALQSGNSQESVTEQDSKDSTYSLSSTL | CL |
| | TLSKADYEKHKVYACEVTHQGLSSPVTKSFNRGEC | |
| 255 | QVQLVQSGAEVKEPGSSVTVSCKASGGTFSNNVISW | FNI17-v19 heavy |
| | VRQAPGQGLEWMGGIIPTSGIANYAQKFQGRVAIIA | chain with M428L |
| | DKSTSTVYMALSSLRSEDSAVYFCARARSDYFNRDL | and N434S |
| | GWEDYYFENWGQGTLVTVSSASTKGPSVFPLAPSSK | mutations in CH3 |
| | STSGGTAALGCLVKDYFPEPVTVSWNSGALTSGVHT | and a C-terminal |
| | FPAVLQSSGLYSLSSVVTVPSSSLGTQTYICNVNHKP | lysine |
| | SNTKVDKRVEPKSCDKTHTCPPCPAPELLGGPSVFLF | |
| | PPKPKDTLMISRTPEVTCVVVDVSHEDPEVKFNWYV | |
| | DGVEVHNAKTKPREEQYNSTYRVVSVLIVLHQDWL | |
| | NGKEYKCKVSNKALPAPIEKTISKAKGQPREPQVYT | |
| | LPPSREEMTKNQVSLTCLVKGFYPSDIAVEWESNGQ | |
| | PENNYKTTPPVLDSDGSFFLYSKLTVDKSRWQQGN | |
| | VFSCSVLHEALHSHYTQKSLSLSPGK | |
| 256 | QVQLVQSGAEVKEPGSSVTVSCKASGGTFSNNVISW | FNI17-v19 heavy |
| | VRQAPGQGLEWMGGIIPTSGIANYAQKFQGRVAIIA | chain with M428L |
| | DKSTSTVYMALSSLRSEDSAVYFCARARSDYFNRDL | and N434S |
| | GWEDYYFENWGQGTLVTVSSASTKGPSVFPLAPSSK | mutations in CH3, |
| | STSGGTAALGCLVKDYFPEPVTVSWNSGALTSGVHT | without C-terminal |
| | FPAVLQSSGLYSLSSVVTVPSSSLGTQTYICNVNHKP | lysine |
| | SNTKVDKRVEPKSCDKTHTCPPCPAPELLGGPSVFLF | |
| | PPKPKDTLMISRTPEVTCVVVDVSHEDPEVKFNWYV | |
| | DGVEVHNAKTKPREEQYNSTYRVVSVLTVLHQDWL | |
| | NGKEYKCKVSNKALPAPIEKTISKAKGQPREPQVYT | |
| | LPPSREEMTKNQVSLTCLVKGFYPSDIAVEWESNGQ | |
| | PENNYKTTPPVLDSDGSFFLYSKLTVDKSRWQQGN | |
| | VFSCSVLHEALHSHYTQKSLSLSPG | |
| 257 | EIVMTQSPATLSVSPGERATLSCRASQSVGSSLAWY | FNI17-v19 light |
| | QQKPGQAPRLLIYGASTRATGVPARFSGSGSGTEFTL | chain |
| | TISSLQSEDFAVYYCQHYNNWPPWTFGQGTKVEIKR | |
| | TVAAPSVFIFPPSDEQLKSGTASVVCLLNNFYPREAK | |
| | VQWKVDNALQSGNSQESVTEQDSKDSTYSLSSTLTL | |
| | SKADYEKHKVYACEVTHQGLSSPVTKSFNRGEC | |
| 258 | QVQLVQSGARVKEPGSSVKVSCKASGGTFSNNVIS | FNI17-v13 VH (aa) |
| | WVRQAPGQGLEWMGGIIPTSGIANYAQKFQGRVAII | |
| | ADKSTSTVYMALSSLRSEDSAVYFCARARSDYFNRD | |
| | LGWEDYYFENWGQGTLVTVSS | |
| 259 | EIVMTQSPATLSVSPGERATLSCRASQSVGSSLAWY | FNI17-v13 VK (aa) |
| | QQKPGQAPRLLIYGASTRATGVPARFSGSGSGTEFTL | |
| | TISSLQSEDFAVYYCQHYNNWPPWTFGQGTKVEIK | |
| 260 | CAGGTGCAGCTGGTGCAGTCTGGCGCCGAGGTGAAGA | FNI-UCA-IGH |
| | AGCCAGGCTCCAGCGTGAAGGTGAGCTGCAAGGCTTC | (wt-nt) |
| | TGGCGGCACCTTCTCTTCCTACGCTATCTCCTGGGTGA | |
| | GGCAGGCTCCAGGACAGGGACTGGAGTGGATGGGCGG | |
| | CATCATCCCTATCTTCGGCACAGCCAACTACGCTCAGA | |
| | AGTTTCAGGGCAGAGTGACCATCACAGCCGACGAGTC | |
| | TACCTCCACAGCTTATATGGAGCTGAGCTCTCTGCGCT | |
| | CCGAGGATACCGCCGTGTACTATTGTGCCAGGGCTGGC | |
| | AGCGACTACTTCAACCGGGATCTGGGCTGGGAGAATT | |
| | ACTATTTTGACTATTGGGGCCAGGGCACCCTGGTGACA | |
| | GTGTCCAGC | |
| 261 | QVQLVQSGAEVKKPGSSVKVSCKASGGTFSSYAIS | FNI-UCA VH (aa) |
| | WVRQAPGQGLEWMGGIIPIFGTANYAQKFQGRVTI | |
| | TADESTSTAYMELSSLRSEDTAVYYCARAGSDYFN | |
| | RDLGWENYYFDYWGQGTLVTVSS | |
| 262 | GAGATCGTGATGACCCAGTCTCCTGCCACACTGA | FNI-UCA-IGK |
| | GCGTGTCTCCAGGAGAGAGGGCCACCCTGTCCTG | (wt-nt) |
| | CAGGGCTTCCCAGAGCGTGTCCAGCAACCTGGCC | |
| | TGGTACCAGCAGAAGCCAGGCCAGGCTCCCAGGC | |
| | TGCTGATCTATGGCGCCAGCACCAGAGCTACAGG | |
| | CATCCCAGCTCGCTTCTCTGGATCCGGAAGCGGCA | |
| | CAGAGTTTACCCTGACAATCTCTTCCCTGCAGTCT | |
| | GAGGACTTCGCCGTGTACTATTGTCAGCAGTACAA | |
| | CAATTGGCCCCCTTGGACCTTTGGCCAGGGCACAA | |
| | AGGTGGAGATCAAG | |
| 263 | EIVMTQSPATLSVSPGERATLSCRASQSVSSNLAWY | FNI-UCA VK (aa) |
| | QQKPGQAPRLLIYGASTRATGIPARFSGSGSGTEFTL | |
| | TISSLQSEDFAVYYCQQYNNWPPWTFGQGTKVEIK | |
| 264 | GGTFSSYA | FNI-UCA CDRH1 |
| | | (aa) |
| 265 | IIPIFGTA | FNI-UCA CDRH2 |
| | | (aa) |
| 266 | ARAGSDYFNRDLGWENYYFDY | FNI-UCA CDRH3 |
| | | (aa) |
| 267 | QSVSSN | FNI-UCA CDRL1 |
| | | (aa) |
| 268 | GAS | FNI-UCA CDRL2 |
| | | (aa) |
| 269 | QQYNNWPPWT | FNI-UCA CDRL3 |
| | | (aa) |
| 270 | QVQLQQSGPGLVKPSQTLSLTCAISGDSVSSYNAVW | FM08_LS Heavy |
| | NWIRQSPSRGLEWLGRTYYRSGWYNDYAESVKSRI | Chain (aa) |
| | TINPDTSKNQFSLQLNSVTPEDTAVYYCARSGHITVF | |
| | GVNVDAFDMWGQGTMVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNVNH | |
| | KPSNTKVDKRVEPKSCDKTHTCPPCPAPELLGGPSV | |
| | FLFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPAPIEKTISKAKGQPREP | |
| | QVYTLPPSREEMTKNQVSLTCLVKGFYPSDIAVEWE | |
| | SNGQPENNYKTTPPVLDSDGSFFLYSKLTVDKSRWQ | |
| | QGNVFSCSVLHEALHSHYTQKSLSLSPGK | |
| 271 | DIQMTQSPSSLSASVGDRVTITCRTSQSLSSYTHWYQ | FM08_LS Light |
| | QKPGKAPKLLIYAASSRGSGVPSRFSGSGSGTDFTLT | Chain (aa) |
| | ISSLQPEDFATYYCQQSRTFGQGTKVEIKRTVAAPSV | |
| | FIFPPSDEQLKSGTASVVCLLNNFYPREAKVQWKVD | |
| | NALQSGNSQESVTEQDSKDSTYSLSSTLTLSKADYE | |
| | KHKVYACEVTHQGLSSPVTKSFNRGEC | |
| 272 | QVQLQQSGPGLVKPSQTLSLTCAISGDSVSSYNAVW | FM08_GAALIE_LS |
| | NWIRQSPSRGLEWLGRTYYRSGWYNDYAESVKSRI | Heavy Chain (aa) |
| | TINPDTSKNQFSLQLNSVTPEDTAVYYCARSGHITVF | |
| | GVNVDAFDMWGQGTMVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNVNH | |
| | KPSNTKVDKRVEPKSCDKTHTCPPCPAPELLAGPSV | |
| | FLFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPLPEEKTISKAKGQPREP | |
| | QVYTLPPSREEMTKNQVSLTCLVKGFYPSDIAVEWE | |
| | SNGQPENNYKTTPPVLDSDGSFFLYSKLTVDKSRWQ | |
| | QGNVFSCSVLHEALHSHYTQKSLSLSPGK | |
| 273 | DIQMTQSPSSLSASVGDRVTITCRTSQSLSSYTHWYQ | FM08_GAALIE_LS |
| | QKPGKAPKLLIYAASSRGSGVPSRFSGSGSGTDFTLT | Light Chain (aa) |
| | ISSLQPEDFATYYCQQSRTFGQGTKVEIKRTVAAPSV | |
| | FIFPPSDEQLKSGTASVVCLLNNFYPREAKVQWKVD | |
| | NALQSGNSQESVTEQDSKDSTYSLSSTLTLSKADYE | |
| | KHKVYACEVTHQGLSSPVTKSFNRGEC | |
| 274 | SYNAVWN | FM08 CDRH1 |
| 275 | RTYYRSGWYNDYAESVKS | FM08 CDRH2 |
| 276 | SGHITVFGVNVDAFDM | FM08 CDRH3 |
| 277 | RTSQSLSSYTH | FM08 CDRL1 |
| 278 | AASSRGS | FM08 CDRL2 |
| 279 | QQSRT | FM08 CDRL3 |
| 280 | ASTKGPSVFPLAPSSKSTSGGTAALGCLVKDYFPEPV | IgG1 GAALIE CH1- |
| | TVSWNSGALTSGVHTFPAVLQSSGLYSLSSVVTVPS | CH3 |
| | SSLGTQTYICNVNHKPSNTKVDKRVEPKSCDKTHTC | |
| | PPCPAPELLAGPSVFLFPPKPKDTLMISRTPEVTCVV | |
| | VDVSHEDPEVKFNWYVDGVEVHNAKTKPREEQYN | |
| | STYRVVSVLTVLHQDWLNGKEYKCKVSNKALPLPE | |
| | EKTISKAKGQPREPQVYTLPPSREEMTKNQVSLTCL | |
| | VKGFYPSDIAVEWESNGQPENNYKTTPPVLDSDGSF | |
| | FLYSKLTVDKSRWQQGNVFSCSVMHEALHNHYTQ | |
| | KSLSLSPGK | |
| 281 | ASTKGPSVFPLAPSSKSTSGGTAALGCLVKDYFPEPV | IgG1 |
| | TVSWNSGALTSGVHTFPAVLQSSGLYSLSSVVTVPS | GAALIE_MLNS |
| | SSLGTQTYICNVNHKPSNTKVDKRVEPKSCDKTHTC | CH1-CH3 |
| | PPCPAPELLAGPSVFLFPPKPKDTLMISRTPEVTCVV | |
| | VDVSHEDPEVKFNWYVDGVEVHNAKTKPREEQYN | |
| | STYRVVSVLTVLHQDWLNGKEYKCKVSNKALPLPE | |
| | EKTISKAKGQPREPQVYTLPPSREEMTKNQVSLTCL | |
| | VKGFYPSDIAVEWESNGQPENNYKTTPPVLDSDGSF | |
| | FLYSKLTVDKSRWQQGNVFSCSVLHEALHSHYTQK | |
| | SLSLSPGK | |

TABLE 2
Neuraminidase Amino Acid Position Comparison (H1N1
California.07.2009 to H3N2 New York.392.2004)

| residue_N1 | position_N1 | residue_N2 | position_N2 |
|---|---|---|---|
| M | 1 | M | 1 |
| N | 2 | N | 2 |
| P | 3 | P | 3 |
| N | 4 | N | 4 |
| Q | 5 | Q | 5 |
| K | 6 | K | 6 |
| I | 7 | I | 7 |
| I | 8 | I | 8 |
| T | 9 | T | 9 |
| I | 10 | I | 10 |
| G | 11 | G | 11 |
| S | 12 | S | 12 |
| V | 13 | V | 13 |
| C | 14 | S | 14 |
| M | 15 | L | 15 |
| T | 16 | T | 16 |
| I | 17 | I | 17 |
| G | 18 | S | 18 |
| M | 19 | T | 19 |
| A | 20 | I | 20 |
| N | 21 | C | 21 |
| L | 22 | F | 22 |
| I | 23 | F | 23 |
| L | 24 | M | 24 |
| Q | 25 | Q | 25 |
| I | 26 | I | 26 |
| G | 27 | A | 27 |
| N | 28 | I | 28 |
| I | 29 | L | 29 |
| I | 30 | I | 30 |
| S | 31 | T | 31 |
| I | 32 | T | 32 |
| W | 33 | V | 33 |
| I | 34 | T | 34 |
| S | 35 | L | 35 |
| H | 36 | H | 36 |
| S | 37 | F | 37 |
| I | 38 | K | 38 |
| Q | 39 | Q | 39 |
| L | 40 | Y | 40 |
| G | 41 | E | 41 |
| N | 42 | F | 42 |
| Q | 43 | N | 43 |
| N | 44 | S | 44 |
| Q | 45 | P | 45 |
| I | 46 | P | 46 |
| E | 47 | N | 47 |
| T | 48 | — | NA |
| C | 49 | — | NA |
| N | 50 | N | 48 |
| Q | 51 | Q | 49 |
| S | 52 | V | 50 |
| V | 53 | M | 51 |
| I | 54 | L | 52 |
| T | 55 | C | 53 |
| Y | 56 | E | 54 |
| E | 57 | P | 55 |
| N | 58 | T | 56 |
| N | 59 | I | 57 |
| T | 60 | I | 58 |
| W | 61 | E | 59 |
| V | 62 | R | 60 |
| N | 63 | N | 61 |
| Q | 64 | I | 62 |
| T | 65 | T | 63 |
| — | NA | E | 64 |
| Y | 66 | I | 65 |
| V | 67 | V | 66 |
| N | 68 | Y | 67 |
| I | 69 | L | 68 |
| S | 70 | T | 69 |
| N | 71 | N | 70 |
| T | 72 | T | 71 |
| N | 73 | T | 72 |
| F | 74 | I | 73 |
| A | 75 | E | 74 |
| A | 76 | K | 75 |
| G | 77 | E | 76 |
| Q | 78 | M | 77 |
| S | 79 | C | 78 |
| V | 80 | P | 79 |
| V | 81 | K | 80 |
| S | 82 | L | 81 |
| V | 83 | A | 82 |
| K | 84 | E | 83 |
| L | 85 | Y | 84 |
| A | 86 | R | 85 |
| G | 87 | N | 86 |
| N | 88 | W | 87 |
| S | 89 | S | 88 |
| S | 90 | K | 89 |
| — | NA | P | 90 |
| L | 91 | Q | 91 |
| C | 92 | C | 92 |
| P | 93 | D | 93 |
| V | 94 | I | 94 |
| S | 95 | T | 95 |
| G | 96 | G | 96 |
| W | 97 | F | 97 |
| A | 98 | A | 98 |
| I | 99 | P | 99 |
| Y | 100 | F | 100 |
| S | 101 | S | 101 |
| K | 102 | K | 102 |
| D | 103 | D | 103 |
| N | 104 | N | 104 |
| S | 105 | S | 105 |
| V | 106 | I | 106 |
| R | 107 | R | 107 |
| I | 108 | L | 108 |
| G | 109 | S | 109 |
| S | 110 | A | 110 |
| K | 111 | G | 111 |
| G | 112 | G | 112 |
| D | 113 | D | 113 |
| V | 114 | I | 114 |
| F | 115 | W | 115 |
| V | 116 | V | 116 |
| I | 117 | T | 117 |
| R | 118 | R | 118 |
| E | 119 | E | 119 |
| P | 120 | P | 120 |
| F | 121 | Y | 121 |
| I | 122 | V | 122 |
| S | 123 | S | 123 |
| C | 124 | C | 124 |
| S | 125 | D | 125 |
| P | 126 | P | 126 |
| L | 127 | D | 127 |
| E | 128 | K | 128 |
| C | 129 | C | 129 |
| R | 130 | Y | 130 |
| T | 131 | Q | 131 |
| F | 132 | F | 132 |
| F | 133 | A | 133 |
| L | 134 | L | 134 |
| T | 135 | G | 135 |
| Q | 136 | Q | 136 |
| G | 137 | G | 137 |
| A | 138 | T | 138 |
| L | 139 | T | 139 |
| L | 140 | L | 140 |
| N | 141 | N | 141 |
| D | 142 | N | 142 |
| K | 143 | V | 143 |
| H | 144 | H | 144 |
| S | 145 | S | 145 |
| N | 146 | N | 146 |
| G | 147 | D | 147 |
| T | 148 | T | 148 |
| I | 149 | V | 149 |
| K | 150 | H | 150 |
| D | 151 | D | 151 |
| R | 152 | R | 152 |
| S | 153 | T | 153 |
| P | 154 | P | 154 |
| Y | 155 | Y | 155 |
| R | 156 | R | 156 |
| T | 157 | T | 157 |
| L | 158 | L | 158 |
| M | 159 | L | 159 |
| S | 160 | M | 160 |
| C | 161 | N | 161 |
| P | 162 | E | 162 |
| I | 163 | L | 163 |
| G | 164 | G | 164 |
| E | 165 | — | NA |
| V | 166 | V | 165 |
| P | 167 | P | 166 |
| S | 168 | F | 167 |
| P | 169 | H | 168 |
| Y | 170 | L | 169 |
| N | 171 | G | 170 |
| S | 172 | T | 171 |
| R | 173 | K | 172 |
| F | 174 | Q | 173 |
| E | 175 | V | 174 |
| S | 176 | C | 175 |
| V | 177 | I | 176 |
| A | 178 | A | 177 |
| W | 179 | W | 178 |
| S | 180 | S | 179 |
| A | 181 | S | 180 |
| S | 182 | S | 181 |
| A | 183 | S | 182 |
| C | 184 | C | 183 |
| H | 185 | H | 184 |
| D | 186 | D | 185 |
| G | 187 | G | 186 |
| I | 188 | K | 187 |
| N | 189 | A | 188 |
| W | 190 | W | 189 |
| L | 191 | L | 190 |
| T | 192 | H | 191 |
| I | 193 | V | 192 |
| G | 194 | C | 193 |
| I | 195 | V | 194 |
| S | 196 | T | 195 |
| G | 197 | G | 196 |
| P | 198 | D | 197 |
| D | 199 | D | 198 |
| N | 200 | K | 199 |
| G | 201 | N | 200 |
| A | 202 | A | 201 |
| V | 203 | T | 202 |
| A | 204 | A | 203 |
| V | 205 | S | 204 |
| L | 206 | F | 205 |
| K | 207 | I | 206 |
| Y | 208 | Y | 207 |
| N | 209 | N | 208 |
| G | 210 | G | 209 |
| I | 211 | R | 210 |
| I | 212 | L | 211 |
| T | 213 | V | 212 |
| D | 214 | D | 213 |
| T | 215 | S | 214 |
| I | 216 | I | 215 |
| K | 217 | V | 216 |
| S | 218 | S | 217 |
| W | 219 | W | 218 |
| R | 220 | S | 219 |
| N | 221 | K | 220 |
| N | 222 | K | 221 |
| I | 223 | I | 222 |
| L | 224 | L | 223 |
| R | 225 | R | 224 |
| T | 226 | T | 225 |
| Q | 227 | Q | 226 |
| E | 228 | E | 227 |
| S | 229 | S | 228 |
| E | 230 | E | 229 |
| C | 231 | C | 230 |
| A | 232 | V | 231 |
| C | 233 | C | 232 |
| V | 234 | I | 233 |
| N | 235 | N | 234 |
| G | 236 | G | 235 |
| S | 237 | T | 236 |
| C | 238 | C | 237 |
| F | 239 | T | 238 |
| T | 240 | V | 239 |
| V | 241 | V | 240 |
| M | 242 | M | 241 |
| T | 243 | T | 242 |
| D | 244 | D | 243 |
| G | 245 | G | 244 |
| P | 246 | S | 245 |
| S | 247 | A | 246 |
| N | 248 | S | 247 |
| G | 249 | G | 248 |
| Q | 250 | K | 249 |
| A | 251 | A | 250 |
| S | 252 | D | 251 |
| Y | 253 | T | 252 |
| K | 254 | K | 253 |
| I | 255 | I | 254 |
| F | 256 | L | 255 |
| R | 257 | F | 256 |
| I | 258 | I | 257 |
| E | 259 | E | 258 |
| K | 260 | E | 259 |
| G | 261 | G | 260 |
| K | 262 | K | 261 |
| I | 263 | I | 262 |
| V | 264 | I | 263 |
| K | 265 | H | 264 |
| S | 266 | T | 265 |
| V | 267 | S | 266 |
| E | 268 | T | 267 |
| M | 269 | L | 268 |
| N | 270 | S | 269 |
| A | 271 | G | 270 |
| P | 272 | S | 271 |
| N | 273 | A | 272 |
| Y | 274 | Q | 273 |
| H | 275 | H | 274 |
| Y | 276 | V | 275 |
| E | 277 | E | 276 |
| E | 278 | E | 277 |
| C | 279 | C | 278 |
| S | 280 | S | 279 |
| C | 281 | C | 280 |
| Y | 282 | Y | 281 |
| P | 283 | P | 282 |
| D | 284 | R | 283 |
| S | 285 | Y | 284 |
| S | 286 | P | 285 |
| E | 287 | G | 286 |
| I | 288 | V | 287 |
| T | 289 | R | 288 |
| C | 290 | C | 289 |
| V | 291 | V | 290 |
| C | 292 | C | 291 |
| R | 293 | R | 292 |
| D | 294 | D | 293 |
| N | 295 | N | 294 |
| W | 296 | W | 295 |
| H | 297 | K | 296 |
| G | 298 | G | 297 |
| S | 299 | S | 298 |
| N | 300 | N | 299 |
| R | 301 | R | 300 |
| P | 302 | P | 301 |
| W | 303 | I | 302 |
| V | 304 | V | 303 |
| S | 305 | D | 304 |
| F | 306 | I | 305 |
| N | 307 | N | 306 |
| — | NA | I | 307 |
| Q | 308 | K | 308 |
| N | 309 | D | 309 |
| L | 310 | Y | 310 |
| E | 311 | S | 311 |
| Y | 312 | I | 312 |
| Q | 313 | V | 313 |
| I | 314 | S | 314 |
| G | 315 | S | 315 |
| Y | 316 | Y | 316 |
| I | 317 | V | 317 |
| C | 318 | C | 318 |
| S | 319 | S | 319 |
| G | 320 | G | 320 |
| I | 321 | L | 321 |
| F | 322 | V | 322 |
| G | 323 | G | 323 |
| D | 324 | D | 324 |
| N | 325 | T | 325 |
| P | 326 | P | 326 |
| R | 327 | R | 327 |
| P | 328 | K | 328 |
| N | 329 | N | 329 |
| D | 330 | D | 330 |
| K | 331 | S | 331 |
| T | 332 | S | 332 |
| G | 333 | S | 333 |
| S | 334 | S | 334 |
| C | 335 | S | 335 |
| — | NA | H | 336 |
| — | NA | C | 337 |
| — | NA | L | 338 |
| G | 336 | D | 339 |
| P | 337 | P | 340 |
| V | 338 | N | 341 |
| S | 339 | N | 342 |
| S | 340 | E | 343 |
| N | 341 | E | 344 |
| G | 342 | G | 345 |
| A | 343 | G | 346 |
| N | 344 | H | 347 |
| G | 345 | G | 348 |
| V | 346 | V | 349 |
| K | 347 | K | 350 |
| G | 348 | G | 351 |
| F | 349 | W | 352 |
| S | 350 | A | 353 |
| F | 351 | F | 354 |
| K | 352 | D | 355 |
| Y | 353 | D | 356 |
| G | 354 | G | 357 |
| N | 355 | N | 358 |
| G | 356 | D | 359 |
| V | 357 | V | 360 |
| W | 358 | W | 361 |
| I | 359 | M | 362 |
| G | 360 | G | 363 |
| R | 361 | R | 364 |
| T | 362 | T | 365 |
| K | 363 | I | 366 |
| S | 364 | S | 367 |
| I | 365 | E | 368 |
| S | 366 | K | 369 |
| S | 367 | L | 370 |
| R | 368 | R | 371 |
| N | 369 | S | 372 |
| G | 370 | G | 373 |
| F | 371 | Y | 374 |
| E | 372 | E | 375 |
| M | 373 | T | 376 |
| I | 374 | F | 377 |
| W | 375 | K | 378 |
| D | 376 | V | 379 |
| P | 377 | I | 380 |
| N | 378 | E | 381 |
| G | 379 | G | 382 |
| W | 380 | W | 383 |
| T | 381 | S | 384 |
| G | 382 | K | 385 |
| T | 383 | P | 386 |
| D | 384 | N | 387 |
| N | 385 | S | 388 |
| N | 386 | K | 389 |
| F | 387 | L | 390 |
| S | 388 | Q | 391 |
| I | 389 | I | 392 |
| — | NA | N | 393 |
| K | 390 | R | 394 |
| Q | 391 | Q | 395 |
| D | 392 | V | 396 |
| I | 393 | I | 397 |
| V | 394 | V | 398 |
| G | 395 | D | 399 |
| I | 396 | R | 400 |
| N | 397 | G | 401 |
| E | 398 | N | 402 |
| W | 399 | R | 403 |
| S | 400 | S | 404 |
| G | 401 | G | 405 |
| Y | 402 | Y | 406 |
| S | 403 | S | 407 |
| G | 404 | G | 408 |
| S | 405 | I | 409 |
| F | 406 | F | 410 |
| V | 407 | — | NA |
| Q | 408 | — | NA |
| H | 409 | — | NA |
| P | 410 | — | NA |
| E | 411 | S | 411 |
| L | 412 | V | 412 |
| T | 413 | E | 413 |
| G | 414 | G | 414 |
| L | 415 | K | 415 |
| D | 416 | S | 416 |
| C | 417 | C | 417 |
| I | 418 | I | 418 |
| R | 419 | N | 419 |
| P | 420 | R | 420 |
| C | 421 | C | 421 |
| F | 422 | F | 422 |
| W | 423 | Y | 423 |
| V | 424 | V | 424 |
| E | 425 | E | 425 |
| L | 426 | L | 426 |
| I | 427 | I | 427 |
| R | 428 | R | 428 |
| G | 429 | G | 429 |
| R | 430 | R | 430 |
| P | 431 | K | 431 |
| K | 432 | E | 432 |
| E | 433 | E | 433 |
| N | 434 | T | 434 |
| T | 435 | E | 435 |
| I | 436 | V | 436 |
| — | NA | L | 437 |
| W | 437 | W | 438 |
| T | 438 | T | 439 |
| S | 439 | S | 440 |
| G | 440 | N | 441 |
| S | 441 | S | 442 |
| S | 442 | I | 443 |
| I | 443 | V | 444 |
| S | 444 | V | 445 |
| F | 445 | F | 446 |
| C | 446 | C | 447 |
| G | 447 | G | 448 |
| V | 448 | T | 449 |
| N | 449 | S | 450 |
| S | 450 | G | 451 |
| D | 451 | T | 452 |
| T | 452 | Y | 453 |
| V | 453 | G | 454 |
| G | 454 | T | 455 |
| W | 455 | G | 456 |
| S | 456 | S | 457 |
| W | 457 | W | 458 |
| P | 458 | P | 459 |
| D | 459 | D | 460 |
| G | 460 | G | 461 |
| A | 461 | A | 462 |
| E | 462 | D | 463 |
| L | 463 | I | 464 |
| P | 464 | N | 465 |
| F | 465 | L | 466 |
| T | 466 | — | NA |
| I | 467 | M | 467 |
| D | 468 | P | 468 |
| K | 469 | I | 469 |

### EXAMPLES

### Example 1

### Anti-NA and Anti-HA

### Monoclonal Antibodies and Functional Testing of Combinations of these

Anti-hemagglutinin (HA) antibodies and anti-neuraminidase (NA) antibodies were isolated from donor tonsillar and PBMC samples.

For HA antibodies, peripheral blood mononuclear cells (PBMCs) from anonymous donors were selected based on neutralization by the corresponding serum against H5 (Group 1) and H7 (Group 2) influenza pseudoviruses. Donors were selected by screening serum from tonsillar donor samples (n=50) for reactivity against hemagglutinin subtype H5 and H7 antigens, and serum from PBMC donor samples (n=124) for reactivity against H5 and H7 subtype pseudoviruses. Binding was evaluated by FACS. B memory cells from five donors were sorted by flow cytometry for input into the discovery workflow. Single sorted B cells (n=6,700) were co-cultured with mesenchymal stromal cells (MSC) in 50 μl culture to stimulate antibody secretion. Secreted antibodies were evaluated using binding and pseudovirus neutralization assays. Binding to HAs from group I influenza A viruses (IAV), group II IAVs, and influenza B viruses was evaluated by enzyme-linked immunosorbent assay (ELISA) to determine breadth. Neutralization—measured as blockade of viral entry and uncoating—was evaluated by monitoring luciferase expression following infection of target cells with H5 or H7 luciferase (Luc)-expressing pseudovirus particles. Antibody sequences from selected B cells were cloned as cDNAs and sequenced.

Clonally related anti-HA antibodies “FHF11” and “FHF12” were selected for further studies, and sequence variants of these with one or more variable domain mutations were generated (see Table 1 SEQ ID NOs.:1-42). FHF11 and FHF12 each bound to several HAs circulating in the animal reservoir by FACS, and to group 1 (H1, H2, H5, H9) and group 2 (H3) HAs by ELISA. These antibodies also bound to H1 A/Swine/Jiangsu/J004/2018 by FACS, and did not exhibit polyreactivity against healthy human epithelial type 2 (HEP-2) cells. FHF11 activated FcγRIIIa (F158) in the presence of H1N1 and H3N2, similar to or to a greater degree than FM08_LS. FHF11 also activated FcγRIIa (H131) in the presence of H1N1 and H3N2, similar to or to a greater degree than FM08_LS.

Various other experiments were performed to characterize FHF11 and FHF12 (parental and variant) antibodies. For example, FHF11v3, FHF11v6, and FHF11v9 bound to panels of H3N2, H1N1, H2N2, H3N1, and H9N2 subtypes by ELISA. FHF11, FHF11v3, FHF11v6, FHF11v9, FY1, and FM08 bound to HA-5 with Kd values of less than 1.0E-12, and with similar or lower affinities to HA-7, by BLI. FHF11, FHF11v3, FHF11v6, and FHF11v9 neutralized H5 pp with 1C50 values between approx. 0.7 and 0.2 ng/mL. Antibodies were examined for neutralization against a number of H1N1 and H3N2 viruses, and for activation of FcγRs in the presence of H1N1 and H3N2. FHF11v9-LS, FHF12-LS, and FM08-LS were assessed for in vivo pharmacokinetics in tg32 mice. FHF11v9 was also investigated for prophylactic effect and pharmacokinetics in BALB/c mice pre-treated with antibody and then infected with H1N1 A/Puerto Rico/8/34 or H3N2 A/Hong Kong/68, and for pharmacokinetics in SCID tg32 mice.

For NA antibodies, peripheral blood mononuclear cells (PBMCs) from anonymous donors were selected based on binding of the corresponding serum against N1 and N4 (G1); and N2, N3 and N9 (G2) influenza pseudoviruses. Donors were selected by screening serum from tonsillar donor samples (n=50) for reactivity against neuraminidase subtype N1 and N2 antigens, and serum from PBMC (peripheral blood mononuclear cell) donor samples (n=124) for reactivity against neuraminidase subtype N4, N3, and N9. Neuraminidase antigens for screening were expressed in mammalian cells and binding was evaluated by FACSflow cytometry.

B memory cells from five donors were sorted by flow cytometry for input into the discovery workflow. Single sorted B cells (n=39,350) were co-cultured with mesenchymal stromal cells (MSC) in 50 μl cultures to stimulate antibody secretion. Secreted antibodies were evaluated by binding and NA inhibition assays. Inhibition of N1 sialidase activity was evaluated using ELLA (enzyme-linked lectin assay), an absorbance-based assay that utilizes a large glycoprotein substrate, fetuin, as a substrate for sialic acid cleavage by NA (Lambre et al. J Immunol Methods. 1990). Inhibition of N1, N2, and N9 sialidase activity was measured using a fluorescence-based assay that measures cleavage of the 2′-(4-Methylumbelliferyl)-α-D-N-acetylneuraminic acid (MUNANA) by the NA enzyme (Potier et al. Anal. Biochem. 1979.).

Binding to NAs from group 1 IAV N1 A/Vietnam/1203/2004, and group 2 IAVs N2 A/Tanzania/205/2010 and N9 A/Hong Kong/56/2015 was evaluated by ELISA to determine breadth. Antibody sequences from selected B cells were cloned as cDNAs and sequenced.

Fourteen clonally related monoclonal antibodies (“FNI”—prefix) resulted from the discovery workflow. These, as well as an antibody containing the unmutated common ancestor (UCA) VH and VL, exhibited binding against a breadth of IAV and IIBV NAs. Antibodies FNI3 and FNI9 demonstrated comparable or even stronger binding to NAs (N1, N2, N9) relative to reference antibody 1G01 (Stadlbauer et al. (Science 366(6464):499-504 (2019)) by ELISA and BLI. FNI3 and FNI9 showed comparable or stronger binding against a panel of Group 1 and Group 2 IAV NAs and stronger binding against a panel of IBV NAs, as compared to 1G01. FNI3 and FNI9 inhibited sialidase activity of H3N2 IAV NAs that include a glycosylation motif at positions 245 (245Gly+) and 247 (247Gly+) (Wan et al. Nat Microbiolog. 2019): A/South Australia/34/2019, A/Switzerland/8060/2017, A/Singapore/INFUIH-16-0019/2016, and A/Switzerland/9715293/2013. FNI3 and FNI9 bound to N2 A/South Australia/34/2019, and N1 A/Swine/Jiangsu/J004/2018, and did not exhibit polyreactivity against healthy Hep2 cells. FNI3 and FNI9 demonstrated strong NAI activity against a panel of IAV G1 NAs, IAV G2 NAs, and IBV NAs. Other experiments demonstrated neutralization of H1N1, H3N2, B/MAL (Victoria lineage), B/JIA (Yamagata lineage), by FNI antibodies, and inhibitory activity by FNI3 against H1N1 and H3N2 engineered to include oseltamivir-resistant mutations, with greater potency than oseltamivir, FM08, and 1G01.

Structural studies showed that CDRH3 of FNI3 interacted with the NA active site that oseltamivir occupies. FNI3 epitope is conserved in N2 NA sequences from H3N2 (n=60,597) and in N1 NA sequences from H1N1 (n=57,597) isolated between the years 2000 and 2020. FNI3 and FNI9 were also tested in mouse models of prophylaxis against LD90 H1N1 PR8 or H3N2 HK/68 influenza; pre-treated mice showed a generally dose-dependent lack of weight loss. Mice treated with FNI3 or FNI9 had improved survival and weight loss versus vehicle over a 15-day study of infection with H3N2 A/HongKong/8/1968. FNI3 and FN19 bearing MLNS Fc mutations had improved half-life (mean values of 12.034 days (SD 1.781 days) and 14.198 days (SD 2.014 days), respectively) versus FM08_MLNS (8.072 days; SD 1.567 days) in tg32 mice. 1G01_MLNS had a calculated half-life of 12.636 days (SD 2.23 days).

Antibodies FNI17 and FNI19 were also investigated. These antibodies had comparable breadth of NA binding to FNI3 and FNI9, and had improved binding versus these antibodies against certain NAs. FNI17 and FNI19 potently neutralized a panel of H3N2 and H1N1 IAVs, as well as a panel of IBVs. FNI3, FN19, FNI17, and FNI19 demonstrated improved neutralization versus FM08 and FHF11 against a pantel of H1N1 and H3N2 viruses, as measured by nucleoprotein staining. FNI3, FNI9, FNI17, and FNI19 neutralized the same panel of viruses with greater potency than oseltamivir, and activated FcγRIIIa, and to lesser extent FcγRIIa, on Jurkat cells with A549 infected with H1N1 PR8 (MOI=6). These antibodies also activated FcγRIIIa in the presence of N1 and N2 IAV NAs and IBV NAs, while FNI3 and FNI9, but not FNI17 and FNI19, activated FcγRIIIa in the presence of a N9 NA. FNI3-LS, FNI9-LS, FNI17-LS, and FNI19-LS showed improved half-life versus FM08-LS in SCID tg32 mice over 30 days post-administration. FNI3-LS, FNI9-LS, FNI17-LS, and FNI19-LS were not polyreactive against Hep-2 cells. Structural studies showed that FNI3, FNI17, and FNI19 have similar docking orientations to NA. The CDRH3s of these antibodies are very similar in their amino acid sequences. Sequence variants of FNI17 and FNI19 were generated. FNI17v19 and FNI19v3 had improved in in vivo half-life in tg32 mice (injected i.v. with 5 mg/kg antibody) as compared to their respective parental antibodies (FNI17v19-LS=14.88 t 3.27 days; FNI17-LS=8.86 t 0.57 days; FNI19v3-LS=14.40±2.13 days; FNI9-LS=11.57±0.63 days). FNI17v19 and FNI19v3 inhibited sialidase activity of a panel of H1N1, H3N2, B/Victoria-lineage, and B/Yamagata-lineage influenza viruses. FNI17-LS improved survival as compared to FM08_LS over twelve days in BALB/c mice infected with H1N1 A/Puerto Rico/8/34. Additional characterization studies using FNI antibodies including FNI17v19 and FNI19v3 were performed.

Anti-HA FHF11 (VH: SEQ ID NO.:2; VL: SEQ ID NO.:8), and anti-NA FNI3 (VH: SEQ ID NO.:72; VL: SEQ ID NO.:78) FNI9 (VH: SEQ ID NO.:132; VL: SEQ ID NO.:138), FNI17 (VH: SEQ ID NO.:192; VL: SEQ ID NO.: 198), FNI17-v19 (VH: SEQ ID NO.:241; VL: SEQ ID NO.:243), FNI19 (VH: SEQ ID NO.:204; VL: SEQ ID NO.:210), and FNI19-v3 (VH: SEQ ID NO.:245; VL: SEQ ID NO.:247) are non-limiting examples of the aforementioned antibodies that bind with high affinity to antigen from, and have robust neutralizing activity against, a variety of influenza viruses.

The ability of anti-NA+anti-HA antibody combinations to inhibit sialidase activity was evaluated in in vitro assays. Anti-NA antibodies FNI3 and FNI9, and anti-HA monoclonal antibodies FHF11 and “FM08” (VH: SEQ ID NO.:43; VL: SEQ ID NO.:44; see also Kallewaard et al. Cell 166(3):596-608 (2016)) were tested using a fluorescence-based assay for sialidase inhibition that measures cleavage of the 2′-(4-Methylumbelliferyl)-α-D-N-acetylneuraminic acid (MUNANA) by the NA enzyme (Potier et al. Anal. Biochem. 1979). Inhibition of H1 Cal/09 sialidase activity by FM08+FNI3 (FIG. 1A), FM08+FNI9 (FIG. 1B), and FHF11+FNI9 (FIG. 1C) was measured. Additionally, inhibition of H3N2 HK/68 sialidase activity by FM08+FNI3 (FIG. 1D), FM08+FNI9 (FIG. 1E), FHF11+FNI9 (FIG. 1F) was also tested. Heatmaps were generated to visualize neutralization (%) at μg/ml (FIGS. 1A-1F, top panels) and Synergy/Antagonism score (FIGS. 1A-1F, bottom panels) between each antibody pair tested. These data show synergistic neutralizing effects of anti-HA+anti-NA antibody combinations.

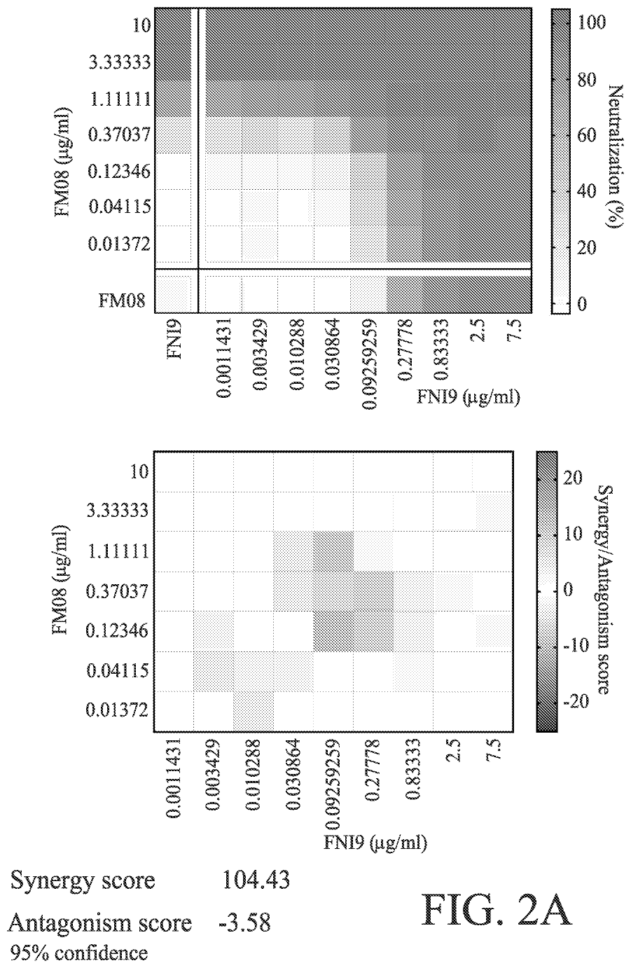

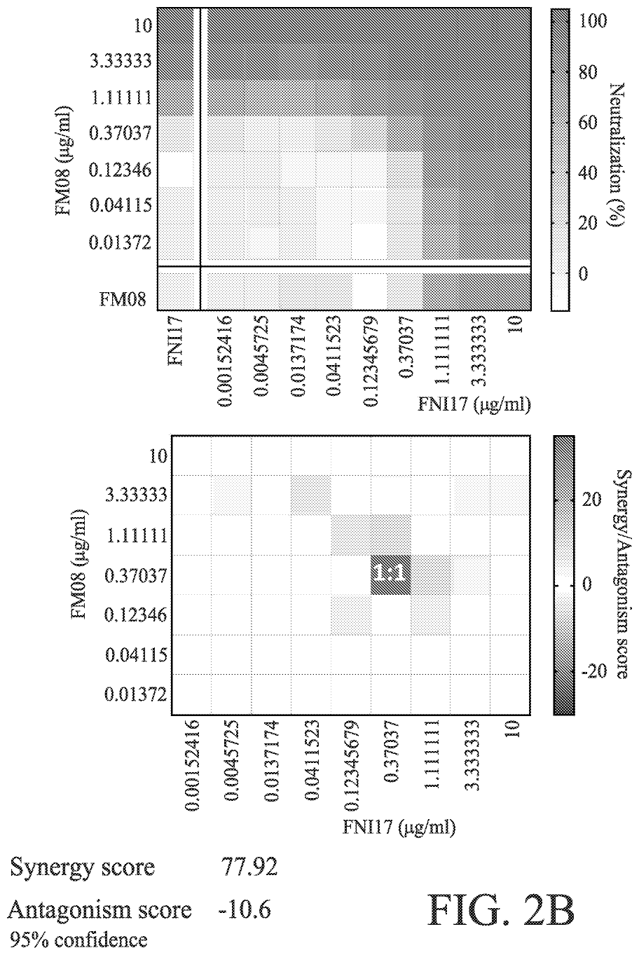

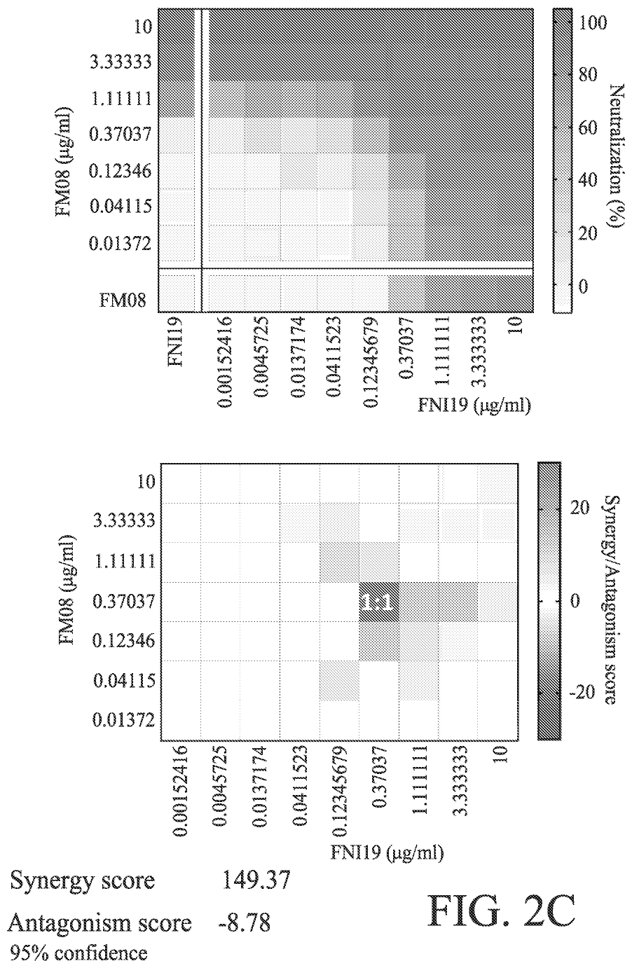

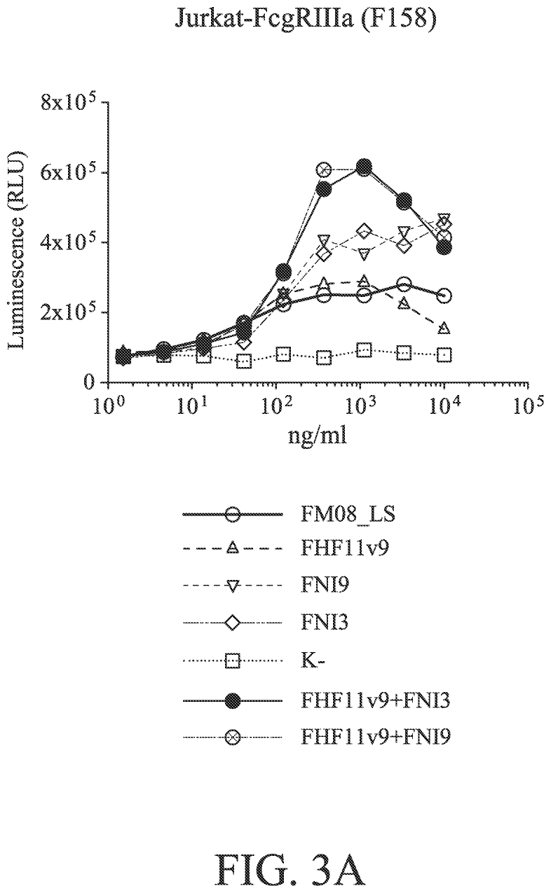

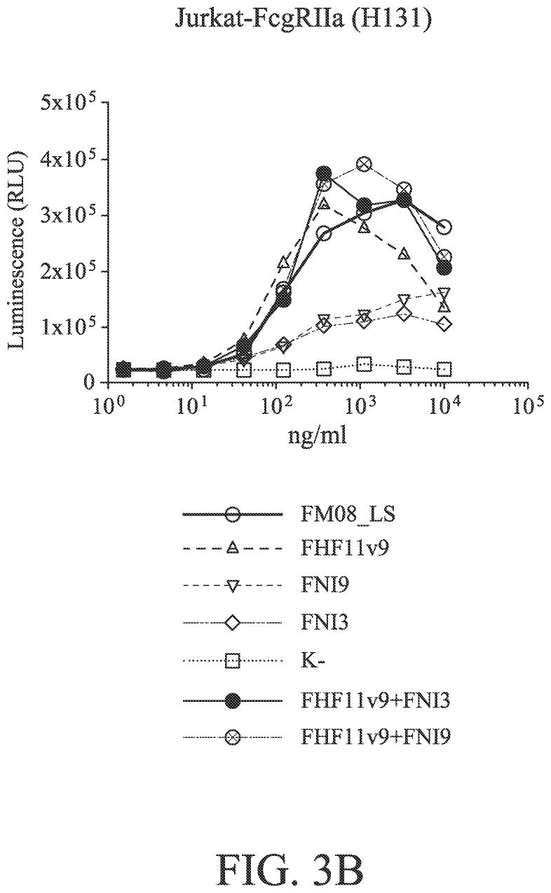

The ability of anti-NA+anti-HA antibody combinations to inhibit sialidase activity was also evaluated using nucleoprotein staining. Anti-NA antibodies FNI9 (VH: SEQ ID NO.:132; VL: SEQ ID NO.:138), FNI17 (VH: SEQ ID NO.:192; VL: SEQ ID NO.:198), and FNI19 (VH: SEQ ID NO.:204; VL: SEQ ID NO.:210) and anti-HA monoclonal antibody FM08 were tested. Inhibition of H3N2 A/Hong Kong/1/1968 sialidase activity by FM08+FN19 (FIG. 2A), FM08+FNI17 (FIG. 2B), and FM08+FNI19 (FIG. 2C) was measured. Heatmaps were generated to visualize neutralization (%) at μg/ml (FIGS. 2A-2C, top panels) and Synergy/Antagonism score (FIGS. 2A-2C, bottom panels) between each antibody pair tested. Synergy matrix and score were generated using MacSynergyII. These data show synergistic neutralizing effects of anti-HA+anti-NA antibody combinations. Anti-NA antibodies, anti-HA antibodies, and combinations thereof were tested for the ability to activate FcγRIIIa (FIG. 3A; F158 allele) and FcγRIIa (FIG. 3B; H131 allele) using a NFAT-driven luciferase reporter assay. Individual anti-NA (FNI3, FNI9) and anti-HA variant antibody FHF11_v9 (VH: SEQ ID NO.:37; VL: SEQ ID NO.:8) antibodies, and combinations thereof were tested using a NFAT-mediated Luciferase reporter in engineered Jurkat cells following contact with A549 cells pre-infected with H1N1 A/PR/8/34. Activation by comparator FM08_LS, comprising M428L and N434S (EU numbering) Fc mutations, and a negative control antibody against an irrelevant antigen, “K-” was also measured. These data show that anti-HA+anti-NA antibody combinations can improve activation of FcγRs in the context of H1N1 influenza infection.

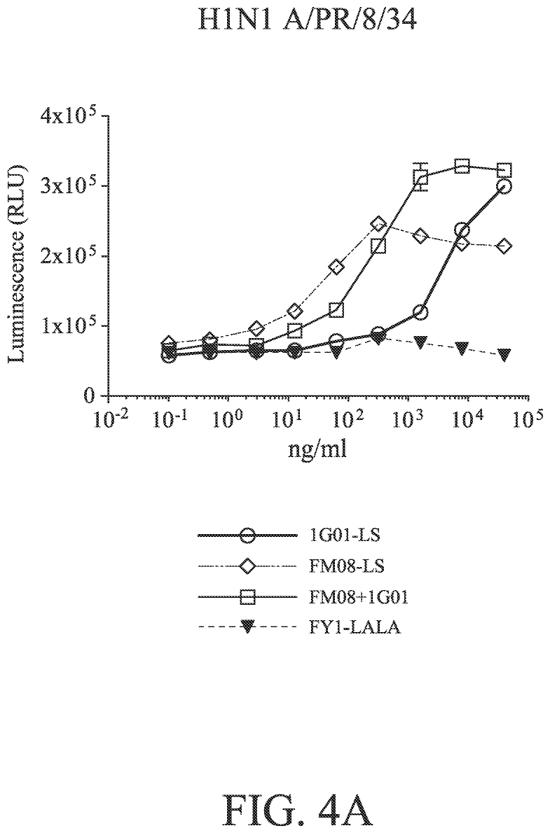

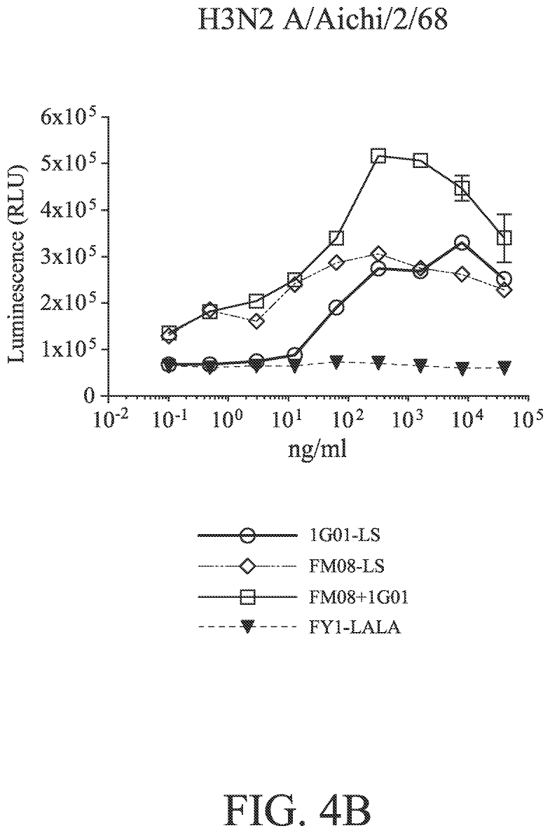

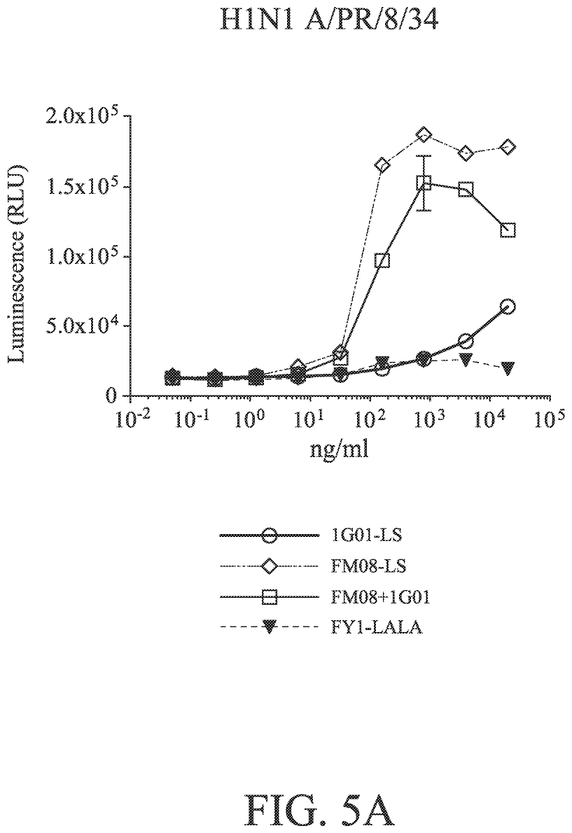

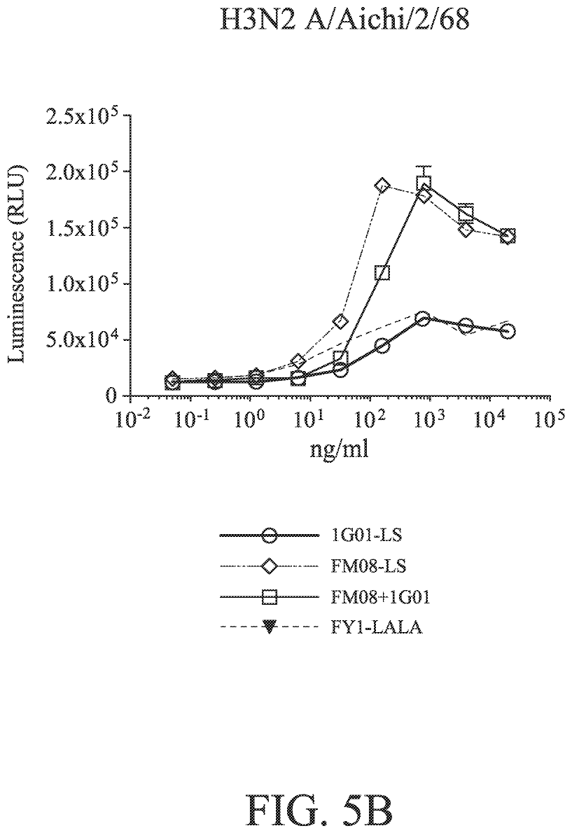

FcγRIIIa- and FcγRIIa-activation by anti-NA 1G01-LS, anti-HA FM08-LS, and the combination of both, was also tested using the luciferase assay. FcγRIIIa activation was measured following contact with A549 cells pre-infected with H1N1 A/PR/8/34 (FIG. 4A) or H3N2 A/Aichi/2/68 (FIG. 4B). FcγRIIa activation was measured following contact with A549 cells pre-infected with H1N1 A/PR/8/34 (FIG. 5A) or H3N2 A/Aichi/2/68 (FIG. 5B). Activation by a negative control antibody (FY1-LALA) was also measured.

### Example 2

### Prophylactic Activity of Anti-NA/Anti-HA Antibody Combinations

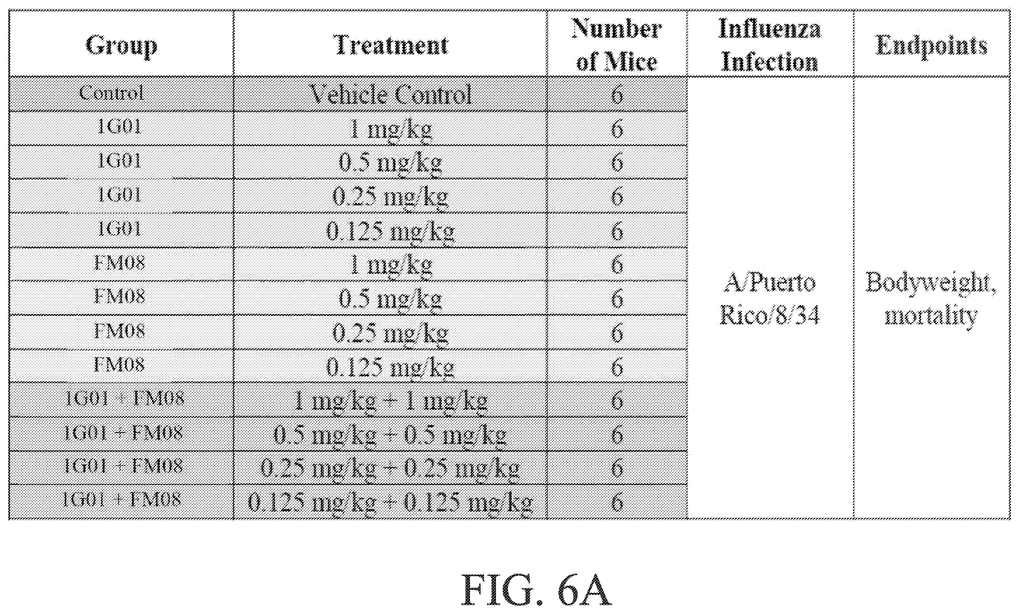

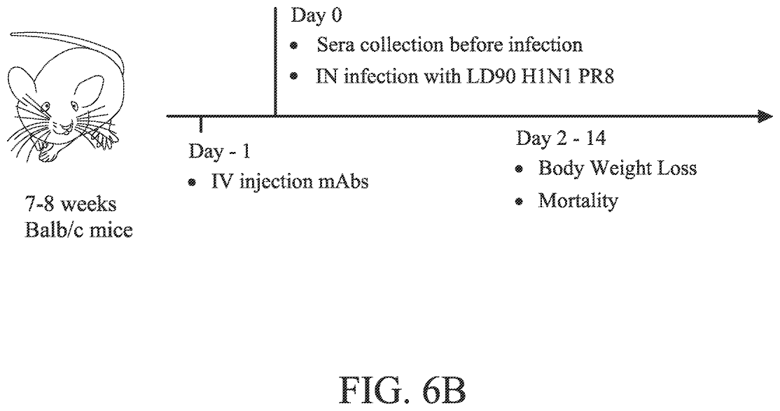

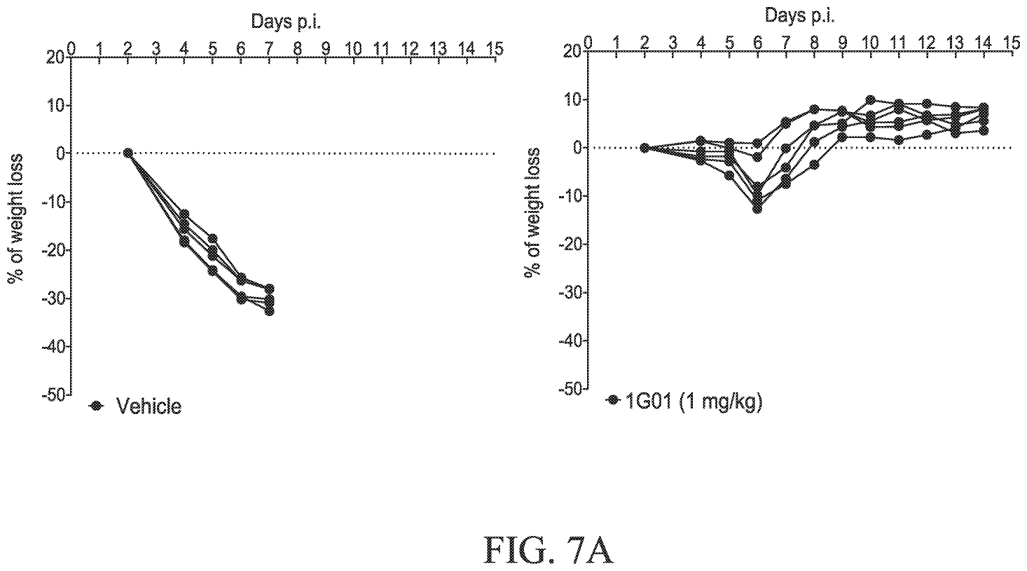

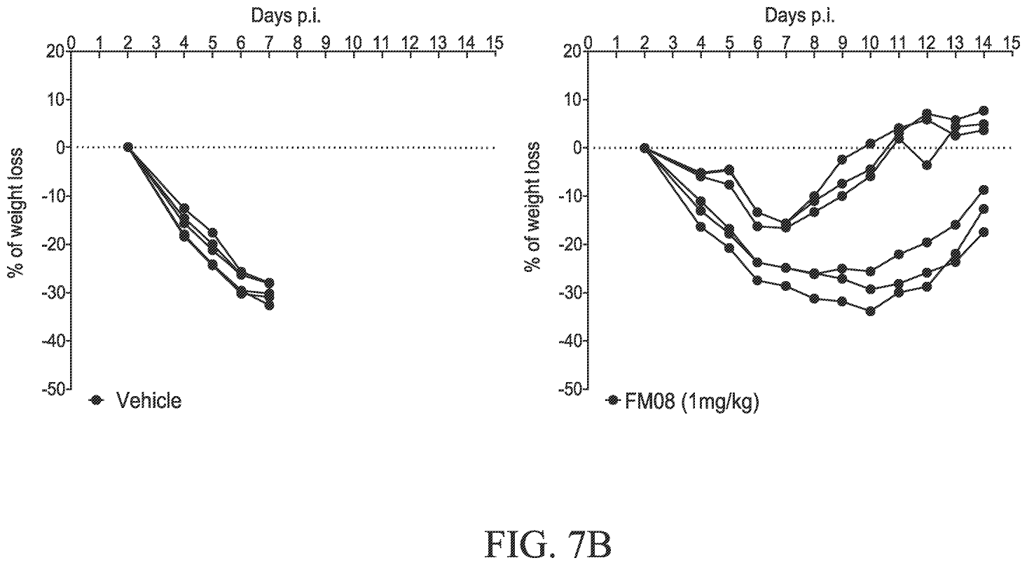

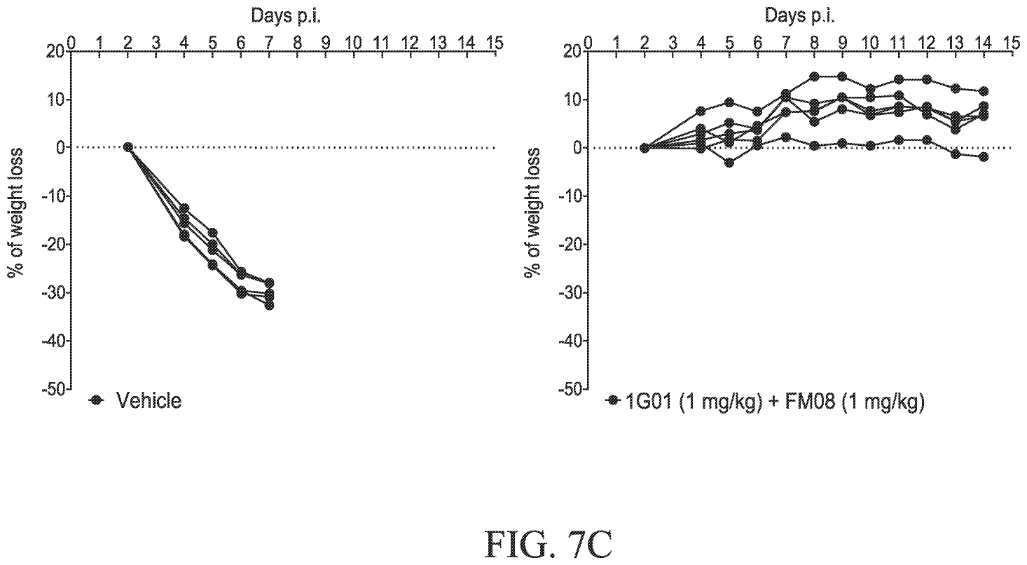

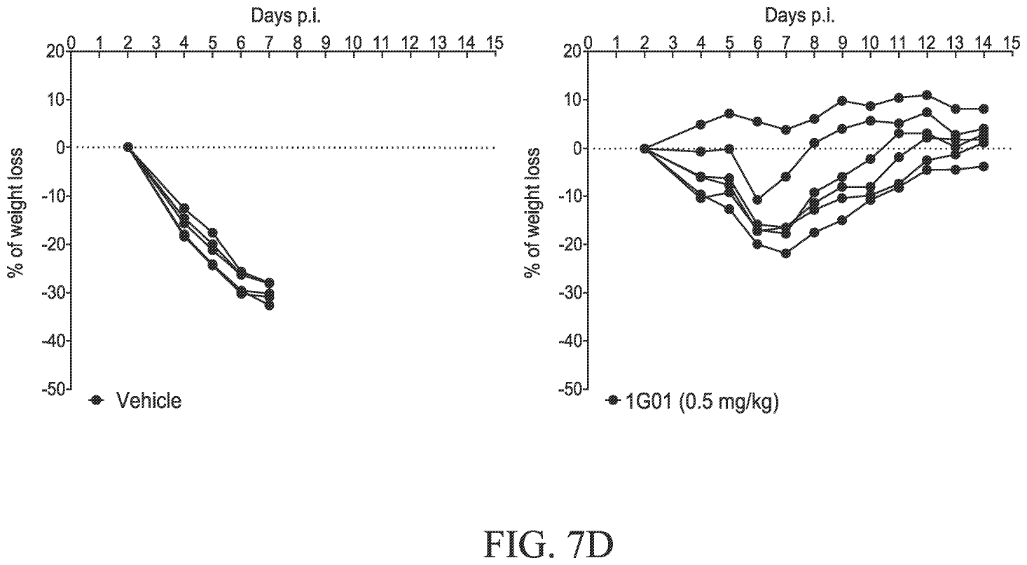

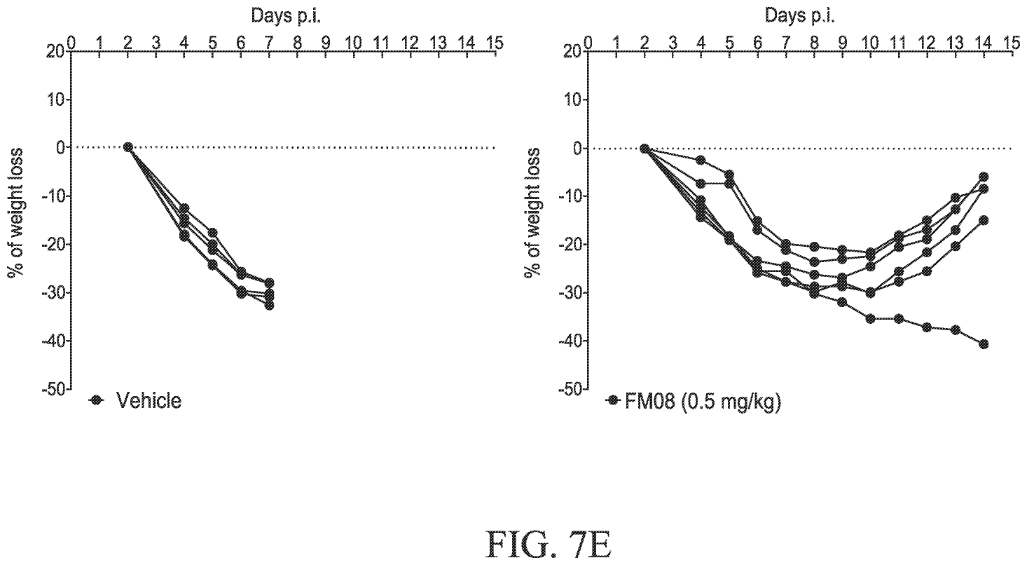

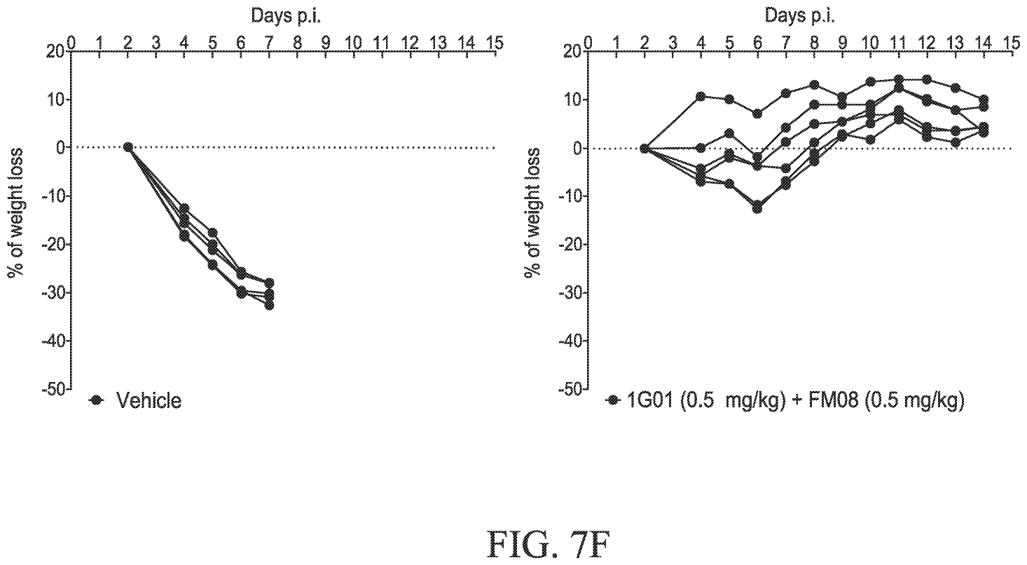

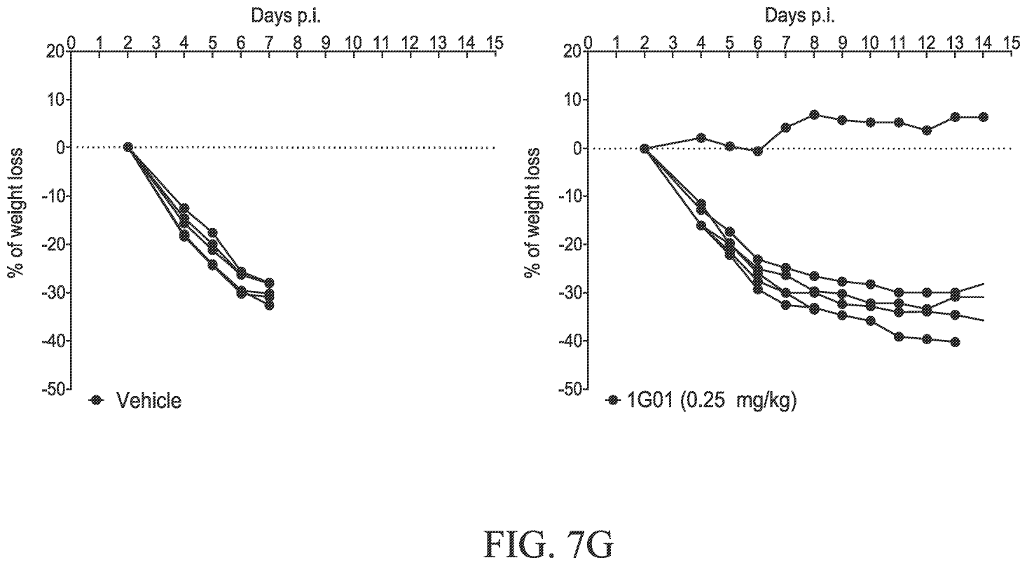

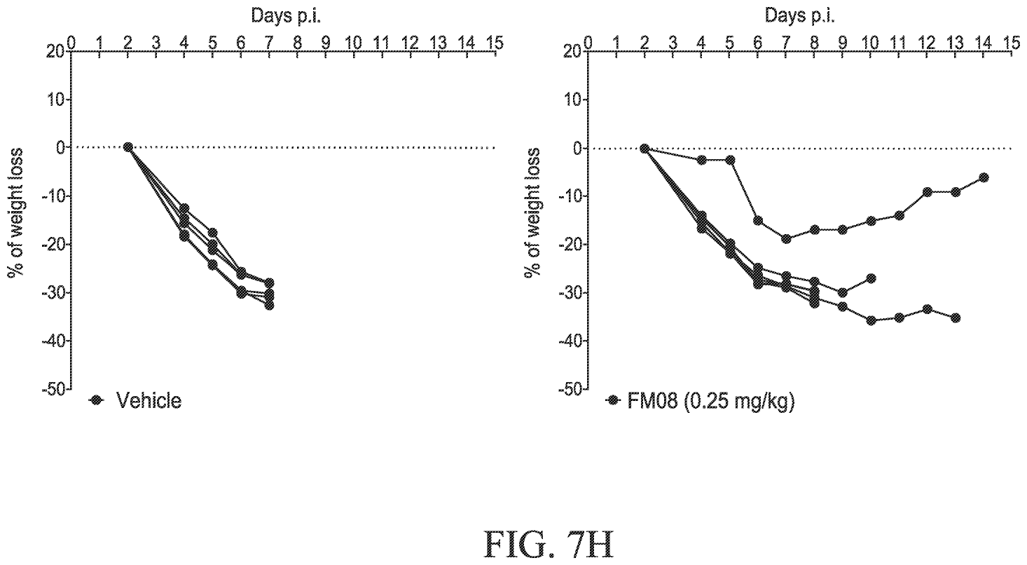

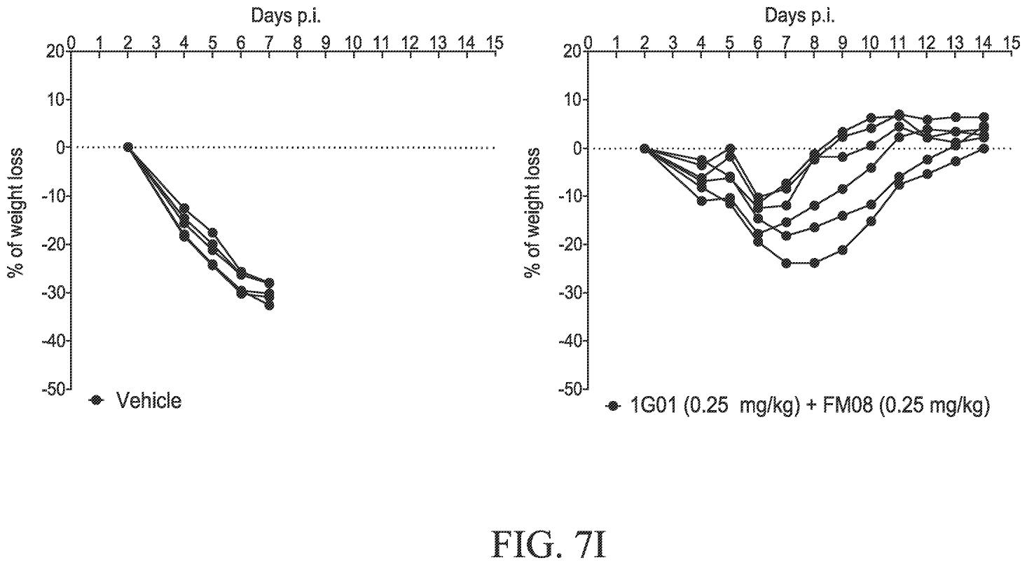

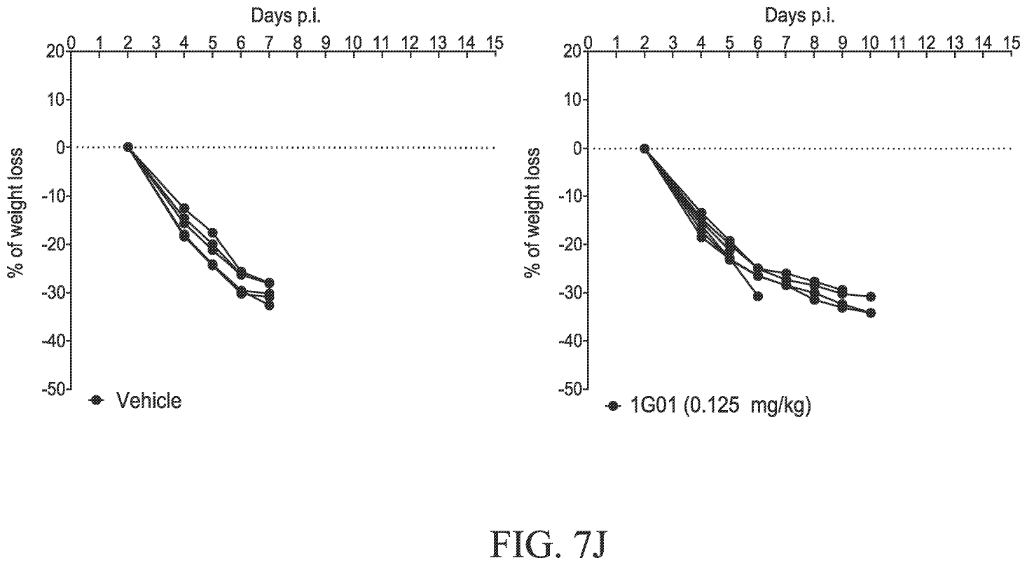

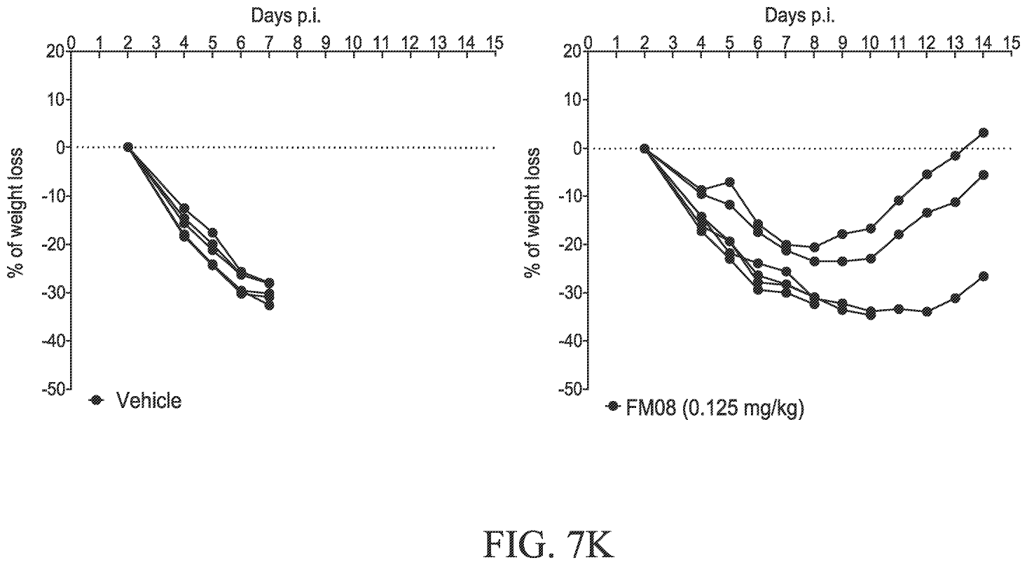

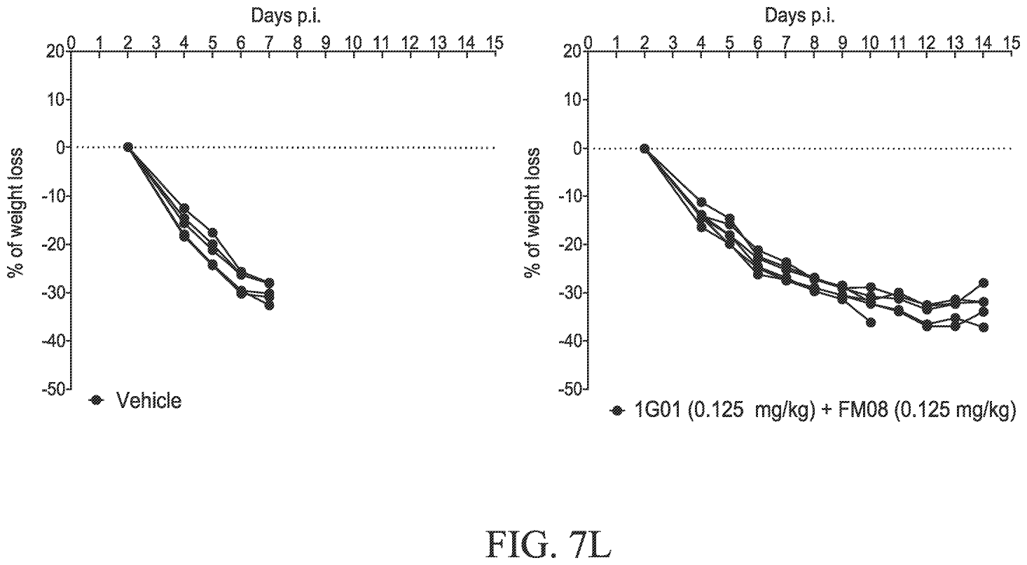

Prophylactic activity of an anti-NA antibody (1G01), an anti-HA antibody (FM08), and the combination of both, was evaluated in a murine BALB/c model of IAV infection. Briefly, BALB/c mice, 7-8 weeks of age, were administered (i.v.) 1G01, FM08, 1G01+FM08, or vehicle control one day prior to intranasal infection at LD90 (90% of a lethal dose) with H1N1 subtype A/Puerto Rico/8/34 (FIGS. 6A and 6B). Each antibody was administered (i.v.) at 1.0, 0.5, 0.25, or 0.125 mg/kg. Baseline serum was collected at the start of infection, and both body weight and mortality were evaluated on each of Days 2-14 post-infection (FIG. 6B). Body weight measurements over fifteen days are shown in FIGS. 7A-7C (1.0 mg/kg test group), FIGS. 7D-7F (0.5 mg/kg test group), FIGS. 7G-7I (0.25 mg/kg test group), and FIGS. 7J-7L (0.125 mg/kg test group). In particular, improvement was observed with the combination at 0.25 mg/kg of each antibody.

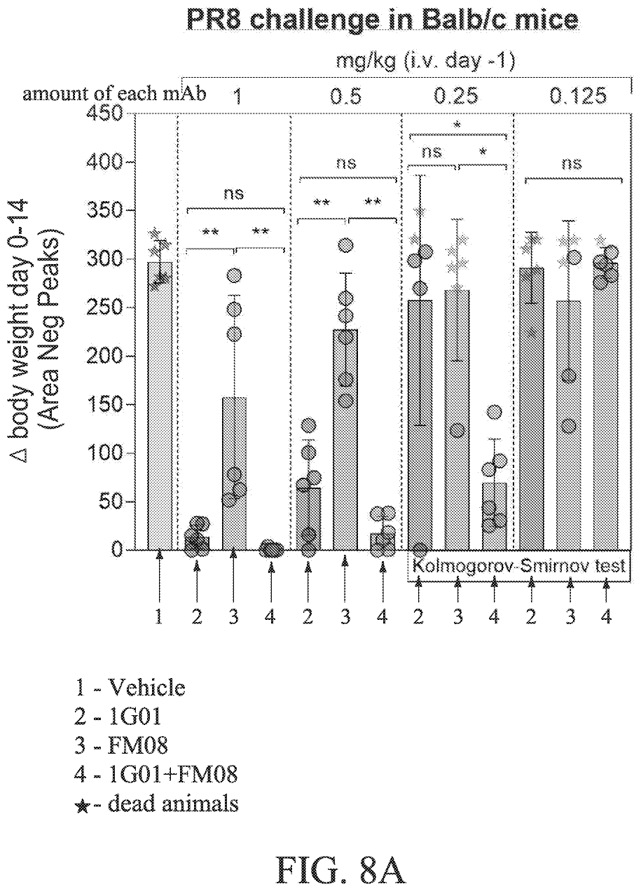

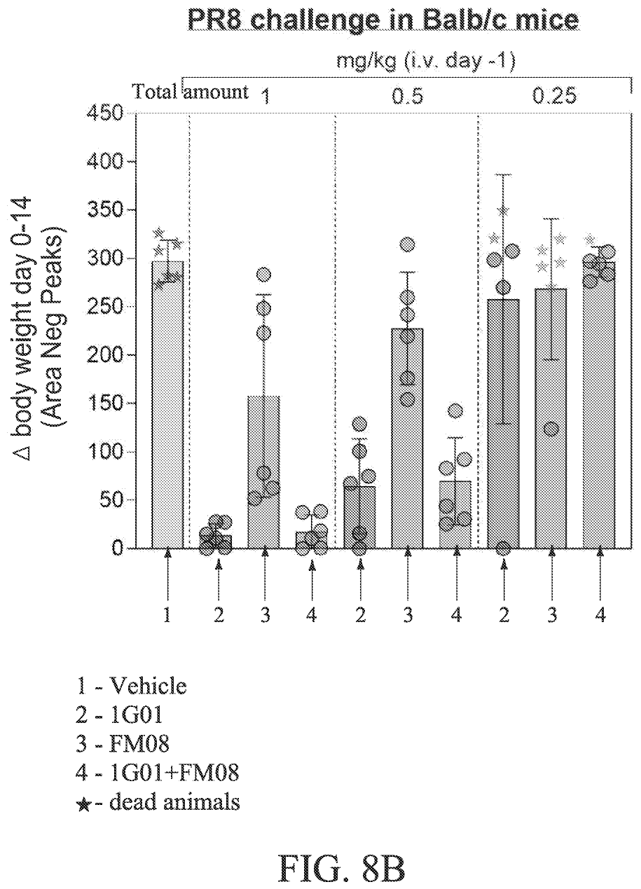

Negative area-under-the-curve peaks compared with IgG in serum from area-under-the-curve analyses of body weight loss in BALB/c mice infected with A/Puerto Rico/8/34 following treatment with 1G01, FM08, or 1G01+FM08 are also shown (FIGS. 8A-8B). Negative area-under-the-curve peaks are graphed by amount of each mAb (FIG. 8A) or amount of total antibody (FIG. 8B) administered in mg/kg. Analysis of area-under-the-curve of body weight loss in BALB/c mice infected with A/Puerto Rico/8/34 following treatment with 1G01+FM08 was analyzed using Compusyn software (combosyn.com).

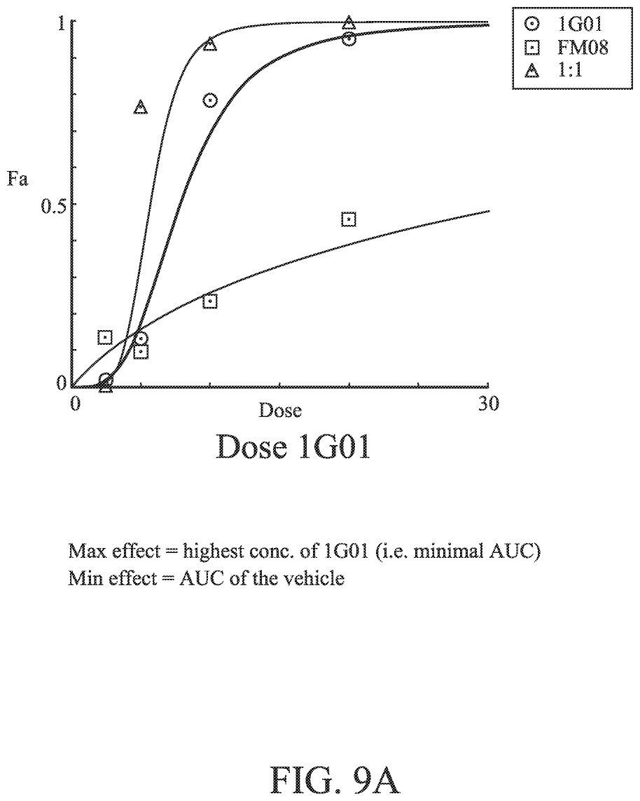

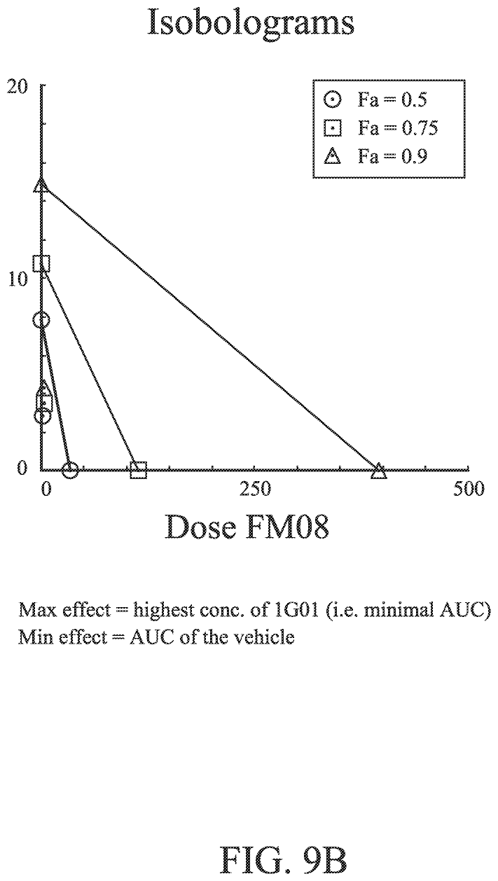

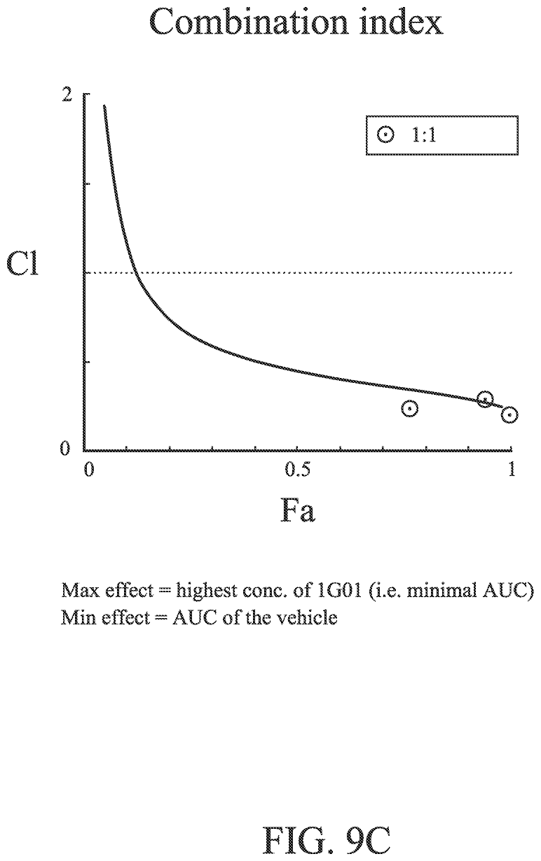

The dose-effect curve (FIG. 9A) is shown, as well as an isobologram (i.e. equi-effective curve, FIG. 9B) for 50%, 75% and 90% inhibition. A combination index (FIG. 9C) was also determined for quantitative definition of synergism, additive effect, and antagonism.

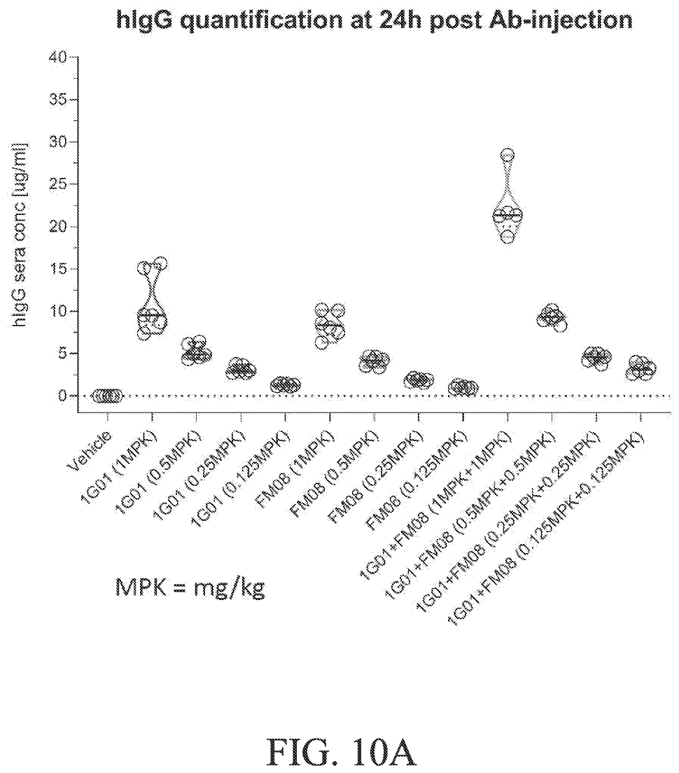

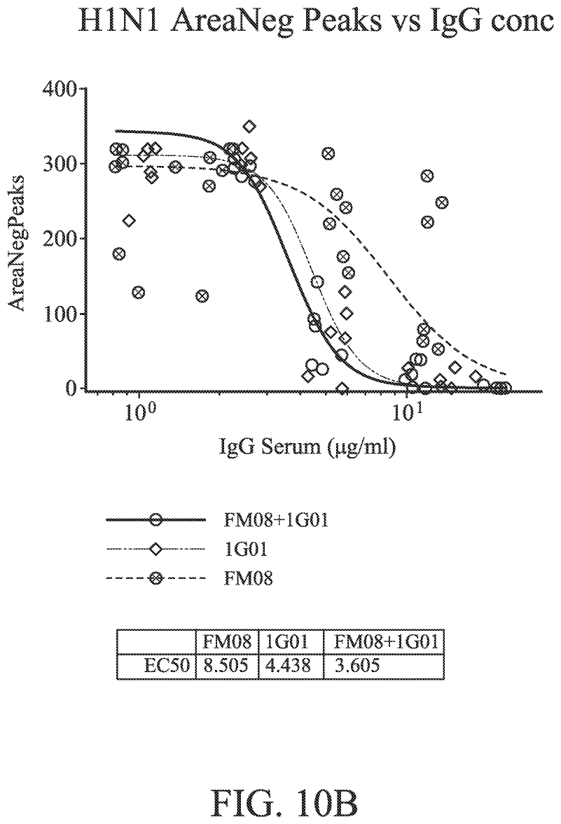

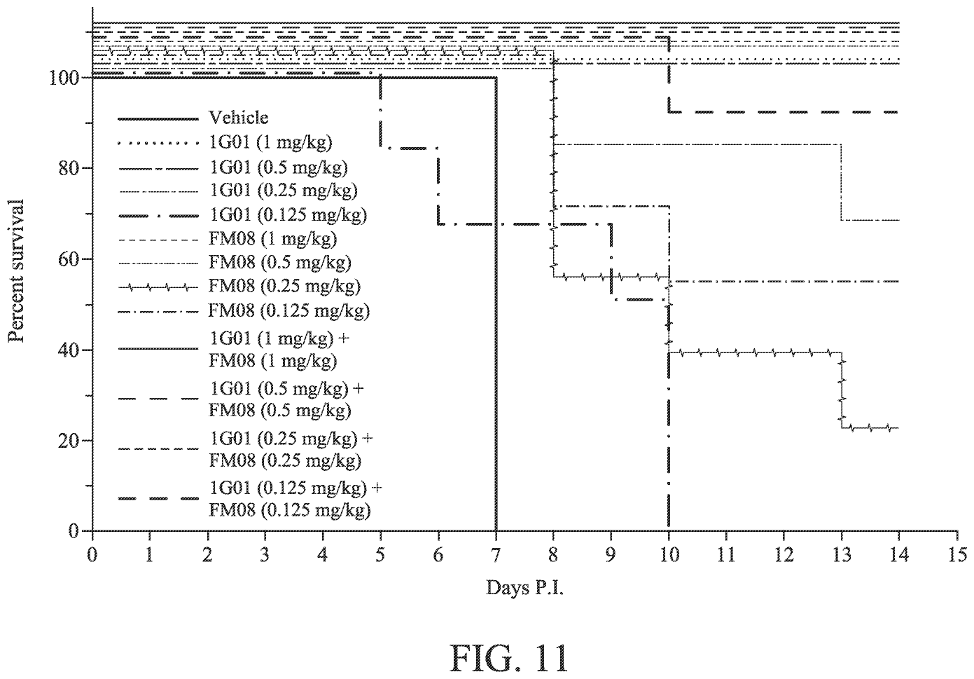

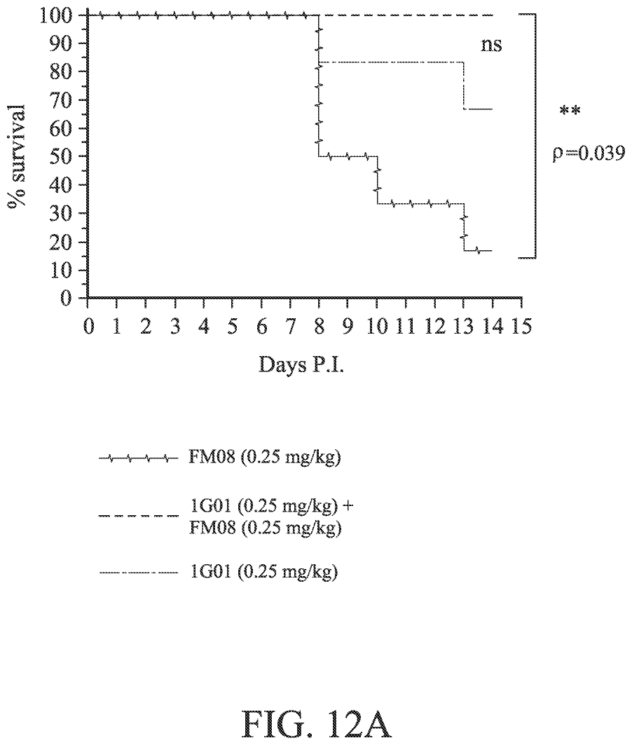

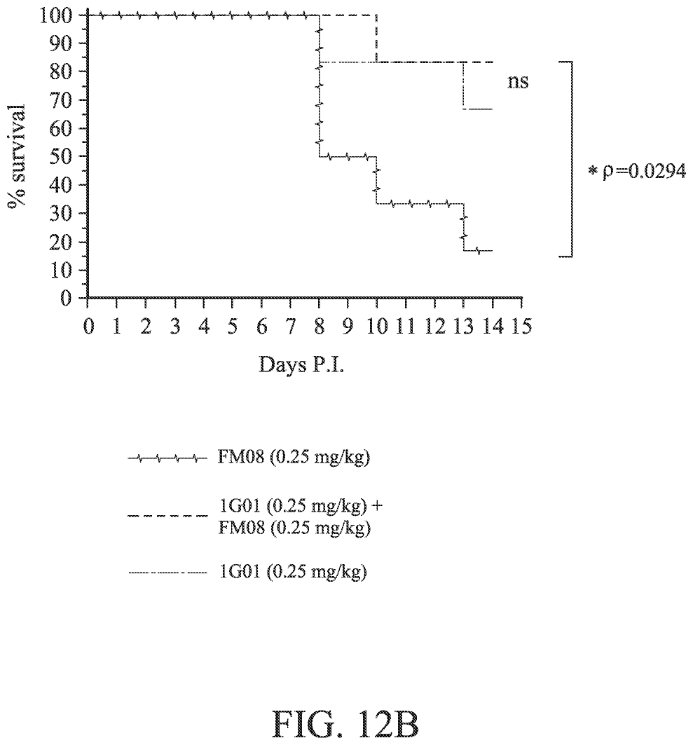

Serum human IgG was measured in mice 24-hours post-antibody injection and immediately prior to infection with a LD90 (90% lethal dose) of A/Puerto Rico/8/34 according to the timeline shown in FIG. 6B. FIG. 10A shows human IgG in serum at 24-hours post-antibody injection reported as μg/ml. FIG. 10B shows H1N1 negative area-under-the-curve peaks compared with IgG in serum and EC50 (half maximal effective concentration) values from area-under-the-curve analyses of body weight loss (FIG. 8A-8B). Overall mortality was also measured (FIG. 11, FIGS. 12A-12B). Animals that received even the lowest dose of the antibody combination (0.125 mg/kg per mAb; FIG. 11) had improved survival versus certain higher doses of single antibodies.

### Example 3

### Additional Studies

Neutralization of anti-HA and anti-NA FNI antibody combinations, including FHF11+FNI, FHF11+1G01, and FM08+FNI, is tested in vitro against additional H1N1 and H3N2 viruses, and against anti-HA or anti-NA monoclonal antibody-resistant mutant (MARM) influenza. In vitro resistance selection assays are performed. Bispecific anti-HA x anti-NA antibodies are generated in DVD-Ig and IEI-Ig formats. In vivo studies using Balb/c mice are performed to test prophylactic activity of anti-HA alone, of anti-NA alone, of anti-HA x anti-NA combinations (e.g., of FHF11+FNI, FHF11+1G01, and FM08+FNI), and of bispecific antibodies, against H1N1 PR8 and H3N2 HK/68 (LD90). Four doses are used for each test article. Study endpoints are body weight loss and survival up to 14 days post-infection.

### Example 4

### Design and In Vitro Testing of Dual Variable Domain (DVD) Bispecific Antibodies

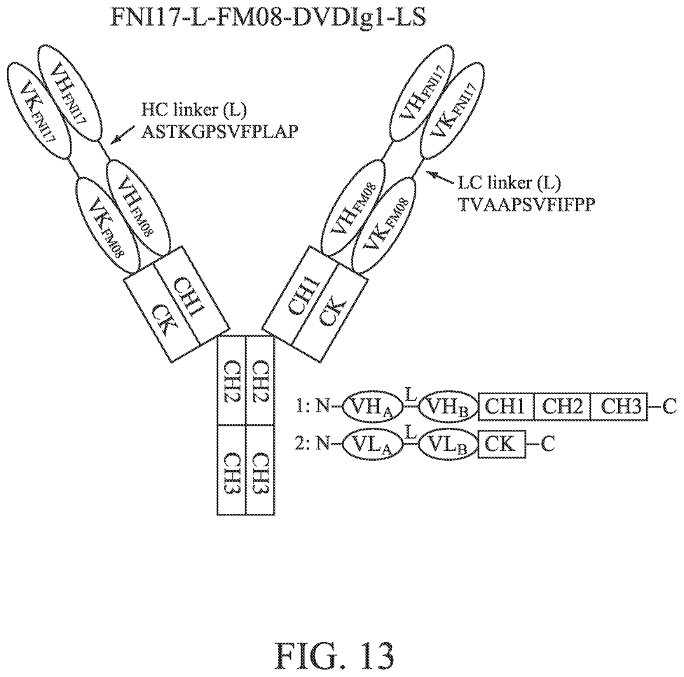

Dual Variable Domain (DVD) bi-specific format antibodies containing anti-NA and anti-HA antibodies were designed and produced. A representative DVD bispecific antibody, FNI17-L-FM08-DVDIg1-LS, containing anti-NA (FNI17) and anti-HA (FM08) antigen-binding domains, is shown in FIG. 13.

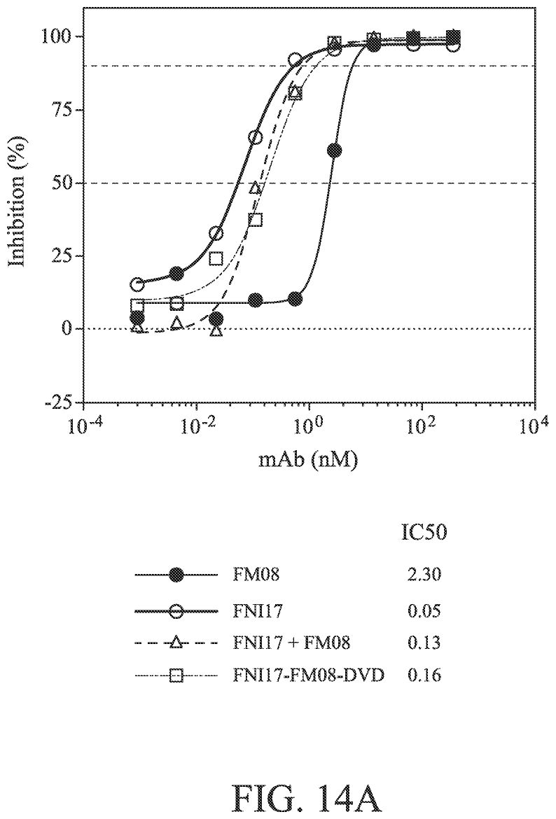

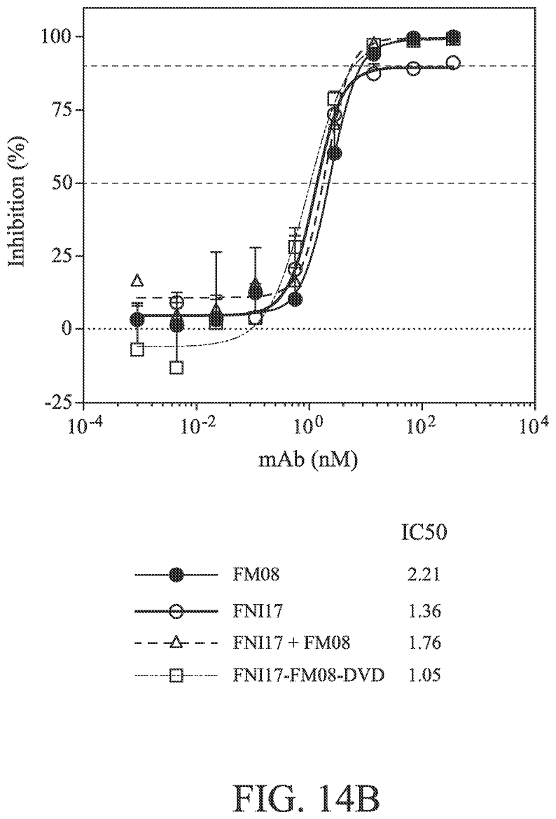

In vitro inhibition of sialidase activity by FNI17-FM08-DVD was evaluated. Comparator test groups included FNI17 mAb alone, FNI17+FM08, mAbs or FM08 mAb alone against H1N1 Cal/09 (FIG. 14A) and H3N2 HK/68 (FIG. 14B). IC50 values (nM) were calculated for each test group.

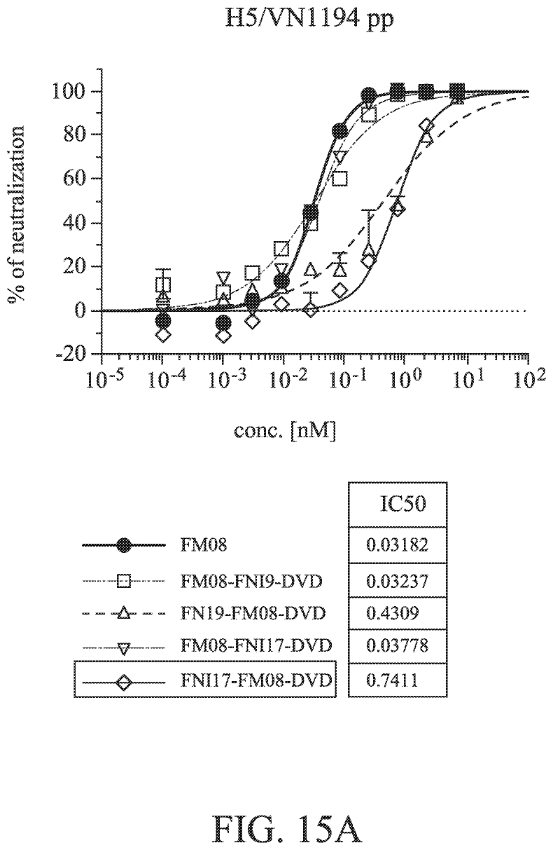

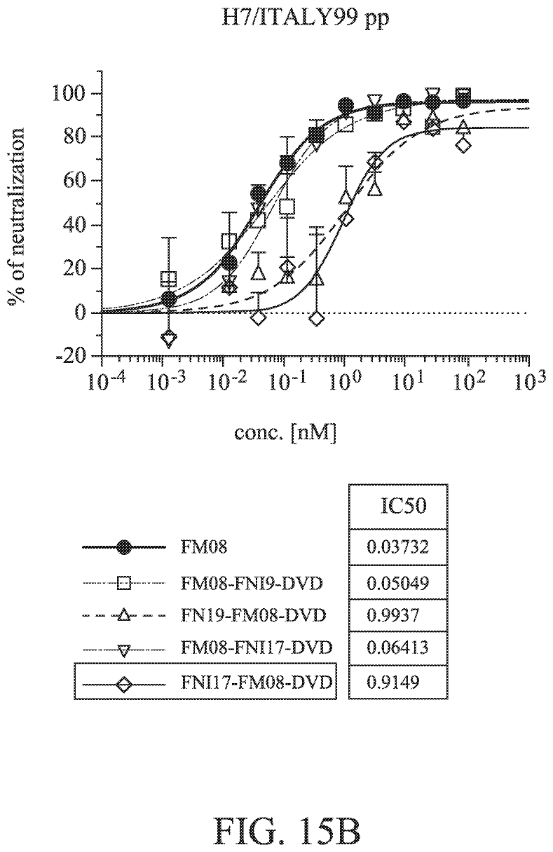

In vitro neutralization of H5 and H7 pseudotyped viruses by FM08-FNI9-DVD, FNI9-FM08-DVD, FM08-FNI17-DVD, and FNI17-FM08-DVD was also evaluated. Comparator antibody FM08 was also tested. FIG. 15A shows neutralization of H5/VN1194 pp. FIG. 15B shows neutralization of H7/IT/99 pp. Calculated 1C50 values (nM) are shown below the graph in each figure.

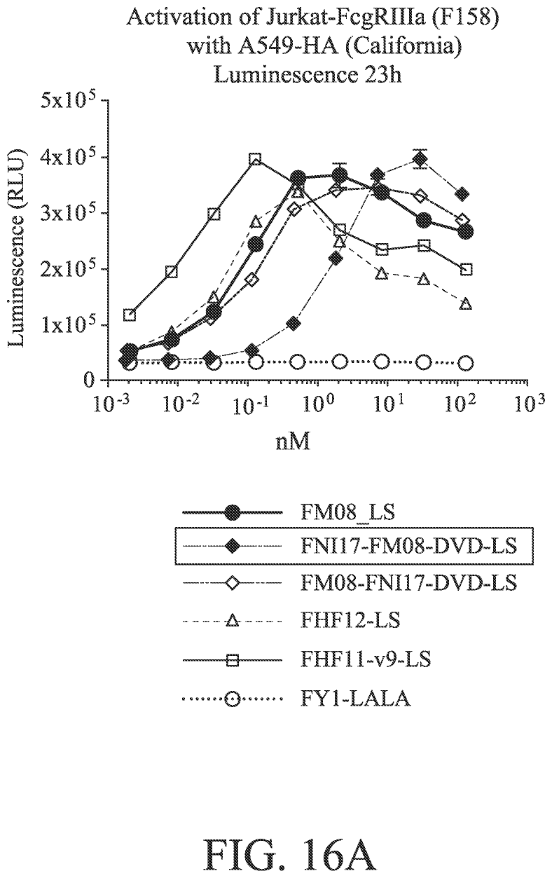

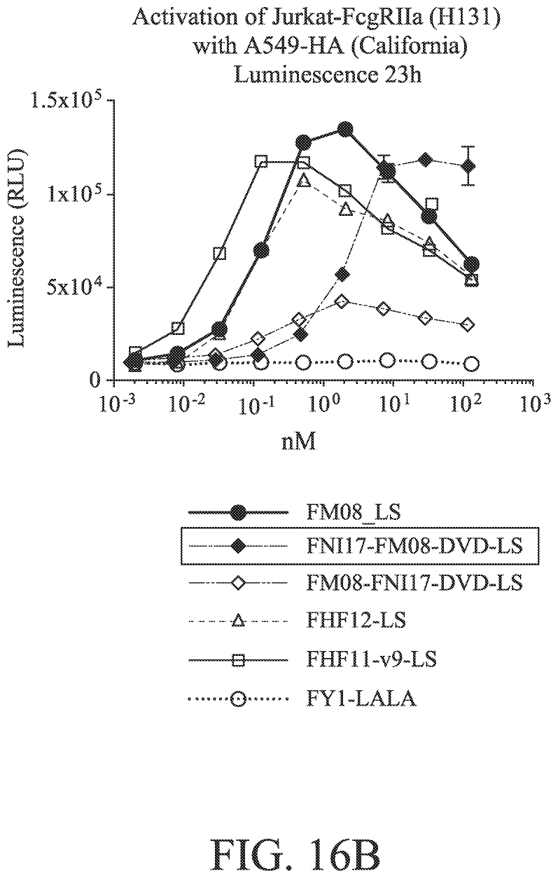

Antibody activation of FcγRIIIa (FIG. 16A; F158 allele) and FcγRIIa (FIG. 16B; H131 allele) was tested for FM08-FNI17-DVD and FNI17-FM08-DVD. Activation was measured using an NFAT-mediated Luciferase reporter in engineered Jurkat cells. Comparator antibodies FM08_LS, FHF12-LS, FHF11-v9-LS, and a negative control antibody (FY1-LALA) were also evaluated.

### Example 5

### In Vivo Testing of Dual Variable Domain (DVD) Bispecific Antibodies

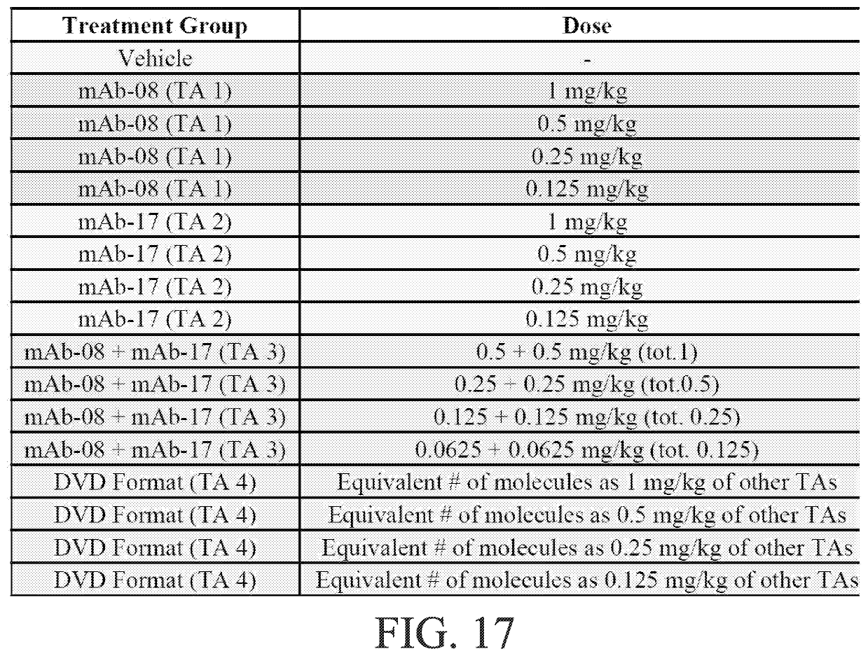

Prophylactic activity of FNI17-FM08-DVD was evaluated in a murine BALB/c model of IAV infection. Briefly, BALB/c mice, 7-8 weeks of age, were administered (i.v.) FM08_LS (TA 1, “mAb-08” in FIG. 17), FNI17_LS (TA-2, “mAb-17” in FIG. 17), FM08_LS+FNI17_LS (TA 3, “mAb-08+mAb-17” in FIG. 17), and FNI17-FM08-DVD-LS (TA 4, “DVD Format” in FIG. 17).

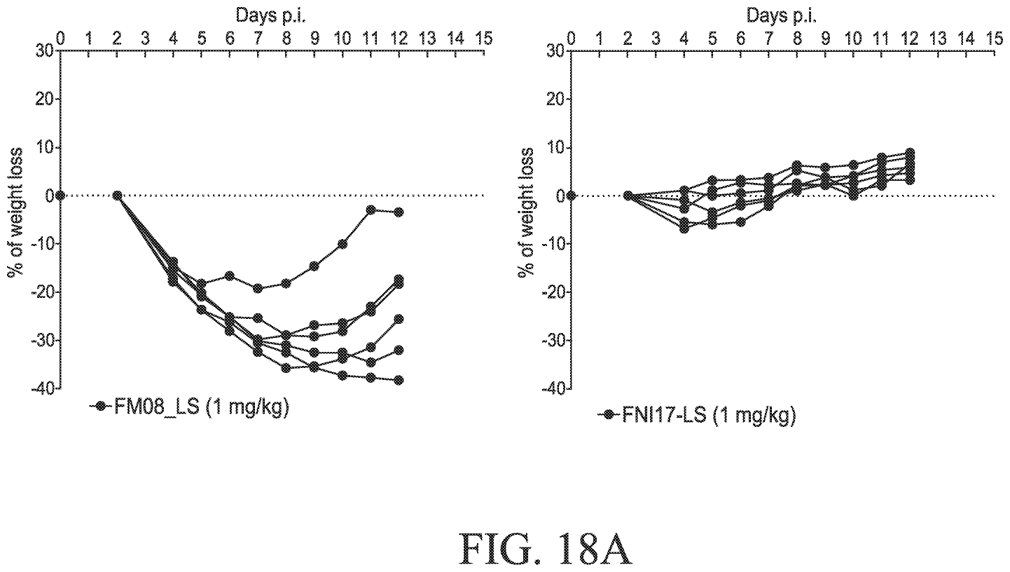

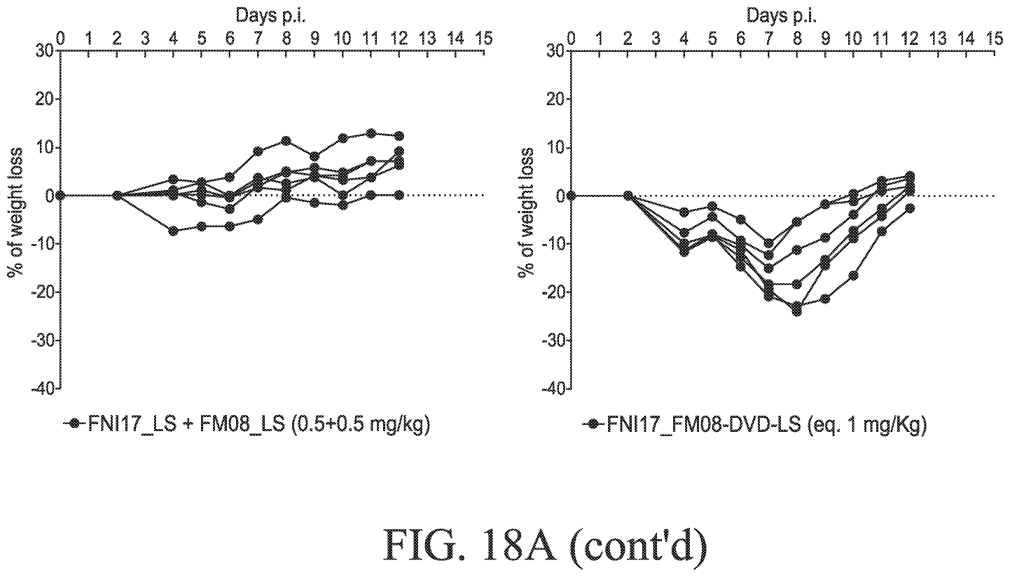

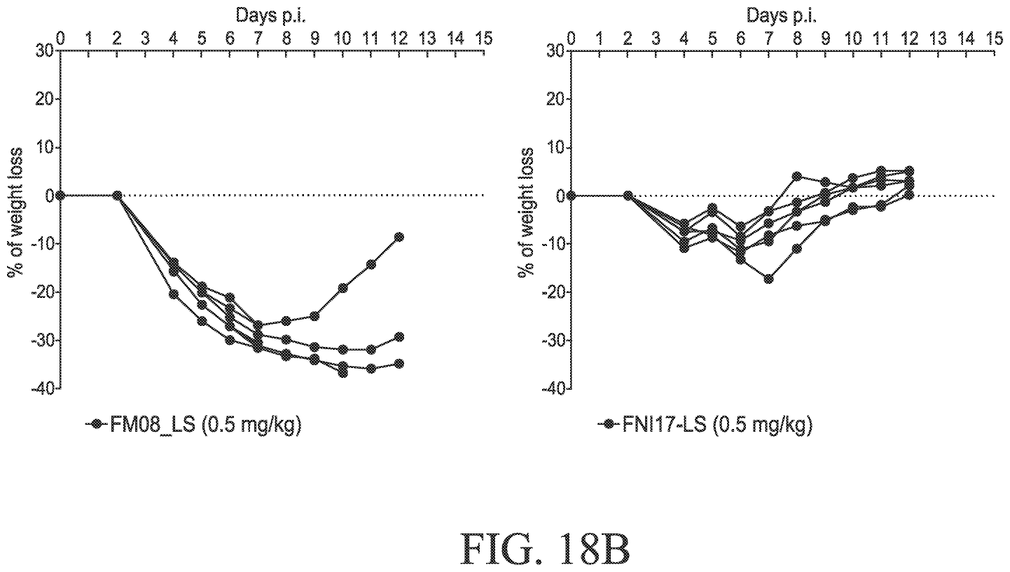

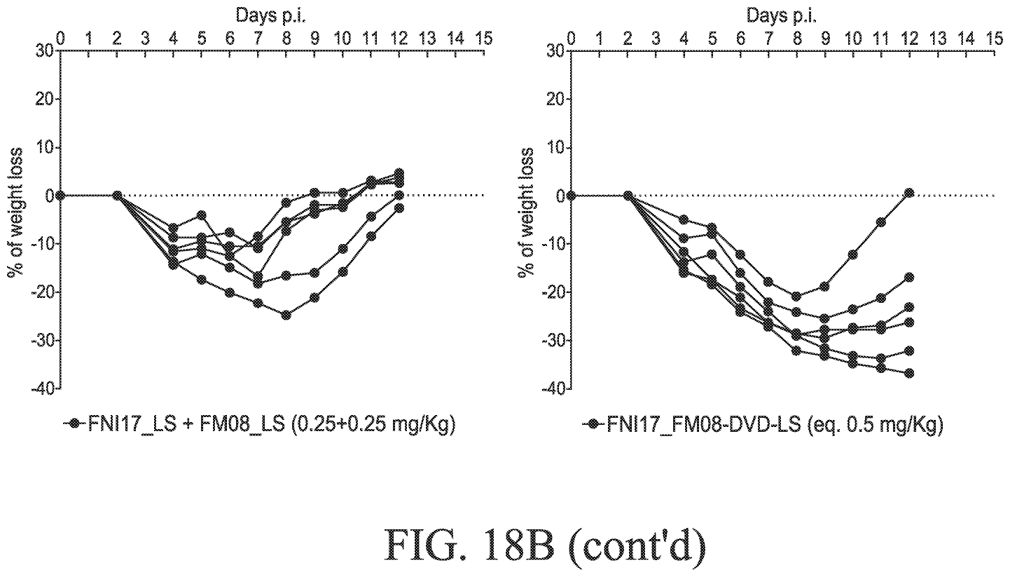

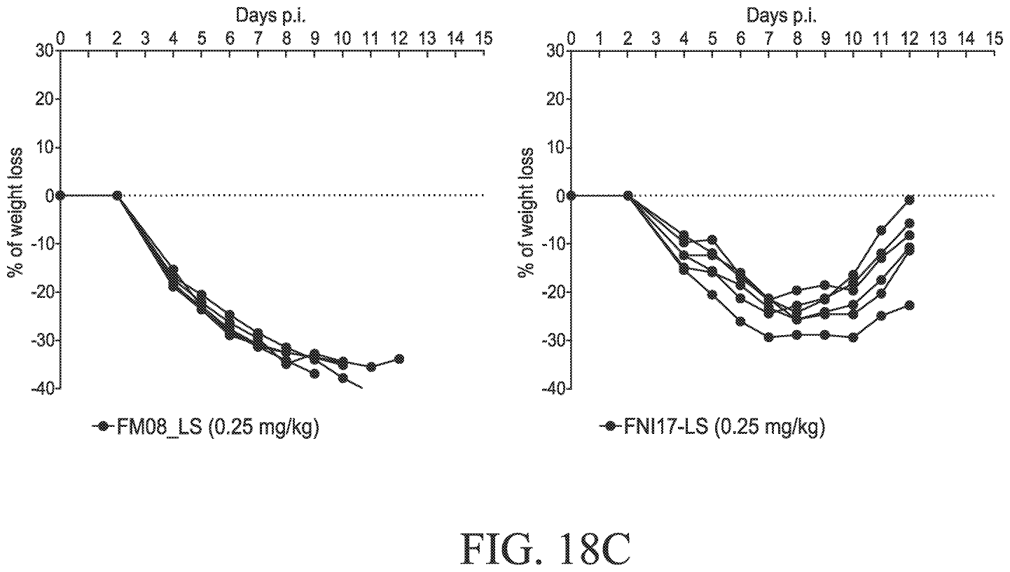

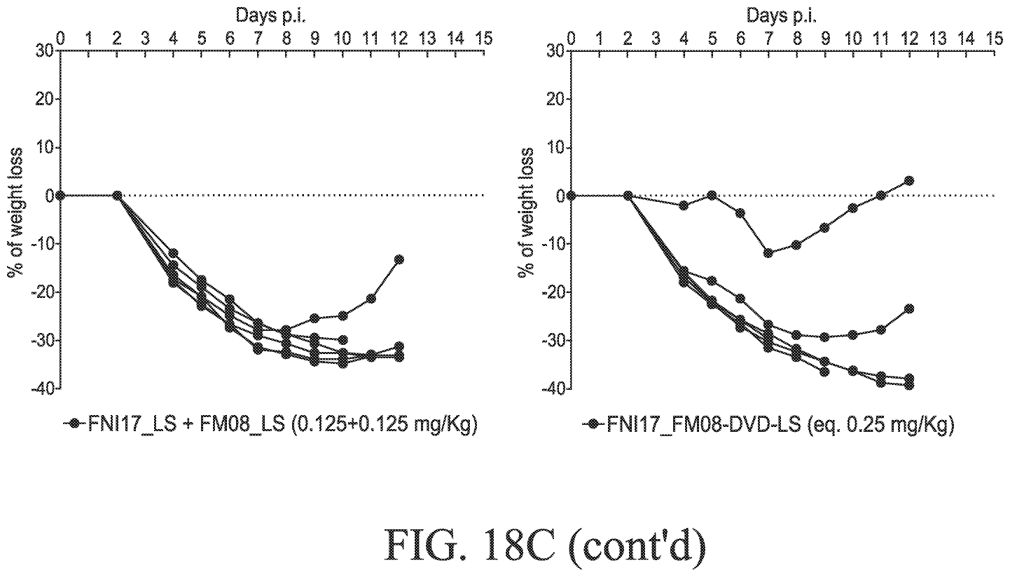

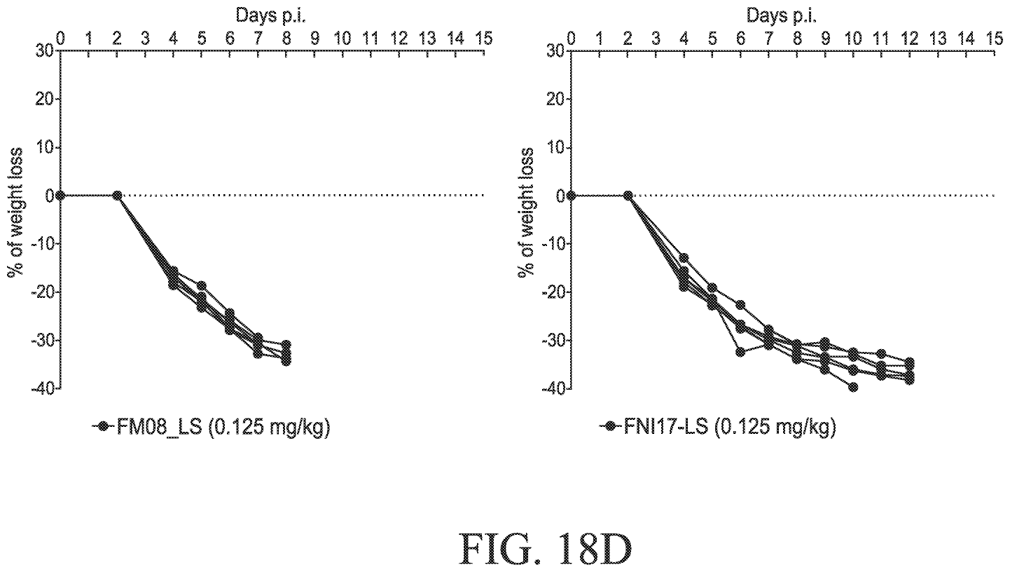

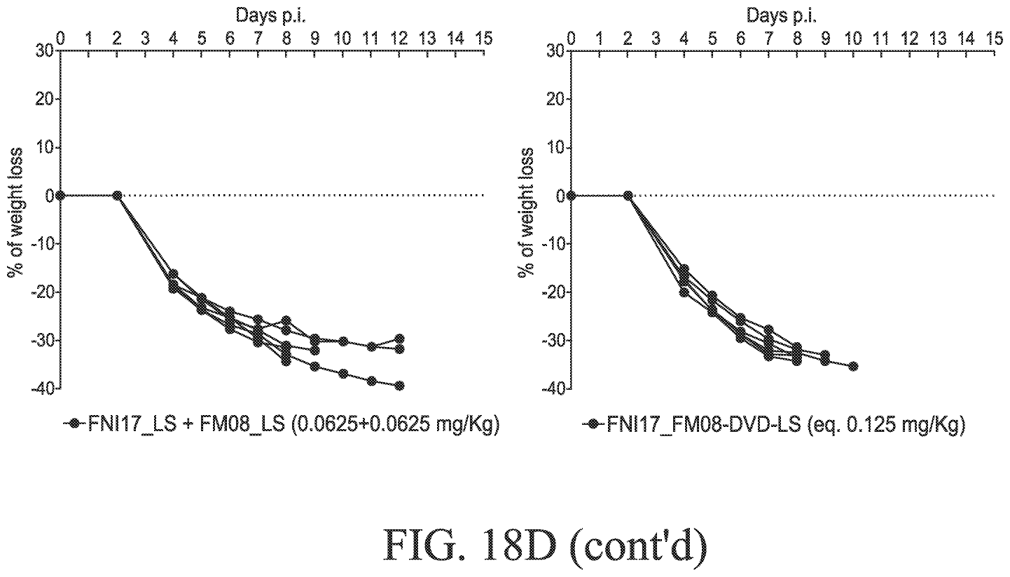

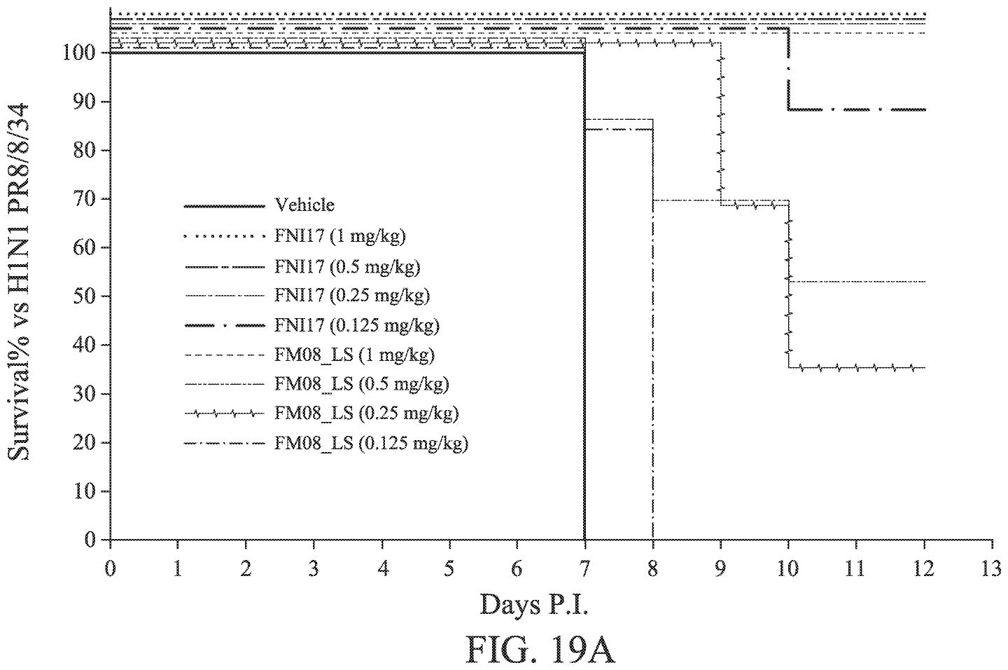

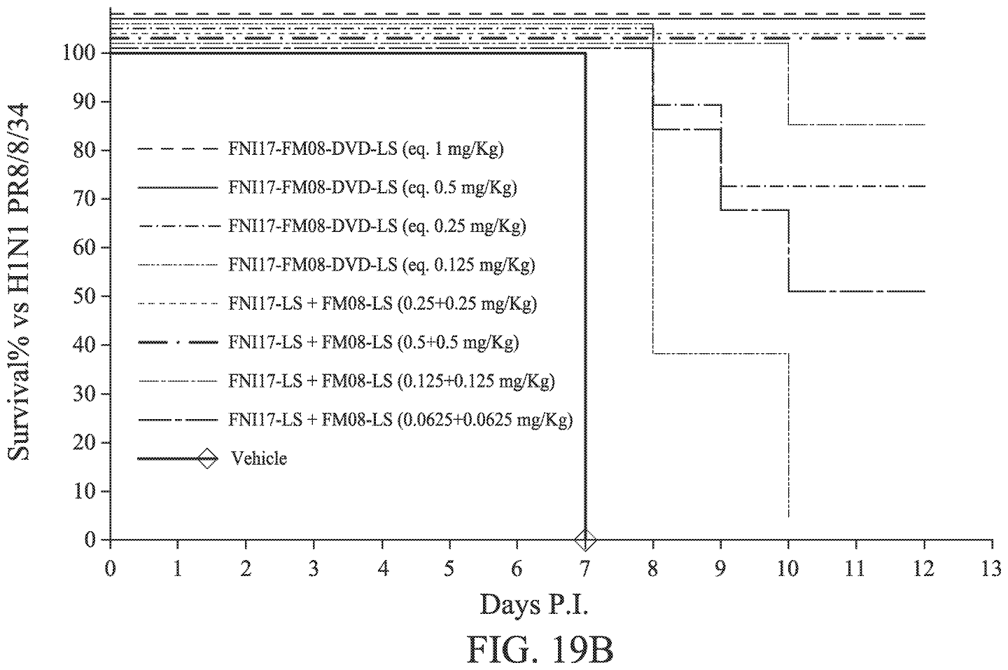

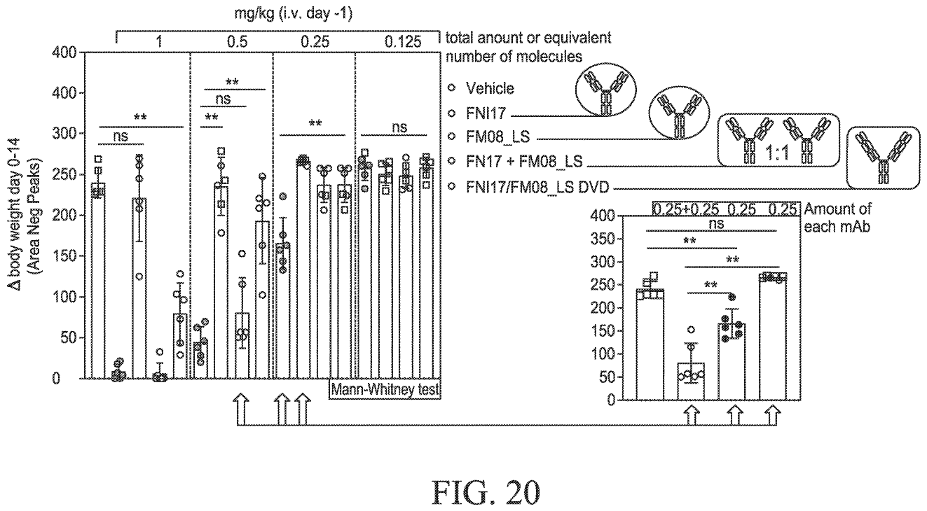

Antibodies were administered one day prior to intranasal infection at LD90 (90% of a lethal dose) with an H1N1 subtype. Antibody was administered (i.v.) at 0.125, 0.25, 0.5, or 1 mg/kg. Baseline serum was collected at the start of infection, and both body weight and mortality were evaluated on each of Days 2-14 post-infection. Body weight was measured over fifteen days following pre-treatment with 1 mg/kg (FIG. 18A), 0.5 mg/kg (FIG. 18B), 0.25 mg/kg (FIG. 18C), or 0.125 mg/kg (FIG. 18D) of the indicated monoclonal antibodies one day prior to infection with a LD90 (90% lethal dose) of H1N1. Mice in the FNI17-FM08-DVD-LS treatment group received an equivalent number of molecules corresponding with the body weight dosage (mg/kg) dosage of the other three treatment groups. Overall mortality was also measured over fifteen days in BALB/c mice infected with H1N1 and pre-treated with FM08_LS or FNI17_LS (FIG. 19A), or FM08_LS+FNI17_LS or FNI17-FM08-DVD-LS (FIG. 19B). Weight change data are summarized in FIG. 20.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/117,454, filed on Nov. 23, 2020, U.S. Provisional Application No. 63/125,892, filed on Dec. 15, 2020, U.S. Provisional Application No. 63/197,254, filed on Jun. 4, 2021 and U.S. Provisional Application No. 63/261,464, filed on Sep. 21, 2021 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A combination comprising:
(1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof; and
(2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof.

2. A composition comprising:
(1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof; and
(2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof,
and a pharmaceutically acceptable carrier, excipient, or diluent.

3.-6. (canceled)

7. A method of treating or preventing an influenza (IAV, IBV, or both) infection in a subject, the method comprising administering to the subject an effective amount of:
(1) (a) an antibody, or an antigen-binding fragment thereof, that is capable of binding to an influenza A virus (IAV) hemagglutinin (HA) and neutralizing infection by the IAV, or (b) a polynucleotide encoding the anti-HA antibody or antigen-binding fragment thereof; and
(2) (a) an antibody, or an antigen-binding fragment thereof, that is capable of binding to a neuraminidase (NA) from: 2(i) an IAV, wherein the IAV comprises a Group 1 IAV, a Group 2 IAV, or both; and 2(ii) an influenza B virus (IBV), and is capable of neutralizing infection and/or inhibiting sialidase activity by the IAV and/or the IBV, or (b) a polynucleotide encoding the anti-NA antibody or antigen-binding fragment thereof.

8.-9. (canceled)

10. The method of claim 7, wherein:
(1) the anti-HA antibody or antigen-binding fragment comprises (1)(i) a VH comprising or consisting of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.:43, 2, 26, 28, 31, 34, 37, 14, 39, and 41; and/or (1)(ii) a VL comprising or consisting of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 44, 8, and 20;
and/or
(2) the anti-NA antibody or antigen-binding fragment comprises (2)(i) a VH comprising or consisting of an amino acid sequence having at least 80% (e.g., 80%, 85%, 9%, 91%, 92%, 93%, 94%, 95%, 96%, 97%, 98%, 99%, or more) identity to the amino acid sequence set forth in any one of SEQ ID NOs.:241, 48, 60, 72, 171, 84, 96, 108, 120, 132, 229, 144, 156, 168, 180, 192, 204, 245, 249, 258, and 261; and/or (2)(ii) a VL comprising or consisting of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.:243, 54, 66, 78, 90, 102, 114, 126, 138, 150, 162, 174, 186, 198, 220, 223, 226, 232, 235, 238, 210, 247, 251, 259, and 263.

11. The method of claim 7, wherein the anti-HA antibody or antigen-binding fragment comprises a VH and a VL comprising an amino acid sequence having at least 80% identity to the amino acid sequences set forth in SEQ ID NOs.: 43 and 44, respectively, and the anti-NA antibody or antigen-binding fragment comprises a VH and a VL comprising or consisting of an amino acid sequence having at least 80% identity to the amino acid sequences set forth in SEQ ID NOs.: 241 and 243, respectively.

12. (canceled)

13. The method of claim 7, wherein:
(1) the anti-HA antibody or antigen-binding fragment comprises a heavy chain variable domain (VH) comprising a complementarity determining region (CDR)H1, a CDRH2, and a CDRH3, and a light chain variable domain (VL) comprising a CDRL1, a CDRL2, and a CDRL3, wherein: (I)(i) the CDRH1 comprises or consists of the amino acid sequence of any one of SEQ ID NOs.: 274, 3, 32, and 15, or a functional variant thereof comprising one, two, or three acid substitutions; (1)(ii) the CDRH2 comprises or consists of the amino acid sequence of any one of SEQ ID NOs.: 275, 4, 29, 35, 16, and 42, or a functional variant thereof comprising one, two, or three amino acid substitutions; (1)(iii) the CDRH3 comprises or consists of the amino acid sequence of any one of SEQ ID NOs.: 276, 5, and 17, or a functional variant thereof comprising one, two, or three amino acid substitutions; (1)(iv) the CDRL1 comprises or consists of the amino acid sequence of any one of SEQ ID NOs.:277, 9, and 21, or a functional variant thereof comprising one, two, or three amino acid substitutions; (1)(v) the CDRL2 comprises or consists of the amino acid sequence of any one of SEQ ID NOs.: 278, 10, and 22, or a functional variant thereof comprising one, two, or three amino acid substitutions; and/or (1)(vi) the CDRL3 comprises or consists of the amino acid sequence of any one of SEQ ID NOs.: 279, 11, and 23, or a functional variant thereof comprising one, two, or three amino acid substitutions;
and/or
(2) the anti-NA antibody or antigen-binding fragment comprises a VH comprising a CDRH1, a CDRH2, and a CDRH3, and a VL comprising a CDRL1, a CDRL2, and a CDRL3, wherein: (2)(i) the CDRH1 comprises or consists of the amino acid sequence set forth in any one of SEQ ID NOs.: 193, 49, 61, 73, 85, 97, 109, 121, 133, 145, 157, 169, 181, 205, and 264, or a functional variant thereof comprising one, two, or three acid substitutions; (2)(ii) the CDRH2 comprises or consists of the amino acid sequence set forth in any one of SEQ ID NOs.: 194, 50, 62, 74, 86, 98, 110, 122, 134, 146, 158, 170, 182, 206, and 265, or a functional variant thereof comprising one, two, or three amino acid substitutions; (2)(iii) the CDRH3 comprises or consists of the amino acid sequence set forth in any one of SEQ ID NOs.: 195, 51, 63, 75, 218, 87, 99, 111, 123, 135, 230, 147, 159, 171, 183, 207, and 266, or a functional variant thereof comprising one, two, or three amino acid substitutions; (2)(iv) the CDRL1 comprises or consists of the amino acid sequence set forth in any one of SEQ ID NOs.: 199, 55, 67, 79, 91, 103, 115, 127, 139, 151, 163, 175, 187, 211, and 267, or a functional variant thereof comprising one, two, or three amino acid substitutions; (2)(v) the CDRL2 comprises or consists of the amino acid sequence set forth in any one of SEQ ID NOs.: 200, 56, 68, 80, 92, 104, 116, 128, 140, 152, 164, 176, 188, 212, and 268, or a functional variant thereof comprising one, two, or three amino acid substitutions; and/or (2)(vi) the CDRL3 comprises or consists of the amino acid sequence set forth in any one of SEQ ID NOs.: 201, 57, 69, 81, 221, 224, 227, 93, 105, 117, 129, 141, 233, 239, 153, 165, 177, 189, 236, 213, and 269, or a functional variant thereof comprising having one, two, or three amino acid substitutions.

14.-15. (canceled)

16. The method of claim 7, wherein: (i) the Group 1 IAV NA comprises a N1, a N4, a N5, and/or a N8; and/or (ii) the Group 2 IAV NA comprises a N2, a N3, a N6, a N7, and/or a N9.

17.-22. (canceled)

23. The method of claim 7, wherein:
(i) the Group 1 IAV NA comprises a H1N1 and/or a H5N1;
(ii) the Group 2 IAV NA comprises a H3N2 and/or a H7N9; and/or
(iii) the IBV NA comprises one or more of: B/Lee/10/1940 (Ancestral);
B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006 (Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

24.-26. (canceled)

27. The method of claim 7, wherein the anti-HA antibody or antigen-binding fragment, the anti-NA antibody or antigen-binding fragment, or both, is capable of activating a human FcγRIIIa, wherein activation is as determined using a host cell comprising: (i) the human FcγRIIIa; and (ii) a NFAT expression control sequence operably linked to a sequence encoding a reporter following incubation of the antibody or antigen-binding fragment with a target cell infected with a IAV and/or a IBV.

28.-33. (canceled)

34. The method of claim 7, wherein the anti-HA antibody or antigen-binding fragment, the anti-NA antibody or antigen binding fragment, or both, is/are capable of preventing a loss in body weight of greater than 25%, 20%, 15%, 10%, or 5% in a subject having an IAV infection and/or an IBV infection, as determined by reference to the subject's body weight just prior to the IAV and/or IBV infection.

35. (canceled)

36. The method of claim 7, wherein the anti-HA antibody or antigen-binding fragment, the anti-NA antibody or antigen binding fragment, or both, is/are a IgG, IgA, IgM, IgE, or IgD isotype.

37. The method of claim 7, wherein the anti-HA antibody or antigen-binding fragment, the anti-NA antibody or antigen binding fragment, or both, is/are an IgG isotype selected from IgG1, IgG2, IgG3, and IgG4.

38. The method of claim 7, wherein the anti-HA antibody or antigen-binding fragment, the anti-NA antibody or antigen binding fragment, or both, comprises or comprise a human antibody, a monoclonal antibody, a purified antibody, a single chain antibody, a Fab, a Fab′, a F(ab′)2, or Fv.

39. (canceled)

40. The method of claim 7, wherein the anti-HA antibody or antigen-binding fragment, the anti-NA antibody or antigen binding fragment, or both, comprises a Fc polypeptide or a fragment thereof.

41. The method of claim 40, wherein the the Fc polypeptide or fragment thereof comprises:
(i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or
(ii) a mutation that increases binding affinity to a human FcγR (e.g., as measured using surface plasmon resonance (SPR) (e.g., Biacore, e.g., T200 instrument, using manufacturer's protocols)) as compared to a reference Fc polypeptide that does not comprise the mutation.

42.-46. (canceled)

47. The method of claim 7, wherein the anti-HA antibody or antigen-binding fragment, the anti-NA antibody or antigen binding fragment, or both, comprises a mutation that alters glycosylation, wherein the mutation that alters glycosylation comprises N297A, N297Q, or N297G, and/or which is aglycosylated and/or afucosylated.

48. The method of claim 7, wherein the treatment and/or prevention comprises post-exposure prophylaxis.

49. The method of claim 7, the wherein subject has received, is receiving, or will receive an antiviral.

50. The method of claim 7, wherein the antibody or antigen-binding fragment comprises:
(i) a CH1-CH3 comprising or consisting of the amino acid sequence set forth in SEQ ID NO.:252;
(ii) a CH1-CH3 comprising or consisting of the amino acid sequence set forth in SEQ ID NO.:253;
(iii) a CL comprising or consisting of the amino acid sequence set forth in SEQ ID NO.:254; or
(iv) any combination of (i)-(iii).

51.-54. (canceled)

55. The method of claim 49, wherein the antiviral comprises oseltamivir, zanamivir, lanimivir, peramivir, baloxavir, or any combination thereof.

56.-98. (canceled)


SEQUENCE LISTING (281 sequences)

SEQ ID NO: 1 - DNA, 381 bp, Artificial Sequence
  FHF11 VH (wt-nt)
caggtacaactgcagcagtcaggtccaggactggtgaagccctcgcagaccctctcagtcacctgtggcatctccggggacagtgtctctagtcacagtgctgcttggaactggatcaggcagtccccatcgagaggccttgagtggctgggaaggacatattacaggtccaagtggtataatgattatgcagtctctgtgaaaagtcgaataaccatcaatccagacacatccaagaaccagttctccctacagttgatctctgtgactcccgaggacacggctgtctattactgtgcaagagtgggtgctatgacttttggacttcttacagggggtatggacgtctggggccaagggaccacggtcaccgtctcctca

SEQ ID NO: 2 - protein, 127 aa, Artificial Sequence
  FHF11 VH (aa)
QVQLQQSGPGLVKPSQTLSVTCGISGDSVSSHSAAWNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 3 - protein, 10 aa, Artificial Sequence
  FHF11 CDR-H1 (aa)
GDSVSSHSAA

SEQ ID NO: 4 - protein, 9 aa, Artificial Sequence
  FHF11 CDR-H2 (aa)
TYYRSKWYN

SEQ ID NO: 5 - protein, 17 aa, Artificial Sequence
  FHF11 CDR-H3 (aa)
ARVGAMTFGLLTGGMDV

SEQ ID NO: 6 - DNA, 381 bp, Artificial Sequence
  FHF11 VH (co-nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 7 - DNA, 328 bp, Artificial Sequence
  FHF11 Vk (wt-nt)
gaaattgtgttgacgcagtctccaggcacccagtctttgtctccaggggaaagagccaccctctcctgcagggccagtcagagtctgagccgcagctacttagcctggtaccagcagagacctggcaagcctcccaggctcctcatctatggtgcatccagcagggccactggcatcccagacaggttcagtggcagtgggtctgggacagacttcagtctcaccatcagcagtctggagcctgaagattctgcaatgtatttctgtcagtactatggtgattcacctctattcagtttcggcccagggaccaaagtggatatcaaac

SEQ ID NO: 8 - protein, 109 aa, Artificial Sequence
  FHF11 Vk (aa)
EIVLTQSPGTQSLSPGERATLSCRASQSLSRSYLAWYQQRPGKPPRLLIYGASSRATGIPDRFSGSGSGTDFSLTISSLEPEDSAMYFCQYYGDSPLFSFGPGTKVDIK

SEQ ID NO: 9 - protein, 7 aa, Artificial Sequence
  FHF11 CDR-L1 (aa)
QSLSRSY

SEQ ID NO: 10 - protein, 3 aa, Artificial Sequence
  FHF11 CDR-L2 (aa)
GAS

SEQ ID NO: 11 - protein, 10 aa, Artificial Sequence
  FHF11 CDR-L3 (aa)
QYYGDSPLFS

SEQ ID NO: 12 - DNA, 327 bp, Artificial Sequence
  FHF11 Vk (co-nt)
gagatcgtgctgacccagtctcctggcacacagagcctgtctccaggagagagggccaccctgtcctgcagggcttcccagagcctgtctaggtcctacctggcctggtatcagcagagaccaggcaagccacctaggctgctgatctacggagcttccagcagggctacaggcatccctgacagattcagcggctctggctccggcaccgatttttccctgacaatctcttccctggagccagaggactccgccatgtatttctgtcagtactatggcgatagcccactgttctcttttggccccggcaccaaggtggacatcaag

SEQ ID NO: 13 - DNA, 381 bp, Artificial Sequence
  FHF12 VH (wt-nt)
caggtacaactgcagcagtcaggtccaggactggtgaagccctcgcagaccctctcagtcacctgtgccatctccggggacagtgtctctagtcacagtgctgcttggaactggatcaggcagtccccatcgagaggccttgagtggctgggaaggacatattacaggtccaagtggtataatgattatgcagtctctgtgaaaagtcgaataaccatcaacccagacacatccaagaaccagttctccctacagctggtctctgtgactcccgaggacacggctgtctattactgtgcaagagtgggtgctgcgacttttggaattcttacagggggtatggacgtctggggccaagggaccacggtcaccgtctcctca

SEQ ID NO: 14 - protein, 127 aa, Artificial Sequence
  FFHF12 VH (aa)
QVQLQQSGPGLVKPSQTLSVTCAISGDSVSSHSAAWNWIRQSPSRGLEWLGRTYYRSKWYNDYAVSVKSRITINPDTSKNQFSLQLVSVTPEDTAVYYCARVGAATFGILTGGMDVWGQGTTVTVSS

SEQ ID NO: 15 - protein, 10 aa, Artificial Sequence
  FHF12 CDR-H1 (aa)
GDSVSSHSAA

SEQ ID NO: 16 - protein, 9 aa, Artificial Sequence
  FHF12 CDR-H2 (aa)
TYYRSKWYN

SEQ ID NO: 17 - protein, 17 aa, Artificial Sequence
  FHF12 CDR-H3 (aa)
ARVGAATFGILTGGMDV

SEQ ID NO: 18 - DNA, 381 bp, Artificial Sequence
  FHF12 VH (co-nt)
caggtgcagctgcagcagtctggaccaggactggtgaagcctagccagaccctgtctgtgacatgcgctatctccggcgacagcgtgtccagccactccgccgcttggaactggatcagacagagcccatctaggggactggagtggctgggaaggacctactatcggagcaagtggtacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 19 - DNA, 328 bp, Artificial Sequence
  FHF12 Vk (wt-nt)
gaaattgtgttgacgcagtctccaggcacccagtctttgtctccaggggatagagccaccctctcctgcagggccagtcagagtctgagcagaagctacttagcctggtaccagcagagacctggcaagcctcccaggctcctcatctatggtgcatccagcagggccactggcatcccagacaggttcagtggcagtgggtctgggacagacttcagtctcaccatcagcagtctggagcctgaagattctgctatgtatttctgtcagtactatggtgattcacctctattcagtttcggccctgggaccaaagtggatatcaaac

SEQ ID NO: 20 - protein, 109 aa, Artificial Sequence
  FHF12 Vk (aa)
EIVLTQSPGTQSLSPGDRATLSCRASQSLSRSYLAWYQQRPGKPPRLLIYGASSRATGIPDRFSGSGSGTDFSLTISSLEPEDSAMYFCQYYGDSPLFSFGPGTKVDIK

SEQ ID NO: 21 - protein, 7 aa, Artificial Sequence
  FHF12 CDR-L1 (aa)
QSLSRSY

SEQ ID NO: 22 - protein, 3 aa, Artificial Sequence
  FHF12 CDR-L2 (aa)
GAS

SEQ ID NO: 23 - protein, 10 aa, Artificial Sequence
  FHF12 CDR-L3 (aa)
QYYGDSPLFS

SEQ ID NO: 24 - DNA, 327 bp, Artificial Sequence
  FHF12 Vk (co-nt)
gagatcgtgctgacccagtctcctggcacacagagcctgtctccaggcgacagggccaccctgtcctgcagggcttcccagagcctgtctaggtcctacctggcctggtatcagcagagaccaggcaagccacctaggctgctgatctacggagcttccagcagggctacaggcatccctgacagattcagcggctctggctccggcaccgatttttccctgacaatctcttccctggagccagaggactccgccatgtatttctgtcagtactatggcgatagcccactgttctcttttggccccggcaccaaggtggatatcaag

SEQ ID NO: 25 - DNA, 381 bp, Artificial Sequence
  FHF11-VH W36F (nt)
caggtgcagctgcagcagtctggaccaggactggtgaagcctagccagaccctgtctgtgacatgcggaatctccggcgacagcgtgtccagccactccgccgctttcaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 26 - protein, 127 aa, Artificial Sequence
  FHF11-VH W36F (aa)
QVQLQQSGPGLVKPSQTLSVTCGISGDSVSSHSAAFNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 27 - DNA, 381 bp, Artificial Sequence
  FHF11-VH W59F (nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcaagttctacaatgactatgccgtgtctgtgaagtccaggatcaccatcaacccagatacatccaagaatcagtttagcctgcagctgatctctgtgacccccgaggacacagccgtgtactattgtgccagagtgggcgctatgaccttcggcctgctgacaggcggaatggacgtgtggggacagggaaccacagtgacagtgtcttcc

SEQ ID NO: 28 - protein, 127 aa, Artificial Sequence
  FHF11-VH W59F (aa)
QVQLQQSGPGLVKPSQTLSVTCGISGDSVSSHSAAWNWIRQSPSRGLEWLGRTYYRSKFYNDYAVSVKSRITINPDTSKNQFSLQLISVTPEDTAVYYCARVGAMTFGLLTGGMDVWGQGTTVTVSS

SEQ ID NO: 29 - protein, 9 aa, Artificial Sequence
  FHF11-VH W59F CDRH2 (aa)
TYYRSKFYN

SEQ ID NO: 30 - DNA, 381 bp, Artificial Sequence
  FHF11v3 VH (nt)
caggtgcagctgcagcagtctggaccaggactggtgaagcctagccagaccctgtctgtgacatgcggcatctccggcgacagcgtgtccagctactccgccgcttggaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 31 - protein, 127 aa, Artificial Sequence
  FHF11v3 VH (aa)
QVQLQQSGPGLVKPSQTLSVTCGISGDSVSSYSAAWNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 32 - protein, 10 aa, Artificial Sequence
  FHF11v3 CDRH1 (aa)
GDSVSSYSAA

SEQ ID NO: 33 - DNA, 381 bp, Artificial Sequence
  FHF11v6 VH (nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcggctggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 34 - protein, 127 aa, Artificial Sequence
  FHF11v6 VH (aa)
QVQLQQSGPGLVKPSQTLSVTCGISGDSVSSHSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSS

SEQ ID NO: 35 - protein, 9 aa, Artificial Sequence
  FHF11v6 CDRH2 (aa)
TYYRSGWYN

SEQ ID NO: 36 - DNA, 381 bp, Artificial Sequence
  FHF11v9 VH (nt)
caggtgcagctgcagcagtctggaccaggactggtgaagcctagccagaccctgtctgtgacatgcggcatctccggcgacagcgtgtccagctactccgccgcttggaactggatcagacagagcccatctaggggactggagtggctgggaaggacctactatcggagcggctggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 37 - protein, 127 aa, Artificial Sequence
  FHF11v9 VH (aa)
QVQLQQSGPGLVKPSQTLSVTCGISGDSVSSYSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSS

SEQ ID NO: 38 - DNA, 381 bp, Artificial Sequence
  FHF12-VH-W36F (nt)
caggtgcagctgcagcagtctggaccaggactggtgaagcctagccagaccctgtctgtgacatgcgctatctccggcgacagcgtgtccagccactccgccgctttcaactggatcagacagagcccatctaggggactggagtggctgggaaggacctactatcggagcaagtggtacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 39 - protein, 127 aa, Artificial Sequence
  FHF12-VH-W36F (aa)
QVQLQQSGPGLVKPSQTLSVTCAISGDSVSSHSAAFNWIRQSPSRGLEWLGRTYYRSKWYNDYAVSVKSRITINPDTSKNQFSLQLVSVTPEDTAVYYCARVGAATFGILTGGMDVWGQGTTVTVSS

SEQ ID NO: 40 - DNA, 381 bp, Artificial Sequence
  FHF12-VH-W59F (nt)
caggtgcagctgcagcagtctggaccaggactggtgaagcctagccagaccctgtctgtgacatgcgctatctccggcgacagcgtgtccagccactccgccgcttggaactggatcagacagagcccatctaggggactggagtggctgggaaggacctactatcggagcaagttctacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 41 - protein, 127 aa, Artificial Sequence
  FHF12-VH-W59F (aa)
QVQLQQSGPGLVKPSQTLSVTCAISGDSVSSHSAAWNWIRQSPSRGLEWLGRTYYRSKFYNDYAVSVKSRITINPDTSKNQFSLQLVSVTPEDTAVYYCARVGAATFGILTGGMDVWGQGTTVTVSS

SEQ ID NO: 42 - protein, 9 aa, Artificial Sequence
  FHF12-CDRH2-W59F (aa)
TYYRSKFYN

SEQ ID NO: 43 - protein, 128 aa, Artificial Sequence
  FM08 VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 44 - protein, 103 aa, Artificial Sequence
  Synthetic sequence FM08 VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 45 - protein, 217 aa, Artificial Sequence
  WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 19 aa, Artificial Sequence
  Chimeric hinge sequence
ESKYGPPCPPCPAPPVAGP

SEQ ID NO: 47 - DNA, 385 bp, Artificial Sequence
  FNI1 VH (wt-nt)
caagttcagctggtgcagtctggggctgaggtgaagaggcctgggtcctcggtgaggatctcctgcaaggcctctggtgacaccttcaacaactatgttctcagctgggtgcgacaggcccctggacaagggcttgagtggatggggggaatcatccctatctctggtatcccacattacgcacagaagttccagggcagagtcgcaattatcgcggacgaatccgcgagcacagtctacatggagttgagcagcctacgatctgaggactcggccgtatattactgtgcgagagcggtttccgattattttaatcgagacctcggctgggatgattactactttcctttgtggggccagggcaccctggtcaccgtctcctcag

SEQ ID NO: 48 - protein, 128 aa, Artificial Sequence
  FNI1 VH (aa)
QVQLVQSGAEVKRPGSSVRISCKASGDTFNNYVLSWVRQAPGQGLEWMGGIIPISGIPHYAQKFQGRVAIIADESASTVYMELSSLRSEDSAVYYCARAVSDYFNRDLGWDDYYFPLWGQGTLVTVSS

SEQ ID NO: 49 - protein, 8 aa, Artificial Sequence
  FNI1 CDRH1 (aa)
GDTFNNYV

SEQ ID NO: 50 - protein, 8 aa, Artificial Sequence
  FNI1 CDRH2 (aa)
IIPISGIP

SEQ ID NO: 51 - protein, 21 aa, Artificial Sequence
  FNI1 CDRH3 (aa)
ARAVSDYFNRDLGWDDYYFPL

SEQ ID NO: 52 - DNA, 384 bp, Artificial Sequence
  FNI1 VH (co-nt)
caggtgcagctggtgcagtctggagctgaggtgaagaggccaggatccagcgtgcggatcagctgcaaggcttctggcgacaccttcaacaattacgtgctgtcctgggtgaggcaggctccaggacagggactggagtggatgggcggcatcatccccatcagcggcatccctcactacgcccagaagtttcagggcagggtggccatcatcgctgacgagtccgctagcacagtgtatatggagctgtcttccctgagatctgaggattccgccgtgtactattgtgccagagccgtgtccgactatttcaaccgcgatctgggctgggacgattactattttccactgtggggacagggcaccctggtgacagtgagctct

SEQ ID NO: 53 - DNA, 325 bp, Artificial Sequence
  FNI1 Vk (wt-nt)
gaaatagtgatgacgcagtctccagccaccctgtctgtgtctccaggggaaagagccaccctcttctgcagggccagtcggagtgttagtgacaacttagcctggtaccagcagaaacctggccaggctcccaggctcctcatctttggtgcctccaccagggccactggtgtcccagccaggttcggtggcagtgggtctgggacacagttcactctcaccatcagcagcctgcagtctgaagattttgcagtttattactgtcagcattataatacctggcctccgtggaccttcggccaagggaccaaggtggaaatcaaac

SEQ ID NO: 54 - protein, 108 aa, Artificial Sequence
  FNI1 VK (aa)
EIVMTQSPATLSVSPGERATLFCRASRSVSDNLAWYQQKPGQAPRLLIFGASTRATGVPARFGGSGSGTQFTLTISSLQSEDFAVYYCQHYNTWPPWTFGQGTKVEIK

SEQ ID NO: 55 - protein, 6 aa, Artificial Sequence
  FNI1 CDRL1(aa)
RSVSDN

SEQ ID NO: 56 - protein, 3 aa, Artificial Sequence
  FNI1 CDRL2(aa)
GAS

SEQ ID NO: 57 - protein, 10 aa, Artificial Sequence
  FNI1 CDRL3(aa)
QHYNTWPPWT

SEQ ID NO: 58 - DNA, 324 bp, Artificial Sequence
  FNI1 Vk (co-nt)
gagatcgtgatgacccagtctcctgccacactgtccgtgtccccaggcgagagggccacactgttctgcagggctagcaggtccgtgtccgacaacctggcctggtaccagcagaagccaggccaggctcccagactgctgatctttggagcttccaccagagctacaggcgtgccagctaggttcggaggaagcggatctggcacccagtttaccctgacaatctccagcctgcagagcgaggatttcgccgtgtactattgtcagcactataatacctggcccccttggacatttggccagggcaccaaggtggagatcaag

SEQ ID NO: 59 - DNA, 385 bp, Artificial Sequence
  FNI2 VH (wt-nt)
caggttcagctggtgcagtctggggctgaggtgaagaggcctgggtcctcggtgagggtctcctgcaaggcttctggagccaccttcaataaccatgttctcacctgggtgcgacaggcccctggacaagggcttgagtggatgggagggatcatccctgtctctggaaaaacaacctacgcacagaagttccagggcagagtcgcgataagcacggacgaatccgcgagcacagcctatatggagttgagcagcctgagatctgaggactcggccatatattactgtgcgagagcggtttccgattactttaatcgagacctcggctgggaagattattactttccgatctggggccagggcaccctggtcaccgtctcttcag

SEQ ID NO: 60 - protein, 128 aa, Artificial Sequence
  FNI2 VH (aa)
QVQLVQSGAEVKRPGSSVRVSCKASGATFNNHVLTWVRQAPGQGLEWMGGIIPVSGKTTYAQKFQGRVAISTDESASTAYMELSSLRSEDSAIYYCARAVSDYFNRDLGWEDYYFPIWGQGTLVTVSS

SEQ ID NO: 61 - protein, 8 aa, Artificial Sequence
  FNI2 CDRH1 (aa)
GATFNNHV

SEQ ID NO: 62 - protein, 8 aa, Artificial Sequence
  FNI2 CDRH2 (aa)
IIPVSGKT

SEQ ID NO: 63 - protein, 21 aa, Artificial Sequence
  FNI2 CDRH3 (aa)
ARAVSDYFNRDLGWEDYYFPI

SEQ ID NO: 64 - DNA, 384 bp, Artificial Sequence
  FNI2 VH (co-nt)
caggtgcagctggtgcagtctggagctgaggtgaagaggccaggatccagcgtgcgggtgagctgcaaggcttctggagctaccttcaacaatcacgtgctgacatgggtgaggcaggctccaggacagggactggagtggatgggcggcatcatccccgtgtccggcaagaccacatacgcccagaagtttcagggcagggtggctatcagcaccgatgagtccgccagcacagcttatatggagctgtcttccctgagatctgaggactccgccatctactattgtgccagagccgtgtccgactacttcaaccgcgatctgggctgggaggactactattttcccatctggggccagggcaccctggtgacagtgagctct

SEQ ID NO: 65 - DNA, 325 bp, Artificial Sequence
  FNI2 Vk (wt-nt)
gacgtagtgatgacgcagtctccagccaccctgtctgtgtctccaggggaaagagccaccctctcctgcagggccagtcagagtgttagtagcaacttggcctggtaccagcagaaacctggccaggctcccaggctcctcatctatggtgcatccaccagggccactggtgtcccagccaggttcagtggcagtgggtctgggacacagttcactctcaccatcagcagcctgcagtctgaagattttgcagtttattactgtcagcactataataactggcctccgtggacgttcggccaagggaccaagttggaaatcaaac

SEQ ID NO: 66 - protein, 108 aa, Artificial Sequence
  FNI2 VK (aa)
DVVMTQSPATLSVSPGERATLSCRASQSVSSNLAWYQQKPGQAPRLLIYGASTRATGVPARFSGSGSGTQFTLTISSLQSEDFAVYYCQHYNNWPPWTFGQGTKLEIK

SEQ ID NO: 67 - protein, 6 aa, Artificial Sequence
  FNI2 CDRL1(aa)
QSVSSN

SEQ ID NO: 68 - protein, 3 aa, Artificial Sequence
  FNI2 CDRL2(aa)
GAS

SEQ ID NO: 69 - protein, 10 aa, Artificial Sequence
  FNI2 CDRL3(aa)
QHYNNWPPWT

SEQ ID NO: 70 - DNA, 324 bp, Artificial Sequence
  FNI2 Vk (co-nt)
gacgtggtcatgacccagtctcctgccacactgagcgtgtctccaggagagagggccaccctgtcctgcagggcttcccagagcgtgtccagcaacctggcctggtaccagcagaagccaggccaggctcccaggctgctgatctatggagctagcaccagagctacaggcgtgccagctcgcttctctggatccggaagcggcacacagtttaccctgacaatctcttccctgcagtctgaggatttcgccgtgtactattgtcagcactacaacaattggcccccttggacctttggccagggcacaaagctggagatcaag

SEQ ID NO: 71 - DNA, 385 bp, Artificial Sequence
  FNI3 VH (wt-nt)
caggttcagctggtgcagtcgggggctgaggtgaagaggcctgggtcctcggtgaaggtctcctgcaaggcttctggagccaccttcagcaacaatgttatagcctgggtgcgacaggcccctggacaagggcttgagtggatgggggggatccaccctatctctgctacagcaacctacgcacagaagttccagggcagagtcgcgattgccgcggacgaattaacgagcacagcctacatggagttgaatggcctgagatctgaggactcggccgtgtattactgtgcgagagcggggtccgattactttaatagagacctcggctgggaaaattactactttgactcctggggccagggaaccctggtcaccgtctcgtcag

SEQ ID NO: 72 - protein, 128 aa, Artificial Sequence
  FNI3 VH (aa)
QVQLVQSGAEVKRPGSSVKVSCKASGATFSNNVIAWVRQAPGQGLEWMGGIHPISATATYAQKFQGRVAIAADELTSTAYMELNGLRSEDSAVYYCARAGSDYFNRDLGWENYYFDSWGQGTLVTVSS

SEQ ID NO: 73 - protein, 8 aa, Artificial Sequence
  FNI3 CDRH1 (aa)
GATFSNNV

SEQ ID NO: 74 - protein, 8 aa, Artificial Sequence
  FNI3 CDRH2 (aa)
IHPISATA

SEQ ID NO: 75 - protein, 21 aa, Artificial Sequence
  FNI3 CDRH3 (aa)
ARAGSDYFNRDLGWENYYFDS

SEQ ID NO: 76 - DNA, 384 bp, Artificial Sequence
  FNI3 VH (co-nt)
caggtgcagctggtgcagtccggagctgaggtgaagaggccaggatccagcgtgaaggtgtcctgcaaggccagcggcgctaccttcagcaacaatgtgatcgcttgggtgagacaggctccaggacagggactggagtggatgggaggaatccaccctatcagcgccaccgctacatacgcccagaagtttcagggcagagtggctatcgccgctgacgagctgacctctacagcctatatggagctgaacggcctgcgcagcgaggattccgccgtgtactattgtgccagggctggctctgactacttcaaccgggatctgggctgggagaattactattttgactcctggggccagggcaccctggtgacagtgtcttcc

SEQ ID NO: 77 - DNA, 325 bp, Artificial Sequence
  FNI3 Vk (wt-nt)
gaaatattgatgacgcagtctccagccaccctgtctgtgtctccaggggaaagagccaccctctcctgcagggccagtcaggatgttagcggcaacttagcctggtaccagcagagacctggccaggctcccaggctccttatctatggtgcatccacgagggccactggtgtcccagccaggttcactggcgctgggtctgggacagagttcactctcaccatcagcagcctgcagtctgaggattttgcactttattactgtcagcactataataactggcctccgtggaccttcggccaagggaccaaggtggaaatcaaac

SEQ ID NO: 78 - protein, 108 aa, Artificial Sequence
  FNI3 Vk (aa)
EILMTQSPATLSVSPGERATLSCRASQDVSGNLAWYQQRPGQAPRLLIYGASTRATGVPARFTGAGSGTEFTLTISSLQSEDFALYYCQHYNNWPPWTFGQGTKVEIK

SEQ ID NO: 79 - protein, 6 aa, Artificial Sequence
  FNI3 CDRL1(aa)
QDVSGN

SEQ ID NO: 80 - protein, 3 aa, Artificial Sequence
  FNI3 CDRL2(aa)
GAS

SEQ ID NO: 81 - protein, 10 aa, Artificial Sequence
  FNI3 CDRL3(aa)
QHYNNWPPWT

SEQ ID NO: 82 - DNA, 324 bp, Artificial Sequence
  FNI3 Vk (co-nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattggcccccttggacctttggccagggcacaaaggtggagatcaag

SEQ ID NO: 83 - DNA, 385 bp, Artificial Sequence
  FNI4 VH (wt-nt)
caggagcagctggtacagtctggggctgaggtgaagaagccggggtcctcggtgagggtctcctgcaaggcctctggagacaccttcagcagatatactatcagctgggttcgacaggcccccggacaaggacttgagtggatgggagggatcatcgctctctctcgaagagcgacatacgcacagaagttccagggcagagttaccattaccgcggacgaatccgcgaccacagcctacatacaactgagcggcctgacatctgacgacacggccgtatattactgtgcgagagcacactccgattactttaatagagacctcggctgggaagattactactttgactactggggccagggaaccctggtcaccgtctcctcag

SEQ ID NO: 84 - protein, 128 aa, Artificial Sequence
  FNI4 VH (aa)
QEQLVQSGAEVKKPGSSVRVSCKASGDTFSRYTISWVRQAPGQGLEWMGGIIALSRRATYAQKFQGRVTITADESATTAYIQLSGLTSDDTAVYYCARAHSDYFNRDLGWEDYYFDYWGQGTLVTVSS

SEQ ID NO: 85 - protein, 8 aa, Artificial Sequence
  FNI4 CDRH1 (aa)
GDTFSRYT

SEQ ID NO: 86 - protein, 8 aa, Artificial Sequence
  FNI4 CDRH2 (aa)
IIALSRRA

SEQ ID NO: 87 - protein, 21 aa, Artificial Sequence
  FNI4 CDRH3 (aa)
ARAHSDYFNRDLGWEDYYFDY

SEQ ID NO: 88 - DNA, 384 bp, Artificial Sequence
  FNI4 VH (co-nt)
caggagcagctggtgcagtccggagctgaggtgaagaagccaggatccagcgtgagagtgagctgcaaggcttctggcgacaccttctctagatacacaatctcctgggtgcgccaggctcctggacagggactggagtggatgggaggaatcatcgctctgagcaggcgggccacctacgctcagaagtttcagggccgcgtgaccatcacagccgatgagtctgccaccacagcttatatccagctgtccggcctgaccagcgacgatacagccgtgtactattgtgccagggctcacagcgactacttcaaccgggatctgggctgggaggactactattttgattattggggccagggcaccctggtgacagtgtcttcc

SEQ ID NO: 89 - DNA, 325 bp, Artificial Sequence
  FNI4 Vk (wt-nt)
gaagtagtgctgacgcagtctccagccaccctgtctgtgtctctaggggaaagagccatcctctcctgcagggccagtcagagtgttagcaccaacttagcctggtaccagcagagacctggccaggctcccaggctcctcatctctggtgcatccaccagggccacgggtatcccagccaggttcagtggcagtgggtctgggacagagttcacgctcaccatcagcagcctgcagtctgaagattttgcagtttattactgtcagcagtataataactggcctccgtggacgttcggccaagggaccaaggtggaaatcagac

SEQ ID NO: 90 - protein, 108 aa, Artificial Sequence
  FNI4 VK (aa)
EVVLTQSPATLSVSLGERAILSCRASQSVSTNLAWYQQRPGQAPRLLISGASTRATGIPARFSGSGSGTEFTLTISSLQSEDFAVYYCQQYNNWPPWTFGQGTKVEIR

SEQ ID NO: 91 - protein, 6 aa, Artificial Sequence
  FNI4 CDRL1 (aa)
QSVSTN

SEQ ID NO: 92 - protein, 3 aa, Artificial Sequence
  FNI4 CDRL2 (aa)
GAS

SEQ ID NO: 93 - protein, 10 aa, Artificial Sequence [sequence omitted]

SEQ ID NO: 94 - DNA, 324 bp, Artificial Sequence
  FNI4 Vk (co-nt)
gaggtggtgctgacccagtcccctgccacactgtccgtgtccctgggagagagggctatcctgagctgcagggctagccagtccgtgtccaccaacctggcctggtaccagcagagaccaggacaggctccaaggctgctgatcagcggagcttctaccagggctacaggcatcccagccagattcagcggctctggctccggcacagagtttaccctgacaatctccagcctgcagtctgaggacttcgccgtgtactattgtcagcagtataacaattggcccccttggacctttggccagggcacaaaggtggagatcagg

SEQ ID NO: 95 - DNA, 385 bp, Artificial Sequence
  FNI5 VH (wt-nt)
caggtgcagctgatacaatctgaggctgaggtgaagaagcctgggtcctcggtgagggtctcctgcaaggcttctggagacaccttcagcaaatatactatcggctgggtgcgacaggcccccggacaagggcttgagtggatgggagggatcatccctctctctcgaacagcgacctacgcacagaagttccagggcagagtcacgattaccgcggacgaatccacgaccacagtttacatgcaactgagcggcctgagatctgacgacacggccgcatattactgtgcgagagcacgctcggattactttaatagagacctcggctgggacgattactactttgattactggggccagggaaccctggtcaccgtctcctcag

SEQ ID NO: 96 - protein, 128 aa, Artificial Sequence
  FNI5 VH (aa)
QVQLIQSEAEVKKPGSSVRVSCKASGDTFSKYTIGWVRQAPGQGLEWMGGIIPLSRTATYAQKFQGRVTITADESTTTVYMQLSGLRSDDTAAYYCARARSDYFNRDLGWDDYYFDYWGQGTLVTVSS

SEQ ID NO: 97 - protein, 8 aa, Artificial Sequence
  FNI5 CDRH1 (aa)
GDTFSKYT

SEQ ID NO: 98 - protein, 8 aa, Artificial Sequence
  FNI5 CDRH2 (aa)
IIPLSRTA

SEQ ID NO: 99 - protein, 21 aa, Artificial Sequence
  FNI5 CDRH3 (aa)
ARARSDYFNRDLGWDDYYFDY

SEQ ID NO: 100 - DNA, 384 bp, Artificial Sequence
  FNI5 VH (co-nt)
caggtgcagctgatccagagcgaggccgaggtgaagaagccaggctccagcgtgagggtgagctgcaaggcttctggcgacacattctctaagtacaccatcggatgggtgcggcaggctccaggacagggcctggagtggatgggcggcatcatccctctgtctagaacagccacctacgctcagaagtttcagggccgcgtgacaatcaccgctgacgagtccaccacaaccgtgtatatgcagctgtccggcctgagaagcgacgatacagccgcttactattgtgccagggctcggtccgactacttcaaccgcgatctgggctgggacgattactattttgattattggggccagggcacactggtgaccgtgtcttcc

SEQ ID NO: 101 - DNA, 325 bp, Artificial Sequence
  FNI5 Vk (wt-nt)
gaaatagtgatgacgcagtctccagccaacctgtctgtgtctccaggggaaagagccaccctctcctgcagggccagtcagactgttagcaccaacttagcctggtaccagcagaagcctggccaggctcccaggctcctcatctctggtgcatccaccagggccactggtatcccagccaggttcagtggcagtgggtctgggacagagttcacgctcaccatcagcagcctgcagtctgaagattttgcagtttattactgtcagcagtataataattggcctccgtggacgttcggccaagggaccaaggtggaaatcagac

SEQ ID NO: 102 - protein, 108 aa, Artificial Sequence
  FNI5 VK (aa)
EIVMTQSPANLSVSPGERATLSCRASQTVSTNLAWYQQKPGQAPRLLISGASTRATGIPARFSGSGSGTEFTLTISSLQSEDFAVYYCQQYNNWPPWTFGQGTKVEIR

SEQ ID NO: 103 - protein, 6 aa, Artificial Sequence
  FNI5 CDRL1(aa)
QTVSTN

SEQ ID NO: 104 - protein, 3 aa, Artificial Sequence
  FNI5 CDRL2(aa)
GAS

SEQ ID NO: 105 - protein, 10 aa, Artificial Sequence [sequence omitted]

SEQ ID NO: 106 - DNA, 324 bp, Artificial Sequence
  FNI5 Vk (co-nt)
gagatcgtgatgacccagtcccctgctaacctgtccgtgtccccaggagagagggccacactgtcctgccgggctagccagaccgtgtctacaaatctggcctggtaccagcagaagccaggacaggctccaaggctgctgatcagcggagcttctaccagagctacaggcatcccagctcgcttcagcggatctggatccggcaccgagtttaccctgacaatctccagcctgcagagcgaggacttcgccgtgtactattgtcagcagtataacaattggcccccttggacctttggccagggcacaaaggtggagatcaga

SEQ ID NO: 107 - DNA, 385 bp, Artificial Sequence
  FNI6 VH (wt-nt)
caggtgcagctggtgcagtctggggctgaggtgaagaagcctgggtcctcggtgaaggtctcctgcaaggcctctggaggcaccttcagtagtcaagttatcagctgggtgcgagaggccccaggacaagggcttgagtggatgggagggatcattcctatcactggaatagcgaacaacgcacagaagttccagggcagagtcacgattaccgcggacggatccacgggcacagtctacatggagttgagcagcctgagatctggggacacggccgtctattactgtgcgagagcgggttcggattattttaatagagacctcggctgggaaaattactactttgaatattggggccagggaaccctggtcaccgtctcctcag

SEQ ID NO: 108 - protein, 128 aa, Artificial Sequence
  FNI6 VH (aa)
QVQLVQSGAEVKKPGSSVKVSCKASGGTFSSQVISWVREAPGQGLEWMGGIIPITGIANNAQKFQGRVTITADGSTGTVYMELSSLRSGDTAVYYCARAGSDYFNRDLGWENYYFEYWGQGTLVTVSS

SEQ ID NO: 109 - protein, 8 aa, Artificial Sequence
  FNI6 CDRH1 (aa)
GGTFSSQV

SEQ ID NO: 110 - protein, 8 aa, Artificial Sequence
  FNI6 CDRH2 (aa)
IIPITGIA

SEQ ID NO: 111 - protein, 21 aa, Artificial Sequence
  FNI6 CDRH3 (aa)
ARAGSDYFNRDLGWENYYFEY

SEQ ID NO: 112 - DNA, 384 bp, Artificial Sequence
  FNI6 VH (co-nt)
caggtgcagctggtgcagagcggagctgaggtgaagaagccaggctccagcgtgaaggtgtcttgcaaggcttccggcggcaccttctcttcccaggtcatctcttgggtgagggaggctccaggacagggactggagtggatgggcggcatcatccctatcacaggcatcgccaacaatgctcagaagtttcagggcagagtgaccatcacagccgacggcagcaccggcacagtgtacatggagctgagctctctgcgctctggcgataccgccgtgtactattgtgccagggctggctccgactacttcaaccgggatctgggctgggagaattactattttgagtattggggccagggcaccctggtgacagtgtccagc

SEQ ID NO: 113 - DNA, 325 bp, Artificial Sequence
  FNI6 Vk (wt-nt)
gaaatcgtgatgacacagtctccagccaccctgtctgtatctccaggggaaagagccatcctctcctgcagggccagtcagagtgttagcacccacttagcctggtaccagcagaaacctggccaggctcccagactcctcgtttttgatgcatccaccagggccactggtgtcccagccagattcggtggcagtgggtctgggacagagttcactctcaccatcagcagcctgcagtctgaagattctgctgtttattactgtcaacactataataactggcctccgtggacgttcggccaagggaccaacgtggaaatcagac

SEQ ID NO: 114 - protein, 108 aa, Artificial Sequence
  FNI6 VK (aa)
EIVMTQSPATLSVSPGERAILSCRASQSVSTHLAWYQQKPGQAPRLLVFDASTRATGVPARFGGSGSGTEFTLTISSLQSEDSAVYYCQHYNNWPPWTFGQGTNVEIR

SEQ ID NO: 115 - protein, 6 aa, Artificial Sequence
  FNI6 CDRL1(aa)
QSVSTH

SEQ ID NO: 116 - protein, 3 aa, Artificial Sequence
  FNI6 CDRL2(aa)
DAS

SEQ ID NO: 117 - protein, 10 aa, Artificial Sequence
  FNI6 CDRL3(aa)
QHYNNWPPWT

SEQ ID NO: 118 - DNA, 324 bp, Artificial Sequence
  FNI6 Vk (co-nt)
gagatcgtgatgacccagtctcctgccacactgtccgtgtccccaggagagagggctatcctgtcctgcagggctagccagtccgtgtccacccacctggcctggtaccagcagaagccaggccaggctcccaggctgctggtgttcgacgctagcaccagagctacaggcgtgccagctaggttcggaggaagcggatctggcacagagtttaccctgacaatctccagcctgcagtccgaggattccgccgtgtactattgtcagcattataacaattggcccccttggacctttggccagggcacaaacgtggagatcaga

SEQ ID NO: 119 - DNA, 385 bp, Artificial Sequence
  FNI7 VH (wt-nt)
caagtgcagctggtgcagtctggggctgaggtgaagaagcctgggtcctcggtgaaagtctcctgtaagacttctggaggcaccttcaataggcaagttatcagctgggtgcgacaggccccaggacaaggacttgagtggatgggagggatcctccctcttactggtagaggggacgaggcagagaggtttcagggcagagtcaccattaccgcggacgaatctgagagtacagtctacatggacttgagcagcctgagatctggggacacggccgtctattactgtgcgagagcgcgttcggattactttaatagagacctcggctgggaaaattactactttgaatcttggggccagggaaccctggtcaccgtctcctcag

SEQ ID NO: 120 - protein, 128 aa, Artificial Sequence
  FNI7 VH (aa)
QVQLVQSGAEVKKPGSSVKVSCKTSGGTFNRQVISWVRQAPGQGLEWMGGILPLTGRGDEAERFQGRVTITADESESTVYMDLSSLRSGDTAVYYCARARSDYFNRDLGWENYYFESWGQGTLVTVSS

SEQ ID NO: 121 - protein, 8 aa, Artificial Sequence
  FNI7 CDRH1 (aa)
GGTFNRQV

SEQ ID NO: 122 - protein, 8 aa, Artificial Sequence
  FNI7 CDRH2 (aa)
ILPLTGRG

SEQ ID NO: 123 - protein, 21 aa, Artificial Sequence
  FNI7 CDRH3 (aa)
ARARSDYFNRDLGWENYYFES

SEQ ID NO: 124 - DNA, 384 bp, Artificial Sequence
  FNI7 VH (co-nt)
caggtgcagctggtgcagtccggagctgaggtgaagaagccaggctccagcgtgaaggtgtcttgcaagacctccggcggcacattcaacaggcaggtcatcagctgggtgcggcaggctccaggacagggactggagtggatgggaggaatcctgcctctgaccggcaggggcgacgaggccgagagatttcagggccgcgtgaccatcacagctgatgagtccgagagcaccgtgtacatggacctgtcttccctgagaagcggcgatacagccgtgtactattgtgccagggctcggtctgactatttcaaccgcgatctgggctgggagaattactattttgagtcttggggccagggcaccctggtgacagtgagctct

SEQ ID NO: 125 - DNA, 325 bp, Artificial Sequence
  FNI7 Vk (wt-nt)
gaaatcgtgatgacgcagtctccagccaccctgtctgtatctccaggggaaagagccaccctctcctgcagggccagtcagagtgttagtaccgacttagtctggtaccagcagaaacctggccaggctccccggctcctcatttatgatgcatccactagggccactggtatcccagccaggttcggtggcagggggtctgggacagagttcactctcaccatcagcagcctgcagtctgaagattctgctgtttattactgtcagcactattcttactggcctccgtggacattcggccaagggaccaaagtggaaatcaatc

SEQ ID NO: 126 - protein, 108 aa, Artificial Sequence
  FNI7 VK (aa)
EIVMTQSPATLSVSPGERATLSCRASQSVSTDLVWYQQKPGQAPRLLIYDASTRATGIPARFGGRGSGTEFTLTISSLQSEDSAVYYCQHYSYWPPWTFGQGTKVEIN

SEQ ID NO: 127 - protein, 6 aa, Artificial Sequence
  FNI7 CDRL1(aa)
QSVSTD

SEQ ID NO: 128 - protein, 3 aa, Artificial Sequence
  FNI7 CDRL2(aa)
DAS

SEQ ID NO: 129 - protein, 10 aa, Artificial Sequence
  FNI7 CDRL3(aa)
QHYSYWPPWT

SEQ ID NO: 130 - DNA, 324 bp, Artificial Sequence
  FNI7 Vk (co-nt)
gagatcgtgatgacccagtcccctgccacactgtccgtgtccccaggagagagagccaccctgagctgcagggctagccagtccgtgtccacagacctggtgtggtaccagcagaagccaggacaggctccaaggctgctgatctatgatgcctctaccagagctacaggcatcccagctaggttcggaggaaggggatccggcaccgagtttaccctgacaatctccagcctgcagagcgaggactccgccgtgtactattgtcagcactacagctattggcccccttggaccttcggccagggcacaaaggtggagatcaac

SEQ ID NO: 131 - DNA, 385 bp, Artificial Sequence
  FNI9 VH (wt-nt)
caggtccacctggtgcagtctggggctgaggtgaaggagcctgggtcctcggtgacggtctcctgcaaggcatctggaggcagcttcaacaaccaggctattagctgggtgcgacaggccccaggacaaggccttgagtggatgggagggatcttccctatctctggcacaccgaccagcgcacagaggttccagggcagagtcacatttaccgcggacgagtccacgaccacagtctacatggatctgagcagcctgagatctgacgacacggccgtctactactgtgcgagagcgggttcggattactttaatagagacctcggctgggaaaactactactttgcgtcctggggccagggaaccctggtcaccgtctcctcag

SEQ ID NO: 132 - protein, 128 aa, Artificial Sequence [sequence omitted]

SEQ ID NO: 133 - protein, 8 aa, Artificial Sequence
  FNI9 CDRH1 (aa)
GGSFNNQA

SEQ ID NO: 134 - protein, 8 aa, Artificial Sequence
  FNI9 CDRH2 (aa)
IFPISGTP

SEQ ID NO: 135 - protein, 21 aa, Artificial Sequence
  FNI9 CDRH3 (aa)
ARAGSDYFNRDLGWENYYFAS

SEQ ID NO: 136 - DNA, 384 bp, Artificial Sequence
  FNI9 VH (co-nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggctgggagaattactattttgcttcctggggccagggcacactggtgaccgtgagctct

SEQ ID NO: 137 - DNA, 325 bp, Artificial Sequence
  FNI9 Vk (wt-nt)
gaaatcgtgatgacgcagtctccagccaccctgtctctatcttcaggggaaagagccaccctctcctgcagggccagtcggagtgttagtagcaacttagcctggtaccagcagaaacctggccaggctcccaggctcctcatttatgatgcatccaccagggccactggtttttcagccaggttcgctggcagtgggtctgggacagagttcactctcaccatcagcagcctgcagtctgaagattctgctatttattactgtcagcagtataataactggcctccgtggacgttcggccaagggaccaaggtggaaatcaaac

SEQ ID NO: 138 - protein, 108 aa, Artificial Sequence
  FNI9 VK (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPWTFGQGTKVEIK

SEQ ID NO: 139 - protein, 6 aa, Artificial Sequence
  FNI9 CDRL1(aa)
RSVSSN

SEQ ID NO: 140 - protein, 3 aa, Artificial Sequence
  FNI9 CDRL2(aa)
DAS

SEQ ID NO: 141 - protein, 10 aa, Artificial Sequence [sequence omitted]

SEQ ID NO: 142 - DNA, 324 bp, Artificial Sequence
  FNI9 Vk (co-nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggcccccttggacctttggccagggcacaaaggtggagatcaag

SEQ ID NO: 143 - DNA, 385 bp, Artificial Sequence
  FNI10 VH (wt-nt)
caggtgcagctggtgcagtctggggctgaggtgaagaagcctgggtcctcggtgaaagtctcctgcaaggcttctggaggcaccttgagtagtcaagttattagctgggtgcgacaggccccaggacaaggactggagtggatcggagggatcatccccaccactggtacagggggcgcggcagaggggttccagggcagagtctccatttccgcggacgaatccaggagcacagtctacatggaactgaccagcctgacttctggggacacggccgtctattattgtgcgagagcggtttcggattactttaatagagacctcggctgggaaaattactactttgaatcttggggccagggaaccctggtcaccgtctcctcag

SEQ ID NO: 144 - protein, 128 aa, Artificial Sequence
  FNI10 VH (aa)
QVQLVQSGAEVKKPGSSVKVSCKASGGTLSSQVISWVRQAPGQGLEWIGGIIPTTGTGGAAEGFQGRVSISADESRSTVYMELTSLTSGDTAVYYCARAVSDYFNRDLGWENYYFESWGQGTLVTVSS

SEQ ID NO: 145 - protein, 8 aa, Artificial Sequence
  FNI10 CDRH1 (aa)
GGTLSSQV

SEQ ID NO: 146 - protein, 8 aa, Artificial Sequence
  FNI10 CDRH2 (aa)
IIPTTGTG

SEQ ID NO: 147 - protein, 21 aa, Artificial Sequence
  FNI10 CDRH3 (aa)
ARAVSDYFNRDLGWENYYFES

SEQ ID NO: 148 - DNA, 384 bp, Artificial Sequence
  FNI10 VH (co-nt)
caggtgcagctggtgcagagcggagctgaggtgaagaagccaggctccagcgtgaaggtgtcctgcaaggctagcggcggcaccctgtcttcccaggtcatctcttgggtgaggcaggctccaggacagggactggagtggatcggcggcatcatccctaccacaggcacaggcggagctgctgagggattccagggcagagtgtccatcagcgccgacgagtctcgctccaccgtgtacatggagctgaccagcctgacatctggcgatacagccgtgtactattgtgccagggccgtgtccgactatttcaaccgggatctgggctgggagaattactattttgagtcctggggccagggcaccctggtgacagtgagctct

SEQ ID NO: 149 - DNA, 325 bp, Artificial Sequence
  FNI10 Vk (wt-nt)
gaaatcgtgatgacgcagtctccagccaccctgtctgtgtctccaggggaaagagccaccctctcttgcagggccagtcggagtgttagtatcaacttagcctggtaccaacagaaacctggccaggctccccggctcctcatttatgatgcatctacgagggccactggcatcccagccaggttcggtggcagggggtctggaacagagttcactctcaccatcagcagcctgcagtctgaagattctgctgtttattactgtcagcactataataactggcctccgtggacattcggccaagggaccagagtggaaatcaaac

SEQ ID NO: 150 - protein, 108 aa, Artificial Sequence
  FNI10 VK (aa)
EIVMTQSPATLSVSPGERATLSCRASRSVSINLAWYQQKPGQAPRLLIYDASTRATGIPARFGGRGSGTEFTLTISSLQSEDSAVYYCQHYNNWPPWTFGQGTRVEIK

SEQ ID NO: 151 - protein, 6 aa, Artificial Sequence
  FNI10 CDRL1(aa)
RSVSIN

SEQ ID NO: 152 - protein, 3 aa, Artificial Sequence
  FNI10 CDRL2(aa)
DAS

SEQ ID NO: 153 - protein, 10 aa, Artificial Sequence
  FNI10 CDRL3(aa)
QHYNNWPPWT

SEQ ID NO: 154 - DNA, 324 bp, Artificial Sequence
  FNI10 Vk (co-nt)
gagatcgtgatgacccagtcccctgccacactgtccgtgtccccaggagagagagccaccctgagctgcagggctagcaggtccgtgtccatcaacctggcctggtaccagcagaagccaggccaggctcccaggctgctgatctatgacgcttctaccagggctacaggcatcccagctagattcggaggaaggggatccggaacagagtttaccctgacaatctccagcctgcagagcgaggattccgccgtgtactattgtcagcactacaacaattggccaccttggaccttcggccagggaacacgcgtggagatcaag

SEQ ID NO: 155 - DNA, 385 bp, Artificial Sequence
  FNI12 VH (wt-nt)
caggtgcacctggtacagtctggggctgaggtgaagaagcctgggtcctcggtgagggtctcctgcaaggcttctggagactccttcaacaaatatgaagtcagctgggtgcgacaggcccccggacatggacttgagtggatgggagggatcatccctctctctcctatagcgaggtacgcagagaaatttcagggcagagtcacgattaccgcggacgaattcacgagcacggtctatatacaactgaccagcctgagatctgacgacacggccgtatactactgtgcgacaacacgttcggattactttaatagagacctcggctgggaagattacttctttgaccactggggccagggaaccctggtcaccgtctcctcag

SEQ ID NO: 156 - protein, 128 aa, Artificial Sequence
  FNI12 VH (aa)
QVHLVQSGAEVKKPGSSVRVSCKASGDSFNKYEVSWVRQAPGHGLEWMGGIIPLSPIARYAEKFQGRVTITADEFTSTVYIQLTSLRSDDTAVYYCATTRSDYFNRDLGWEDYFFDHWGQGTLVTVSS

SEQ ID NO: 157 - protein, 8 aa, Artificial Sequence
  FNI12 CDRH1 (aa)
GDSFNKYE

SEQ ID NO: 158 - protein, 8 aa, Artificial Sequence
  FNI12 CDRH2 (aa)
IIPLSPIA

SEQ ID NO: 159 - protein, 21 aa, Artificial Sequence
  FNI12 CDRH3 (aa)
ATTRSDYFNRDLGWEDYFFDH

SEQ ID NO: 160 - DNA, 384 bp, Artificial Sequence
  FNI12 VH (co-nt)
caggtgcacctggtgcagtctggcgccgaggtgaagaagccaggctccagcgtgagggtgtcctgcaaggctagcggcgactctttcaacaagtacgaggtgagctgggtgagacaggctccaggacatggactggagtggatgggcggcatcatccccctgtctcctatcgccagatacgctgagaagttccagggccgcgtgaccatcacagctgatgagtttacctccacagtgtatatccagctgacctccctgaggagcgacgatacagccgtgtactattgtgctaccacaaggagcgactactttaatcgggatctgggctgggaggactatttctttgatcactggggccagggcaccctggtgacagtgtcttcc

SEQ ID NO: 161 - DNA, 325 bp, Artificial Sequence
  FNI12 Vk (wt-nt)
gaaatagtgatgacgcagtctccagccaccctgtctgtgtctccaggggaaagagccaccctctcctgcagggccagtcagagtattagcaccaacttagcctggtaccagcagaaacctggccaggctcccaggctcctcatctctggtgcatccaccagggccactggtatcccagccaggttcagtggcagtgggtctgggacagagttcactctcaccatcagcagcctgcagtctgaagattttggagtttattactgtcagcactataataactggcctccgtggacgttcggccaagggaccaaggtggaaatcaaac

SEQ ID NO: 162 - protein, 108 aa, Artificial Sequence
  FNI12 VK (aa)
EIVMTQSPATLSVSPGERATLSCRASQSISTNLAWYQQKPGQAPRLLISGASTRATGIPARFSGSGSGTEFTLTISSLQSEDFGVYYCQHYNNWPPWTFGQGTKVEIK

SEQ ID NO: 163 - protein, 6 aa, Artificial Sequence
  FNI12 CDRL1(aa)
QSISTN

SEQ ID NO: 164 - protein, 3 aa, Artificial Sequence
  FNI12 CDRL2(aa)
GAS

SEQ ID NO: 165 - protein, 10 aa, Artificial Sequence
  FNI12 CDRL3(aa)
QHYNNWPPWT

SEQ ID NO: 166 - DNA, 324 bp, Artificial Sequence
  FNI12 Vk (co-nt)
gagatcgtgatgacccagtcccctgccacactgtccgtgtccccaggagagagggccaccctgagctgccgggctagccagtctatctccacaaacctggcctggtaccagcagaagccaggacaggctccaaggctgctgatcagcggagcttctaccagagctacaggcatcccagctcgcttcagcggatctggatccggaaccgagtttaccctgacaatctccagcctgcagtctgaggacttcggcgtgtactattgtcagcactataacaattggcccccttggacctttggccagggcacaaaggtggagatcaag

SEQ ID NO: 167 - DNA, 385 bp, Artificial Sequence
  FNI13 VH (wt-nt)
caggttcagctggtgcaatctggggctgaggtgaagaggcctgggtcctcggtgagggtctcctgcaagggttctggagacaccttcaacaactatgttatcagttgggtgcgacaggcccctggccaagggcttgagtggatgggggggatcatccctatctttcaaacaccaaactacgcagagaagttccagggcagagtcgcgattaccgcggacgaatccacgagcacggcctacatggagttgagcagcctgagatctgaggactcggccatttattactgtgcgagagcgaattccgattactttaatagagacctcggctgggaaaattactactttgaagactggggccagggaaccctggtcaccgtctcctcag

SEQ ID NO: 168 - protein, 128 aa, Artificial Sequence
  FNI13 VH (aa)
QVQLVQSGAEVKRPGSSVRVSCKGSGDTFNNYVISWVRQAPGQGLEWMGGIIPIFQTPNYAEKFQGRVAITADESTSTAYMELSSLRSEDSAIYYCARANSDYFNRDLGWENYYFEDWGQGTLVTVSS

SEQ ID NO: 169 - protein, 8 aa, Artificial Sequence
  FNI13 CDRH1 (aa)
GDTFNNYV

SEQ ID NO: 170 - protein, 8 aa, Artificial Sequence
  FNI13 CDRH2 (aa)
IIPIFQTP

SEQ ID NO: 171 - protein, 21 aa, Artificial Sequence
  FNI13 CDRH3 (aa)
ARANSDYFNRDLGWENYYFED

SEQ ID NO: 172 - DNA, 384 bp, Artificial Sequence
  FNI13 VH (co-nt)
caggtgcagctggtgcagtccggagctgaggtgaagaggccaggatccagcgtgcgggtgagctgcaagggatctggcgacaccttcaacaattacgtgatcagctgggtgaggcaggctccaggacagggactggagtggatgggcggcatcatccccatcttccagacccctaactacgctgagaagtttcagggcagggtggccatcacagctgacgagtccaccagcacagcctatatggagctgtcttccctgagatctgaggattccgctatctactattgtgccagagctaactctgactatttcaatcgcgatctgggctgggagaattactattttgaggattggggccagggcaccctggtgacagtgagctct

SEQ ID NO: 173 - DNA, 325 bp, Artificial Sequence
  FNI13 Vk (wt-nt)
gaaagagtgatgacgcagtctccagccaccctttctgtgtctccagggggaagagccaccctctcctgcagggccagtcagagtgttggtagcaacttagcctggtaccagcagaaacctggccaggctcccaggctcctcatctatgatgcttctgccagggccactggtgtcccagccaggttcagtggcagtgggtctgggacagagttctctctctccatcaacagcctgcagtctgaagattctgcagtttattactgtcagcactataatatctggccgccgtggacgttcggccaagggaccaaggtggaaatcaaac

SEQ ID NO: 174 - protein, 108 aa, Artificial Sequence
  FNI13 VK (aa)
ERVMTQSPATLSVSPGGRATLSCRASQSVGSNLAWYQQKPGQAPRLLIYDASARATGVPARFSGSGSGTEFSLSINSLQSEDSAVYYCQHYNIWPPWTFGQGTKVEIK

SEQ ID NO: 175 - protein, 6 aa, Artificial Sequence
  FNI13 CDRL1(aa)
QSVGSN

SEQ ID NO: 176 - protein, 3 aa, Artificial Sequence
  FNI13 CDRL2(aa)
DAS

SEQ ID NO: 177 - protein, 10 aa, Artificial Sequence
  FNI13 CDRL3(aa)
QHYNIWPPWT

SEQ ID NO: 178 - DNA, 324 bp, Artificial Sequence
  FNI13 Vk (co-nt)
gagagagtgatgacccagtctcctgctacactgtccgtgagcccaggaggaagggctaccctgtcctgcagggcttctcagtccgtgggaagcaacctggcttggtaccagcagaagccaggccaggcccccagactgctgatctatgacgcttccgctagagctaccggcgtgccagctcgcttcagcggatctggctccggcacagagtttagcctgtctatcaactccctgcagagcgaggattctgccgtgtactattgtcagcactacaatatctggccaccttggaccttcggccagggaacaaaggtggagatcaag

SEQ ID NO: 179 - DNA, 385 bp, Artificial Sequence
  FNI14 VH (wt-nt)
caagttcagttggtgcagtctggggctgagctgaagcggcctgggtcctcggtgaggatctcctgcaaggcctctggtgtcaccttcaacaagtatgttctcagctgggtgcgactggcccctggacaagggcttgagtggatgggaggaatcatccctatttctggtataccacattacgcagagaagttccagggcagagtcgcgattaccgcggacgaatccacgagcacagtctacatggagttgagcagcctacgatctgaggactcggccctatattactgtgcgagagcggtctccgattattttaatcgggacctcggctgggatgattactactttcctttgtggggccacggcaccctggtcaccgtctcctcag

SEQ ID NO: 180 - protein, 128 aa, Artificial Sequence
  FNI14 VH (aa)
QVQLVQSGAELKRPGSSVRISCKASGVTFNKYVLSWVRLAPGQGLEWMGGIIPISGIPHYAEKFQGRVAITADESTSTVYMELSSLRSEDSALYYCARAVSDYFNRDLGWDDYYFPLWGHGTLVTVSS

SEQ ID NO: 181 - protein, 8 aa, Artificial Sequence
  FNI14 CDRH1 (aa)
GVTFNKYV

SEQ ID NO: 182 - protein, 8 aa, Artificial Sequence
  FNI14 CDRH2 (aa)
IIPISGIP

SEQ ID NO: 183 - protein, 21 aa, Artificial Sequence
  FNI14 CDRH3 (aa)
ARAVSDYFNRDLGWDDYYFPL

SEQ ID NO: 184 - DNA, 384 bp, Artificial Sequence
  FNI14 VH (co-nt)
caggtgcagctggtgcagtctggagctgagctgaagaggccaggatccagcgtgcggatcagctgcaaggcttctggcgtgaccttcaacaagtacgtgctgtcctgggtgaggctggctccaggacagggactggagtggatgggcggcatcatccccatcagcggcatccctcactacgctgagaagtttcagggcagggtggccatcacagctgacgagtccaccagcacagtgtatatggagctgtcttccctgagatctgaggattccgccctgtactattgtgccagagccgtgtccgactatttcaatcgcgatctgggctgggacgattactattttcccctgtggggccatggcaccctggtgacagtgagctct

SEQ ID NO: 185 - DNA, 325 bp, Artificial Sequence
  FNI14 Vk (wt-nt)
gaaatagtgatgacgcagtctccagccaccctgtctgtgtctccaggggaaagcgccaccctcttctgcagggccagtcggagtgttagtgacaacttagcctggtaccagcagaaacctggccaggctcccaggctcctcatctttggtgcttccaccagggccactggtgtcccagccaggttcggtggcagtgggtctgggacacagttcactctcaccatcagcagcctgcagtctgaagattttgcagtttattactgtcagcattataataactggcctccgtggacgttcggccaagggaccaaggtggagatcaaac

SEQ ID NO: 186 - protein, 108 aa, Artificial Sequence
  FNI14 VK (aa)
EIVMTQSPATLSVSPGESATLFCRASRSVSDNLAWYQQKPGQAPRLLIFGASTRATGVPARFGGSGSGTQFTLTISSLQSEDFAVYYCQHYNNWPPWTFGQGTKVEIK

SEQ ID NO: 187 - protein, 6 aa, Artificial Sequence
  FNI14 CDRL1(aa)
RSVSDN

SEQ ID NO: 188 - protein, 3 aa, Artificial Sequence
  FNI14 CDRL2(aa)
GAS

SEQ ID NO: 189 - protein, 10 aa, Artificial Sequence
  FNI14 CDRL3(aa)
QHYNNWPPWT

SEQ ID NO: 190 - DNA, 324 bp, Artificial Sequence
  FNI14 Vk (co-nt)
gagatcgtgatgacccagtcccctgccacactgtccgtgtccccaggagagagcgccaccctgttctgcagggctagcaggtccgtgtccgacaacctggcctggtaccagcagaagccaggccaggctcccaggctgctgatctttggcgcctctaccagagctacaggcgtgccagctaggttcggaggaagcggatctggcacacagtttaccctgacaatctccagcctgcagtccgaggatttcgccgtgtactattgtcagcactataacaattggcccccttggacctttggccagggcacaaaggtggagatcaag

SEQ ID NO: 191 - DNA, 385 bp, Artificial Sequence
  FNI17 VH (wt-nt)
caggttcaactggtgcagtctggggctgaggtgaagaggcctgggtcctcggtgaaggtctcctgcaagccttccggaggcaccttcagcaacaatgttatcagctgggtgcgacaggcccctggacaagggcttgagtggatgggagggatcatccccacctctggtatagcaaactacgcgcagaagttccagggcagagtcgcgattattgcggacaaatctacgagcacagtctacatggcgttgagcagcctgagatctgaggactcggccgtgtatttctgtgccagagcgcggtccgactacttcaatagagacctcggctgggaagattactactttgagaactggggccagggaaccctggtcaccgtctcctcag

SEQ ID NO: 192 - protein, 128 aa, Artificial Sequence
  FNI17 VH (aa)
QVQLVQSGAEVKRPGSSVKVSCKPSGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 193 - protein, 8 aa, Artificial Sequence
  FNI17 CDRH1 (aa)
GGTFSNNV

SEQ ID NO: 194 - protein, 8 aa, Artificial Sequence
  FNI17 CDRH2 (aa)
IIPTSGIA

SEQ ID NO: 195 - protein, 21 aa, Artificial Sequence
  FNI17 CDRH3 (aa)
ARARSDYFNRDLGWEDYYFEN

SEQ ID NO: 196 - DNA, 384 bp, Artificial Sequence
  FNI17 VH (co-nt)
caggtgcagctggtgcagtccggagctgaggtgaagaggccaggctccagcgtgaaggtgagctgcaagccttctggcggcaccttctccaacaatgtgatcagctgggtgagacaggctccaggacagggactggagtggatgggaggaatcatccccacatctggcatcgccaactacgctcagaagtttcagggcagggtggccatcatcgctgataagtccaccagcacagtgtatatggccctgtcttccctgagatctgaggactccgccgtgtacttctgtgccagggctcggtccgactacttcaaccgcgatctgggctgggaggactactatttcgagaattggggccagggcaccctggtgacagtgagctct

SEQ ID NO: 197 - DNA, 325 bp, Artificial Sequence
  FNI17 Vk (wt-nt)
gaaatagtgatgacgcagtctccagccaccctgtctgtgtctccaggggaaagagccaccctctcctgcagggccagtcagagtgttggcagcagcttagtctggtaccagcagaaacctggccaggctcccaggctcctcatctatggtgcatccaccagggccactggtgtcccagccaggttcagtggcagtgggtctgggacagagttcactctcaccatcagcagcctgcagtctgaagattttgcagtttattactgtcagcactataataactggcctccgtggacgttcggccaagggaccaaggtggaaatcaaac

SEQ ID NO: 198 - protein, 108 aa, Artificial Sequence
  FNI17 VK (aa)
EIVMTQSPATLSVSPGERATLSCRASQSVGSSLVWYQQKPGQAPRLLIYGASTRATGVPARFSGSGSGTEFTLTISSLQSEDFAVYYCQHYNNWPPWTFGQGTKVEIK

SEQ ID NO: 199 - protein, 6 aa, Artificial Sequence
  FNI17 CDRL1(aa)
QSVGSS

SEQ ID NO: 200 - protein, 3 aa, Artificial Sequence
  FNI17 CDRL2(aa)
GAS

SEQ ID NO: 201 - protein, 10 aa, Artificial Sequence
  FNI17 CDRL3(aa)
QHYNNWPPWT

SEQ ID NO: 202 - DNA, 324 bp, Artificial Sequence
  FNI17 Vk (co-nt)
gagatcgtgatgacccagtctcctgccacactgagcgtgtctccaggagagagggccaccctgtcctgcagggcttcccagagcgtgggatccagcctggtgtggtaccagcagaagccaggacaggctccaaggctgctgatctatggagctagcaccagagctacaggcgtgccagctcgcttctctggatccggaagcggcacagagtttaccctgacaatctcttccctgcagtctgaggacttcgccgtgtactattgtcagcactacaacaattggcccccttggacctttggccagggcacaaaggtggagatcaag

SEQ ID NO: 203 - DNA, 385 bp, Artificial Sequence
  FNI19 VH (wt-nt)
caagttcagctggtgcagtctggggctgaggtgaagaggcctgggtcctcggtgagggtctcctgcaaggcttctgaaggcaccttcaacaagtatactctcacctgggtgcgacaggcccctggacagggacttgagtggatgggaggaatcatccctatctccggtatagcaaactacgcacagaagttccagggcagagtcgcgattaccgcggacgaatccacgaccacagcctacatggaattgagcagcctaagatctgaagactcggccgtatattactgtgcgacagcggtctccgattattttaatcgagacctcggctgggaagattactactttccgttctggggccagggcaccctggtcaccgtcgcctcag

SEQ ID NO: 204 - protein, 128 aa, Artificial Sequence
  FNI19 VH (aa)
QVQLVQSGAEVKRPGSSVRVSCKASEGTFNKYTLTWVRQAPGQGLEWMGGIIPISGIANYAQKFQGRVAITADESTTTAYMELSSLRSEDSAVYYCATAVSDYFNRDLGWEDYYFPFWGQGTLVTVAS

SEQ ID NO: 205 - protein, 8 aa, Artificial Sequence
  FNI19 CDRH1 (aa)
EGTFNKYT

SEQ ID NO: 206 - protein, 8 aa, Artificial Sequence
  FNI19 CDRH2 (aa)
IIPISGIA

SEQ ID NO: 207 - protein, 21 aa, Artificial Sequence
  FNI19 CDRH3 (aa)
ATAVSDYFNRDLGWEDYYFPF

SEQ ID NO: 208 - DNA, 384 bp, Artificial Sequence
  FNI19 VH (co-nt)
caggtgcagctggtgcagtccggagctgaggtgaagaggccaggatccagcgtgcgggtgtcctgcaaggctagcgagggcacattcaacaagtacacactgacctgggtgaggcaggctccaggacagggactggagtggatgggcggcatcatccctatctctggcatcgccaattacgctcagaagtttcagggcagagtggccatcacagctgatgagtccaccacaaccgcctatatggagctgtcttccctgagaagcgaggactccgccgtgtactattgtgccaccgctgtgagcgactatttcaaccgcgatctgggctgggaggactactatttccccttttggggccagggcacactggtgaccgtggcttct

SEQ ID NO: 209 - DNA, 325 bp, Artificial Sequence
  FNI19 Vk (wt-nt)
gaaatagtgatgacgcagtctccagccaccctgtctgtgtctccgggggccagagccaccctcttctgcagggccagtcggagtgttagtgacaacttagcctggtaccagcagaaacctggccaggctcccaggctcctcatctttggtgcatccaccagggccactggtgtcccagccaggttcagtggaagtgggtctgggacacagttcactctcaccatcagcagcctgcagtccgaagattttgcagtttattactgtcagcattataatatttggcctccgtggacgttcggccaagggaccaaggtggagatcaaac

SEQ ID NO: 210 - protein, 108 aa, Artificial Sequence
  FNI19 VK (aa)
EIVMTQSPATLSVSPGARATLFCRASRSVSDNLAWYQQKPGQAPRLLIFGASTRATGVPARFSGSGSGTQFTLTISSLQSEDFAVYYCQHYNIWPPWTFGQGTKVEIK

SEQ ID NO: 211 - protein, 6 aa, Artificial Sequence
  FNI19 CDRL1(aa)
RSVSDN

SEQ ID NO: 212 - protein, 3 aa, Artificial Sequence
  FNI19 CDRL2(aa)
GAS

SEQ ID NO: 213 - protein, 10 aa, Artificial Sequence
  FNI19 CDRL3(aa)
QHYNIWPPWT

SEQ ID NO: 214 - DNA, 324 bp, Artificial Sequence
  FNI19 Vk (co-nt)
gagatcgtgatgacccagtcccctgctacactgtccgtgtccccaggagctagggctaccctgttctgcagggctagcaggtccgtgtccgacaacctggcttggtaccagcagaagccaggccaggcccccagactgctgatctttggagctagcaccagagctacaggcgtgccagctcgcttcagcggatctggatccggcacacagtttaccctgacaatctccagcctgcagtctgaggatttcgccgtgtactattgtcagcactataatatctggcccccttggacctttggccagggcacaaaggtggagatcaag

SEQ ID NO: 215 - skipped

SEQ ID NO: 216 - DNA, 384 bp, Artificial Sequence
  FNI3-VH-W110F (nt)
caggtgcagctggtgcagtccggagctgaggtgaagaggccaggatccagcgtgaaggtgtcctgcaaggccagcggcgctaccttcagcaacaatgtgatcgcttgggtgagacaggctccaggacagggactggagtggatgggaggaatccaccctatcagcgccaccgctacatacgcccagaagtttcagggcagagtggctatcgccgctgacgagctgacctctacagcctatatggagctgaacggcctgcgcagcgaggattccgccgtgtactattgtgccagggctggctctgactacttcaaccgggatctgggcttcgagaattactattttgactcctggggccagggcaccctggtgacagtgtcttcc

SEQ ID NO: 217 - protein, 128 aa, Artificial Sequence
  FNI3-VH-W110F (aa)
QVQLVQSGAEVKRPGSSVKVSCKASGATFSNNVIAWVRQAPGQGLEWMGGIHPISATATYAQKFQGRVAIAADELTSTAYMELNGLRSEDSAVYYCARAGSDYFNRDLGFENYYFDSWGQGTLVTVSS

SEQ ID NO: 218 - protein, 21 aa, Artificial Sequence
  FNI3-VH-W110F CDRH3 (aa)
ARAGSDYFNRDLGFENYYFDS

SEQ ID NO: 219 - DNA, 324 bp, Artificial Sequence
  FNI3-VK-W94F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattttcccccttggacctttggccagggcacaaaggtggagatcaag

SEQ ID NO: 220 - protein, 108 aa, Artificial Sequence
  FNI3-VK-W94F (aa)
EILMTQSPATLSVSPGERATLSCRASQDVSGNLAWYQQRPGQAPRLLIYGASTRATGVPARFTGAGSGTEFTLTISSLQSEDFALYYCQHYNNFPPWTFGQGTKVEIK

SEQ ID NO: 221 - protein, 10 aa, Artificial Sequence
  FNI3-VK-W94F CDRL3 (aa)
QHYNNFPPWT

SEQ ID NO: 222 - DNA, 324 bp, Artificial Sequence
  FNI3-VK-W97F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattggccccctttcacctttggccagggcacaaaggtggagatcaag

SEQ ID NO: 223 - protein, 108 aa, Artificial Sequence
  FNI3-VK-W97F (aa)
EILMTQSPATLSVSPGERATLSCRASQDVSGNLAWYQQRPGQAPRLLIYGASTRATGVPARFTGAGSGTEFTLTISSLQSEDFALYYCQHYNNWPPFTFGQGTKVEIK

SEQ ID NO: 224 - protein, 10 aa, Artificial Sequence
  FNI3-VK-W97F CDRL3 (aa)
QHYNNWPPFT

SEQ ID NO: 225 - DNA, 324 bp, Artificial Sequence
  FNI3-VK-W94F-W97F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattttccccctttcacctttggccagggcacaaaggtggagatcaag

SEQ ID NO: 226 - protein, 108 aa, Artificial Sequence
  FNI3-VK-W94F-W97F (aa)
EILMTQSPATLSVSPGERATLSCRASQDVSGNLAWYQQRPGQAPRLLIYGASTRATGVPARFTGAGSGTEFTLTISSLQSEDFALYYCQHYNNFPPFTFGQGTKVEIK

SEQ ID NO: 227 - protein, 10 aa, Artificial Sequence
  FNI3-VK-W94F-W97F CDRL3 (aa)
QHYNNFPPFT

SEQ ID NO: 228 - DNA, 384 bp, Artificial Sequence
  FNI9-VH-W110F (nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggcttcgagaattactattttgcttcctggggccagggcacactggtgaccgtgagctct

SEQ ID NO: 229 - protein, 128 aa, Artificial Sequence
  FNI9-VH-W110F (aa)
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGFENYYFASWGQGTLVTVSS

SEQ ID NO: 230 - protein, 21 aa, Artificial Sequence
  FNI9-VH-W110F CDRH3 (aa)
ARAGSDYFNRDLGFENYYFAS

SEQ ID NO: 231 - DNA, 324 bp, Artificial Sequence
  FNI9-VK-W94F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaatttccccccttggacctttggccagggcacaaaggtggagatcaag

SEQ ID NO: 232 - protein, 108 aa, Artificial Sequence
  FNI9-VK-W94F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPWTFGQGTKVEIK

SEQ ID NO: 233 - protein, 10 aa, Artificial Sequence
  FNI9-VK-W94F CDRL3 (aa)
QQYNNFPPWT

SEQ ID NO: 234 - DNA, 324 bp, Artificial Sequence
  FNI9-VK-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggccccctttcacctttggccagggcacaaaggtggagatcaag

SEQ ID NO: 235 - protein, 108 aa, Artificial Sequence
  FNI9-VK-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPFTFGQGTKVEIK

SEQ ID NO: 236 - protein, 10 aa, Artificial Sequence
  FNI9-VK-W97F CDRL3 (aa)
QQYNNWPPFT

SEQ ID NO: 237 - DNA, 324 bp, Artificial Sequence
  FNI9-VK-W94F-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattttccccctttcacctttggccagggcacaaaggtggagatcaag

SEQ ID NO: 238 - protein, 108 aa, Artificial Sequence
  FNI9-VK-W94F-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPFTFGQGTKVEIK

SEQ ID NO: 239 - protein, 10 aa, Artificial Sequence
  FNI9-VK-W94F-W97F CDRL3 (aa)
QQYNNFPPFT

SEQ ID NO: 240 - DNA, 386 bp, Artificial Sequence
  FNI17-v19-VH (co-nt)
caggtccagctggtccagagtggggcagaggtcaaagagccagggtcttcagtcacagtctcatgcaaagcaagcggaggaacattttccaacaatgtgatcagctgggtgaggcaggctccaggacagggactggagtggatgggcggcatcatccctacctctggcatcgccaactacgctcagaagttccagggcagagtggccatcatcgctgacaagtctacctccacagtgtatatggccctgtccagcctgagaagcgaggattccgccgtgtacttctgcgccagggctcggtccgactacttcaaccgcgatctgggttgggaggactattactttgaaaactgggggcagggcacactggtcactgtctcatcagc

SEQ ID NO: 241 - protein, 128 aa, Artificial Sequence
  FNI17-v19-VH (aa)
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 242 - DNA, 324 bp, Artificial Sequence
  FNI17-v19-VK (co-nt)
gaaattgtgatgacccagtctccagccactctgtcagtctctccaggggaacgagccactctgtcatgtcgggcctctcagtccgtcggctccagcctggcttggtaccagcagaagccaggacaggctcctaggctgctgatctatggagctagcaccagggctacaggcgtgccagctcggttcagcggatctggatccggcaccgagtttaccctgacaatctcttccctgcagtctgaggacttcgccgtgtactattgccagcactacaataactggcctccttggacattcgggcaggggacaaaagtcgagattaag

SEQ ID NO: 243 - protein, 108 aa, Artificial Sequence
  FNI17-v19-VK (aa)
EIVMTQSPATLSVSPGERATLSCRASQSVGSSLAWYQQKPGQAPRLLIYGASTRATGVPARFSGSGSGTEFTLTISSLQSEDFAVYYCQHYNNWPPWTFGQGTKVEIK

SEQ ID NO: 244 - DNA, 384 bp, Artificial Sequence
  FNI19-v3-VH (co-nt)
caggtccagctggtgcagagtggtgccgaggtcaaaaagccagggtcaagtgtcaaagtcagttgtaaagcatcagagggaacattcaacaagtacacaatcagctgggtgagacaggctccaggacagggactggagtggatgggcggcatcatccctatctctggcatcgccaattacgctcagaagttccagggccgcgtggccatcacagctgacgagtccaccacaaccgcctatatggagctgtccagcctgaggtctgaggattccgccgtgtactattgcgccaccgctgtgagcgactacttcaaccgggatctgggctgggaggactattattttccattctggggtcaggggacactggtcaccgtctcttcc

SEQ ID NO: 245 - protein, 128 aa, Artificial Sequence
  FNI19-v3-VH (aa)
QVQLVQSGAEVKKPGSSVKVSCKASEGTFNKYTISWVRQAPGQGLEWMGGIIPISGIANYAQKFQGRVAITADESTTTAYMELSSLRSEDSAVYYCATAVSDYFNRDLGWEDYYFPFWGQGTLVTVSS

SEQ ID NO: 246 - DNA, 324 bp, Artificial Sequence; identical to SEQ ID NO: 214
  FNI19-v3-VK (co-nt)

SEQ ID NO: 247 - protein, 108 aa, Artificial Sequence; identical to SEQ ID NO: 210
  FNI19-v3-VK (aa)

SEQ ID NO: 248 - DNA, 384 bp, Artificial Sequence; identical to SEQ ID NO: 136
  FNI9-v5-VH (co-nt)

SEQ ID NO: 249 - protein, 128 aa, Artificial Sequence [sequence omitted]

SEQ ID NO: 250 - DNA, 324 bp, Artificial Sequence
  FNI9-v5-VK (co-nt)
gagattgtgatgacccagtcccctgctaccctgagcgtgtcccccggagagagagctaccctgagttgccgcgccagccgcagtgtctctgacaacctggcttggtaccagcagaagccaggacaggctcctaggctgctgatctatggcgcctccaccagggctacaggcatcccagctcggttctctggatccggaagcggcaccgagtttaccctgacaatctccagcctgcagagcgaggatttcgccgtgtactattgccagcattacaacatctggcctccttggacattcggtcagggaactaaagtggaaattaag

SEQ ID NO: 251 - protein, 108 aa, Artificial Sequence
  FNI9-v5-VK (aa)
EIVMTQSPATLSVSPGERATLSCRASRSVSDNLAWYQQKPGQAPRLLIYGASTRATGIPARFSGSGSGTEFTLTISSLQSEDFAVYYCQHYNIWPPWTFGQGTKVEIK

SEQ ID NO: 252 - protein, 330 aa, Artificial Sequence
  IgHG1*01, G1m3 CH1-CH3 with M428L and N434S mutations and C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 253 - protein, 329 aa, Artificial Sequence
  IgHG1*01, G1m3 CH1-CH3 with M428L and N434S mutations, without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 254 - protein, 107 aa, Artificial Sequence [sequence omitted]

SEQ ID NO: 255 - protein, 458 aa, Artificial Sequence
  FNI17-v19 heavy chain with M428L and N434S mutations in CH3 and a C-terminal lysine
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 256 - protein, 457 aa, Artificial Sequence
  FNI17-v19 heavy chain with M428L and N434S mutations in CH3, without C-terminal lysine
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 257 - protein, 215 aa, Artificial Sequence
  FNI17-v19 light chain
EIVMTQSPATLSVSPGERATLSCRASQSVGSSLAWYQQKPGQAPRLLIYGASTRATGVPARFSGSGSGTEFTLTISSLQSEDFAVYYCQHYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 128 aa, Artificial Sequence
  Synthetic sequence FNI17-v13 VH
QVQLVQSGARVKEPGSSVKVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 259 - protein, 108 aa, Artificial Sequence; identical to SEQ ID NO: 243
  Synthetic sequence FNI17-v13 VK

SEQ ID NO: 260 - DNA, 384 bp, Artificial Sequence
  Synthetic sequence FNI-UCA-IGH
caggtgcagctggtgcagtctggcgccgaggtgaagaagccaggctccagcgtgaaggtgagctgcaaggcttctggcggcaccttctcttcctacgctatctcctgggtgaggcaggctccaggacagggactggagtggatgggcggcatcatccctatcttcggcacagccaactacgctcagaagtttcagggcagagtgaccatcacagccgacgagtctacctccacagcttatatggagctgagctctctgcgctccgaggataccgccgtgtactattgtgccagggctggcagcgactacttcaaccgggatctgggctgggagaattactattttgactattggggccagggcaccctggtgacagtgtccagc

SEQ ID NO: 261 - protein, 128 aa, Artificial Sequence
  Synthetic sequence FNI-UCA VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAGSDYFNRDLGWENYYFDYWGQGTLVTVSS

SEQ ID NO: 262 - DNA, 324 bp, Artificial Sequence
  Synthetic sequence FNI-UCA-IGK
gagatcgtgatgacccagtctcctgccacactgagcgtgtctccaggagagagggccaccctgtcctgcagggcttcccagagcgtgtccagcaacctggcctggtaccagcagaagccaggccaggctcccaggctgctgatctatggcgccagcaccagagctacaggcatcccagctcgcttctctggatccggaagcggcacagagtttaccctgacaatctcttccctgcagtctgaggacttcgccgtgtactattgtcagcagtacaacaattggcccccttggacctttggccagggcacaaaggtggagatcaag

SEQ ID NO: 263 - protein, 108 aa, Artificial Sequence
  Synthetic sequence FNI-UCA VK
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 264 - protein, 8 aa, Artificial Sequence
  Synthetic sequence FNI-UCA CDRH1
GGTFSSYA

SEQ ID NO: 265 - protein, 8 aa, Artificial Sequence
  Synthetic sequence FNI-UCA CDRH2
IIPIFGTA

SEQ ID NO: 266 - protein, 21 aa, Artificial Sequence
  Synthetic sequence FNI-UCA CDRH3
ARAGSDYFNRDLGWENYYFDY

SEQ ID NO: 267 - protein, 6 aa, Artificial Sequence
  Synthetic sequence FNI-UCA CDRL1
QSVSSN

SEQ ID NO: 268 - protein, 3 aa, Artificial Sequence
  Synthetic sequence FNI-UCA CDRL2
GAS

SEQ ID NO: 269 - protein, 10 aa, Artificial Sequence [sequence omitted]

SEQ ID NO: 270 - protein, 458 aa, Artificial Sequence
  Synthetic sequence FM08_LS Heavy Chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 271 - protein, 210 aa, Artificial Sequence
  Synthetic sequence FM08_LS Light Chain
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 458 aa, Artificial Sequence
  Synthetic sequence FM08_GAALIE_LS Heavy Chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 273 - protein, 210 aa, Artificial Sequence; identical to SEQ ID NO: 271
  Synthetic sequence FM08_GAALIE_LS Light Chain

SEQ ID NO: 274 - protein, 7 aa, Artificial Sequence
  Synthetic sequence FM08 CDRH1
SYNAVWN

SEQ ID NO: 275 - protein, 18 aa, Artificial Sequence
  Synthetic sequence FM08 CDRH2
RTYYRSGWYNDYAESVKS

SEQ ID NO: 276 - protein, 16 aa, Artificial Sequence
  Synthetic sequence FM08 CDRH3
SGHITVFGVNVDAFDM

SEQ ID NO: 277 - protein, 11 aa, Artificial Sequence
  Synthetic sequence FM08 CDRL1
RTSQSLSSYTH

SEQ ID NO: 278 - protein, 7 aa, Artificial Sequence
  Synthetic sequence FM08 CDRL2
AASSRGS

SEQ ID NO: 279 - protein, 5 aa, Artificial Sequence
  Synthetic sequence FM08 CDRL3
QQSRT

SEQ ID NO: 280 - protein, 330 aa, Artificial Sequence
  Synthetic sequence IgG1 GAALIE CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 330 aa, Artificial Sequence
  Synthetic sequence IgG1 GAALIE_MLNS CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK